(19) United States
(12) Patent Application Publication
(10) Pub. No.: US 2004/0019053 A1
(43) Pub. Date: Jan. 29, 2004

(54) Combination of an allosteric carboxylic inhibitor of matrix metalloproteinase-13 with celecoxib or valdecoxib

(76) Inventor: William Howard Roark, Ann Arbor, MI (US)

Correspondence Address: WARNER-LAMBERT COMPANY, 2800 PLYMOUTH RD, ANN ARBOR, MI, 48105, US

(21) Appl. No.: 10/619,662

(22) Filed: Jul. 15, 2003

Related U.S. Application Data
(60) Provisional application No. 60/396,903, filed on Jul. 17, 2002.

(51) Int. Cl. A61K 31/519; A61K 31/415

(52) U.S. Cl. 514/248; 514/262.1; 514/406

(57) ABSTRACT

This invention provides a combination, comprising an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof. This invention also provides a method of treating a disease that is responsive to inhibition of MMP-13 and cyclooxygenase-2, comprising administering to a patient suffering from such a disease the invention combination comprising an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof. This invention also provides a pharmaceutical composition, comprising the invention combination comprising an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof, and a pharmaceutically acceptable carrier, diluent, or excipient.

The invention combination may also be further combined with other pharmaceutical agents depending on the disease being treated.

### CROSS-REFERENCE TO RELATED APPLICATIONS

This application claims benefit of priority from U.S. Provisional Patent Application No. 60/396,903, filed Jul. 17, 2002.

### FIELD OF THE INVENTION

This invention provides a combination of an allosteric carboxylic inhibitor of matrix metalloproteinase-13 with celecoxib or valdecoxib, a pharmaceutical composition comprising the combination, and methods of using the combination to treat diseases characterized by connective tissue breakdown, including cartilage damage, and inflammation or pain. Such diseases include arthritis, heart failure, multiple sclerosis, atherosclerosis, and osteoporosis.

### BACKGROUND OF THE INVENTION

More than 23 million Americans have some form of arthritis. Among the various forms of arthritis, osteoarthritis (“OA”) is the most prevalent, affecting 21 million Americans. Characterized by the degeneration of joint cartilage and adjacent bone, OA is a chronic disorder that can cause pain and stiffness. Rheumatoid arthritis (“RA”), which affects more than 2.1 million Americans, is an autoimmune disease that affects joint lining, cartilage and bones.

Aspirin and conventional nonsteroidal anti-inflammatory drugs (NSAIDs) such as ibuprofen, diclofenac, and naproxen are the primary agents used to treat OA- and RA-related pain. These agents inhibit prostaglandin release by blocking cyclooxygenase-mediated conversion of cell membrane lipids from arachidonic acid.

Two forms of COX are now known, a constitutive isoform usually named cyclooxygenase-1 (“COX-1”) and an inducible isoform usually named cyclooxygenase-2 (“COX-2”), the latter of which expression is upregulated at sites of inflammation. COX-1 appears to play a physiological role and to be responsible for gastrointestinal and renal protection. On the other hand, COX-2 appears to play a pathological role and is believed to be the predominant isoform present in inflammation conditions. The therapeutic use of conventional COX inhibitors, which are typically nonselective inhibitors of both COX-1 and COX-2, is limited due to drug associated side effects, including life threatening ulceration and renal toxicity. Compounds that selectively inhibit COX-2 would exert anti-inflammatory effects without the adverse side effects associated with COX-1 inhibition.

Valdecoxib is a COX-2 specific inhibitor that was approved in 2001 by the United States Food and Drug Administration (“FDA”) for treating the signs and symptoms of osteoarthritis (OA) and adult rheumatoid arthritis (RA); and the treatment of pain associated with menstrual cramping. Valdecoxib tablets are marketed under the tradename BEXTRA®. In a combined analysis of various clinical studies with valdecoxib, valdecoxib was well tolerated with an overall upper gastrointestinal safety profile (ulcers, perforations, obstructions and GI bleeds) significantly better than the conventional NSAIDs studied such as ibuprofen, diclofenac and naproxen.

Matrix metalloproteinases (“MMPs”) are naturally occurring enzymes found in most mammals. Stromelysin-1 and gelatinase A are members of the matrix metalloproteinases (MMP) family. Other members include fibroblast collagenase (MMP-1), neutrophil collagenase (MMP-8), gelatinase B (92 kDa gelatinase) (MMP-9), stromelysin-2 (MMP-10), stromelysin-3 (MMP-11), matrilysin (MMP-7), collagenase 3 (MMP-13), and other newly discovered membrane-associated matrix metalloproteinases.

Over-expression or activation of MMPs, or an imbalance between MMPs and their endogenous inhibitors, namely tissue inhibitors of metalloproteinases (“TIMPs”), have been suggested as factors in the pathogenesis of diseases characterized by the breakdown of extracellular matrix or connective tissues. These diseases include rheumatoid arthritis, osteoarthritis, osteoporosis, periodontitis, multiple sclerosis, gingivitis, corneal epidermal and gastric ulceration, atherosclerosis, neointimal proliferation which leads to restenosis and ischemic heart failure, and tumor metastasis.

A major limitation on the use of currently known MMP inhibitors is their lack of specificity for any particular MMP enzyme. Recent data has established that specific MMP enzymes are associated with some diseases, with no effect on others. The MMPs are generally categorized based on their substrate specificity, and indeed the collagenase subfamily of MMP-1, MMP-8, and MMP-13 selectively cleave native interstitial collagens, and thus are associated only with diseases linked to such interstitial collagen tissue. This is evidenced by the recent discovery that MMP-13 alone is over expressed in breast carcinoma, while MMP-1 alone is over expressed in papillary carcinoma (see Chen et al., J. Am. Chem. Soc., 2000;122:9648-9654).

Another major limitation of currently known MMP inhibitors related to their lack of specificity for any particular MMP enzyme is their production of undesirable side effects related to inhibition of multiple MMP enzymes and/or tumor necrosis factor-alpha converting enzyme (“TACE”). One example of such a side effect is musculoskeletal syndrome (“MSS”).

There appears to be few selective inhibitors of MMP-13 reported. A compound named WAY-170523 has been reported by Chen et al., supra., 2000, and a few other compounds are reported in PCT International Patent Application Publication Number WO 01/63244 A1, as allegedly selective inhibitors of MMP-13. Further, U.S. Pat. No. 6,008,243 discloses inhibitors of MMP-13. These inhibitors contain functional groups that ligate, coordinate, or bind the catalytic zinc cation on MMP-13. However, selectivity in these cases can mean only a 5-fold or 10-fold greater inhibition of MMP-13 versus as few as one other MMP enzyme. Further, no selective or non-allosteric carboxylic inhibitor of MMP-13 has been marketed for the treatment of any disease in any mammal.

Applicant has previously discovered highly selective inhibitors of MMP-13 that show promising pharmacological and pharmacokinetic activity in vivo. These inhibitors have been the subjects of previously filed patent applications.

Applicant's inhibitors are more selective than prior art inhibitors for MMP-13 versus other MMP enzymes, both in terms of relative potencies and in terms of the numbers of the other MMP enzymes. For example, some of Applicant's inhibitors have shown 100-fold or greater selectivity with MMP-13 versus five or more other MMP enzymes, and further have shown efficacy in animal models of osteoarthritis.

The observed selectivity of Applicant's inhibitors may be attributed to the inhibitors' binding to MMP-13 at an allosteric site and, further, to a binding mode which does not involve binding to the enzyme's catalytic zinc. Prior to Applicant's allosteric MMP-13 inhibitors, it is believed that all prior art MMP-13 inhibitors bound to an MMP enzyme's catalytic zinc and occupied the MMP enzyme's substrate binding site. This latter binding mode was erroneously believed by others to be necessary for MMP-13 inhibitor potency.

Applicant's discovery that a combination of an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof, is particularly useful for treating diseases characterized by damage to connective tissue such as cartilage damage. All that is required to treat diseases characterized by damage to connective tissue such as cartilage damage, including osteoarthritis, heart failure, multiple sclerosis, atherosclerosis, or osteoporosis in a mammal according to the invention is to administer to the mammal in need of treatment a therapeutically effective amount of the combination, wherein the combination comprises an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof. As will be discussed below, the instant combination of an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof, possesses many advantages over any combination of a prior art selective inhibitor of MMP-13 with a COX-2 inhibitor.

### SUMMARY OF THE INVENTION

This invention provides a combination, comprising an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof.

Another invention embodiment is a combination, comprising celecoxib, or a pharmaceutically acceptable salt thereof, and an allosteric carboxylic inhibitor of MMP-13, or a pharmaceutically acceptable salt thereof.

Other invention embodiments are described below:

1. A combination, comprising valdecoxib, or a pharmaceutically acceptable salt thereof, and an allosteric carboxylic inhibitor of MMP-13 of Formula I

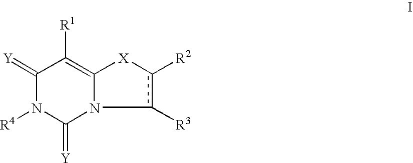

or a pharmaceutically acceptable salt thereof,

wherein:

“- - -” is absent or is a bond;

X is O, S, SO, SO₂, CH₂, C═O, CHOH, NH, or NR⁵;

Y is O or S;

R¹ is H, (O)ₙC₁-C₆ alkyl, (O)ₙ substituted C₁-C₆ alkyl, NO₂, NR⁵R⁶, CHO, or halo;

R², R³, and R⁴ independently are hydrogen, halo, C₁-C₆ alkyl, substituted C₁-C₆ alkyl, C₂-C₆ alkenyl, substituted C₂-C₆ alkenyl, C₂-C₁₀alkynyl, substituted C₂-C₁₀alkynyl, (CH₂)ₘ OH, (CH₂)ₘ OR⁵, (CH₂)ₘ cycloalkyl, (CH₂)ₘ substituted cycloalkyl, CHOH(CH₂)ₘ aryl, CHOH (CH₂)ₘ substituted aryl, CHOH(CH₂)ₘ heteroaryl, CHOH(CH₂)ₘ substituted heteroaryl, (CO₂)ₙ(CH₂)ₘ aryl, (CO₂)ₙ(CH₂)ₘ substituted aryl, (CO₂)ₙ(CH₂)ₘ heteroaryl, (CO₂)ₙ(CH₂)ₘ substituted heteroaryl, (CO₂)ₙ(CH₂)ₘ carbocycle, (CO₂)ₙ(CH₂)ₘ substituted carbocycle, (CO₂)ₙ(CH₂)ₘ heterocycle, (CO₂)ₙ(CH₂)ₘ substituted heterocycle, (CO₂)ₙ(CH₂)ₘ NR⁵R⁶, CH(C₁₋₆ alkyl)-aryl, (CH₂)ₘ N(H)C(═O)aryl, (CH₂)ₘ—S(O)₀₋₂—(CH₂)ₙ-aryl, CH(C₁-C₆ alkyl)-substituted aryl, (CH₂)ₘN(H)C(═O) substituted aryl, (CH₂)ₘ—S(O)₀₋₂—(CH₂)ₙ substituted aryl,

C(═O)N(R⁵)—(CH₂)ₘ aryl, C(═O)N(R⁵)—(CH₂)ₘ substituted aryl, C(═O)N(R⁵)—(CH₂)ₘ heteroaryl, C(═O)N(R⁵)—(CH₂)ₘ substituted heteroaryl, C≡C—(CH₂)ₘ aryl, C≡C—(CH₂)ₘ substituted aryl, C≡C—(CH₂)ₘ-heteroaryl, C≡C—(CH₂)ₘ substituted heteroaryl, C≡C—(CH₂)ₘ carbocycle, C≡C—(CH₂)ₘ substituted carbocycle, (CH₂)ₘ—O-aryl, (CH₂)ₘ—O-substituted aryl, (CH₂)ₘ COR⁵,

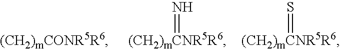

or (CH₂)ₘ CO₂R⁵;

m is an integer from 0 to 6;

R⁵ and R⁶ independently are hydrogen, C₁-C₆ alkyl, substituted C₁-C₆ alkyl, (CH₂)ₘ aryl, (CH₂)ₘ substituted aryl, (CH₂)ₘ heteroaryl or (CH₂)ₘ substituted heteroaryl, or R⁵ and R⁶ are taken together with the nitrogen atom to which they are attached complete a 3- to 7-membered ring; containing carbon atoms, the nitrogen atom bearing R⁵ and R⁶, and optionally 1 or 2 heteroatoms independently selected form O, S, and NR², wherein R² is as defined above and;

n is 0 or 1; with the proviso that R² and R⁴ are not both selected from hydrogen and C₁-C₆ alkyl.

2. The combination according to Embodiment 1, wherein the compound of Formula I is a compound of Formula III

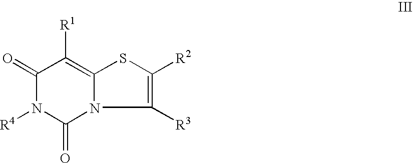

or a pharmaceutically acceptable salt thereof, wherein R¹, R², R³, and R⁴ are as defined above for Embodiment 1.

3. The combination according to Embodiment 2, wherein the compound of Formula III is selected from:

6-Benzyl-8-methyl-5,7-dioxo-6,7-dihydro-5H-thiazolo[3,2-c]pyrimidine-2-carbothioic acid benzylamide;

6-Benzyl-8-methyl-5,7-dioxo-6,7-dihydro-5H-thiazolo[3,2-c]pyrimidine-2-carbothioic acid 4-methoxy-benzylamide;

6-Benzyl-2-(3-phenyl-propionyl)-thiazolo[3,2-c]pyrimidine-5,7-dione;

6-Benzyl-8-methyl-5,7-dioxo-6,7-dihydro-5H-thiazolo[3,2-c]pyrimidine-2-carboxylic acid prop-2-ynylamide;

6-Benzyl-8-methyl-5,7-dioxo-6,7-dihydro-5H-thiazolo[3,2-c]pyrimidine-2-carboxylic acid (piperidin-4-ylmethyl)-amide hydrochloride;

6-Benzyl-2-(1-hydroxy-3-phenyl-allyl)-8-methyl-[3,2-c]pyrimidine-5,7-dione;

6-Benzyl-2-(1-hydroxy-3-phenyl-prop-2-ynyl)-8-methyl-[3,2-c]pyrimidine-5,7-dione;

6-Benzyl-2-(hydroxy-phenyl-methyl)-thiazolo[3,2-c]pyrimidine-5,7-dione; and

6-Benzyl-2-(1-hydroxy-3-phenyl-propyl)-thiazolo[3,2-c]pyrimidine-5,7-dione, or a pharmaceutically acceptable salt thereof.

4. The combination according to Embodiment 2, wherein the compound of Formula III is selected from:

6-Benzyl-8-methyl-5,7-dioxo-6,7-dihydro-5H-thiazolo[3,2-c]pyrimidine-2-carboxylic acid prop-2-ynylamide;

6-Benzyl-8-methyl-5,7-dioxo-6,7-dihydro-5H-thiazolo[3,2-c]pyrimidine-2-carboxylic acid (piperidin-4-ylmethyl)-amide hydrochloride;

6-(4-Bromo-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(3-Bromo-4-fluoro-benzyl)-8-methyl-5,7-dioxo-6,7-dihydro-5H-[3,2-c]pyrimidine-2-carboxylic acid (2-amino-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(4-Bromo-3-fluoro-benzyl)-8-methyl-5,7-dioxo-6,7-dihydro-5H-[3,2-c]pyrimidine-2-carboxylic acid (2-amino-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(3,4-Dibromo-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(4-Bromo-3-chloro-benzyl)-8-methyl-5,7-dioxo-6,7-dihydro-5H-[3,2-c]pyrimidine-2-carboxylic acid (2-amino-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid (2-amino-pyridin-4-ylmethyl)-amide;

6-(3-Bromo-4-chloro-benzyl)-8-methyl-5,7-dioxo-6,7-dihydro-5H-[3,2-c]pyrimidine-2-carboxylic acid (2-amino-pyridin-4-ylmethyl)-amide;

6-(3-Chloro-4-fluoro-benzyl)-8-methyl-5,7-dioxo-6,7-dihydro-5H-[3,2-c]pyrimidine-2-carboxylic acid (2-amino-pyridin-4-ylmethyl)-amide;

6-(4-Chloro-3-fluoro-benzyl)-8-methyl-5,7-dioxo-6,7-dihydro-5H-[3,2-c]pyrimidine-2-carboxylic acid (2-amino-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid (2-ethoxy-pyridin-4-ylmethyl)-amide;

6-(4-Chloro-benzyl)-8-methyl-5,7-dioxo-6,7-dihydro-SH-thiazolo[3,2-c]pyrimidine-2-carboxylic acid (2-ethoxy-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid (2-ethoxy-pyridin-4-ylmethyl)-amide;

6-(3-Bromo-4-fluoro-benzyl)-8-methyl-5,7-dioxo-6,7-dihydro-5H-[3,2-c]pyrimidine-2-carboxylic acid (2-ethoxy-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid (2-ethoxy-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid (2-ethoxy-pyridin-4-ylmethyl)-amide;

6-(4-Bromo-3-fluoro-benzyl)-8-methyl-5,7-dioxo-6,7-dihydro-5H-[3,2-c]pyrimidine-2-carboxylic acid (2-ethoxy-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid (2-ethoxy-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid (2-ethoxy-pyridin-4-ylmethyl)-amide;

6-(3,4-Dibromo-benzyl)-8-methyl-5,7-dioxo-6,7-dihydro-5H-thiazolo[3,2-c]pyrimidine-2-carboxylic acid (2-ethoxy-pyridin-4-ylmethyl)-amide;

6-(4-Bromo-3-chloro-benzyl)-8-methyl-5,7-dioxo-6,7-dihydro-5H-[3,2-c]pyrimidine-2-carboxylic acid (2-ethoxy-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid (2-ethoxy-pyridin-4-ylmethyl)-amide;

6-(3-Bromo-4-chloro-benzyl)-8-methyl-5,7-dioxo-6,7-dihydro-5H-[3,2-c]pyrimidine-2-carboxylic acid (2-ethoxy-pyridin-4-ylmethyl)-amide;

6-(3-Chloro-4-fluoro-benzyl)-8-methyl-5,7-dioxo-6,7-dihydro-5H-[3,2-c]pyrimidine-2-carboxylic acid (2-ethoxy-pyridin-4-ylmethyl)-amide;

6-(4-Chloro-3-fluoro-benzyl)-8-methyl-5,7-dioxo-6,7-dihydro-5H-[3,2-c]pyrimidine-2-carboxylic acid (2-ethoxy-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(3-Bromo-4-fluoro-benzyl)-8-methyl-5,7-dioxo-6,7-dihydro-5H-[3,2-c]pyrimidine-2-carboxylic acid (6-hydroxy-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(4-Bromo-3-fluoro-benzyl)-8-methyl-5,7-dioxo-6,7-dihydro-5H-[3,2-c]pyrimidine-2-carboxylic acid (6-hydroxy-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(3,4-Dibromo-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(4-Bromo-3-chloro-benzyl)-8-methyl-5,7-dioxo-6,7-dihydro-5H-[3,2-c]pyrimidine-2-carboxylic acid (6-hydroxy-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid (6-hydroxy-pyridin-3-ylmethyl)-amide;

6-(3-Bromo-4-chloro-benzyl)-8-methyl-5,7-dioxo-6,7-dihydro-5H-[3,2-c]pyrimidine-2-carboxylic acid (6-hydroxy-pyridin-3-ylmethyl)-amide;

6-(3-Chloro-4-fluoro-benzyl)-8-methyl-5,7-dioxo-6,7-dihydro-5H-[3,2-c]pyrimidine-2-carboxylic acid (6-hydroxy-pyridin-3-ylmethyl)-amide;

6-(4-Chloro-3-fluoro-benzyl)-8-methyl-5,7-dioxo-6,7-dihydro-5H-[3,2-c]pyrimidine-2-carboxylic acid (6-hydroxy-pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(3-Bromo-4-fluoro-benzyl)-8-methyl-5,7-dioxo-6,7-dihydro-5H-[3,2-c]pyrimidine-2-carboxylic acid (2-methoxy-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(4-Bromo-3-fluoro-benzyl)-8-methyl-5,7-dioxo-6,7-dihydro-5H-[3,2-c]pyrimidine-2-carboxylic acid (2-methoxy-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(3,4-Dibromo-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(4-Bromo-3-chloro-benzyl)-8-methyl-5,7-dioxo-6,7-dihydro-5H-[3,2-c]pyrimidine-2-carboxylic acid (2-methoxy-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid (2-methoxy-pyridin-4-ylmethyl)-amide;

6-(3-Bromo-4-chloro-benzyl)-8-methyl-5,7-dioxo-6,7-dihydro-5H-[3,2-c]pyrimidine-2-carboxylic acid (2-methoxy-pyridin-4-ylmethyl)-amide;

6-(3-Chloro-4-fluoro-benzyl)-8-methyl-5,7-dioxo-6,7-dihydro-5H-[3,2-c]pyrimidine-2-carboxylic acid (2-methoxy-pyridin-4-ylmethyl)-amide;

6-(4-Chloro-3-fluoro-benzyl)-8-methyl-5,7-dioxo-6,7-dihydro-5H-[3,2-c]pyrimidine-2-carboxylic acid (2-methoxy-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(3-Bromo-4-fluoro-benzyl)-8-methyl-5,7-dioxo-6;7-dihydro-5H-[3,2-c]pyrimidine-2-carboxylic acid (2-methyl-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(4-Bromo-3-fluoro-benzyl)-8-methyl-5,7-dioxo-6,7-dihydro-5H-[3,2-c]pyrimidine-2-carboxylic acid (2-methyl-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(3,4-Dibromo-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(4-Bromo-3-chloro-benzyl)-8-methyl-5,7-dioxo-6,7-dihydro-5H-[3,2-c]pyrimidine-2-carboxylic acid (2-methyl-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid (2-methyl-pyridin-4-ylmethyl)-amide;

6-(3-Bromo-4-chloro-benzyl)-8-methyl-5,7-dioxo-6,7-dihydro-5H-[3,2-c]pyrimidine-2-carboxylic acid (2-methyl-pyridin-4-ylmethyl)-amide;

6-(3-Chloro-4-fluoro-benzyl)-8-methyl-5,7-dioxo-6,7-dihydro-5H-[3,2-c]pyrimidine-2-carboxylic acid (2-methyl-pyridin-4-ylmethyl)-amide;

6-(4-Chloro-3-fluoro-benzyl)-8-methyl-5,7-dioxo-6,7-dihydro-5H-[3,2-c]pyrimidine-2-carboxylic acid (2-methyl-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(3-Bromo-4-fluoro-benzyl)-8-methyl-5,7-dioxo-6,7-dihydro-5H-[3,2-c]pyrimidine-2-carboxylic acid (2-methylamino-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(4-Bromo-3-fluoro-benzyl)-8-methyl-5,7-dioxo-6,7-dihydro-5H-[3,2-c]pyrimidine-2-carboxylic acid (2-methylamino-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(3,4-Dibromo-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(4-Bromo-3-chloro-benzyl)-8-methyl-5,7-dioxo-6,7-dihydro-5H-[3,2-c]pyrimidine-2-carboxylic acid (2-methylamino-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid (2-methylamino-pyridin-4-ylmethyl)-amide;

6-(3-Chloro-4-fluoro-benzyl)-8-methyl-5,7-dioxo-6,7-dihydro-5H-[3,2-c]pyrimidine-2-carboxylic acid (2-methylamino-pyridin-4-ylmethyl)-amide;

6-(4-Chloro-3-fluoro-benzyl)-8-methyl-5,7-dioxo-6,7-dihydro-5H-[3,2-c]pyrimidine-2-carboxylic acid (2-methylamino-pyridin-4-ylmethyl)-amide;

6-(4-Chloro-3-bromo-benzyl)-8-methyl-5,7-dioxo-6,7-dihydro-5H-[3,2-c]pyrimidine-2-carboxylic acid (2-methylamino-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid (pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid (pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid (pyridin-3-ylmethyl)-amide;

6-(3-Bromo-4-fluoro-benzyl)-8-methyl-5,7-dioxo-6,7-dihydro-5H-[3,2-c]pyrimidine-2-carboxylic acid (pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid (pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid (pyridin-3-ylmethyl)-amide;

6-(4-Bromo-3-fluoro-benzyl)-8-methyl-5,7-dioxo-6,7-dihydro-5H-[3,2-c]pyrimidine-2-carboxylic acid (pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid (pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid (pyridin-3-ylmethyl)-amide;

6-(3,4-Dibromo-benzyl)-8-methyl-5,7-dioxo-6,7-dihydro-5H-thiazolo[3,2-c]pyrimidine-2-carboxylic acid (pyridin-3-ylmethyl)-amide;

6-(4-Bromo-3-chloro-benzyl)-8-methyl-5,7-dioxo-6,7-dihydro-5H-[3,2-c]pyrimidine-2-carboxylic acid (pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid (pyridin-3-ylmethyl)-amide;

6-(3-Bromo-4-chloro-benzyl)-8-methyl-5,7-dioxo-6,7-dihydro-5H-[3,2-c]pyrimidine-2-carboxylic acid (pyridin-3-ylmethyl)-amide;

6-(3-Chloro-4-fluoro-benzyl)-8-methyl-5,7-dioxo-6,7-dihydro-5H-[3,2-c]pyrimidine-2-carboxylic acid (pyridin-3-ylmethyl)-amide;

6-(4-Chloro-3-fluoro-benzyl)-8-methyl-5,7-dioxo-6,7-dihydro-5H-[3,2-c]pyrimidine-2-carboxylic acid (pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(3-Bromo-4-fluoro-benzyl)-8-methyl-5,7-dioxo-6,7-dihydro-5H-[3,2-c]pyrimidine-2-carboxylic acid (6-amino-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(4-Bromo-3-fluoro-benzyl)-8-methyl-5,7-dioxo-6,7-dihydro-5H-[3,2-c]pyrimidine-2-carboxylic acid (6-amino-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(3,4-Dibromo-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(4-Bromo-3-chloro-benzyl)-8-methyl-5,7-dioxo-6,7-dihydro-5H-[3,2-c]pyrimidine-2-carboxylic acid (6-amino-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid (6-amino-pyridin-3-ylmethyl)-amide;

6-(3-Bromo-4-chloro-benzyl)-8-methyl-5,7-dioxo-6,7-dihydro-5H-[3,2-c]pyrimidine-2-carboxylic acid (6-amino-pyridin-3-ylmethyl)-amide;

6-(3-Chloro-4-fluoro-benzyl)-8-methyl-5,7-dioxo-6,7-dihydro-5H-[3,2-c]pyrimidine-2-carboxylic acid (6-amino-pyridin-3-ylmethyl)-amide;

6-(4-Chloro-3-fluoro-benzyl)-8-methyl-5,7-dioxo-6,7-dihydro-5H-[3,2-c]pyrimidine-2-carboxylic acid (6-amino-pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(3-Bromo-4-fluoro-benzyl)-8-methyl-5,7-dioxo-6,7-dihydro-5H-[3,2-c]pyrimidine-2-carboxylic acid (6-ethoxy-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(4-Bromo-3-fluoro-benzyl)-8-methyl-5,7-dioxo-6,7-dihydro-5H-[3,2-c]pyrimidine-2-carboxylic acid (6-ethoxy-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(3,4-Dibromo-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(4-Bromo-3-chloro-benzyl)-8-methyl-5,7-dioxo-6,7-dihydro-5H-[3,2-c]pyrimidine-2-carboxylic acid (6-ethoxy-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid (6-ethoxy-pyridin-3-ylmethyl)-amide;

6-(3-Bromo-4-chloro-benzyl)-8-methyl-5,7-dioxo-6,7-dihydro-5H-[3,2-c]pyrimidine-2-carboxylic acid (6-ethoxy-pyridin-3-ylmethyl)-amide;

6-(3-Chloro-4-fluoro-benzyl)-8-methyl-5,7-dioxo-6,7-dihydro-5H-[3,2-c]pyrimidine-2-carboxylic acid (6-ethoxy-pyridin-3-ylmethyl)-amide;

6-(4-Chloro-3-fluoro-benzyl)-8-methyl-5,7-dioxo-6,7-dihydro-5H-[3,2-c]pyrimidine-2-carboxylic acid (6-ethoxy-pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(3-Bromo-4-fluoro-benzyl)-8-methyl-5,7-dioxo-6,7-dihydro-5H-[3,2-c]pyrimidine-2-carboxylic acid (6-methoxy-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(4-Bromo-3-fluoro-benzyl)-8-methyl-5,7-dioxo-6,7-dihydro-5H-[3,2-c]pyrimidine-2-carboxylic acid (6-methoxy-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(3,4-Dibromo-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(4-Bromo-3-chloro-benzyl)-8-methyl-5,7-dioxo-6,7-dihydro-5H-[3,2-c]pyrimidine-2-carboxylic acid (6-methoxy-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid (6-methoxy-pyridin-3-ylmethyl)-amide;

6-(3-Bromo-4-chloro-benzyl)-8-methyl-5,7-dioxo-6,7-dihydro-5H-[3,2-c]pyrimidine-2-carboxylic acid (6-methoxy-pyridin-3-ylmethyl)-amide;

6-(3-Chloro-4-fluoro-benzyl)-8-methyl-5,7-dioxo-6,7-dihydro-5H-[3,2-c]pyrimidine-2-carboxylic acid (6-methoxy-pyridin-3-ylmethyl)-amide;

6-(4-Chloro-3-fluoro-benzyl)-8-methyl-5,7-dioxo-6,7-dihydro-5H-[3,2-c]pyrimidine-2-carboxylic acid (6-methoxy-pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(3-Bromo-4-fluoro-benzyl)-8-methyl-5,7-dioxo-6,7-dihydro-5H-[3,2-c]pyrimidine-2-carboxylic acid (6-methyl-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(4-Bromo-3-fluoro-benzyl)-8-methyl-5,7-dioxo-6,7-dihydro-5H-[3,2-c]pyrimidine-2-carboxylic acid (6-methyl-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(3,4-Dibromo-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(4-Bromo-3-chloro-benzyl)-8-methyl-5,7-dioxo-6,7-dihydro-5H-[3,2-c]pyrimidine-2-carboxylic acid (6-methyl-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid (6-methyl-pyridin-3-ylmethyl)-amide;

6-(3-Bromo-4-chloro-benzyl)-8-methyl-5,7-dioxo-6,7-dihydro-5H-[3,2-c]pyrimidine-2-carboxylic acid (6-methyl-pyridin-3-ylmethyl)-amide;

6-(3-Chloro-4-fluoro-benzyl)-8-methyl-5,7-dioxo-6,7-dihydro-5H-[3,2-c]pyrimidine-2-carboxylic acid (6-methyl-pyridin-3-ylmethyl)-amide;

6-(4-Chloro-3-fluoro-benzyl)-8-methyl-5,7-dioxo-6,7-dihydro-5H-[3,2-c]pyrimidine-2-carboxylic acid (6-methyl-pyridin-3-ylmethyl)-amide;

6-(4-Cyano-benzyl)-8-methyl-5,7-dioxo-6,7-dihydro-5H-thiazolo[3,2-c]pyrimidine-2-carboxylic acid (2-methoxy-pyridin-4-ylmethyl)-amide; and

6-(4-Isopropylsulfamoyl-benzyl)-8-methyl-5,7-dioxo-6,7-dihydro-5H-[3,2-c]pyrimidine-2-carboxylic acid (2-methoxy-pyridin-4-ylmethyl)-amide, or a pharmaceutically acceptable salt thereof.

5. The combination according to Embodiment 1, wherein the compound of Formula I is a compound of Formula IV

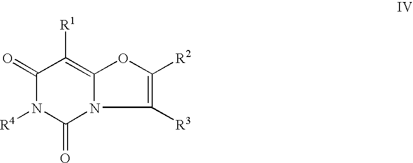

or a pharmaceutically acceptable salt thereof, wherein R¹, R², R³, and R⁴ are as defined above for Embodiment 1.

6. The combination according to Embodiment 5, wherein the compound of Formula IV is selected from:

6-Benzyl-8-methyl-5,7-dioxo-6,7-dihydro-5H-oxazolo[3,2-c]pyrimidine-2-carboxylic acid benzyl ester;

6-Benzyl-5,7-dioxo-6,7-dihydro-5H-oxazolo[3,2-c]pyrimidine-2-carboxylic acid benzyl ester;

6-Benzyl-5,7-dioxo-6,7-dihydro-5H-oxazolo[3,2-c]pyrimidine-2-carboxylic acid benzylamide;

6-Benzyl-8-methyl-5,7-dioxo-6,7-dihydro-5H-oxazolo[3,2-c]pyrimidine-2-carboxylic acid 4-methoxy-benzylamide;

6-Benzyl-8-methyl-5,7-dioxo-6,7-dihydro-5H-oxazolo[3,2-c]pyrimidine-2-carboxylic acid (pyridin-4-ylmethyl)-amide; and

6-Benzyl-8-methyl-5,7-dioxo-6,7-dihydro-5H-oxazolo[3,2-c]pyrimidine-2-carboxylic acid (benzo[1,3]dioxol-5-ylmethyl)-amide, or a pharmaceutically acceptable salt thereof.

7. The combination according to Embodiment 1, wherein the compound of Formula I is a compound of Formula V

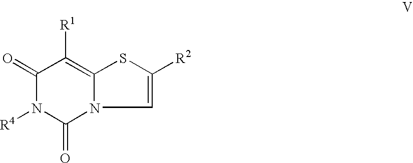

or a pharmaceutically acceptable salt thereof, wherein R¹ is hydrogen, (O)ₙ C₁-C₆ alkyl, or (O)ₙ substituted C₁-C₆ alkyl, R² is CO₂(CH₂)ₘ aryl, CO₂(CH₂)ₘ substituted aryl,

R⁴ is (CH₂)ₘ CO₂R⁵, (CH₂)ₘ CONR⁵R⁶, (CH₂)ₘ CNR⁵R⁶,

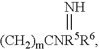

CHOH(CH₂)ₘ aryl, CHOH(CH₂)ₘ substituted aryl, CHOH(CH₂)ₘ heteroaryl, CHOH(CH₂)ₘ substituted aryl.

8. The combination according to Embodiment 1, wherein the compound of Formula I is a compound of Formula VI:

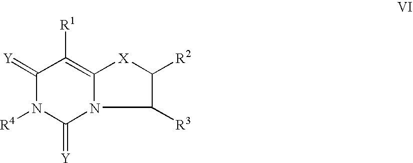

or a pharmaceutically acceptable salt thereof, wherein R¹, R², R³, R⁴, Y, and X are as defined above for Embodiment 1.

9. The combination according to Embodiment 8, wherein the compound of Formula VI is selected from:

6-Benzyl-8-methyl-5,7-dioxo-1,5,6,7-tetrahydro-imidazo[1,2-c]pyrimidine-2-carboxylic acid (benzo[1,3]dioxol-5-ylmethyl)-amide;

6-Benzyl-1,8-dimethyl-5,7-dioxo-1,5,6,7-tetrahydro-imidazo[1,2-c]pyrimidine-2-carboxylic acid (benzo[1,3]dioxol-5-ylmethyl)-amide;

6-Benzyl-1,8-dimethyl-5,7-dioxo-1,5,6,7-tetrahydro-imidazo[1,2-c]pyrimidine-2-carboxylic acid benzylamide;

6-Benzyl-1,8-dimethyl-5,7-dioxo-1,5,6,7-tetrahydro-imidazo[1,2-c]pyrimidine-2-carboxylic acid 4-methoxy-benzylamide;

6-Benzyl-1-methyl-5,7-dioxo-1,5,6,7-tetrahydro-imidazo[1,2-c]pyrimidine-2-carboxylic acid 4-methoxy-benzylamide;

6-(4-Methoxy-benzyl)-1-methyl-5,7-dioxo-1,5,6,7-tetrahydro-imidazo[1,2-c]pyrimidine-2-carboxylic acid 4-methoxy-benzylamide;

6-(4-Methoxy-benzyl)-1,8-dimethyl-5,7-dioxo-1,5,6,7-tetrahydro-[1,2-c]pyrimidine-2-carboxylic acid (pyridin-4-ylmethyl)-amide;

6-Benzyl-5,7-dioxo-2,3,6,7-tetrahydro-5H-thiazolo[3,2-c]pyrimidine-2-carboxylic acid benzyl ester 2,3-Dihydroxypropionic acid benzyl ester;

6-Benzyl-5,7-dioxo-2,3,6,7-tetrahydro-5H-thiazolo[3,2-c]pyrimidine-2-carboxylic acid pyridin-4-ylmethyl ester hydrochloride;

6-Benzyl-1,5,7-trioxo-1,2,3,5,6,7-hexahydro-11⁴-thiazolo[3,2-c]pyrimidine-3-carboxylic acid benzyl ester;

6-Benzyl-1,8-dimethyl-5,7-dioxo-1,5,6,7-tetrahydro-imidazo-[1,2-c]pyrimidine-2-carboxylic acid 4-methoxy-benzyl ester; and

6-benzyl-3-ethoxy-2,3-dihydro-oxazolo[3,2-c]pyrimidine-5,7-dione, or a pharmaceutically acceptable salt thereof.

10. The combination according to Embodiment 1, wherein the compound of Formula I is a compound of Formula VII

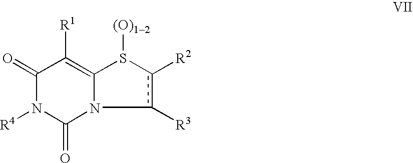

or a pharmaceutically acceptable salt thereof, wherein R¹, R², R³, and R⁴ are as defined above for Embodiment 1.

11. The combination according to Embodiment 1, wherein the compound of Formula I is a compound of Formula VIII

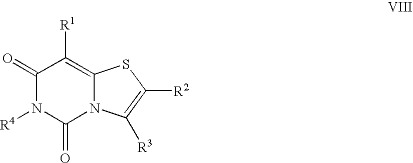

or a pharmaceutically acceptable salt thereof, wherein:

R¹ is H, CH₃, CH₂OH, or CHO;

R² is (CO₂)(CH₂)ₘ aryl, (CO₂)(CH₂)ₘ substituted aryl, (CO₂)(CH₂)ₘ heteroaryl, (CO₂)(CH₂)ₘ substituted heteroaryl, C(═O)N(R⁵)—(CH₂)ₘ-aryl, C(═O)N(R⁵)—(CH₂)ₘ substituted aryl, C(═O)N(R⁵)—(CH₂)ₘ heteroaryl, C(═O)N(R⁵)—(CH₂)ₘ substituted heteroaryl, C≡C—(CH₂)maryl, C≡C—(CH₂)ₘ substituted aryl, C≡C—(CH₂)ₘ heteroaryl, or C≡C—(CH₂)ₘ substituted heteroaryl, wherein R⁵ is hydrogen or methyl;

R³ is hydrogen or fluoro;

R⁴ is C₂-C₆ alkenyl, substituted C₂-C₆ alkenyl, C₁-C₆ alkyl, substituted C₁-C₆ alkyl, C₂-C₁₀alkenyl, substituted C₂-C₁₀alkynyl, (CH₂)ₘCOR⁵, (CH₂)ₘS(O)₀₋₂—(CH₂)naryl, C(═O)N(R⁵)—CH₂)maryl, (CH₂)ₘ—O-aryl, (CH₂)ₘS(O)₀₋₂—(CH₂)ₙ substituted aryl, C(═O)N(R⁵)—CH₂)ₘ substituted aryl, (CH₂)ₘ—O-substituted aryl, (CO₂)ₙ(CH₂)ₘ aryl, (CO₂)ₙ(CH₂)ₘ substituted aryl, (CO₂)ₙ(CH₂)ₘ heteroaryl, (CO₂)ₙ(CH₂)ₘ substituted heteroaryl, (CO₂)ₙ(CH₂)ₘ carbocycle, or (CO₂)ₙ(CH₂)ₘ substituted carbocycle, wherein

n is 0 or 1;

m is an integer of from 0 to 6; and

R⁵ is as defined above for Embodiment 1.

12. The combination according to Embodiment 11, wherein the compound of Formula VIII is a compound selected from:

4-[8-Methyl-5,7-dioxo-2-(3-phenyl-prop-1-ynyl)-7H-thiazolo[3,2-c]pyrimidin-6-ylmethyl]-benzoic acid;

4-{2-[3-(4-Methoxy-phenyl)-prop-1-ynyl]-8-methyl-5,7-dioxo-7H-[3,2-c]pyrimidin-6-ylmethyl}-benzoic acid;

4-{2-[3-(4-Fluoro-phenyl)-prop-1-ynyl]-8-methyl-5,7-dioxo-7H-[3,2-c]pyrimidin-6-ylmethyl}-benzoic acid;

4-{2-[3-(3-Methoxy-phenyl)-prop-1-ynyl]-8-methyl-5,7-dioxo-7H-[3,2-c]pyrimidin-6-ylmethyl}-benzoic acid;

4-{2-[3-(3,4-Difluoro-phenyl)-prop-1-ynyl]-8-methyl-5,7-dioxo-7H-[3,2-c]pyrimidin-6-ylmethyl}-benzoic acid;

6-Benzyl-8-methyl-2-(3-pyridin-4-yl-prop-1-ynyl)-thiazolo[3,2-c]pyrimidine-5,7-dione;

6-(3,4-Dichloro-benzyl)-8-methyl-2-(3-pyridin-4-yl-prop-1-ynyl)-[3,2-c]pyrimidine-5,7-dione;

6-(3,4-Dichloro-benzyl)-2-[3-(2-methoxy-pyridin-4-yl)-prop-1-ynyl]-8-methyl-thiazolo[3,2-c]pyrimidine-5,7-dione;

6-Benzyl-8-methyl-2-phenylethynyl-thiazolo[3,2-c]pyrimidine-5,7-dione;

6-(4-Bromo-benzyl)-2-[3-(3-methoxy-phenyl)-prop-1-ynyl]-8-methyl-[3,2-c]pyrimidine-5,7-dione;

4-{2-[3-(3-Methoxy-phenyl)-prop-1-ynyl]-8-methyl-5,7-dioxo-7H-[3,2-c]pyrimidin-6-ylmethyl}-benzenesulfonamide;

4-{2-[3-(3-Fluoro-4-methoxy-phenyl)-prop-1-ynyl]-8-methyl-5,7-dioxo-7H-thiazolo[3,2-c]pyrimidin-6-ylmethyl}-benzoic acid;

6-(4-Fluoro-benzyl)-8-methyl-2-(3-phenyl-prop-1-ynyl)-thiazolo[3,2-c]pyrimidine-5,7-dione;

6-Benzyl-8-methyl-2-(3-phenyl-prop-1-ynyl)-thiazolo[3,2-c]pyrimidine-5,7-dione;

6-(3,4-Dichloro-benzyl)-2-[3-(3-methoxy-phenyl)-prop-1-ynyl]-8-methyl-[3,2-c]pyrimidine-5,7-dione;

6-(4-Methanesulfonyl-benzyl)-8-methyl-2-(3-pyridin-4-yl-prop-1-ynyl)-[3,2-c]pyrimidine-5,7-dione;

4-{2-[3-(3-Methoxy-phenyl)-prop-1-ynyl]-8-methyl-5,7-dioxo-7H-[3,2-c]pyrimidin-6-ylmethyl}-benzonitrile;

4-[8-Methyl-5,7-dioxo-2-(3-phenyl-prop-1-ynyl)-7H-thiazolo[3,2-c]pyrimidin-6-ylmethyl]-benzoic acid;

2-[3-(3-Methoxy-phenyl)-prop-1-ynyl]-8-methyl-6-[4-(2H-tetrazol-5-yl)-benzyl]-thiazolo[3,2-c]pyrimidine-5,7-dione;

6-Benzyl-2-[3-(3-methoxy-phenyl)-prop-1-ynyl]-8-methyl-thiazolo[3,2-c]pyrimidine-5,7-dione;

6-Benzyl-8-methyl-2-(3-phenyl-prop-1-ynyl)-thiazolo[3,2-c]pyrimidine-5,7-dione;

2-[3-(3-Methoxy-phenyl)-prop-1-ynyl]-8-methyl-6-[4-(morpholine-4-carbonyl)-benzyl]-thiazolo[3,2-c]pyrimidine-5,7-dione;

8-Methyl-6-[4-(morpholine-4-sulfonyl)-benzyl]-2-(3-pyridin-4-yl-prop-1-ynyl)-thiazolo[3,2-c]pyrimidine-5,7-dione;

2-[3-(4-Fluoro-phenyl)-prop-1-ynyl]-8-methyl-6-(2-oxo-2H-1-benzopyran-6-ylmethyl)-thiazolo[3,2-c]pyrimidine-5,7-dione;

2-[3-(3-Methoxy-phenyl)-prop-1-ynyl]-8-methyl-6-(2-oxo-2H-1-benzopyran-6-ylmethyl)-thiazolo[3,2-c]pyrimidine-5,7-dione;

4-[8-Methyl-5,7-dioxo-2-(4-phenyl-but-1-ynyl)-7H-thiazolo[3,2-c]pyrimidin-6-ylmethyl]-benzoic acid;

4-[8-Methyl-5,7-dioxo-2-(6-phenyl-hex-1-ynyl)-7H-thiazolo[3,2-c]pyrimidin-6-ylmethyl]-benzoic acid;

4-[8-Methyl-5,7-dioxo-2-(5-phenyl-pent-1-ynyl)-7H-thiazolo[3,2-c]pyrimidin-6-ylmethyl]-benzoic acid;

4-[8-Methyl-5,7-dioxo-2-(7-phenyl-hept-1-ynyl)-7H-thiazolo[3,2-c]pyrimidin-6-ylmethyl]-benzoic acid;

(4-{2-[3-(3,4-Difluoro-phenyl)-prop-1-ynyl]-8-methyl-5,7-dioxo-7H-[3,2-c]pyrimidin-6-ylmethyl}-phenyl)-acetic acid;

6-(3-Fluoro-benzyl)-8-methyl-2-(3-pyridin-4-yl-prop-1-ynyl)-thiazolo[3,2-c]pyrimidine-5,7-dione;

6-(3,4-Difluoro-benzyl)-8-methyl-2-(3-pyridin-4-yl-prop-1-ynyl)-[3,2-c]pyrimidine-5,7-dione;

6-(3-Fluoro-benzyl)-2-[3-(2-methoxy-pyridin-4-yl)-prop-1-ynyl]-8-methyl-thiazolo[3,2-c]pyrimidine-5,7-dione;

[3-(8-Methyl-5,7-dioxo-2-phenylethynyl-7H-thiazolo[3,2-c]pyrimidin-6-ylmethyl)-phenyl]-acetic acid;

6-(4-Bromo-benzyl)-2-[3-(4-fluoro-3-methoxy-phenyl)-prop-1-ynyl]-8-methyl-thiazolo[3,2-c]pyrimidine-5,7-dione;

4-{2-[3-(3-Methoxy-phenyl)-prop-1-ynyl]-8-methyl-5,7-dioxo-7H-[3,2-c]pyrimidin-6-ylmethyl}-N,N-dimethyl-benzenesulfonamide;

4-{2-[3-(3-Fluoro-4-methoxy-phenyl)-prop-1-ynyl]-8-methyl-5,7-dioxo-7H-thiazolo[3,2-c]pyrimidin-6-ylmethyl}-cyclohexanecarboxylic acid;

6-(3,4-Difluoro-benzyl)-2-[3-(3,4-difluoro-phenyl)-prop-1-ynyl]-8-methyl-[3,2-c]pyrimidine-5,7-dione;

4-[8-Methyl-5,7-dioxo-2-(3-phenyl-prop-1-ynyl)-7H-thiazolo[3,2-c]pyrimidin-6-ylmethyl]-cyclohexanecarboxylic acid;

2-Chloro-4-{2-[3-(3-methoxy-phenyl)-prop-1-ynyl]-8-methyl-5,7-dioxo-7H-thiazolo[3,2-c]pyrimidin-6-ylmethyl}-benzoic acid;

2-[3-(4-Fluoro-phenyl)-prop-1-ynyl]-6-(4-methanesulfonyl-benzyl)-8-methyl-thiazolo[3,2-c]pyrimidine-5,7-dione;

4-{2-[3-(4-Fluoro-3-methoxy-phenyl)-prop-1-ynyl]-8-methyl-5,7-dioxo-7H-thiazolo[3,2-c]pyrimidin-6-ylmethyl}-benzonitrile;

(3-{2-[3-(4-Fluoro-3-methoxy-phenyl)-prop-1-ynyl]-8-methyl-5,7-dioxo-7H-thiazolo[3,2-c]pyrimidin-6-ylmethyl}-phenyl)-acetic acid;

(4-{2-[3-(3-Methoxy-phenyl)-prop-1-ynyl]-8-methyl-5,7-dioxo-7H-[3,2-c]pyrimidin-6-ylmethyl}-phenyl)-acetic acid;

6-(3,4-Difluoro-benzyl)-8-methyl-2-(3-phenyl-prop-1-ynyl)-thiazolo[3,2-c]pyrimidine-5,7-dione;

2-[3-(3-Methoxy-phenyl)-prop-1-ynyl]-8-methyl-6-[4-(thiomorpholine-4-carbonyl)-benzyl]-thiazolo[3,2-c]pyrimidine-5,7-dione;

8-Methyl-2-(3-pyridin-4-yl-prop-1-ynyl)-6-[4-(thiomorpholine-4-sulfonyl)-benzyl]-thiazolo[3,2-c]pyrimidine-5,7-dione;

2-[3-(4-Fluoro-3-methoxy-phenyl)-prop-1-ynyl]-8-methyl-6-(2-oxo-2H-1-benzopyran-6-ylmethyl)-thiazolo[3,2-c]pyrimidine-5,7-dione; and

2-[3-(3-Methoxy-4-methyl-phenyl)-prop-1-ynyl]-8-methyl-6-(2-oxo-2H-1-benzopyran-6-ylmethyl)-thiazolo[3,2-c]pyrimidine-5,7-dione, or a pharmaceutically acceptable salt thereof.

13. The combination according to Embodiment 11, wherein the compound of Formula VIII is a compound selected from:

4-[8-Methyl-5,7-dioxo-2-(3-phenyl-prop-1-ynyl)-7H-thiazolo[3,2-c]pyrimidin-6-ylmethyl]-benzoic acid;

4-{2-[3-(4-Methoxy-phenyl)-prop-1-ynyl]-8-methyl-5,7-dioxo-7H-[3,2-c]pyrimidin-6-ylmethyl}-benzoic acid;

4-{2-[3-(4-Fluoro-phenyl)-prop-1-ynyl]-8-methyl-5,7-dioxo-7H-[3,2-c]pyrimidin-6-ylmethyl}-benzoic acid;

4-{2-[3-(3-Methoxy-phenyl)-prop-1-ynyl]-8-methyl-5,7-dioxo-7H-[3,2-c]pyrimidin-6-ylmethyl}-benzoic acid;

4-{2-[3-(3,4-Difluoro-phenyl)-prop-1-ynyl]-8-methyl-5,7-dioxo-7H-[3,2-c]pyrimidin-6-ylmethyl}-benzoic acid;

6-Benzyl-8-methyl-2-(3-pyridin-4-yl-prop-1-ynyl)-thiazolo[3,2-c]pyrimidine-5,7-dione;

6-(3,4-Dichloro-benzyl)-8-methyl-2-(3-pyridin-4-yl-prop-1-ynyl)-[3,2-c]pyrimidine-5,7-dione;

6-(3,4-Dichloro-benzyl)-2-[3-(2-methoxy-pyridin-4-yl)-prop-1-ynyl]-8-methyl-thiazolo[3,2-c]pyrimidine-5,7-dione;

6-Benzyl-8-methyl-2-phenylethynyl-thiazolo[3,2-c]pyrimidine-5,7-dione;

6-(4-Bromo-benzyl)-2-[3-(3-methoxy-phenyl)-prop-1-ynyl]-8-methyl-[3,2-c]pyrimidine-5,7-dione;

4-{2-[3-(3-Methoxy-phenyl)-prop-1-ynyl]-8-methyl-5,7-dioxo-7H-[3,2-c]pyrimidin-6-ylmethyl}-benzenesulfonamide;

4-{2-[3-(3-Fluoro-4-methoxy-phenyl)-prop-1-ynyl]-8-methyl-5,7-dioxo-7H-thiazolo[3,2-c]pyrimidin-6-ylmethyl}-benzoic acid;

6-(4-Fluoro-benzyl)-8-methyl-2-(3-phenyl-prop-1-ynyl)-thiazolo[3,2-c]pyrimidine-5,7-dione;

6-Benzyl-8-methyl-2-(3-phenyl-prop-1-ynyl)-thiazolo[3,2-c]pyrimidine-5,7-dione;

6-(3,4-Dichloro-benzyl)-2-[3-(3-methoxy-phenyl)-prop-1-ynyl]-8-methyl-[3,2-c]pyrimidine-5,7-dione;

6-(4-Methanesulfonyl-benzyl)-8-methyl-2-(3-pyridin-4-yl-prop-1-ynyl)-[3,2-c]pyrimidine-5,7-dione;

4-{2-[3-(3-Methoxy-phenyl)-prop-1-ynyl]-8-methyl-5,7-dioxo-7H-[3,2-c]pyrimidin-6-ylmethyl}-benzonitrile;

4-[8-Methyl-5,7-dioxo-2-(3-phenyl-prop-1-ynyl)-7H-thiazolo[3,2-c]pyrimidin-6-ylmethyl]-benzoic acid;

2-[3-(3-Methoxy-phenyl)-prop-1-ynyl]-8-methyl-6-[4-(2H-tetrazol-5-yl)-benzyl]-thiazolo[3,2-c]pyrimidine-5,7-dione;

6-Benzyl-2-[3-(3-methoxy-phenyl)-prop-1-ynyl]-8-methyl-thiazolo[3,2-c]pyrimidine-5,7-dione;

6-Benzyl-8-methyl-2-(3-phenyl-prop-1-ynyl)-thiazolo[3,2-c]pyrimidine-5,7-dione;

2-[3-(3-Methoxy-phenyl)-prop-1-ynyl]-8-methyl-6-[4-(morpholine-4-carbonyl)-benzyl]-thiazolo[3,2-c]pyrimidine-5,7-dione;

8-Methyl-6-[4-(morpholine-4-sulfonyl)-benzyl]-2-(3-pyridin-4-yl-prop-1-ynyl)-thiazolo[3,2-c]pyrimidine-5,7-dione;

2-[3-(4-Fluoro-phenyl)-prop-1-ynyl]-8-methyl-6-(2-oxo-2H-1-benzopyran-6-ylmethyl)-thiazolo[3,2-c]pyrimidine-5,7-dione;

2-[3-(3-Methoxy-phenyl)-prop-1-ynyl]-8-methyl-6-(2-oxo-2H-1-benzopyran-6-ylmethyl)-thiazolo[3,2-c]pyrimidine-5,7-dione;

4-[8-Methyl-5,7-dioxo-2-(4-phenyl-but-1-ynyl)-7H-thiazolo[3,2-c]pyrimidin-6-ylmethyl]-benzoic acid;

4-[8-Methyl-5,7-dioxo-2-(6-phenyl-hex-1-ynyl)-7H-thiazolo[3,2-c]pyrimidin-6-ylmethyl]-benzoic acid;

4-[8-Methyl-5,7-dioxo-2-(5-phenyl-pent-1-ynyl)-7H-thiazolo[3,2-c]pyrimidin-6-ylmethyl]-benzoic acid;

4-[8-Methyl-5,7-dioxo-2-(7-phenyl-hept-1-ynyl)-7H-thiazolo[3,2-c]pyrimidin-6-ylmethyl]-benzoic acid;

(4-{2-[3-(3,4-Difluoro-phenyl)-prop-1-ynyl]-8-methyl-5,7-dioxo-7H-[3,2-c]pyrimidin-6-ylmethyl}-phenyl)-acetic acid;

6-(3-Fluoro-benzyl)-8-methyl-2-(3-pyridin-4-yl-prop-1-ynyl)-thiazolo[3,2-c]pyrimidine-5,7-dione;

6-(3,4-Difluoro-benzyl)-8-methyl-2-(3-pyridin-4-yl-prop-1-ynyl)-[3,2-c]pyrimidine-5,7-dione;

6-(3-Fluoro-benzyl)-2-[3-(2-methoxy-pyridin-4-yl)-prop-1-ynyl]-8-methyl-thiazolo[3,2-c]pyrimidine-5,7-dione;

[3-(8-Methyl-5,7-dioxo-2-phenylethynyl-7H-thiazolo[3,2-c]pyrimidin-6-ylmethyl)-phenyl]-acetic acid;

6-(4-Bromo-benzyl)-2-[3-(4-fluoro-3-methoxy-phenyl)-prop-1-ynyl]-8-methyl-thiazolo[3,2-c]pyrimidine-5,7-dione;

4-{2-[3-(3-Methoxy-phenyl)-prop-1-ynyl]-8-methyl-5,7-dioxo-7H-[3,2-c]pyrimidin-6-ylmethyl}-N,N-dimethyl-benzenesulfonamide;

4-{2-[3-(3-Fluoro-4-methoxy-phenyl)-prop-1-ynyl]-8-methyl-5,7-dioxo-7H-thiazolo[3,2-c]pyrimidin-6-ylmethyl}-cyclohexanecarboxylic acid;

6-(3,4-Difluoro-benzyl)-2-[3-(3,4-difluoro-phenyl)-prop-1-ynyl]-8-methyl-[3,2-c]pyrimidine-5,7-dione;

4-[8-Methyl-5,7-dioxo-2-(3-phenyl-prop-1-ynyl)-7H-thiazolo[3,2-c]pyrimidin-6-ylmethyl]-cyclohexanecarboxylic acid;

2-Chloro-4-{2-[3-(3-methoxy-phenyl)-prop-1-ynyl]-8-methyl-5,7-dioxo-7H-thiazolo[3,2-c]pyrimidin-6-ylmethyl}-benzoic acid;

2-[3-(4-Fluoro-phenyl)-prop-1-ynyl]-6-(4-methanesulfonyl-benzyl)-8-methyl-thiazolo[3,2-c]pyrimidine-5,7-dione;

4-{2-[3-(4-Fluoro-3-methoxy-phenyl)-prop-1-ynyl]-8-methyl-5,7-dioxo-7H-thiazolo[3,2-c]pyrimidin-6-ylmethyl}-benzonitrile;

(3-{2-[3-(4-Fluoro-3-methoxy-phenyl)-prop-1-ynyl]-8-methyl-5,7-dioxo-7H-thiazolo[3,2-c]pyrimidin-6-ylmethyl}-phenyl)-acetic acid;

(4-{2-[3-(3-Methoxy-phenyl)-prop-1-ynyl]-8-methyl-5,7-dioxo-7H-[3,2-c]pyrimidin-6-ylmethyl}-phenyl)-acetic acid;

6-(3,4-Difluoro-benzyl)-8-methyl-2-(3-phenyl-prop-1-ynyl)-thiazolo[3,2-c]pyrimidine-5,7-dione;

2-[3-(3-Methoxy-phenyl)-prop-1-ynyl]-8-methyl-6-[4-(thiomorpholine-4-carbonyl)-benzyl]-thiazolo[3,2-c]pyrimidine-5,7-dione;

8-Methyl-2-(3-pyridin-4-yl-prop-1-ynyl)-6-[4-(thiomorpholine-4-sulfonyl)-benzyl]-thiazolo[3,2-c]pyrimidine-5,7-dione;

2-[3-(4-Fluoro-3-methoxy-phenyl)-prop-1-ynyl]-8-methyl-6-(2-oxo-2H-1-benzopyran-6-ylmethyl)-thiazolo[3,2-c]pyrimidine-5,7-dione; and

2-[3-(3-Methoxy-4-methyl-phenyl)-prop-1-ynyl]-8-methyl-6-(2-oxo-2H-1-benzopyran-6-ylmethyl)-thiazolo[3,2-c]pyrimidine-5,7-dione, or a pharmaceutically acceptable salt thereof.

14. The combination according to Embodiment 1, wherein the compound of Formula I is a compound selected from:

6-Benzoyl-thiazolo[3,2-c]pyrimidine-5,7-dione;

6-(4-Chlorobenzyl)-thiazolo[3,2-c]pyrimidine-5,7-dione;

6-Pyridin-4-ylmethyl-thiazolo]3,2-c]pyrimidine-5,7-dione;

8-Methyl-thiazolo[3,2-c]pyrimidine-5,7-dione;

8-Methyl-5,7-dioxo-6,7-dihydro-5H-thiazolo[3,2-c]pyrimidine-2-carboxylic acid;

6-Benzyl-8-methyl-5,7-dioxo-6,7-dihydro-5H-thiazolo[3,2-c]pyrimidine-2-carboxylic acid;

4-(8-methyl-5,7-dioxo-7H-thiazolo[3,2-c]pyrimidin-6-yl-methyl)-benzoic acid tert-butyl ester; and

8-Methyl-6-[4-(Morpholine-4-sulfonyl)benzyl]-thiazolo[3,2-c]pyrimidine-5,7-dione, or a pharmaceutically acceptable salt thereof.

15. The combination according to Embodiment 1, wherein the compound of Formula I is selected from:

8-Methyl-5,7-dioxo-6-(3-oxo-3-phenyl-propyl)-6,7-dihydro-5H-[3,2-c]pyrimidine-2-carboxylic acid 4-fluoro-benzylamide;

8-Methyl-6-(1-phenylethyl) 5,7-dioxo-6,7-dihydro-SH-thiazolo[3,2-c]pyrimidine-2-carboxylic acid 4-fluorobenzylamide;

8-Methyl-5,7-dioxo-6-(2-phenylmethanesulfonyl-ethyl)-6,7-dihydro-5H-[3,2-c]pyrimidine-2-carboxylic acid 4-fluorobenzylamide;

6-(5-Cyano-pentyl)-8-Methyl-5,7-dioxo-6,7-dihydro-5H-thiazolo[3,2-c]pyrimidine-2-carboxylic acid 4-fluorobenzylamide;

6-(E)-But-2-enyl-8-Methyl-5,7-dioxo-6,7-dihydro-SH-thiazolo[3,2-c]pyrimidine-2-carboxylic acid 4-fluorobenzylamide;

8-Methyl-5,7-dioxo-6-(E)-pent-2-enyl-6,7-dihydro-SH-thiazolo[3,2-c]pyrimidine-2-carboxylic acid 4-fluorobenzylamide;

6-sec-Butyl-8-Methyl-5,7-dioxo-6,7-dihydro-5H-thiazolo[3,2-c]pyrimidine-2-carboxylic acid 4-fluorobenzylamide;

8-Methyl-6-(2-methyl-allyl)-5,7-dioxo-6,7-dihydro-5H-thiazolo[3,2-c]pyrimidine-2-carboxylic acid 4-fluorobenzylamide;

6-(1-Ethyl-propyl)-8-Methyl-5,7-dioxo-6,7-dihydro-SH-thiazolo[3,2-c]pyrimidine-2-carboxylic acid 4-fluorobenzylamide;

8-Methyl-5,7-dioxo-6-pent-2-ynyl-6,7-dihydro-5H-thiazolo[3,2-c]pyrimidine-2-carboxylic acid 4-fluorobenzylamide;

6-(2-Benzensulfonyl-ethyl)-8-Methyl-5,7-dioxo-6,7-dihydro-5H-[3,2-c]pyrimidine-2-carboxylic acid 4-fluorobenzylamide;

8-Methyl-6-(3-methyl-but-2-enyl)-5,7-dioxo-6,7-dihydro-5H-thiazolo[3,2-c]pyrimidine-2-carboxylic acid 4-fluorobenzylamide;

6-[2-(4-Fluoro-benzensulfonyl)-ethyl]-8-Methyl-5,7-dioxo-6,7-dihydro-[3,2-c]pyrimidine-2-carboxylic acid 4-fluorobenzylamide;

6-[3-(4-Fluoro-phenyl)-3-oxo-propyl]-8-Methyl-5,7-dioxo-6,7-dihydro-5H-thiazolo[3,2-c]pyrimidine-2-carboxylic acid 4-fluorobenzylamide;

8-Methyl-5,7-dioxo-6-{2-[(1-phenyl-methanoyl)-amino]-ethyl}-6,7-dihydro-5H-thiazolo[3,2-c]pyrimidine-2-carboxylic acid 4-fluorobenzylamide;

8-Methyl-5,7-dioxo-6-(2-phenoxy-ethyl)-6,7-dihydro-5H-thiazolo[3,2-c]pyrimidine-2-carboxylic acid 4-fluorobenzylamide; and

{5-[2-(4-Fluoro-benzylcarbamoyl)-8-methyl-5,7-dioxo-7H-thiazolo[3,2-c]pyrimidine-6-ylmethyl]-isoxazol-3-yl]}-carbamic acid methyl ester, or a pharmaceutically acceptable salt thereof.

16. A combination, comprising valdecoxib, or a pharmaceutically acceptable salt thereof, and an allosteric carboxylic inhibitor of MMP-13 of Formula IA

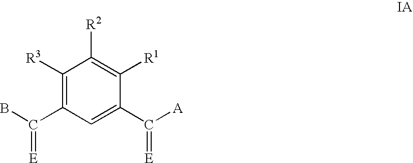

wherein R¹, R², and R³ independently are hydrogen, halo, hydroxy, C₁-C₆ alkyl, C₁-C₆ alkoxy, C₂-C₆ alkenyl, C₂-C₆ alkynyl, NO₂, NR⁴R⁵, CN, or CF₃;

E is independently O or S;

A and B independently are OR⁴ or NR⁴R⁵;

each R⁴ and R⁵ independently are H, C₁-C₆ alkyl, C₂-C₆ alkenyl, C₂-C₆ alkynyl, (CH₂)ₙ aryl, (CH₂)ₙ cycloalkyl, (CH₂)ₙ heteroaryl, or R⁴ and R⁵ when taken together with the nitrogen to which they are attached complete a 3- to 8-membered ring, optionally containing a heteroatom selected from O, S, or NH, and optionally substituted or unsubstituted;

n is an integer from 0 to 6;

or a pharmaceutically acceptable salt thereof.

17. The combination according to Embodiment 16, wherein the compound of Formula IA is a compound of Formula IIA

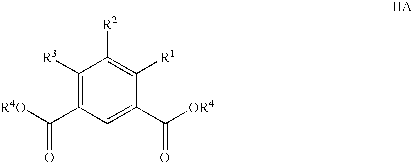

or a pharmaceutically acceptable salt thereof,

wherein R¹, R², and R³ are as defined above for Embodiment 16, and each R⁴ independently is as defined above for Embodiment 16.

18. The combination according to Embodiment 16, wherein the compound of Formula IA is a compound of Formula IIIA

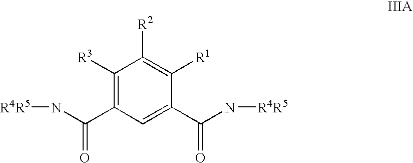

or a pharmaceutically acceptable salt thereof,

wherein R¹, R², and R³ are as defined above for Embedment 16, and each R⁴ and R⁵ independently are as defined above.

19. The combination according to Embodiment 16, wherein the compound of Formula IA is a compound of Formula IVA

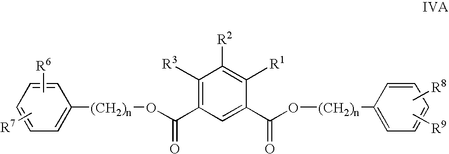

or a pharmaceutically acceptable salt thereof,

wherein n, R¹, R², and R³ are as defined above for Embodiment 16, and R⁶, R⁷, R⁸, and R⁹ independently are hydrogen, halo, C₁-C₆ alkyl, C₁-C₆ alkoxy, nitro, or NH_{2.}

20. The combination according to Embodiment 16, wherein the compound of Formula IA is a compound of Formula VA

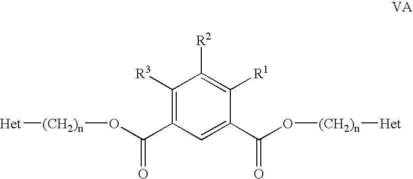

or a pharmaceutically acceptable salt thereof,

wherein n, R¹, R², and R³ are as defined above for Embodiment 16, and Het is an unsubstituted or substituted heteroaryl group.

21. The combination according to Embodiment 16, wherein the compound of Formula IA is a compound of Formula VIA

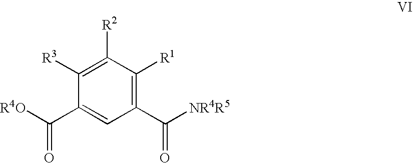

or a pharmaceutically acceptable salt thereof,

wherein R¹, R², and R³ are as defined above for Embodiment 16, and each R⁴ and R⁵ independently are as defined above for Embodiment 16.

22. The combination according to Embodiment 16, wherein the compound of Formula IA is selected from:

4-Methoxy-N,N′-bis-(4-methoxybenzyl)-isophthalamide;

Isophthalic acid di-(2,1,3-benzothiadiazol-5-yl) methyl ester;

4-Methoxy-isophthalic acid dibenzyl ester;

4-Methoxy-isophthalic acid dipyridin-4-ylmethyl ester;

Isophthalic acid bis-(4-fluoro-benzyl) ester;

Isophthalic acid bis-(3-fluoro-benzyl) ester;

Isophthalic acid bis-(4-methoxy-benzyl) ester;

Isophthalic acid bis-(3-methoxy-benzyl) ester;

Isophthalic acid bis-(1,3-benzodioxol-5-ylmethyl) ester;

N,N′-Bis-(3-fluoro-benzyl)-isophthalamide;

4-Acetyl-isophthalic acid dibenzyl ester;

4-Methoxycarbonylmethoxy-isophthalic acid dibenzyl ester;

N,N′-Bis-1,3-benzodioxol-5-ylmethyl-4-methoxy-isophthalamide;

N-1,3-Benzodioxol-5-ylmethyl-4-methoxy-N′-(4-methoxy-benzyl)-isophthalamide;

4-Methoxy-N,N′-bis-(4-methoxy-benzyl)-isophthalamide;

N-1,3-Benzodioxol-5-ylmethyl-N′-(4-chloro-benzyl)-4-methoxy-isophthalamide;

N-Benzyl-4-methoxy-N′-(4-methoxy-benzyl)-isophthalamide;

N′-Benzyl-4-methoxy-N-(4-methoxy-benzyl)-isophthalamide;

4-Methoxy-N-(4-methoxy-benzyl)-N′-pyridin-4-ylmethyl-isophthalamide;

N′-1,3-Benzodioxol-5-ylmethyl-4-methoxy-N-(2-phenoxy-ethyl)-isophthalamide;

N-1,3-Benzodioxol-5-ylmethyl-4-methoxy-N′-(2-phenoxy-ethyl)-isophthalamide;

N-1,3-Benzodioxol-5-ylmethyl-N′-furan-2-ylmethyl-isophthalamide;

N′-1,3-Benzodioxol-5-ylmethyl-N-(2-ethoxy-ethyl)-4-methoxy-isophthalamide;

N,N′-Bis-(3-hydroxymethyl-phenyl)-isophthalamide;

N-Benzyl-4-methoxy-N′-(2-phenoxy-ethyl)-isophthalamide;

4-Methoxy-N,N′-bis-(4-methyl-benzyl)-isophthalamide;

4-Methoxy-N,N′-bis-(3-methoxy-benzyl)-isophthalamide;

N-1,3-Benzodioxol-5-ylmethyl-4-methoxy-N′-(4-methoxy-benzyl)-isophthalamide;

N-1,3-Benzodioxol-5-ylmethyl-isophthalamic acid, (4-carboxyphenyl) methyl ester;

4-{[3-(3-Methoxy-benzylcarbamoyl)-benzoylamino]-methyl}-benzoic acid;

4-Methoxy-isophthalic acid di-2,1,3-benzothiadiazol-5-ylmethyl ester;

4-{[3-(3-Methoxy-benzylcarbamoyl)-benzoylamino]-methyl}-benzoic acid methyl ester;

N-(3-Methoxy-benzyl)-N′-(4-nitro-benzyl)-isophthalamide;

N-(3,4-Dichloro-benzyl)-N′-pyridin-4-ylmethyl-isophthalamide;

N-1,3-Bis-1,3-benzodioxol-5-ylmethyl-4-ethoxy-isophthalamide;

N-(4-Chloro-benzyl)-N′-(3-methoxy-benzyl)-isophthalamide;

N-(3,4-Dichloro-benzyl)-N′-(3-methoxy-benzyl)-isophthalamide;

N-(4-Methoxy-benzyl)-N′-(3-methoxy-benzyl)-isophthalamide;

N,N′-Bis-(4-fluoro-3-methoxy-benzyl)-isophthalamide;

4-Ethoxy-N1,N3-bis-(3-methoxy-benzyl)-isophthalamide;

N1,N3-Bis-1,3-benzodioxol-5-ylmethyl-4-ethoxy-isophthalamide;

N-(3-Methoxy-benzyl)-N′-pyridin-3-ylmethyl-isophthalamide;

N-(3-Methoxy-benzyl)-N′-pyridin-4-ylmethyl-isophthalamide;

N1-1,3-Benzodioxol-5-ylmethyl-N-3-pyridin-3-ylmethyl-isophthalamide;

N-(3-Methoxy-benzyl)-N′-(3-trifluoromethoxy-benzyl)-isophthalamide;

N1,N3-Bis-1,3-benzodioxol-5-ylmethyl-4-isopropoxy-isophthalamide;

4-Isopropoxy-N1,N3-bis-(3-methoxy-benzyl)-isophthalamide;

N1-Benzyl-4-methoxy-N-3-(4-methoxy-benzyl)-isophthalamide;

N1-1,3-Benzodioxol-5-ylmethyl-4-methoxy-N-3-(4-methoxy-benzyl)-isophthalamide;

N1-1,3-Benzodioxol-5-ylmethyl-4-methoxy-N-3-(2-phenoxy-ethyl)-isophthalamide;

N1-Benzyl-4-methoxy-N-3-(2-phenoxy-ethyl)-isophthalamide;

N1-1,3-Benzodioxol-5-ylmethyl-N-3-(4-chloro-benzyl)-4-methoxy-isophthalamide;

N3-1,3-Benzodioxol-5-ylmethyl-4-methoxy-N 1-(4-methoxy-benzyl)-isophthalamide;

N3-Benzyl-4-methoxy-N-1-(4-methoxy-benzyl)-isophthalamide;

N3-1,3-Benzodioxol-5-ylmethyl-4-methoxy-N 1-(2-phenoxy-ethyl)-isophthalamide;

N3-1,3-Benzodioxol-5-ylmethyl-N 1-(2-ethoxy-ethyl)-4-methoxy-isophthalamide;

4-Methoxy-N1-(4-methoxy-benzyl)-N-3-pyridin-4-ylmethyl-isophthalamide;

4-Amino-N1,N3-bis-1,3-benzodioxol-5-ylmethyl-isophthalamide;

4-Acetylamino-N1,N3-bis-1,3-benzodioxol-5-ylmethyl-isophthalamide;

N-(3-Methoxy-benzyl)-N′-pyridin-3-ylmethyl-isophthalamide;

N-(3-Methoxy-benzyl)-N′-pyridin-4-ylmethyl-isophthalamide;

N1-1,3-Benzodioxol-5-ylmethyl-N-3-pyridin-3-ylmethyl-isophthalamide;

N-(4-Chloro-benzyl)-N′-(3-methoxy-benzyl)-isophthalamide;

N-(3,4-Dichloro-benzyl)-N′-(3-methoxy-benzyl)-isophthalamide;

N-(4-Methoxy-benzyl)-N′-(3-methoxy-benzyl)-isophthalamide;

N-(3-Methoxy-benzyl)-N′-(4-methyl-benzyl)-isophthalamide;

N,N′-Bis-(4-fluoro-3-methoxy-benzyl)-isophthalamide;

({3-[(1,3-Benzodioxol-5-ylmethyl)-carbamoyl]-benzoyl}-benzyl-amino)-acetic acid;

N-Benzo[1,3]dioxol-5-ylmethyl-isophthalamic(4-hydroxymethyl-benzoic acid) ester;

N-(3,4-Dichloro-benzyl)-N′-pyridin-4-ylmethyl-isophthalamide;

N-(3-Methoxy-benzyl)-N′-(4-nitro-benzyl)-isophthalamide;

4-{[3-(3-Methoxy-benzylcarbamoyl)-benzoylamino]-methyl}-benzoic acid methyl ester;

N-3-Methoxybenzyl-isophthalamic(4-hydroxymethyl-benzoic acid) ester;

4-{[3-(3-Methoxy-benzylcarbamoyl)-benzoylamino]-methyl}-benzoic acid;

N-(3-Amino-benzyl)-N′-(3-methoxy-benzyl)-isophthalamide;

N-(3-Methoxy-benzyl)-N′-(3-nitro-benzyl)-isophthalamide;

4-Ethoxy-N′1,N″3-bis-(3-methoxy-benzyl)-isophthalamide;

N1,N3-Bis-1,3-benzodioxol-5-ylmethyl-4-ethoxy-isophthalamide;

N1,N3-Bis-1,3-benzodioxol-5-ylmethyl-4-propoxy-isophthalamide;

N1,N3-Bis-1,3-benzodioxol-5-ylmethyl-4-isopropoxy-isophthalamide;

N1,N3-Bis-2,1,3-benzothiadiazol-5-ylmethyl-4-methoxy-isophthalamide; and

4-Methoxy-isophthalic acid di-2,1,3-benzothiadiazol-5-ylmethyl ester, or a pharmaceutically acceptable salt thereof.

23. A combination, comprising valdecoxib, or a pharmaceutically acceptable salt thereof, and an allosteric carboxylic inhibitor of MMP-13 of Formula IB

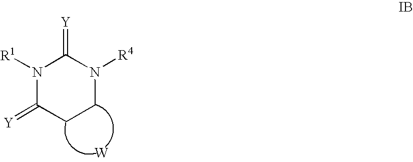

or a pharmaceutically acceptable salt thereof;

wherein:

W, together with the carbon atoms to which it is attached, form a 5-membered ring diradical

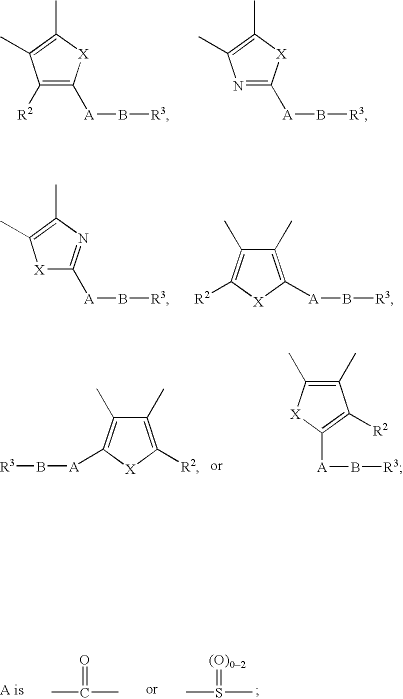

B is O or NR⁵; or

A and B are taken together to form —C≡C—;

X is O, S, SO, SO₂, NR⁵, or CH₂;

each Y independently is O or S;

R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;

R² and R³ independently are hydrogen, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl; CONR⁴R⁵, or COR⁶;

R² may further be halo;

n is an integer of from 0 to 5;

R⁴ and R⁵ when taken together with the nitrogen to which they are attached complete a 3- to 8-membered ring containing carbon atoms and optionally containing O, S, or N, and substituted or unsubstituted;

wherein R¹ and R³ are not both selected from: hydrogen and C₁-C₆ alkyl.

24. The combination according to Embodiment 23, wherein the compound of Formula IB is a compound of Formula IIB

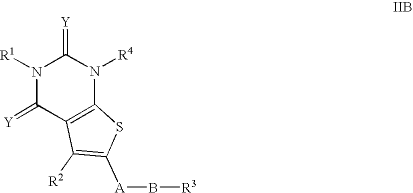

or a pharmaceutically acceptable salt thereof, wherein A, B, R¹, R², R³, R⁴, and Y are as defined above for Embodiment 23.

25. The combination according to Embodiment 23, wherein the compound of Formula IB is a compound of Formula IIIB

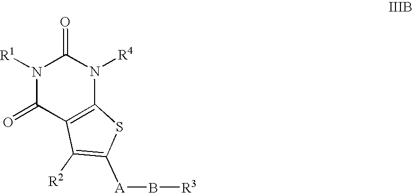

or a pharmaceutically acceptable salt thereof, wherein A, B, R¹, R², and R⁴ are as defined above for Embodiment 23, and R³ is (CH₂)ₙ aryl, (CH₂)ₙ cycloalkyl, or (CH₂)ₙ heteroaryl.

26. The combination according to Embodiment 23, wherein the compound of Formula IB is a compound of Formula IVB

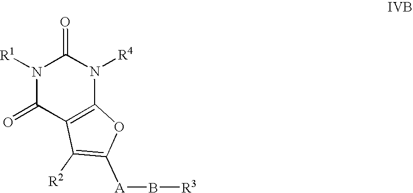

or a pharmaceutically acceptable salt thereof, wherein A, B, R¹, R², R³, and R⁴ are as defined above for Embodiment 23.

27. The combination according to Embodiment 23, wherein the compound of Formula IB is a compound of Formula VB

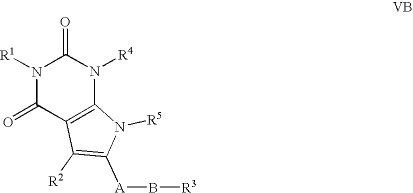

or a pharmaceutically acceptable salt thereof, wherein A, B, R¹, R², R³, R⁴, and R⁵ are as defined above for Embodiment 23.

28. The combination according to Embodiment 23, wherein the compound of Formula IB is a compound of Formulas VIIB, VIIB, VIIIB, or IXB:

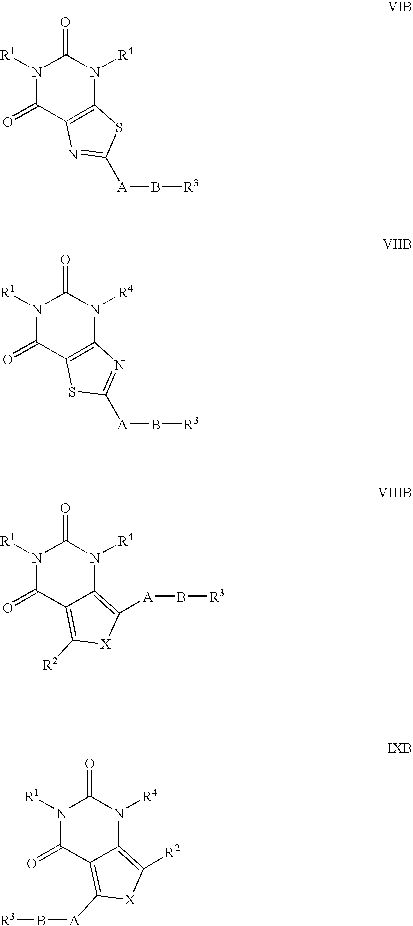

or a pharmaceutically acceptable salt thereof, wherein A, B, X, R¹, R², R³, and R⁴ are as defined above for Embodiment 23.

29. The combination according to Embodiment 23, wherein the compound of Formula IB is a compound of Formula XB

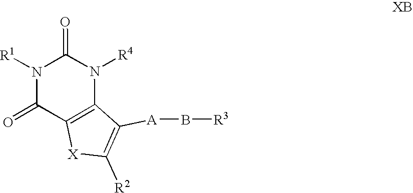

or a pharmaceutically acceptable salt thereof, wherein R¹—R⁴, A, B, and X are as defined above for Embodiment 23.

30. The combination according to Embodiment 23, wherein the compound of Formula IB is a compound of Formula XIB

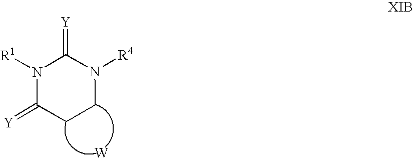

or a pharmaceutically acceptable salt thereof,

wherein:

W, together with the carbon atoms to which it is attached, form a 5-membered ring diradical

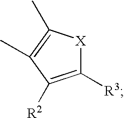

each Y independently is O or S;

X is S, O, or NR⁵;

R¹, R⁴, and R⁵ independently are hydrogen, C₁-C₆ alkyl, C₂-C₆ alkenyl, C₂-C₆ alkynyl, (CH₂)ₙ cycloalkyl, (CH₂)ₙ heterocyclic, C₁-C₆ alkanoyl, (CH₂)ₙ aryl, or (CH₂)ₙ heteroaryl;

R² is hydrogen, C₁-C₆ alkyl, C₂-C₆ alkenyl, C₂-C₆ alkynyl, CN, NO₂, NR⁴R⁵, (CH₂)ₙ cycloalkyl, (CH₂)ₙ aryl, or (CH₂)ₙ heteroaryl; R³ is hydrogen, halo, C₁-C₆ alkyl, C₂-C₆ alkenyl, CN, NO₂, NR⁴R⁵, (CH₂)_{q} cycloalkyl, (CH₂)_{q} aryl, or (CH₂)_{q} heteroaryl;

n is 0, 1, or 2;

q is 2, 3, or 4; and

R⁴ and R⁵ when taken together with the nitrogen to which they are attached complete a 3- to 8-membered ring containing carbon atoms and optionally containing O, S, or N, and substituted or unsubstituted;

wherein R¹ and R³ are not both selected from: hydrogen and C₁-C₆ alkyl.

31. The combination according to Embodiment 23, wherein the compound of Formula 1B is selected from:

3-Benzyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzyl ester;

3-(4-Pyridyl)-2,4-dioxo-1,2,3,4-tetrahydro-thieno[2,3-d]pyrimidine-6-carboxylic acid benzyl ester;

3-(4-Pyridyl)-1-methyl-2,4-dioxo-1,2,3,4-tetrahydro-thieno[2,3-d]-pyrimidine-6-carboxylic acid benzyl ester;

3-Benzyl-2,4-dioxo-1,2,3,4-tetrahydro-thieno[2,3-d]pyrimidine-6-carboxylic acid (4-pyridyl) ester;

3-Benzyl-1-methyl-2,4-dioxo-1,2,3,4-tetrahydro-thieno[2,3-d]pyrimidine-6-carboxylic acid (4-pyridyl) ester;

3-(4-Pyridyl)-2,4-dioxo-1,2,3,4-tetrahydro-thieno[2,3-d]pyrimidine-6-carboxylic acid (4-pyridyl) ester;

3-(4-Pyridyl)-1-methyl-2,4-dioxo-1,2,3,4-tetrahydro-thieno[2,3-d]-pyrimidine-6-carboxylic acid (4-pyridyl) ester;

3-Benzyl-2,4-dioxo-1,2,3,4-tetrahydro-thieno[2,3-d]pyrimidine-6-carboxylic acid piperoyl ester;

3-Benzyl-1-methyl-2,4-dioxo-1,2,3,4-tetrahydro-thieno[2,3-d]pyrimidine-6-carboxylic acid piperoyl ester;

3-Piperoyl-2,4-dioxo-1,2,3,4-tetrahydro-thieno[2,3-d]pyrimidine-6-carboxylic acid piperoyl ester;

3-Piperoyl-1-methyl-2,4-dioxo-1,2,3,4-tetrahydro-thieno[2,3-d]-pyrimidine-6-carboxylic acid piperoyl ester;

3-Benzyl-1-methyl-2,4-dioxo-1,2,3,4-tetrahydro-furo[2,3-d]pyrimidine-6-carboxylic acid benzyl ester;

3-Benzyl-1-methyl-2,4-dioxo-1,2,3,4-tetrahydro-1H-pyrrolo[2,3-d]-pyrimidine-6-carboxylic acid benzyl ester;

3-Benzyl-1,7-dimethyl-2,4-dioxo-1,2,3,4-tetrahydro-1H-pyrrolo[2,3-d]-pyrimidine-6-carboxylic acid benzyl ester;

3-Benzyl-1,7-dimethyl-2,4-dioxo-1,2,3,4-tetrahydro-1H-pyrrolo[2,3-d]-pyrimidine-6-carboxylic acid benzofuran-6-ylmethyl ester;

3-Benzyl-1-dimethyl-2,4-dioxo-1,2,3,4-tetrahydro-1H-pyrrolo[2,3-d]-pyrimidine-6-carboxylic acid benzofuran-6-ylmethyl ester;

3-Benzyl-1-methyl-2,4-dioxo-1,2,3,4-tetrahydro-furo[2,3-d]pyrimidine-6-carboxylic acid benzofuran-6-ylmethyl ester;

3-Benzyl-1-methyl-2,4-dioxo-1,2,3,4-tetrahydro-thieno[2,3-d]pyrimidine-6-carboxylic acid benzofuran-6-ylmethyl ester;

3-Benzyl-1,7-dimethyl-2,4-dioxo-1,2,3,4-tetrahydro-1H-pyrrolo[2,3-d]-pyrimidine-6-carboxylic acid benzothiophene-6-ylmethyl ester;

3-Benzyl-1-dimethyl-2,4-dioxo-1,2,3,4-tetrahydro-1H-pyrrolo[2,3-d]-pyrimidine-6-carboxylic acid benzothiophene-6-ylmethyl ester;

3-Benzyl-1-methyl-2,4-dioxo-1,2,3,4-tetrahydro-furo[2,3-d]pyrimidine-6-carboxylic acid benzothiophene-6-ylmethyl ester;

3-Benzyl-1-methyl-2,4-dioxo-1,2,3,4-tetrahydro-thieno[2,3-d]pyrimidine-6-carboxylic acid benzothiophene-6-ylmethyl ester;

3-(3-Methoxy-benzyl)-1-methyl-2,4-dioxo-1,2,3,4-tetrahydro-thieno [2,3-d]pyrimidine-6-carboxylic acid benzyl ester;

3-(4-Chloro-benzyl)-1-methyl-2,4-dioxo-1,2,3,4-tetrahydro-thieno[2,3-d]pyrimidine-6-carboxylic acid benzyl ester;

3-(4-Cyano-benzyl)-1-methyl-2,4-dioxo-1,2,3,4-tetrahydro-thieno[2,3-d]pyrimidine-6-carboxylic acid benzyl ester;

3-(4-Fluoro-benzyl)-1-methyl-2,4-dioxo-1,2,3,4-tetrahydro-thieno[2,3-d]pyrimidine-6-carboxylic acid benzyl ester;

3-(3-Chloro-benzyl)-1-methyl-2,4-dioxo-1,2,3,4-tetrahydro-thieno[2,3-d]pyrimidine-6-carboxylic acid benzyl ester;

1-Methyl-3-(2-methyl-benzyl)-2,4-dioxo-1,2,3,4-tetrahydro-thieno[2,3-d]pyrimidine-6-carboxylic acid benzyl ester;

1-Methyl-3-(4-methyl-benzyl)-2,4-dioxo-1,2,3,4-tetrahydro-thieno[2,3-d]pyrimidine-6-carboxylic acid benzyl ester;

3-(4-Carboxy-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(3-trifluoromethyl-benzyl)-1,2,3,4-tetrahydro-[2,3-d]pyrimidine-6-carboxylic acid benzyl ester;

3-Biphenyl-4-ylmethyl-1-methyl-2,4-dioxo-1,2,3,4-tetrahydro-thieno[2,3-d]pyrimidine-6-carboxylic acid benzyl ester;

1-Methyl-2,4-dioxo-3-(2-trifluoromethyl-benzyl)-1,2,3,4-tetrahydro-[2,3-d]pyrimidine-6-carboxylic acid benzyl ester;

3-(3-Cyano-benzyl)-1-methyl-2,4-dioxo-1,2,3,4-tetrahydro-thieno[2,3-d]pyrimidine-6-carboxylic acid benzyl ester;

3-(2-Cyano-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethyl-benzyl)-1,2,3,4-tetrahydro-[2,3-d]pyrimidine-6-carboxylic acid benzyl ester;

3-[2-Hydroxy-3-(naphthalen-1-yloxy)-propyl]-1-methyl-2,4-dioxo-1,2,3,4-tetrahydro-thieno[2,3-d]pyrimidine-6-carboxylic acid benzyl ester;

3-(3-Chloro-benzyl)-1-methyl-2,4-dioxo-1,2,3,4-tetrahydro-thieno[2,3-d]pyrimidine-6-carboxylic acid benzyl ester;

1-Methyl-3-naphthalen-1-ylmethyl-2,4-dioxo-1,2,3,4-tetrahydro-[2,3-d]pyrimidine-6-carboxylic acid benzyl ester;

3-(6-Chloro-benzo[1,3]dioxol-5-ylmethyl)-1-methyl-2,4-dioxo-1,2,3,4-tetrahydro-thieno[2,3-d]pyrimidine-6-carboxylic acid benzyl ester;

1-Methyl-2,4-dioxo-3-(4-oxo-4-thiophen-2-yl-butyl)-1,2,3,4-tetrahydro-[2,3-d]pyrimidine-6-carboxylic acid benzyl ester;

1-Methyl-2,4-dioxo-3-pyridin-4-ylmethyl-1,2,3,4-tetrahydro-thieno[2,3-d]pyrimidine-6-carboxylic acid benzyl ester;

1-Methyl-2,4-dioxo-3-(4-m-tolyloxy-butyl)-1,2,3,4-tetrahydro-thieno[2,3-d]pyrimidine-6-carboxylic acid benzyl ester;

3-(3,5-Dimethyl-isoxazol-4-ylmethyl)-1-methyl-2,4-dioxo-1,2,3,4-tetrahydro-thieno[2,3-d]pyrimidine-6-carboxylic acid benzyl ester;

3-Dioxo-1,3-dihydro-isoindol-2-ylmethyl)-1-methyl-2,4-dioxo-1,2,3,4-tetrahydro-thieno[2,3-d]pyrimidine-6-carboxylic acid benzyl ester;

3-Dihydro-benzo[1,4]dioxin-2-ylmethyl)-1-methyl-2,4-dioxo-1,2,3,4-tetrahydro-thieno[2,3-d]pyrimidine-6-carboxylic acid benzyl ester;

1-Methyl-2,4-dioxo-3-pyridin-2-ylmethyl-1,2,3,4-tetrahydro-thieno[2,3-d]pyrimidine-6-carboxylic acid benzyl ester;

3-[2-(2,5-Dimethoxy-phenyl)-2-oxo-ethyl]-1-methyl-2,4-dioxo-1,2,3,4-tetrahydro-thieno[2,3-d]pyrimidine-6-carboxylic acid benzyl ester;

3-Benzyloxymethyl-1-methyl-2,4-dioxo-1,2,3,4-tetrahydro-thieno [2,3-d]pyrimidine-6-carboxylic acid benzyl ester;

1-Methyl-2,4-dioxo-3-(4-m-tolyloxy-butyl)-1,2,3,4-tetrahydro-thieno[2,3-d]pyrimidine-6-carboxylic acid benzyl ester;

1-Methyl-2,4-dioxo-3-(2-phenylmethanesulfonyl-ethyl)-1,2,3,4-tetrahydro-thieno[2,3-d]pyrimidine-6-carboxylic acid benzyl ester;

3-(4-Amino-6-phenylamino-[1,3,5]triazin-2-ylmethyl)-1-methyl-2,4-dioxo-1,2,3,4-tetrahydro-thieno[2,3-d]pyrimidine-6-carboxylic acid benzyl ester;

3-[4-(4-Fluoro-phenyl)-4-oxo-butyl]-11-methyl-2,4-dioxo-1,2,3,4-tetrahydro-thieno[2,3-d]pyrimidine-6-carboxylic acid benzyl ester;

3-[4-(1,3-Dioxo-1,3-dihydro-isoindol-2-yl)-butyl]-11-methyl-2,4-dioxo-1,2,3,4-tetrahydro-thieno[2,3-d]pyrimidine-6-carboxylic acid benzyl ester;

1-Methyl-2,4-dioxo-3-(4-phenoxy-butyl)-1,2,3,4-tetrahydro-thieno[2,3-d]pyrimidine-6-carboxylic acid benzyl ester;

1-Methyl-2,4-dioxo-3-(4-oxo-4-phenyl-butyl)-1,2,3,4-tetrahydro-[2,3-d]pyrimidine-6-carboxylic acid benzyl ester;

1-Methyl-2,4-dioxo-3-(2-phenoxy-ethyl)-1,2,3,4-tetrahydro-thieno[2,3-d]pyrimidine-6-carboxylic acid benzyl ester;

3-{3-[4-(3-Chloro-phenyl)-piperazin-1-yl]-propyl}-1-methyl-2,4-dioxo-1,2,3,4-tetrahydro-thieno[2,3-d]pyrimidine-6-carboxylic acid benzyl ester;

3-[1-Bromo-2-(1H-indol-3-yl)-ethyl]-1-methyl-2,4-dioxo-1,2,3,4-tetrahydro-thieno[2,3-d]pyrimidine-6-carboxylic acid benzyl ester;

3-(2-Benzenesulfinyl-ethyl)-1-methyl-2,4-dioxo-1,2,3,4-tetrahydro-[2,3-d]pyrimidine-6-carboxylic acid benzyl ester;

3-[3-(3-Fluoro-phenylcarbamoyl)-propyl]-1-methyl-2,4-dioxo-1,2,3,4-tetrahydro-thieno[2,3-d]pyrimidine-6-carboxylic acid benzyl ester;

1-Methyl-2,4-dioxo-3-[2-(2-trifluoromethyl-phenylcarbamoyl)-ethyl]-1,2,3,4-tetrahydro-thieno[2,3-d]pyrimidine-6-carboxylic acid benzyl ester;

3-[2-(4-Methoxy-phenyl)-ethyl]-1-methyl-2,4-dioxo-1,2,3,4-tetrahydro-[2,3-d]pyrimidine-6-carboxylic acid benzyl ester;

3-[2-(4-Chloro-2-nitro-phenoxy)-ethyl]-1-methyl-2,4-dioxo-1,2,3,4-tetrahydro-thieno[2,3-d]pyrimidine-6-carboxylic acid benzyl ester;

1-Methyl-3-(5-nitro-furan-2-ylmethyl)-2,4-dioxo-1,2,3,4-tetrahydro-[2,3-d]pyrimidine-6-carboxylic acid benzyl ester;

3-(1-Benzyl-1H-imidazol-2-ylmethyl)-1-methyl-2,4-dioxo-1,2,3,4-tetrahydro-thieno[2,3-d]pyrimidine-6-carboxylic acid benzyl ester;

3-[3-(Benzyl-methyl-amino)-propyl]-1-methyl-2,4-dioxo-1,2,3,4-tetrahydro-thieno[2,3-d]pyrimidine-6-carboxylic acid benzyl ester;

3-(Bis-trifluoromethyl-benzyl)-1-methyl-2,4-dioxo-1,2,3,4-tetrahydro-[2,3-d]pyrimidine-6-carboxylic acid benzyl ester;

3-[3-(2-Bromo-4-methyl-phenoxy)-propyl]-1-methyl-2,4-dioxo-1,2,3,4-tetrahydro-thieno[2,3-d]pyrimidine-6-carboxylic acid benzyl ester;

3-Benzenesulfonylmethyl-1-methyl-2,4-dioxo-1,2,3,4-tetrahydro-[2,3-d]pyrimidine-6-carboxylic acid benzyl ester;

3-[2-(4-Chloro-benzenesulfonyl)-ethyl]-1-methyl-2,4-dioxo-1,2,3,4-tetrahydro-thieno[2,3-d]pyrimidine-6-carboxylic acid benzyl ester;

3-Benzo[1,3]dioxol-5-ylmethyl-1-methyl-2,4-dioxo-1,2,3,4-tetrahydro-[2,3-d]pyrimidine-6-carboxylic acid benzyl ester;

3-(3-Iodo-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethoxy-benzyl)-1,2,3,4-tetrahydro-[2,3-d]pyrimidine-6-carboxylic acid benzyl ester;

3-(4-Acetoxy-butyl)-1-methyl-2,4-dioxo-1,2,3,4-tetrahydro-thieno[2,3-d]pyrimidine-6-carboxylic acid benzyl ester;

3-(4-Methanesulfonyl-benzyl)-1-methyl-2,4-dioxo-1,2,3,4-tetrahydro-[2,3-d]pyrimidine-6-carboxylic acid benzyl ester;

1-Methyl-2,4-dioxo-3-(4-[1,2,3]thiadiazol-4-yl-benzyl)-1,2,3,4-tetrahydro-[2,3-d]pyrimidine-6-carboxylic acid benzyl ester;

3-(5-Methoxycarbonyl-furan-2-ylmethyl)-1-methyl-2,4-dioxo-1,2,3,4-tetrahydro-thieno[2,3-d]pyrimidine-6-carboxylic acid benzyl ester;

3-(2-Carboxy-ethyl)-1-methyl-2,4-dioxo-1,2,3,4-tetrahydro-thieno[2,3-d]pyrimidine-6-carboxylic acid benzyl ester;

1-Methyl-2,4-dioxo-3-(3-pyrrol-1-yl-propyl)-1,2,3,4-tetrahydro-[2,3-d]pyrimidine-6-carboxylic acid benzyl ester;

3-(3-Carboxy-propyl)-1-methyl-2,4-dioxo-1,2,3,4-tetrahydro-thieno[2,3-d]pyrimidine-6-carboxylic acid benzyl ester;

3-(2-Cyano-ethyl)-1-methyl-2,4-dioxo-1,2,3,4-tetrahydro-thieno[2,3-d]pyrimidine-6-carboxylic acid benzyl ester;

3-(3-Ethoxycarbonyl-furan-2-ylmethyl)-1-methyl-2,4-dioxo-1,2,3,4-tetrahydro-thieno[2,3-d]pyrimidine-6-carboxylic acid benzyl ester;

3-(3-Amino-propyl)-1-methyl-2,4-dioxo-1,2,3,4-tetrahydro-thieno[2,3-d]pyrimidine-6-carboxylic acid benzyl ester;

3-(3-Cyano-propyl)-1-methyl-2,4-dioxo-1,2,3,4-tetrahydro-thieno[2,3-d]pyrimidine-6-carboxylic acid benzyl ester;

3-(2-Hydroxy-ethyl)-1-methyl-2,4-dioxo-1,2,3,4-tetrahydro-thieno[2,3-d]pyrimidine-6-carboxylic acid benzyl ester;

3-(2-Carboxy-hexyl)-1-methyl-2,4-dioxo-1,2,3,4-tetrahydro-thieno[2,3-d]pyrimidine-6-carboxylic acid benzyl ester;

1-Methyl-2,4-dioxo-3-(2,2,2-trifluoro-ethyl)-1,2,3,4-tetrahydro-thieno[2,3-d]pyrimidine-6-carboxylic acid benzyl ester;

1-Methyl-2,4-dioxo-3-(2,2,2-trifluoro-ethyl)-1,2,3,4-tetrahydro-thieno[2,3-d]pyrimidine-6-carboxyl carboxylic acid benzyl ester;

Iodomethyl-1-methyl-2,4-dioxo-1,2,3,4-tetrahydro-thieno[2,3-d]pyrimidine-6-carboxylic acid benzyl ester;

3-(2-Fluoro-ethyl)-1-methyl-2,4-dioxo-1,2,3,4-tetrahydro-thieno[2,3-d]pyrimidine-6-carboxylic acid benzyl ester;

Methyl-2,4-dioxo-3-(tetrahydro-furan-2-ylmethyl)-1,2,3,4-tetrahydro-[2,3-d]pyrimidine-6-carboxylic acid benzyl ester;

3-[1-(4-Carboxy-phenyl)-ethyl]-1-methyl-2,4-dioxo-1,2,3,4-tetrahydro-[2,3-d]pyrimidine-6-carboxylic acid benzyl ester;

3-(Hex-5-enyl)-1-methyl-2,4-dioxo-1,2,3,4-tetrahydro-thieno[2,3-d]pyrimidine-6-carboxylic acid benzyl ester;

3-(2-Ethyl-butyl)-1-methyl-2,4-dioxo-1,2,3,4-tetrahydro-thieno[2,3-d]pyrimidine-6-carboxylic acid benzyl ester;

1-Methyl-2,4-dioxo-3-(2,2,2-trifluoro-ethyl)-1,2,3,4-tetrahydro-thieno[2,3-d]pyrimidine-6-carboxyl pyrimidine acid benzyl ester;

3-(Diethoxy-phosphorylmethyl)-1-methyl-2,4-dioxo-1,2,3,4-tetrahydro-[2,3-d]pyrimidine-6-carboxylic acid benzyl ester;

3-But-2-ynyl-1-methyl-2,4-dioxo-1,2,3,4-tetrahydro-thieno[2,3-d]pyrimidine-6-carboxylic acid benzyl ester;

Bromo-ethyl)-1-methyl-2,4-dioxo-1,2,3,4-tetrahydro-thieno[2,3-d]pyrimidine-6-carboxylic acid;

1-Methyl-2,4-dioxo-3-[2-(tetrahydro-pyran-2-yloxy)-ethyl]-1,2,3,4-tetrahydro-thieno[2,3-d]pyrimidine-6-carboxylic acid benzyl ester;

1-Methyl-2,4-dioxo-3-propyl-1,2,3,4-tetrahydro-thieno[2,3-d]pyrimidine-6-carboxylic acid benzyl ester;

3-(2-Acetoxy-ethyl)-1-methyl-2,4-dioxo-1,2,3,4-tetrahydro-thieno[2,3-d]pyrimidine-6-carboxylic acid;

3-Butyl-1-methyl-2,4-dioxo-1,2,3,4-tetrahydro-thieno[2,3-d]pyrimidine-6-carboxylic acid benzyl ester;

3-Isobutyl-1-methyl-2,4-dioxo-1,2,3,4-tetrahydro-thieno[2,3-d]pyrimidine-6-carboxylic acid benzyl ester;

3-Ethyl-1-methyl-2,4-dioxo-1,2,3,4-tetrahydro-thieno[2,3-d]pyrimidine-6-carboxylic acid benzyl ester;

3-(3-Bromo-propyl)-1-methyl-2,4-dioxo-1,2,3,4-tetrahydro-thieno[2,3-d]pyrimidine-6-carboxylic acid benzyl ester;

3-Cyclohexylmethyl-1-methyl-2,4-dioxo-1,2,3,4-tetrahydro-thieno[2,3-d]pyrimidine-6-carboxylic acid benzyl ester;

3-(2-Ethylamino-ethyl)-1-methyl-2,4-dioxo-1,2,3,4-tetrahydro-thieno[2,3-d]pyrimidine-6-carboxylic acid benzyl ester;

3-Cyclobutylmethyl-1-methyl-2,4-dioxo-1,2,3,4-tetrahydro-thieno[2,3-d]pyrimidine-6-carboxylic acid benzyl ester;

3-((R)-3-Hydroxy-2-methyl-propyl)-1-methyl-2,4-dioxo-1,2,3,4-tetrahydro-thieno[2,3-d]pyrimidine-6-carboxylic acid benzyl ester;

3-(4-Hydroxy-butyl)-1-methyl-2,4-dioxo-1,2,3,4-tetrahydro-thieno[2,3-d]pyrimidine-6-carboxylic acid benzyl ester;

3-(2-Ethoxy-ethyl)-1-methyl-2,4-dioxo-1,2,3,4-tetrahydro-thieno[2,3-d]pyrimidine-6-carboxylic acid benzyl ester;

3-Isobutyl-1-methyl-2,4-dioxo-1,2,3,4-tetrahydro-thieno [2,3-d]pyrimidine-6-carboxylic acid benzyl ester;

3-(2-Chloro-ethyl)-1-methyl-2,4-dioxo-1,2,3,4-tetrahydro-thieno[2,3-d]pyrimidine-6-carboxylic acid benzyl ester;

1-Methyl-3-(3-methyl-but-2-enyl)-2,4-dioxo-1,2,3,4-tetrahydro-[2,3-d]pyrimidine-6-carboxylic acid benzyl ester;

3-Allyl-1-methyl-2,4-dioxo-1,2,3,4-tetrahydro-thieno[2,3-d]pyrimidine-6-carboxylic acid benzyl ester;

3-(2,2-Dimethoxy-ethyl)-1-methyl-2,4-dioxo-1,2,3,4-tetrahydro-[2,3-d]pyrimidine-6-carboxylic acid benzyl ester;

1-Methyl-3-oxiranylmethyl-2,4-dioxo-1,2,3,4-tetrahydro-thieno [2,3-d]pyrimidine-6-carboxylic acid benzyl ester;

1-Methyl-2,4-dioxo-3-propyl-1,2,3,4-tetrahydro-thieno[2,3-d]pyrimidine-6-carboxylic acid benzyl ester;

3-Benzo[1,2,5]oxadiazol-5-ylmethyl-1-methyl-2,4-dioxo-1,2,3,4-tetrahydro-thieno[2,3-d]pyrimidine-6-carboxylic acid benzyl ester;

3-(3-Hydroxy-2,2-dimethyl-propyl)-1-methyl-2,4-dioxo-1,2,3,4-tetrahydro-thieno[2,3-d]pyrimidine-6-carboxylic acid benzyl ester;

3-(2-Carboxy-ethyl)-1-methyl-2,4-dioxo-1,2,3,4-tetrahydro-thieno[2,3-d]pyrimidine-6-carboxylic acid benzyl ester;

3-Propyl-1-methyl-2,4-dioxo-1,2,3,4-tetrahydro-thieno[2,3-d]pyrimidine-6-carboxylic benzyl ester;

1-Methyl-2,4-dioxo-3-(4-sulfamoyl-benzyl)-1,2,3,4-tetrahydro-thieno [2,3-d]pyrimidine-6-carboxylic acid benzyl ester;

1-Methyl-3-(4-methylsulfamoyl-benzyl)-2,4-dioxo-1,2,3,4-tetrahydro-[2,3-d]pyrimidine-6-carboxylic acid benzyl ester;

3-(4-Dimethylsulfamoyl-benzyl)-1-methyl-2,4-dioxo-1,2,3,4-tetrahydro-[2,3-d]pyrimidine-6-carboxylic acid benzyl ester;

3-(4-Methanesulfonylamino-benzyl)-1-methyl-2,4-dioxo-1,2,3,4-tetrahydro-thieno[2,3-d]pyrimidine-6-carboxylic acid benzyl ester;

3-[4-(Methanesulfonyl-methyl-amino)-benzyl]-1-methyl-2,4-dioxo-1,2,3,4-tetrahydro-thieno[2,3-d]pyrimidine-6-carboxylic acid benzyl ester;

3-(4-Acetylamino-benzyl)-1-methyl-2,4-dioxo-1,2,3,4-tetrahydro-[2,3-d]pyrimidine-6-carboxylic acid benzyl ester;

3-[4-(Acetyl-methyl-amino)-benzyl]-1-methyl-2,4-dioxo-1,2,3,4-tetrahydro-thieno[2,3-d]pyrimidine-6-carboxylic acid benzyl ester;

3-(4-Dimethylamino-benzyl)-1-methyl-2,4-dioxo-1,2,3,4-tetrahydro-[2,3-d]pyrimidine-6-carboxylic acid benzyl ester;

1-Methyl-3-(4-methylamino-benzyl)-2,4-dioxo-1,2,3,4-tetrahydro-[2,3-d]pyrimidine-6-carboxylic acid benzyl ester;

3-(4-Carbamoyl-benzyl)-1-methyl-2,4-dioxo-1,2,3,4-tetrahydro-[2,3-d]pyrimidine-6-carboxylic acid benzyl ester;

3-(4-Dimethylcarbamoyl-benzyl)-1-methyl-2,4-dioxo-1,2,3,4-tetrahydro-[2,3-d]pyrimidine-6-carboxylic acid benzyl ester;

3-(4-Carboxy-benzyl)-1-methyl-2,4-dioxo-1,2,3,4-tetrahydro-thieno [2,3-d]pyrimidine-6-carboxylic acid benzyl ester;

3-(4-Methoxycarbonyl-benzyl)-1-methyl-2,4-dioxo-1,2,3,4-tetrahydro-[2,3-d]pyrimidine-6-carboxylic acid benzyl ester;

3-{4-[Bis-(2-hydroxy-ethyl)-amino]-benzyl}-1-methyl-2,4-dioxo-1,2,3,4-tetrahydro-thieno[2,3-d]pyrimidine-6-carboxylic acid benzyl ester;

3-(3,5-Dimethoxy-benzyl)-1-methyl-2,4-dioxo-1,2,3,4-tetrahydro-[2,3-d]pyrimidine-6-carboxylic acid benzyl ester;

3-(4-tert-Butyl-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethoxy-benzyl)-1,2,3,4-tetrahydro-[2,3-d]pyrimidine-6-carboxylic acid benzyl ester;

3-(4-Methanesulfonyl-benzyl)-1-methyl-2,4-dioxo-1,2,3,4-tetrahydro-[2,3-d]pyrimidine-6-carboxylic acid benzyl ester;

2,4-Dioxo-3-[1,3,4]thiadiazol-2-ylmethyl-1,2,3,4-tetrahydro-thieno[2,3-d]pyrimidine-6-carboxylic acid benzyl ester;

3-Isoxazol-3-ylmethyl-2,4-dioxo-1,2,3,4-tetrahydro-thieno[2,3-d]pyrimidine-6-carboxylic acid benzyl ester;

3-Oxazol-2-ylmethyl-2,4-dioxo-1,2,3,4-tetrahydro-thieno [2,3-d]pyrimidine-6-carboxylic acid benzyl ester;

2,4-Dioxo-3-thiazol-2-ylmethyl-1,2,3,4-tetrahydro-thieno[2,3-d]pyrimidine-6-carboxylic acid benzyl ester;

3-(1H-imidazol-2-ylmethyl)-2,4-dioxo-1,2,3,4-tetrahydro-thieno[2,3-d]pyrimidine-6-carboxylic acid benzyl ester;

3-(1-Methyl-1H-imidazol-2-ylmethyl)-2,4-dioxo-1,2,3,4-tetrahydro-[2,3-d]pyrimidine-6-carboxylic acid benzyl ester;

3-(1-Methyl-1H-pyrrol-2-ylmethyl)-2,4-dioxo-1,2,3,4-tetrahydro-[2,3-d]pyrimidine-6-carboxylic acid benzyl ester;

2,4-Dioxo-3-(1H-pyrrol-2-ylmethyl)-1,2,3,4-tetrahydro-thieno[2,3-d]pyrimidine-6-carboxylic acid benzyl ester;

2,4-Dioxo-3-(1H-pyrrol-2-ylmethyl)-1,2,3,4-tetrahydro-thieno[2,3-d]pyrimidine-6-carboxylic acid benzyl ester;

2,4-Dioxo-3-thiophen-2-ylmethyl-1,2,3,4-tetrahydro-thieno[2,3-d]pyrimidine-6-carboxylic acid benzyl ester;

2,4-Dioxo-3-[1,2,3,4]tetrazin-5-ylmethyl-1,2,3,4-tetrahydro-thieno[2,3-d]pyrimidine-6-carboxylic acid benzyl ester;

2,4-Dioxo-3-[1,2,4,5]tetrazin-3-ylmethyl-1,2,3,4-tetrahydro-thieno[2,3-d]pyrimidine-6-carboxylic acid benzyl ester;

3-(1-Methyl-piperidin-4-ylmethyl)-2,4-dioxo-1,2,3,4-tetrahydro-[2,3-d]pyrimidine-6-carboxylic acid benzyl ester;

2,4-Dioxo-3-pyrimidin-2-ylmethyl-1,2,3,4-tetrahydro-thieno[2,3-d]pyrimidine-6-carboxylic acid benzyl ester;

2,4-Dioxo-3-(2H-pyran-2-ylmethyl)-1,2,3,4-tetrahydro-thieno[2,3-d]pyrimidine-6-carboxylic acid benzyl ester;

3-(1H-Imidazo[4,5-b]pyridin-2-ylmethyl)-2,4-dioxo-1,2,3,4-tetrahydro-[2,3-d]pyrimidine-6-carboxylic acid benzyl ester;

3-(1H-Benzoimidazol-2-ylmethyl)-2,4-dioxo-1,2,3,4-tetrahydro-[2,3-d]pyrimidine-6-carboxylic acid benzyl ester;

3-Benzo[b]thiophen-2-ylmethyl-2,4-dioxo-1,2,3,4-tetrahydro-thieno[2,3-d]pyrimidine-6-carboxylic acid benzyl ester;

2,4-Dioxo-3-quinolin-2-ylmethyl-1,2,3,4-tetrahydro-thieno[2,3-d]pyrimidine-6-carboxylic acid benzyl ester;

3-(2H-Chromen-2-ylmethyl)-2,4-dioxo-1,2,3,4-tetrahydro-thieno[2,3-d]pyrimidine-6-carboxylic acid benzyl ester;

3-(1H-Benzoimidazol-2-ylmethyl)-2,4-dioxo-1,2,3,4-tetrahydro-[2,3-d]pyrimidine-6-carboxylic acid benzyl ester;

3-(1-Methyl-1H-benzoimidazol-2-ylmethyl)-2,4-dioxo-1,2,3,4-tetrahydro-[2,3-d]pyrimidine-6-carboxylic acid benzyl ester;

3-(1H-Indol-2-ylmethyl)-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid furan-3-ylmethyl ester;

3-Benzyl-1-methyl-2,4-dioxo-1,2,3,4-tetrahydro-thieno[2,3-d]pyrimidine-6-carboxylic acid 1-ethyl-propyl ester;

3-Benzyl-1-methyl-2,4-dioxo-1,2,3,4-tetrahydro-thieno[2,3-d]pyrimidine-6-carboxylic acid 1,1-dioxo-tetrahydro-116-thiophen-3-yl ester;

3-Benzyl-1-methyl-2,4-dioxo-1,2,3,4-tetrahydro-thieno[2,3-d]pyrimidine-6-carboxylic acid 4-hydroxy-benzyl ester;

3-Benzyl-1-methyl-2,4-dioxo-1,2,3,4-tetrahydro-thieno[2,3-d]pyrimidine-6-carboxylic acid 1-oxy-pyridin-4-ylmethyl ester;

3-Benzyl-1-methyl-2,4-dioxo-1,2,3,4-tetrahydro-thieno[2,3-d]pyrimidine-6-carboxylic acid but-3-enyl ester;

3-Benzyl-1-methyl-2,4-dioxo-1,2,3,4-tetrahydro-thieno[2,3-d]pyrimidine-6-carboxylic acid 3-diethylamino-propyl ester;

3-Benzyl-1-methyl-2,4-dioxo-1,2,3,4-tetrahydro-thieno[2,3-d]pyrimidine-6-carboxylic acid 1-cyano-1-phenyl-methyl ester;

3-Benzyl-1-methyl-2,4-dioxo-1,2,3,4-tetrahydro-thieno[2,3-d]pyrimidine-6-carboxylic acid 3-amino-benzyl ester;

3-Benzyl-1-methyl-2,4-dioxo-1,2,3,4-tetrahydro-thieno[2,3-d]pyrimidine-6-carboxylic acid 3-methoxy-benzyl ester;

3-Benzyl-1-methyl-2,4-dioxo-1,2,3,4-tetrahydro-thieno[2,3-d]pyrimidine-6-carboxylic acid 1-oxy-pyridin-3-ylmethyl ester;

3-Benzyl-1-methyl-2,4-dioxo-1,2,3,4-tetrahydro-thieno[2,3-d]pyrimidine-6-carboxylic acid 2-ethoxy-ethyl ester;

3-Benzyl-1-methyl-2,4-dioxo-1,2,3,4-tetrahydro-thieno[2,3-d]pyrimidine-6-carboxylic acid thiophen-2-ylmethyl ester;

3-Benzyl-1-methyl-2,4-dioxo-1,2,3,4-tetrahydro-thieno[2,3-d]pyrimidine-6-carboxylic acid 2,6-dichloro-benzyl ester;

3-Benzyl-1-methyl-2,4-dioxo-1,2,3,4-tetrahydro-thieno[2,3-d]pyrimidine-6-carboxylic acid dimethylamino-methyl-ethyl ester;

3-Benzyl-1-methyl-2,4-dioxo-1,2,3,4-tetrahydro-thieno[2,3-d]pyrimidine-6-carboxylic acid 2,2-diphenyl-ethyl ester;

3-Benzyl-1-methyl-2,4-dioxo-1,2,3,4-tetrahydro-thieno[2,3-d]pyrimidine-6-carboxylic acid 2-pyridin-2-yl-ethyl ester;

3-Benzyl-1-methyl-2,4-dioxo-1,2,3,4-tetrahydro-thieno[2,3-d]pyrimidine-6-carboxylic acid 2-ethanesulfonyl-ethyl ester;

3-Benzyl-1-methyl-2,4-dioxo-1,2,3,4-tetrahydro-thieno[2,3-d]pyrimidine-6-carboxylic acid diethylamino-methyl-ethyl ester;

3-Benzyl-1-methyl-2,4-dioxo-1,2,3,4-tetrahydro-thieno[2,3-d]pyrimidine-6-carboxylic acid dimethylamino-methyl-propyl ester;

3-Benzyl-1-methyl-2,4-dioxo-1,2,3,4-tetrahydro-thieno[2,3-d]pyrimidine-6-carboxylic acid 2-(2-chloro-phenoxy)-ethyl ester;

3-Benzyl-1-methyl-2,4-dioxo-1,2,3,4-tetrahydro-thieno[2,3-d]pyrimidine-6-carboxylic acid 2-(2-ethoxy-ethoxy)-ethyl ester;

3-Benzyl-1-methyl-2,4-dioxo-1,2,3,4-tetrahydro-thieno[2,3-d]pyrimidine-6-carboxylic acid 2-hydroxy-benzyl ester;

1-Methyl-2,4-dioxo-1,2,3,4-tetrahydro-thieno[2,3-d]pyrimidine-6-carboxylic acid 2-morpholin-4-yl-ethyl ester;

3-Benzyl-1-methyl-2,4-dioxo-1,2,3,4-tetrahydro-thieno[2,3-d]pyrimidine-6-carboxylic acid 2-(1,3-dioxo-1,3-dihydro-isoindol-2-yl)-ethyl ester;

3-Benzyl-1-methyl-2,4-dioxo-1,2,3,4-tetrahydro-thieno[2,3-d]pyrimidine-6-carboxylic acid 2,3-dihydro-benzo[1,4]dioxin-2-ylmethyl ester;

3-Benzyl-1-methyl-2,4-dioxo-1,2,3,4-tetrahydro-thieno[2,3-d]pyrimidine-6-carboxylic acid 1-methyl-piperidin-4-yl ester;

3-Benzyl-1-methyl-2,4-dioxo-1,2,3,4-tetrahydro-thieno[2,3-d]pyrimidine-6-carboxylic acid 2-(4-hydroxy-phenyl)-ethyl ester;

3-Benzyl-1-methyl-2,4-dioxo-1,2,3,4-tetrahydro-thieno[2,3-d]pyrimidine-6-carboxylic acid 2-cyano-ethyl ester;

3-Benzyl-1-methyl-2,4-dioxo-1,2,3,4-tetrahydro-thieno[2,3-d]pyrimidine-6-carboxylic acid hexyl ester;

3-Benzyl-1-methyl-2,4-dioxo-1,2,3,4-tetrahydro-thieno[2,3-d]pyrimidine-6-carboxylic acid 4-fluoro-benzyl ester;

3-Benzyl-1-methyl-2,4-dioxo-1,2,3,4-tetrahydro-thieno[2,3-d]pyrimidine-6-carboxylic acid 3-hydroxy-6-methyl-pyridin-2-ylmethyl ester;

3-Benzyl-1-methyl-2,4-dioxo-1,2,3,4-tetrahydro-thieno[2,3-d]pyrimidine-6-carboxylic acid 2-benzyloxy-ethyl ester;

3-Benzyl-1-methyl-2,4-dioxo-1,2,3,4-tetrahydro-thieno[2,3-d]pyrimidine-6-carboxylic acid 2-methoxy-benzyl ester;

3-Benzyl-1-methyl-2,4-dioxo-1,2,3,4-tetrahydro-thieno[2,3-d]pyrimidine-6-carboxylic acid 4-methoxy-benzyl ester;

3-Benzyl-1-methyl-2,4-dioxo-1,2,3,4-tetrahydro-thieno[2,3-d]pyrimidine-6-carboxylic acid 2,2,2-trifluoro-ethyl ester;

3-Benzyl-1-methyl-2,4-dioxo-1,2,3,4-tetrahydro-thieno[2,3-d]pyrimidine-6-carboxylic acid 2,2,2-trichloro-ethyl ester;

3-Benzyl-1-methyl-2,4-dioxo-1,2,3,4-tetrahydro-thieno[2,3-d]pyrimidine-6-carboxylic acid pyridin-3-ylmethyl ester;

3-Benzyl-1-methyl-2,4-dioxo-1,2,3,4-tetrahydro-thieno[2,3-d]pyrimidine-6-carboxylic acid pyridin-4-ylmethyl ester;

3-Benzyl-1-methyl-2,4-dioxo-1,2,3,4-tetrahydro-thieno[2,3-d]pyrimidine-6-carboxylic acid 3-pyridin-3-yl-propyl ester;

3-Benzyl-1-methyl-2,4-dioxo-1,2,3,4-tetrahydro-thieno[2,3-d]pyrimidine-6-carboxylic acid;

3-Benzyl-1-methyl-2,4-dioxo-1,2,3,4-tetrahydro-thieno[2,3-d]pyrimidine-6-carboxylic acid 2-phenoxy-ethyl ester;

3-Benzyl-1-methyl-2,4-dioxo-1,2,3,4-tetrahydro-thieno[2,3-d]pyrimidine-6-carboxylic acid 1,3-dimethyl-butyl ester;

3-Benzyl-1-methyl-2,4-dioxo-1,2,3,4-tetrahydro-thieno [2,3-d]pyrimidine-6-carboxylic acid 2-methyl-benzyl ester;

3-Benzyl-1-methyl-2,4-dioxo-1,2,3,4-tetrahydro-thieno[2,3-d]pyrimidine-6-carboxylic acid 1-phenyl-ethyl ester;

3-Benzyl-1-methyl-2,4-dioxo-1,2,3,4-tetrahydro-thieno[2,3-d]pyrimidine-6-carboxylic acid 1-benzyl-piperidin-4-yl ester;

3-Benzyl-1-methyl-2,4-dioxo-1,2,3,4-tetrahydro-thieno[2,3-d]pyrimidine-6-carboxylic acid propyl ester;

3-Benzyl-1-methyl-2,4-dioxo-1,2,3,4-tetrahydro-thieno[2,3-d]pyrimidine-6-carboxylic acid methyl ester;

3-Benzyl-1-methyl-2,4-dioxo-1,2,3,4-tetrahydro-thieno[2,3-d]pyrimidine-6-carboxylic acid 2-trifluoromethyl-benzyl ester;

3-Benzyl-1-methyl-2,4-dioxo-1,2,3,4-tetrahydro-thieno[2,3-d]pyrimidine-6-carboxylic acid 2-p-tolyl-ethyl ester;

3-Benzyl-1-methyl-2,4-dioxo-1,2,3,4-tetrahydro-thieno[2,3-d]pyrimidine-6-carboxylic acid 4-trifluoromethyl-benzyl ester;

3-Benzyl-1-methyl-2,4-dioxo-1,2,3,4-tetrahydro-thieno[2,3-d]pyrimidine-6-carboxylic acid tetrahydro-furan-2-ylmethyl ester;

3-Benzyl-1-methyl-2,4-dioxo-1,2,3,4-tetrahydro-thieno[2,3-d]pyrimidine-6-carboxylic acid octahydro-inden-5-yl ester;

3-Benzyl-1-methyl-2,4-dioxo-1,2,3,4-tetrahydro-thieno[2,3-d]pyrimidine-6-carboxylic acid 4-amino-benzyl ester;

3-Benzyl-1-methyl-2,4-dioxo-1,2,3,4-tetrahydro-thieno[2,3-d]pyrimidine-6-carboxylic acid 2-aziridin-1-yl-ethyl ester;

3-Benzyl-1-methyl-2,4-dioxo-1,2,3,4-tetrahydro-thieno[2,3-d]pyrimidine-6-carboxylic acid 3-methyl-but-2-enyl ester;

3-Benzyl-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid trifluoro-trifluoromethyl-ethyl ester;

3-Benzyl-1-methyl-2,4-dioxo-1,2,3,4-tetrahydro-thieno[2,3-d]pyrimidine-6-carboxylic acid phenethyl ester;

3-Benzyl-1-methyl-2,4-dioxo-1,2,3,4-tetrahydro-thieno[2,3-d]pyrimidine-6-carboxylic acid 2-methoxy-ethyl ester;

3-Benzyl-1-methyl-2,4-dioxo-1,2,3,4-tetrahydro-thieno[2,3-d]pyrimidine-6-carboxylic acid biphenyl-4-ylmethyl ester;

3-Benzyl-1-methyl-2,4-dioxo-1,2,3,4-tetrahydro-thieno [2,3-d]pyrimidine-6-carboxylic acid 2-chloro-6-fluoro-benzyl ester;

3-Benzyl-1-methyl-2,4-dioxo-1,2,3,4-tetrahydro-thieno[2,3-d]pyrimidine-6-carboxylic acid tetrahydro-pyran-4-yl ester;

3-Benzyl-1-methyl-2,4-dioxo-1,2,3,4-tetrahydro-thieno[2,3-d]pyrimidine-6-carboxylic acid 3-ethyl-oxetan-3-ylmethyl ester;

3-Benzyl-1-methyl-2,4-dioxo-1,2,3,4-tetrahydro-thieno[2,3-d]pyrimidine-6-carboxylic acid butyl ester;

3-Benzyl-1-methyl-2,4-dioxo-1,2,3,4-tetrahydro-thieno[2,3-d]pyrimidine-6-carboxylic acid 2-(2-hydroxy-phenyl)-ethyl ester;

3-Benzyl-1-methyl-2,4-dioxo-1,2,3,4-tetrahydro-thieno[2,3-d]pyrimidine-6-carboxylic acid 2-(4-fluoro-phenyl)-ethyl ester;

3-Benzyl-1-methyl-2,4-dioxo-1,2,3,4-tetrahydro-thieno[2,3-d]pyrimidine-6-carboxylic acid cyclopropylmethyl ester;

3-Benzyl-1-methyl-2,4-dioxo-1,2,3,4-tetrahydro-thieno[2,3-d]pyrimidine-6-carboxylic acid 4-ethyl-benzyl ester;

3-Benzyl-1-methyl-2,4-dioxo-1,2,3,4-tetrahydro-thieno[2,3-d]pyrimidine-6-carboxylic acid (S)-1-phenyl-ethyl ester;

3-Benzyl-1-methyl-2,4-dioxo-1,2,3,4-tetrahydro-thieno[2,3-d]pyrimidine-6-carboxylic acid 2,6-difluoro-benzyl ester;

3-Benzyl-1-methyl-2,4-dioxo-1,2,3,4-tetrahydro-thieno[2,3-d]pyrimidine-6-carboxylic acid cyclobutyl methyl ester;

3-Benzyl-1-methyl-2,4-dioxo-1,2,3,4-tetrahydro-thieno[2,3-d]pyrimidine-6-carboxylic acid 2-pyridin-4-yl-ethyl ester;

3-Benzyl-1-methyl-2,4-dioxo-1,2,3,4-tetrahydro-thieno[2,3-d]pyrimidine-6-carboxylic acid 3-hydroxy-cyclopentyl ester;

3-Benzyl-1-methyl-2,4-dioxo-1,2,3,4-tetrahydro-thieno[2,3-d]pyrimidine-6-carboxylic acid 1-pentafluorophenyl-ethyl ester;

3-Benzyl-1-methyl-2,4-dioxo-1,2,3,4-tetrahydro-thieno[2,3-d]pyrimidine-6-carboxylic acid 2-benzyloxycarbonylamino-ethyl ester; and

3-Benzyl-1-methyl-2,4-dioxo-1,2,3,4-tetrahydro-thieno[2,3-d]pyrimidine-6-carboxylic acid ethyl ester; or a pharmaceutically acceptable salt thereof.

32. The combination according to Embodiment 23, wherein the compound of Formula 1B is selected from:

3-Benzyl-1-methyl-2,4-dioxo-1,2,3,4-tetrahydro-thieno[2,3-d]pyrimidine-6-carboxylic acid (2-pyridin-4-yl-ethyl)-amide;

3-Benzyl-1-methyl-2,4-dioxo-1,2,3,4-tetrahydro-thieno [2,3-d]pyrimidine-6-carboxylic acid (2-morpholin-4-yl-ethyl)-amide;

3-Benzyl-1-methyl-2,4-dioxo-1,2,3,4-tetrahydro-thieno[2,3-d]pyrimidine-6-carboxylic acid 4-methyl-benzylamide;

3-Benzyl-1-methyl-2,4-dioxo-1,2,3,4-tetrahydro-thieno[2,3-d]pyrimidine-6-carboxylic acid sec-butylamide;

3-Benzyl-1-methyl-2,4-dioxo-1,2,3,4-tetrahydro-thieno[2,3-d]pyrimidine-6-carboxylic acid cyclopentylamide;

3-Benzyl-1-methyl-2,4-dioxo-1,2,3,4-tetrahydro-thieno[2,3-d]pyrimidine-6-carboxylic acid cyclopropylamide;

3-Benzyl-1-methyl-2,4-dioxo-1,2,3,4-tetrahydro-thieno[2,3-d]pyrimidine-6-carboxylic acid cyanomethyl-amide;

3-Benzyl-1-methyl-2,4-dioxo-1,2,3,4-tetrahydro-thieno[2,3-d]pyrimidine-6-carboxylic acid cyclohexylamide;

3-Benzyl-1-methyl-2,4-dioxo-1,2,3,4-tetrahydro-thieno[2,3-d]pyrimidine-6-carboxylic acid 3-methyl-benzylamide;

3-Benzyl-1-methyl-2,4-dioxo-1,2,3,4-tetrahydro-thieno[2,3-d]pyrimidine-6-carboxylic acid (3-ethoxy-propyl)-amide;

3-Benzyl-1-methyl-2,4-dioxo-1,2,3,4-tetrahydro-thieno[2,3-d]pyrimidine-6-carboxylic acid 2-chloro-benzylamide;

3-Benzyl-1-methyl-2,4-dioxo-1,2,3,4-tetrahydro-thieno[2,3-d]pyrimidine-6-carboxylic acid 2-methyl-benzylamide;

3-Benzyl-1-methyl-2,4-dioxo-1,2,3,4-tetrahydro-thieno[2,3-d]pyrimidine-6-carboxylic acid (2,2-diphenyl-ethyl)-amide;

3-Benzyl-1-methyl-2,4-dioxo-1,2,3,4-tetrahydro-thieno[2,3-d]pyrimidine-6-carboxylic acid (pyridin-3-ylmethyl)-amide;

3-Benzyl-1-methyl-2,4-dioxo-1,2,3,4-tetrahydro-thieno[2,3-d]pyrimidine-6-carboxylic acid cyclopropylmethyl-amide;

3-Benzyl-1-methyl-2,4-dioxo-1,2,3,4-tetrahydro-thieno[2,3-d]pyrimidine-6-carboxylic acid (1-ethyl-pyrrolidin-2-ylmethyl)-amide;

3-Benzyl-1-methyl-2,4-dioxo-1,2,3,4-tetrahydro-thieno[2,3-d]pyrimidine-6-carboxylic acid (pyridin-2-ylmethyl)-amide;

3-Benzyl-1-methyl-2,4-dioxo-1,2,3,4-tetrahydro-thieno[2,3-d]pyrimidine-6-carboxylic acid [2-(3,4-dimethoxy-phenyl)-ethyl]-amide;

3-Benzyl-1-methyl-2,4-dioxo-1,2,3,4-tetrahydro-thieno[2,3-d]pyrimidine-6-carboxylic acid (furan-2-ylmethyl)-amide;

3-Benzyl-1-methyl-2,4-dioxo-1,2,3,4-tetrahydro-thieno[2,3-d]pyrimidine-6-carboxylic acid 2-fluoro-benzylamide;

3-Benzyl-1-methyl-2,4-dioxo-1,2,3,4-tetrahydro-thieno[2,3-d]pyrimidine-6-carboxylic acid (2-bromo-ethyl)-amide;

3-Benzyl-1-methyl-2,4-dioxo-1,2,3,4-tetrahydro-thieno[2,3-d]pyrimidine-6-carboxylic acid 4-sulfamoyl-benzylamide;

3-Benzyl-1-methyl-2,4-dioxo-1,2,3,4-tetrahydro-thieno [2,3-d]pyrimidine-6-carboxylic acid 3-methoxy-benzylamide;

3-Benzyl-1-methyl-2,4-dioxo-1,2,3,4-tetrahydro-thieno[2,3-d]pyrimidine-6-carboxylic acid 4-methoxy-benzylamide;

3-Benzyl-1-methyl-2,4-dioxo-1,2,3,4-tetrahydro-thieno[2,3-d]pyrimidine-6-carboxylic acid phenethyl-amide;

(S)-2-{[1-(3-Benzyl-1-methyl-2,4-dioxo-1,2,3,4-tetrahydro-thieno[2,3-d]pyrimidin-6-yl)-methanoyl]-amino}-propionic acid;

3-Benzyl-1-methyl-2,4-dioxo-1,2,3,4-tetrahydro-thieno[2,3-d]pyrimidine-6-carboxylic acid (1-phenyl-ethyl)-amide;

3-Benzyl-1-methyl-2,4-dioxo-1,2,3,4-tetrahydro-thieno[2,3-d]pyrimidine-6-carboxylic acid 2-methoxy-benzylamide;

3-Benzyl-1-methyl-2,4-dioxo-1,2,3,4-tetrahydro-thieno[2,3-d]pyrimidine-6-carboxylic acid benzylamide;

3-Benzyl-1-methyl-2,4-dioxo-1,2,3,4-tetrahydro-thieno[2,3-d pyrimidine-6-carboxylic acid 3-bromo-benzylamide;

3-Benzyl-1-methyl-2,4-dioxo-1,2,3,4-tetrahydro-thieno[2,3-d]pyrimidine-6-carboxylic acid [2-(4-sulfamoyl-phenyl)-ethyl]-amide;

2-{[1-(3-Benzyl-1-methyl-2,4-dioxo-1,2,3,4-tetrahydro-thieno[2,3-d]pyrimidin-6-yl)-methanoyl]-amino}-3-phenyl-propionic acid methyl ester;

3-Benzyl-1-methyl-2,4-dioxo-1,2,3,4-tetrahydro-thieno[2,3-d]pyrimidine-6-carboxylic acid (3-imidazol-1-yl-propyl)-amide;

3-Benzyl-1-methyl-2,4-dioxo-1,2,3,4-tetrahydro-thieno[2,3-d]pyrimidine-6-carboxylic acid [2-(2-methoxy-phenyl)-ethyl]-amide;

3-Benzyl-1-methyl-2,4-dioxo-1,2,3,4-tetrahydro-thieno[2,3-d]pyrimidine-6-carboxylic acid 4-trifluoromethyl-benzylamide;

3-Benzyl-1-methyl-2,4-dioxo-1,2,3,4-tetrahydro-thieno[2,3-d]pyrimidine-6-carboxylic acid 4-amino-benzylamide;

3-Benzyl-1-methyl-2,4-dioxo-1,2,3,4-tetrahydro-thieno[2,3-d]pyrimidine-6-carboxylic acid [2-(4-fluoro-phenyl)-ethyl]-amide; and

3-Benzyl-1-methyl-2,4-dioxo-1,2,3,4-tetrahydro-thieno[2,3-d]pyrimidine-6-carboxylic acid ((R)-2-hydroxy-1-methyl-ethyl)-amide; or a pharmaceutically acceptable salt thereof.

33. The combination according to Embodiment 23, wherein the compound of Formula IB is selected from:

3-Benzyl-1-methyl-2,4-dioxo-1,2,3,4-tetrahydro-thieno[2,3-d]pyrimidine-6-carboxylic acid benzofuran-5-ylmethyl ester;

(3-1{[1-(3-Benzyl-1-methyl-2,4-dioxo-1,2,3,4-tetrahydro-thieno[2,3-d]pyrimidin-6-yl)-methanoyl]-amino}-propyl)-carbamic acid tert-butyl ester;

3-Benzyl-1-methyl-2,4-dioxo-1,2,3,4-tetrahydro-thieno[2,3-d]pyrimidine-6-carboxylic acid benzofuran-2-ylmethyl ester;

3-Benzyl-2,4-dioxo-1,2,3,4-tetrahydro-thieno[2,3-d]pyrimidine-6-carboxylic acid thiophen-3-ylmethyl ester;

3-Benzyl-1-methyl-2,4-dioxo-1,2,3,4-tetrahydro-thieno[2,3-d]pyrimidine-6-carboxylic acid 3H-[1,2,3]oxathiazol-5-ylmethyl ester;

3-Benzyl-2,4-dioxo-1,2,3,4-tetrahydro-thieno[2,3-d]pyrimidine-6-carboxylic acid 3H-[1,2,3]oxathiazol-5-ylmethyl ester;

3-Benzyl-2,4-dioxo-1,2,3,4-tetrahydro-thieno[2,3-d]pyrimidine-6-carboxylic acid [1,4,2]dioxazol-3-ylmethyl ester;

3-Benzyl-1-methyl-2,4-dioxo-1,2,3,4-tetrahydro-thieno[2,3-d]pyrimidine-6-carboxylic acid [1,4,2]dioxazol-3-ylmethyl ester;

3-Benzyl-2,4-dioxo-1,2,3,4-tetrahydro-thieno[2,3-d]pyrimidine-6-carboxylic acid furazan-3-ylmethyl ester;

3-Benzyl-1-methyl-2,4-dioxo-1,2,3,4-tetrahydro-thieno[2,3-d]pyrimidine-6-carboxylic acid furazan-3-ylmethyl ester;

3-Benzyl-1-methyl-2,4-dioxo-1,2,3,4-tetrahydro-thieno[2,3-d]pyrimidine-6-carboxylic acid [1,2,4]oxadiazol-5-ylmethyl ester;

3-Benzyl-2,4-dioxo-1,2,3,4-tetrahydro-thieno[2,3-d]pyrimidine-6-carboxylic acid [1,2,4]oxadiazol-5-ylmethyl ester;

3-Benzyl-2,4-dioxo-1,2,3,4-tetrahydro-thieno[2,3-d]pyrimidine-6-carboxylic acid 3H-[1,2,3]triazol-4-ylmethyl ester;

3-Benzyl-1-methyl-2,4-dioxo-1,2,3,4-tetrahydro-thieno[2,3-d]pyrimidine-6-carboxylic acid 3H-[1,2,3]triazol-4-ylmethyl ester;

3-Benzyl-1-methyl-2,4-dioxo-1,2,3,4-tetrahydro-thieno[2,3-d]pyrimidine-6-carboxylic acid 2H-[1,2,4]triazol-3-ylmethyl ester;

3-Benzyl-2,4-dioxo-1,2,3,4-tetrahydro-thieno[2,3-d]pyrimidine-6-carboxylic acid 2H-[1,2,4]triazol-3-ylmethyl ester;

3-Benzyl-2,4-dioxo-1,2,3,4-tetrahydro-thieno[2,3-d]pyrimidine-6-carboxylic acid isoxazol-5-ylmethyl ester;

3-Benzyl-1-methyl-2,4-dioxo-1,2,3,4-tetrahydro-thieno[2,3-d]pyrimidine-6-carboxylic acid isoxazol-5-ylmethyl ester;

3-Benzyl-1-methyl-2,4-dioxo-1,2,3,4-tetrahydro-thieno[2,3-d]pyrimidine-6-carboxylic acid oxazol-2-ylmethyl ester;

3-Benzyl-2,4-dioxo-1,2,3,4-tetrahydro-thieno[2,3-d]pyrimidine-6-carboxylic acid oxazol-2-ylmethyl ester;

3-Benzyl-2,4-dioxo-1,2,3,4-tetrahydro-thieno[2,3-d]pyrimidine-6-carboxylic acid isothiazol-5-ylmethyl ester;

3-Benzyl-1-methyl-2,4-dioxo-1,2,3,4-tetrahydro-thieno[2,3-d]pyrimidine-6-carboxylic acid isothiazol-5-ylmethyl ester;

3-Benzyl-2,4-dioxo-1,2,3,4-tetrahydro-thieno[2,3-d]pyrimidine-6-carboxylic acid thiazol-2-ylmethyl ester;

3-Benzyl-1-methyl-2,4-dioxo-1,2,3,4-tetrahydro-thieno[2,3-d]pyrimidine-6-carboxylic acid thiazol-2-ylmethyl ester;

3-Benzyl-1-methyl-2,4-dioxo-1,2,3,4-tetrahydro-thieno[2,3-d]pyrimidine-6-carboxylic acid 1H-imidazol-2-ylmethyl ester;

3-Benzyl-2,4-dioxo-1,2,3,4-tetrahydro-thieno[2,3-d]pyrimidine-6-carboxylic acid 2H-imidazol-2-ylmethyl ester;

3-Benzyl-2,4-dioxo-1,2,3,4-tetrahydro-thieno[2,3-d]pyrimidine-6-carboxylic acid 1H-pyrazol-3-ylmethyl ester;

3-Benzyl-1-methyl-2,4-dioxo-1,2,3,4-tetrahydro-thieno[2,3-d]pyrimidine-6-carboxylic acid 2H-pyrazol-3-ylmethyl ester;

3-Benzyl-1-methyl-2,4-dioxo-1,2,3,4-tetrahydro-thieno[2,3-d]pyrimidine-6-carboxylic acid 1H-pyrrol-2-ylmethyl ester;

3-Benzyl-2,4-dioxo-1,2,3,4-tetrahydro-thieno[2,3-d]pyrimidine-6-carboxylic acid 2H-pyrrol-2-ylmethyl ester;

3-Furazan-3-ylmethyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid 2H-chromen-2-ylmethyl ester;

3-Benzyl-1-methyl-2,4-dioxo-1,2,3,4-tetrahydro-thieno[2,3-d]pyrimidine-6-carboxylic acid 2H-thiochromen-2-ylmethyl ester;

3-Benzyl-2,4-dioxo-1,2,3,4-tetrahydro-thieno[2,3-d]pyrimidine-6-carboxylic acid 2H-thiochromen-2-ylmethyl ester;

3-Benzyl-2,4-dioxo-1,2,3,4-tetrahydro-thieno[2,3-d]pyrimidine-6-carboxylic acid [1,3,4]thiadiazol-2-ylmethyl ester;

3-Benzyl-1-methyl-2,4-dioxo-1,2,3,4-tetrahydro-thieno[2,3-d]pyrimidine-6-carboxylic acid [1,3,4]thiadiazol-2-ylmethyl ester;

3-Benzyl-2,4-dioxo-1,2,3,4-tetrahydro-thieno[2,3-d]pyrimidine-6-carboxylic acid 1H-benzoimidazol-5-ylmethyl ester;

3-Benzyl-1-methyl-2,4-dioxo-1,2,3,4-tetrahydro-thieno[2,3-d]pyrimidine-6-carboxylic acid 1H-benzoimidazol-5-ylmethyl ester;

3-Benzyl-1-methyl-2,4-dioxo-1,2,3,4-tetrahydro-thieno[2,3-d]pyrimidine-6-carboxylic acid 1H-benzoimidazol-2-ylmethyl ester;

3-Benzyl-2,4-dioxo-1,2,3,4-tetrahydro-thieno[2,3-d]pyrimidine-6-carboxylic acid 1H-benzoimidazol-2-ylmethyl ester;

3-Benzyl-2,4-dioxo-1,2,3,4-tetrahydro-thieno[2,3-d]pyrimidine-6-carboxylic acid 1H-indol-2-ylmethyl ester;

3-Benzyl-1-methyl-2,4-dioxo-1,2,3,4-tetrahydro-thieno[2,3-d]pyrimidine-6-carboxylic acid 1H-indol-2-ylmethyl ester;

3-Benzyl-1-methyl-2,4-dioxo-1,2,3,4-tetrahydro-thieno[2,3-d]pyrimidine-6-carboxylic acid 1H-indol-5-ylmethyl ester;

3-Benzyl-2,4-dioxo-1,2,3,4-tetrahydro-thieno[2,3-d]pyrimidine-6-carboxylic acid 1H-indol-5-ylmethyl ester;

3-Benzyl-2,4-dioxo-1,2,3,4-tetrahydro-thieno[2,3-d]pyrimidine-6-carboxylic acid 2,3-dihydro-benzofuran-5-ylmethyl ester; and

3-Benzyl-1-methyl-2,4-dioxo-1,2,3,4-tetrahydro-thieno[2,3-d]pyrimidine-6-carboxylic acid 2,3-dihydro-benzofuran-5-ylmethyl ester; or a pharmaceutically acceptable salt thereof.

34. The combination according to Embodiment 23, wherein the compound of Formula IB is selected from:

4-{6-[3-(4-Methoxy-phenyl)-prop-1-ynyl]-1-methyl-2,4-dioxo-1,4-dihydro-2H-thieno[2,3-d]pyrimidine-3-ylmehtyl}-benzoic acid;

3-(4-Methanesulfonyl-benzyl)-6-[3-(4-methoxy-phenyl)-prop-1-ynyl]-1-methyl-1H-thieno[2,3-d]pyrimidine-2,4-dione;

4-{6-[3-(3-Methoxy-phenyl)-prop-1-ynyl]-1-methyl-2,4-dioxo-1,4-dihydro-2H-thieno[2,3-d]pyrimidine-3-ylmehtyl}-benzoic acid;

3-(4-Methanesulfonyl-benzyl)-6-[3-(3-methoxy-phenyl)-prop-1-ynyl]-1-methyl-1H-thieno[2,3-d]pyrimidine-2,4-dione;

4-[1-Methyl-2,4-dioxo-6-(3-pyridine-4-yl-prop-1-ynyl)-1,4-dihydro-2H-[2,3-d]pyrimidine-3-ylmehtyl]-benzoic acid;

3-(4-Methanesulfonyl-benzyl)-1⁻⁶-(3-pyridin-4-yl-prop-1-ynyl)-1H-[2,3-d]pyrimidine-2,4-dione;

4-[1-Methyl-2,4-dioxo-6-(3-pyridine-3-yl-prop-1-ynyl)-1,4-dihydro-2H-[2,3-d]pyrimidine-3-ylmehtyl]-benzoic acid;

3-(4-Methanesulfonyl-benzyl)-6-(3-pyrimidin-3-yl-prop-1-ynyl)-1H-[2,3-d]pyrimidine-2,4-dione;

4-{6-[3-(4-Fluoro-phenyl)-prop-1-ynyl]-1-methyl-2,4-dioxo-1,4-dihydro-2H-thieno[2,3-d]pyrimidine-3-ylmehtyl}-benzoic acid 6-[3-(4-Fluoro-phenyl)-prop-1-ynyl]-3-(4-methanesulfonyl-benzyl)-1-methyl-1H-thieno[2,3-d]pyrimidine-2,4-dione;

4-{6-[3-(3-Fluoro-phenyl)-prop-1-ynyl]-1-methyl-2,4-dioxo-1,4-dihydro-2H-thieno[2,3-d]pyrimidine-3-ylmehtyl}-benzoic acid;

6-[3-(3-Fluoro-phenyl)-prop-1-ynyl]-3-(4-methanesulfonyl-benzyl)-1-methyl-1H-thieno[2,3-d]pyrimidine-2,4-dione;

4-{6-[3-(4-Chloro-phenyl)-prop-1-ynyl]-1-methyl-2,4-dioxo-1,4-dihydro-2H-thieno[2,3-d]pyrimidine-3-ylmehtyl}-benzoic acid;

6-[3-(4-Chloro-phenyl)-prop-1-ynyl]-3-(4-methanesulfonyl-benzyl)-1-methyl-1H-thieno[2,3-d]pyrimidine-2,4-dione;

4-{6-[3-(3-Chloro-phenyl)-prop-1-ynyl]-1-methyl-2,4-dioxo-1,4-dihydro-2H-thieno[2,3-d]pyrmidine-3-ylmehtyl}-benzoic acid;

6-[3-(3-Chloro-phenyl)-prop-1-ynyl]-3-(4-methanesulfonyl-benzyl)-1-methyl-1H-thieno[2,3-d]pyrimidine-2,4-dione;

4-{6-[3-(4-Bromo-phenyl)-prop-1-ynyl]-1-methyl-2,4-dioxo-1,4-dihydro-2H-thieno[2,3-d]pyrimidine-3-ylmehtyl}-benzoic acid;

⁶-[3-(4-Bromo-phenyl)-prop-1-ynyl]-3-(4-methanesulfonyl-benzyl)-1-methyl-1H-thieno[2,3-d]pyrimidine-2,4-dione;

4-{6-[3-(3-Bromo-phenyl)-prop-1-ynyl]-1-methyl-2,4-dioxo-1,4-dihydro-2H-thieno[2,3-d]pyrimidine-3-ylmehtyl}-benzoic acid;

6-[3-(3-Bromo-phenyl)-prop-1-ynyl]-3-(4-methanesulfonyl-benzyl)-1-methyl-1H-thieno[2,3-d]pyrimidine-2,4-dione;

4-{1-Methyl-6-[3-(4-nitro-phenyl)-prop-1-ynyl]-2,4-dioxo-1,4-dihydro-2H-thieno[2,3-d]pyrimidine-3-ylmehtyl}-benzoic acid;

3-(4-Methanesulfonyl-benzyl)-1-methyl-6-[3-(4-nitro-phenyl)-prop-1-ynyl)-1H-thieno[2,3-d]pyrimidine-2,4-dione;

4-{6-[3-(2-Methoxy-pyridin-4-yl)-prop-1-ynyl]-1-methyl-2,4-dioxo-1,4-dihydro-2H-thieno[2,3-d]pyrimidine-3-ylmehtyl}-benzoic acid;

3-(4-Methanesulfonyl-benzyl)-6-[3-(2-methoxy-pyridin-4-yl)-prop-1-ynyl]-1-methyl-1H-thieno[2,3-d]pyrimidine-2,4-dione;

4-{1-Methyl-6-[3-(4-methylsulfanyl-phenyl)-prop-1-ynyl]-2,4-dioxo-1,4-dihydro-2H-thieno[2,3-d]pyrimidine-3-ylmehtyl}-benzoic acid;

3-(4-Methanesulfonyl-benzyl)-1-methyl-6-[3-(4-methylsulfanyl-phenyl)-prop-1-ynyl]-1H-thieno[2,3-d]pyrimidine-2,4-dione;

4-{1-Methyl-6-[3-(3-methylsulfanyl-phenyl)-prop-1-ynyl]-2,4-dioxo-1,4-dihydro-2H-thieno[2,3-d]pyrimidine-3-ylmehtyl}-benzoic acid;

3-(4-Methanesulfonyl-benzyl)-1-methyl-6-[3-(3-methylsulfanyl-phenyl)-prop-1-ynyl]-1H-thieno[2,3-d]pyrimidine-2,4-dione;

4-[1-Methyl-2,4-dioxo-6-(3-p-tolyl-prop-1-ynyl)-1,4-dihydro-2H-[2,3-d]pyrimidin-3-ylmethyl]benzoic acid;

3-(4-Methanesulfonyl-benzyl)-1-methyl-6-(3-p-tolyl-prop-1-ynyl)-1H-[2,3-d]pyrimidine-2,4-dione;

4-[1-Methyl-2,4-dioxo-6-(3-m-tolyl-prop-1-ynyl)-1,4-dihydro-2H-[2,3-d]pyrimidin-3-ylmethyl]benzoic acid;

3-(4-Methanesulfonyl-benzyl)-1-methyl-6-(3-m-tolyl-prop-1-ynyl)-1H-[2,3-d]pyrimidine-2,4-dione;

3-Benzyl-6-[3-(4-methoxy-phenyl)-prop-1-ynyl]-1-methyl-1H-thieno[2,3-d]pyrimidine-2,4-dione;

3-Benzyl-6-[3-(3-methoxy-phenyl)-prop-1-ynyl]-1-methyl-1H-thieno[2,3-d]pyrimidine-2,4-dione;

3-Benzyl-1-methyl-6-(3-pyridin-4-yl-prop-1-ynyl)-1H-thieno[2,3-d]pyrimidine-2,4-dione;

3-Benzyl-1-methyl-6-(3-pyridin-3-yl-prop-1-ynyl)-1H-thieno[2,3-d]pyrimidine-2,4-dione;

3-Benzyl-6-[3-(4-fluoro-phenyl)-prop-1-ynyl]-11-methyl-1H-thieno[2,3-d]pyrimidine-2,4-dione;

3-Benzyl-6-[3-(3-fluoro-phenyl)-prop-1-ynyl]-1-methyl-1H-thieno[2,3-d]pyrimidine-2,4-dione;

3-Benzyl-6-[3-(4-chloro-phenyl)-prop-1-ynyl]-1-methyl-1H-thieno[2,3-d]pyrimidine-2,4-dione;

3-Benzyl-6-[3-(3-chloro-phenyl)-prop-1-ynyl]-1-methyl-1H-thieno [2,3-d]pyrimidine-2,4-dione;

3-Benzyl-6-[3-(4-bromo-phenyl)-prop-1-ynyl]-1-methyl-1H-thieno[2,3-d]pyrimidine-2,4-dione;

3-Benzyl-6-[3-(3-bromo-phenyl)-prop-1-ynyl]-1-methyl-1H-thieno[2,3-d]pyrimidine-2,4-dione;

3-Benzyl-6-[3-(2-methoxy-pyridin-4-yl)-prop-1-ynyl]-1-methyl-1H-[2,3-d]pyrimidine-2,4-dione;

3-Benzyl-1-methyl-6-[3-(4-methylsulfanyl-phenyl)-prop-1-ynyl]-1H-[2,3-d]pyrimidine-2,4-dione;

3-Benzyl-1-methyl-6-[3-(3-methylsulfanyl-phenyl)-prop-1-ynyl]-1H-[2,3-d]pyrimidine-2,4-dione;

3-Benzyl-1-methyl-6-(3-p-tolyl-prop-1-ynyl)-1H-thieno [2,3-d]pyrimidine-2,4-dione;

3-Benzyl-1-methyl-6-(3-m-tolyl-prop-1-ynyl)-1H-thieno[2,3-d]pyrimidine-2,4-dione;

3-(3-Fluoro-benzyl)-6-[3-(4-methoxy-phenyl)-prop-1-ynyl]-1-methyl-1H-[2,3-d]pyrimidine-2,4-dione;

3-(3-Fluoro-benzyl)-6-[3-(3-methoxy-phenyl)-prop-1-ynyl]-1-methyl-1H-[2,3-d]pyrimidine-2,4-dione;

3-(3-Fluoro-benzyl)-1-methyl-6-(3-pyridine-4-yl-prop-1-ynyl)-1H-[2,3-d]pyrimidine-2,4-dione;

3-(3-Fluoro-benzyl)-1-methyl-6-(3-pyridine-3-yl-prop-1-ynyl)-1H-[2,3-d]pyrimidine-2,4-dione;

3-(3-Fluoro-benzyl)-6-[3-(4-fluoro-phenyl)-prop-1-ynyl]-1-methyl-1H-[2,3-d]pyrimidine-2,4-dione;

3-(3-Fluoro-benzyl)-6-[3-(3-fluoro-phenyl)-prop-1-ynyl]-1-methyl-1H-[2,3-d]pyrimidine-2,4-dione;

6-[3-(4-Chloro-phenyl)-prop-1-ynyl]-3-(3-fluoro-benzyl)-1-methyl-1H-[2,3-d]pyrimidine-2,4-dione;

6-[3-(3-Chloro-phenyl)-prop-1-ynyl]-3-(3-fluoro-benzyl)-1-methyl-1H-[2,3-d]pyrimidine-2,4-dione;

6-[3-(4-Bromo-phenyl)-prop-1-ynyl]-3-(3-fluoro-benzyl)-1-methyl-1H-[2,3-d]pyrimidine-2,4-dione;

6-[3-(3-Bromo-phenyl)-prop-1-ynyl]-3-(3-fluoro-benzyl)-1-methyl-1H-[2,3-d]pyrimidine-2,4-dione;

3-(3-Fluoro-benzyl)-6-[3-(2-methoxy-pyridin-4-yl)-prop-1-ynyl]-1-methyl-1H-thieno[2,3-d]pyrimidine-2,4-dione;

3-(3-Fluoro-benzyl)-1-methyl-6-[3-(4-methylsulfanyl-phenyl)-prop-1-ynyl]-1H-thieno[2,3-d]pyrimidine-2,4-dione;

3-(3-Fluoro-benzyl)-1-methyl-6-[3-(3-methylsulfanyl-phenyl)-prop-1-ynyl]-1H-thieno[2,3-d]pyrimidine-2,4-dione;

3-(3-Fluoro-benzyl)-1-methyl-6-(3-p-tolyl-prop-1-ynyl)-1H-thieno[2,3-d]pyrimidine-2,4-dione; and

3-(3-Fluoro-benzyl)-1-methyl-6-(3-m-tolyl-prop-1-ynyl)-1H-thieno[2,3-d]pyrimidine-2,4-dione; or a pharmaceutically acceptable salt thereof.

35. The combination according to Embodiment 23, wherein the compound of Formula 1B is selected from:

3-(3-Methoxycarbonyl-benzyl)-1-methyl-2,4-dioxo-1,2,3,4-tetrahydro-[2,3-d]pyrimidine-6-carboxylic acid benzyl ester;

3-(3-Methoxycarbonyl-benzyl)-1-methyl-2,4-dioxo-1,2,3,4-tetrahydro-[2,3-d]pyrimidine-6-carboxylic acid benzyl ester;

3-Benzofuran-5-ylmethyl-1-methyl-2,4-dioxo-1,2,3,4-tetrahydro-[2,3-d]pyrimidine-6-carboxylic acid benzyl ester;

1-Methyl-3-(4-methyl-benzyl)-2,4-dioxo-1,2,3,4-tetrahydro-thieno[2,3-d]pyrimidine-6-carboxylic acid benzyl ester;

3-(4-Acetylamino-benzyl)-1-methyl-2,4-dioxo-1,2,3,4-tetrahydro-[2,3-d]pyrimidine-6-carboxylic acid benzyl ester;

1-Methyl-2,4-dioxo-3-(4-vinyl-benzyl)-1,2,3,4-tetrahydro-thieno[2,3-d]pyrimidine-6-carboxylic acid benzyl ester;

1-Methyl-2,4-dioxo-3-(4-sulfamoyl-benzyl)-1,2,3,4-tetrahydro-thieno[2,3-d]pyrimidine-6-carboxylic acid benzyl ester;

3-(4-Bromo-benzyl)-2,4-dioxo-1,2,3,4-tetrahydro-thieno[2,3-d]pyrimidine-6-carboxylic acid pyridin-4-ylmethyl ester;

1-Methyl-2,4-dioxo-3-phenethyl-1,2,3,4-tetrahydro-thieno[2,3-d]pyrimidine-6-carboxylic acid benzyl ester;

1-Methyl-2,4-dioxo-3-[4-(2H-tetrazol-5-yl)-benzyl]-1,2,3,4-tetrahydro-[2,3-d]pyrimidine-6-carboxylic acid benzyl ester;

3-(4-Fluoro-benzyl)-2,4-dioxo-1,2,3,4-tetrahydro-thieno[2,3-d]pyrimidine-6-carboxylic acid pyridin-4-ylmethyl ester;

3-(4-tert-Butyoxycarbonyl-benzyl)-1-methyl2,4-dioxo-1,2,3,4-tetrahydro-[2,3-d]pyrimidine-6-carboxylic acid benzyl ester;

3-(4-tert-Butyoxycarbonyl-benzyl)-1-methyl-2,4-dioxo-1,2,3,4-tetrahydro-[2,3-d]pyrimidine-6-carboxylic acid;

4-[6-(4-Fluoro-benzylcarbamoyl)-1-methyl-2,4-dioxo-1,4-dihydro-2H-[2,3-d]pyrimidin-3-ylmethyl]-benzoic acid;

4-[6-(4-Dimethylamino-benzylcarbamoyl)-1-methyl-2,4-dioxo-1,4-dihydro-2H-thieno[2,3-d]pyrimidin-3-ylmethyl]-benzoic acid, compound with trifluoro-acetic acid;

4-[6-(2-Ethoxy-benzylcarbamoyl)-1-methyl-2,4-dioxo-1,2,3,4-tetrahydro-[2,3-d]pyrimidine-3-ylmethyl]-benzoic acid;

1-Methyl-2,4-dioxo-1,2,3,4-tetrahydro-thieno[2,3-d]pyrimidine-6-carboxylic acid;

1-Methyl-2,4-dioxo-1,2,3,4-tetrahydro-thieno[2,3-d]pyrimidine-6-carboxylic acid 3-methoxy-benzylamide;

1-Methyl-2,4-dioxo-3-[4-(1H-tetrazol-5-yl)-benzyl]-1,2,3,4-tetrahydro-[2,3-d]pyrimidine-6-carboxylic acid 3-methoxy-benzylamide;

1-Methyl-3-[4-(morpholine-4-sulfonyl)-benzyl]-2,4-dioxo-1,2,3,4-tetrahydro-thieno[2,3-d]pyrimidine-6-carboxylic acid 3-methoxy-benzylamide;

1-Methyl-3-[4-(morpholine-4-carbonyl)-benzyl]-2,4-dioxo-1,2,3,4-tetrahydro-thieno[2,3-d]pyrimidine-6-carboxylic acid 3-methoxy-benzylamide;

3-But-2-ynyl-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-[3-(1H-tetrazol-5-yl)-benzyl]-1,2,3,4-tetrahydro-[2,3-d]pyrimidine-6-carboxylic acid 3-methoxy-benzylamide;

3-(4-Cyano-benzyl)-1-methyl-2,4-dioxo-1,2,3,4-tetrahydro-thieno[2,3-d]pyrimidine-6-carboxylic acid 3-methoxy-benzylamide;

{4-[6-(3-Methoxy-benzylcarbamoyl)-1-methyl-2,4-dioxo-1,4-dihydro-2H-[2,3-d]pyrimidin-3-ylmethyl]-phenyl}-acetic acid;

3-[2-(2,4-Dichloro-benzenesulfonyl)-ethyl]-1-methyl-2,4-dioxo-1,2,3,4-tetrahydro-thieno[2,3-d]pyrimidine-6-carboxylic acid 3-methoxy-benzylamide;

3-(4-Methanesulfonyl-benzyl)-1-methyl-2,4-dioxo-1,2,3,4-tetrahydro-[2,3-d]pyrimidine-6-carboxylic acid 3-methoxy-benzylamide;

1-Methyl-2,4-dioxo-3-(4-sulfamoyl-benzyl)-1,2,3,4-tetrahydro-thieno[2,3-d]-pyrimidine-6-carboxylic acid 3-methoxy-benzylamide;

3-Benzyl-1-methyl-2,4-dioxo-1,2,3,4-tetrahydro-thieno[2,3-d]pyrimidine-6-carboxylic acid (2-methoxy-pyridin-4-ylmethyl)-amide;

1-Methyl-3-(4-methylsulfamoyl-benzyl)-2,4-dioxo-1,2,3,4-tetrahydro-[2,3-d]pyrimidine-6-carboxylic acid 3-methoxy-benzylamide;

3-(4-Isopropylsulfamoyl-benzyl)-1-methyl-2,4-dioxo-1,2,3,4-tetrahydro-[2,3-d]pyrimidine-6-carboxylic acid 3-methoxy-benzylamide;

1-Methyl-2,4-dioxo-3-[4-(pyrrolidine-1-sulfonyl)-benzyl]-1,2,3,4-tetrahydro-thieno[2,3-d]pyrimidine-6-carboxylic acid 3-methoxy-benzylamide; and

1-Methyl-3-[4-(4-methyl-piperidine-1-sulfonyl)-benzyl]-2,4-dioxo-1,2,3,4-tetrahydro-thieno[2,3-d]pyrimidine-6-carboxylic acid 3-methoxy-benzylamide; or a pharmaceutically acceptable salt thereof.

36. The combination according to Embodiment 23, wherein the compound of Formula 1B is selected from:

3-Benzyl-1-methyl-2,4-dioxo-1,2,3,4-tetrahydro-thieno[2,3-d]pyrimidine-6-carboxylic acid benzofuran-2-ylmethyl ester;

3-(4-Bromo-benzyl)-2,4-dioxo-1,2,3,4-tetrahydro-thieno[2,3-d]pyrimidine-6-carboxylic acid pyridin-4-ylmethyl ester;

3-Benzyl-2,4-dioxo-1,2,3,4-tetrahydro-thieno[2,3-d]pyrimidine-6-carboxylic acid 4-methoxy-benzyl ester;

4-{1-Methyl-2,4-dioxo-6-[(pyridin-4-ylmethyl)-carbamoyl]-1,4-dihydro-2H-thieno[2,3-d]pyrimidin-3-ylmethyl}-benzoic acid, compound with trifluoro-acetic acid;

4-[6-(4-Methoxy-benzylcarbamoyl)-1-methyl-2,4-dioxo-1,4-dihydro-2H-[2,3-d]pyrimidin-3-ylmethyl]-benzoic acid;

4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid tert-butyl ester;

4-[6-(3,4-Dimethoxy-benzylcarbamoyl)-1-methyl-2,4-dioxo-1,4-dihydro-2H-thieno[2,3-d]pyrimidin-3-ylmethyl]-benzoic acid;

4-[6-(4-Bromo-benzylcarbamoyl)-1-methyl-2,4-dioxo-1,4-dihydro-2H-[2,3-d]pyrimidin-3-ylmethyl]-benzoic acid;

4-[6-(4-Bromo-benzylcarbamoyl)-1-methyl-2,4-dioxo-1,4-dihydro-2H-[2,3-d]pyrimidin-3-ylmethyl]-benzoic acid tert-butyl ester;

4-[6-(3,5-Bis-trifluoromethyl-benzylcarbamoyl)-1-methyl-2,4-dioxo-1,4-dihydro-2H-thieno[2,3-d]pyrimidin-3-ylmethyl]-benzoic acid;

4-[6-(4-Chloro-benzylcarbamoyl)-1-methyl-2,4-dioxo-1,4-dihydro-2H-[2,3-d]pyrimidin-3-ylmethyl]-benzoic acid;

4-[1-Methyl-2,4-dioxo-6-(4-sulfamoyl-benzylcarbamoyl)-1,4-dihydro-2H-[2,3-d]pyrimidin-3-ylmethyl]-benzoic acid;

3-(4-Fluoro-benzyl)-1-methyl-2,4-dioxo-1,2,3,4-tetrahydro-thieno[2,3-d]pyrimidine-6-carboxylic acid 4-methoxy-benzylamide;

3-(4-Iodo-benzyl)-1-methyl-2,4-dioxo-1,2,3,4-tetrahydro-thieno[2,3-d]pyrimidine-6-carboxylic acid 3-methoxy-benzylamide;

3-(4-Dimethylsulfamoyl-benzyl)-1-methyl-2,4-dioxo-1,2,3,4-tetrahydro-[2,3-d]pyrimidine-6-carboxylic acid 4-methoxy-benzylamide;

3-(3-Methoxy-benzyl)-1-methyl-2,4-dioxo-1,2,3,4-tetrahydro-thieno[2,3-d]pyrimidine-6-carboxylic acid 4-methoxy-benzylamide;

3-(4-Cyano-benzyl)-1-methyl-2,4-dioxo-1,2,3,4-tetrahydro-thieno[2,3-d]pyrimidine-6-carboxylic acid 4-methoxy-benzylamide;

3-(4-Acetylamino-benzyl)-1-methyl-2,4-dioxo-1,2,3,4-tetrahydro-[2,3-d]pyrimidine-6-carboxylic acid 3-methoxy-benzylamide;

5-[6-(3-Methoxy-benzylcarbamoyl)-1-methyl-2,4-dioxo-1,4-dihydro-2H-[2,3-d]pyrimidin-3-ylmethyl]-furan-2-carboxylic acid ethyl ester;

3-(4-Cyano-benzyl)-2,4-dioxo-1,2,3,4-tetrahydro-thieno[2,3-d]pyrimidine-6-carboxylic acid 3-methoxy-benzyl ester;

1-Methyl-2,4-dioxo-3-[4-(5-thioxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;

4-[6-(3-Methoxy-benzylcarbamoyl)-1-methyl-2,4-dioxo-1,4-dihydro-2H-[2,3-d]pyrimidin-3-ylmethyl]-benzoic acid 2-dimethylamino-ethyl ester;

3-Cyclohexylmethyl-1-methyl-2,4-dioxo-1,2,3,4-tetrahydro-thieno [2,3-d]pyrimidine-6-carboxylic acid 4-methoxy-benzylamide;

3-Cyclohexylmethyl-1-methyl-2,4-dioxo-1,2,3,4-tetrahydro-thieno [2,3-d]pyrimidine-6-carboxylic acid 4-methoxy-benzylamide;

3-Benzyl-2,4-dioxo-1,2,3,4-tetrahydro-thieno [2,3-d]pyrimidine-6-carboxylic acid furan-3-ylmethyl ester;

3-Benzyl-1-methyl-2,4-dioxo-1,2,3,4-tetrahydro-thieno[2,3-d]pyrimidine-6-carboxylic acid pentafluorophenylmethyl ester;

3-Benzyl-1-ethyl-2,4-dioxo-1,2,3,4-tetrahydro-thieno[2,3-d]pyrimidine-6-carboxylic acid benzyl ester;

3-Benzyl-1-cyclopropylmethyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid (pyridin-4-ylmethyl)-amide;

3-Benzyl-1-methyl-2,4-dioxo-1,2,3,4-tetrahydro-thieno[2,3-d]pyrimidine-6-carboxylic acid 3-bromo-benzyl ester;

4-[6-(3-Difluoromethoxy-benzylcarbamoyl)-1-methyl-2,4-dioxo-1,4-dihydro-2H-thieno[2,3-d]pyrimidin-3-ylmethyl]-benzoic acid;

4-[6-(3-Difluoromethoxy-benzylcarbamoyl)-1-methyl-2,4-dioxo-1,4-dihydro-2H-thieno[2,3-d]pyrimidin-3-ylmethyl]-benzoic acid tert-butyl ester;

4-[6-(3-Methoxy-benzylcarbamoyl)-1-methyl-2,4-dioxo-1,4-dihydro-2H-[2,3-d]pyrimidin-3-ylmethyl]-benzoic acid;

4-[6-(4-Methanesulfonyl-benzylcarbamoyl)-1-methyl-2,4-dioxo-1,4-dihydro-2H-thieno[2,3-d]pyrimidin-3-ylmethyl]-benzoic acid tert-butyl ester;

4-[6-(4-Methanesulfonyl-benzylcarbamoyl)-1-methyl-2,4-dioxo-1,4-dihydro-2H-thieno[2,3-d]pyrimidin-3-ylmethyl]-benzoic acid;

4-[1-Methyl-2,4-dioxo-6-(2-pyridin-4-yl-ethylcarbamoyl)-1,4-dihydro-2H-[2,3-d]pyrimidin-3-ylmethyl]-benzoic acid;

1-Methyl-2,4-dioxo-3-(4-trifluoromethoxy-benzyl)-1,2,3,4-tetrahydro-[2,3-d]pyrimidine-6-carboxylic acid 3-methoxy-benzylamide;

4-[6-(3-Methoxy-benzylcarbamoyl)-1-methyl-2,4-dioxo-1,4-dihydro-2H-[2,3-d]pyrimidin-3-ylmethyl]-benzoic acid methyl ester;

3-(2,3-Dihydro-benzofuran-6-ylmethyl)-1-methyl-2,4-dioxo-1,2,3,4-tetrahydro-thieno[2,3-d]pyrimidine-6-carboxylic acid 3-methoxy-benzylamide;

1-Methyl-3-(2-methyl-thiazol-5-ylmethyl)-2,4-dioxo-1,2,3,4-tetrahydro-[2,3-d]pyrimidine-6-carboxylic acid 3-methoxy-benzylamide;

1-Methyl-2,4-dioxo-3-[4-(1H-tetrazol-5-yl)-benzyl]-1,2,3,4-tetrahydro-[2,3-d]pyrimidine-6-carboxylic acid 4-fluoro-benzylamide;

3-Benzyl-2-methoxy-4-oxo-3,4-dihydro-thieno[2,3-d]pyrimidine-6-carboxylic acid benzyl ester;

4-[6-(3-Methoxy-benzylcarbamoyl)-1-methyl-2,4-dioxo-1,4-dihydro-2H-[2,3-d]pyrimidin-3-ylmethyl]-benzoic acid 2,2-dimethyl-propionyloxymethyl ester;

4-[6-(3-Methoxy-benzylcarbamoyl)-1-methyl-2,4-dioxo-1,4-dihydro-2H-[2,3-d]pyrimidin-3-ylmethyl]-cyclohexanecarboxylic acid;

4-[6-(3-Methoxy-benzylcarbamoyl)-1-methyl-2,4-dioxo-1,4-dihydro-2H-[2,3-d]pyrimidin-3-ylmethyl]-cyclohexanecarboxylic acid methyl ester;

1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid methyl ester;

1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid tert-butyl ester;

1-{4-[6-(3-Methoxy-benzylcarbamoyl)-1-methyl-2,4-dioxo-1,4-dihydro-2H-thieno[2,3-d]pyrimidin-3-ylmethyl]-phenyl}-cyclopropanecarboxylic acid;

2-{4-[6-(3-Methoxy-benzylcarbamoyl)-1-methyl-2,4-dioxo-1,4-dihydro-2H-thieno[2,3-d]pyrimidin-3-ylmethyl]-phenoxy}-2-methyl-propionic acid tert-butyl ester;

2-{4-[6-(3-Methoxy-benzylcarbamoyl)-1-methyl-2,4-dioxo-1,4-dihydro-2H-thieno[2,3-d]pyrimidin-3-ylmethyl]-phenoxy}-2-methyl-propionic acid;

3-Benzyl-1-methyl-2,4-dioxo-1,2,3,4-tetrahydro-furo[2,3-d]pyrimidine-6-carboxylic acid benzyl ester;

3-(3-Methoxy-benzyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzyl ester;

3-Biphenyl-4-ylmethyl-1-methyl-2,4-dioxo-1,2,3,4-tetrahydro-thieno[2,3-d]pyrimidine-6-carboxylic acid benzyl ester;

3-(4-Methanesulfonyl-benzyl)-1-methyl-2,4-dioxo-1,2,3,4-tetrahydro-[2,3-d]pyrimidine-6-carboxylic acid benzyl ester;

3-(4-Methanesulfonyl-benzyl)-1-methyl-2,4-dioxo-1,2,3,4-tetrahydro-[2,3-d]pyrimidine-6-carboxylic acid benzyl ester;

1-Methyl-3-(4-methyl-benzyl)-2,4-dioxo-1,2,3,4-tetrahydro-thieno[2,3-d]pyrimidine-6-carboxylic acid benzyl ester;

1-Methyl-2,4-dioxo-3-phenethyl-1,2,3,4-tetrahydro-thieno[2,3-d]pyrimidine-6-carboxylic acid benzyl ester;

3-(4-Amino-6-phenylamino-1,3,5-triazin-2-ylmethyl)-1-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethyl-benzyl)-1,2,3,4-tetrahydro-[2,3-d]pyrimidine-6-carboxylic acid benzyl ester;

3-(6-Cyano-hexyl)-1-methyl-2,4-dioxo-1,2,3,4-tetrahydro-thieno[2,3-d]pyrimidine-6-carboxylic acid benzyl ester;

3-[2-(2,5-Dimethoxy-phenyl)-2-oxo-ethyl]-1-methyl-2,4-dioxo-1,2,3,4-tetrahydro-thieno[2,3-d]pyrimidine-6-carboxylic acid benzyl ester;

3-(3-Iodo-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(3-trifluoromethyl-benzyl)-1,2,3,4-tetrahydro-[2,3-d]pyrimidine-6-carboxylic acid benzyl ester;

3-(2,4-Bis-trifluoromethyl-benzyl)-1-methyl-2,4-dioxo-1,2,3,4-tetrahydro-[2,3-d]pyrimidine-6-carboxylic acid benzyl ester;

3-[2-(1,3-Dioxo-1,3-dihydro-isoindol-2-yl)-ethyl]-1-methyl-2,4-dioxo-1,2,3,4-tetrahydro-thieno[2,3-d]pyrimidine-6-carboxylic acid benzyl ester;

3-[2-(1,3-Dioxo-1,3-dihydro-isoindol-2-yl)-ethyl]-1-methyl-2,4-dioxo-1,2,3,4-tetrahydro-thieno[2,3-d]pyrimidine-6-carboxylic acid benzyl ester;

3-(2-Carboxy-allyl)-1-methyl-2,4-dioxo-1,2,3,4-tetrahydro-thieno[2,3-d]pyrimidine-6-carboxylic acid benzyl ester;

3-(2-Carboxy-allyl)-1-methyl-2,4-dioxo-1,2,3,4-tetrahydro-thieno[2,3-d]pyrimidine-6-carboxylic acid benzyl ester;

3-(3-Amino-propyl)-1-methyl-2,4-dioxo-1,2,3,4-tetrahydro-thieno[2,3-d]pyrimidine-6-carboxylic acid benzyl ester;

3-(1,3-Dioxo-1,3-dihydro-isoindol-2-ylmethyl)-1-methyl-2,4-dioxo-1,2,3,4-tetrahydro-thieno[2,3-d]pyrimidine-6-carboxylic acid benzyl ester;

3-(4-Fluoro-benzyl)-1-methyl-2,4-dioxo-1,2,3,4-tetrahydro-thieno[2,3-d]pyrimidine-6-carboxylic acid benzyl ester;

1-Methyl-3-oxiranylmethyl-2,4-dioxo-1,2,3,4-tetrahydro-thieno[2,3-d]pyrimidine-6-carboxylic acid benzyl ester;

1-Methyl-3-((S)-2-methyl-butyl)-2,4-dioxo-1,2,3,4-tetrahydro-thieno[2,3-d]pyrimidine-6-carboxylic acid benzyl ester;

1-Methyl-2,4-dioxo-3-(4-phenoxy-butyl)-1,2,3,4-tetrahydro-thieno[2,3-d]pyrimidine-6-carboxylic acid benzyl ester;

3-(2-Cyano-benzyl)-1-methyl-2,4-dioxo-1,2,3,4-tetrahydro-thieno[2,3-d]pyrimidine-6-carboxylic acid benzyl ester;

1-Methyl-2,4-dioxo-3-(3-phenoxy-propyl)-1,2,3,4-tetrahydro-thieno [2,3-d]pyrimidine-6-carboxylic acid benzyl ester;

3-Hex-5-enyl-1-methyl-2,4-dioxo-1,2,3,4-tetrahydro-thieno[2,3-d]pyrimidine-6-carboxylic acid benzyl ester;

1-Methyl-2,4-dioxo-3-pyridin-3-ylmethyl-1,2,3,4-tetrahydro-thieno[2,3-d]pyrimidine-6-carboxylic acid benzyl ester;

3-[2-Hydroxy-3-(naphthalen-1-yloxy)-propyl]-1-methyl-2,4-dioxo-1,2,3,4-tetrahydro-thieno[2,3-d]pyrimidine-6-carboxylic acid benzyl ester;

1,3-Dimethyl-2,4-dioxo-1,2,3,4-tetrahydro-thieno [2,3-d]pyrimidine-6-carboxylic acid benzyl ester;

3-Cyclobutylmethyl-1-methyl-2,4-dioxo-1,2,3,4-tetrahydro-thieno[2,3-d]pyrimidine-6-carboxylic acid benzyl ester;

3-Allyl-1-methyl-2,4-dioxo-1,2,3,4-tetrahydro-thieno[2,3-d]pyrimidine-6-carboxylic acid benzyl ester;

1-Methyl-2,4-dioxo-3-prop-2-ynyl-1,2,3,4-tetrahydro-thieno [2,3-d]pyrimidine-6-carboxylic acid benzyl ester;

3-But-2-ynyl-1-methyl-2,4-dioxo-1,2,3,4-tetrahydro-thieno[2,3-d]pyrimidine-6-carboxylic acid benzyl ester;

1-Methyl-2,4-dioxo-3-(2-phenoxy-ethyl)-1,2,3,4-tetrahydro-thieno[2,3-d]pyrimidine-6-carboxylic acid benzyl ester;

1-Methyl-2,4-dioxo-3-(2-phenoxy-ethyl)-1,2,3,4-tetrahydro-thieno[2,3-d]pyrimidine-6-carboxylic acid benzyl ester;

3-((R)-3-Hydroxy-2-methyl-propyl)-1-methyl-2,4-dioxo-1,2,3,4-tetrahydro-thieno[2,3-d]pyrimidine-6-carboxylic acid benzyl ester;

3-Isobutyl-1-methyl-2,4-dioxo-1,2,3,4-tetrahydro-thieno[2,3-d]pyrimidine-6-carboxylic acid benzyl ester;

3-(6-Chloro-pyridin-3-ylmethyl)-1-methyl-2,4-dioxo-1,2,3,4-tetrahydro-[2,3-d]pyrimidine-6-carboxylic acid benzyl ester;

3-(2-Benzenesulfonylmethyl-benzyl)-1-methyl-2,4-dioxo-1,2,3,4-tetrahydro-thieno[2,3-d]pyrimidine-6-carboxylic acid benzyl ester;

1-Methyl-3-naphthalen-1-ylmethyl-2,4-dioxo-1,2,3,4-tetrahydro-[2,3-d]pyrimidine-6-carboxylic acid benzyl ester;

1-Methyl-2,4-dioxo-3-(2-trifluoromethyl-benzyl)-1,2,3,4-tetrahydro-[2,3-d]pyrimidine-6-carboxylic acid benzyl ester;

3-(3-Chloro-benzyl)-1-methyl-2,4-dioxo-1,2,3,4-tetrahydro-thieno[2,3-d]pyrimidine-6-carboxylic acid benzyl ester;

3-(4-Methoxycarbonyl-butyl)-1-methyl-2,4-dioxo-1,2,3,4-tetrahydro-[2,3-d]pyrimidine-6-carboxylic acid benzyl ester;

3-Ethyl-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid 4-fluoro-benzyl ester;

3-[2-(4-Chloro-benzenesulfonyl)-ethyl]-1-methyl-2,4-dioxo-1,2,3,4-tetrahydro-thieno[2,3-d]pyrimidine-6-carboxylic acid benzyl ester;

3-(2-Acetoxy-ethyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid 2-phenoxy-ethyl ester;

3-Benzyl-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzylamide;

3-Benzyl-1-methyl-2,4-dioxo-1,2,3,4-tetrahydro-thieno[2,3-d]pyrimidine-6-carboxylic acid 2,6-dichloro-benzyl ester;

3-Benzyl-1-methyl-2,4-dioxo-1,2,3,4-tetrahydro-thieno[2,3-d]pyrimidine-6-carboxylic acid butyl ester;

3-Benzyl-1-methyl-2,4-dioxo-1,2,3,4-tetrahydro-thieno[2,3-d]pyrimidine-6-carboxylic acid 2,3-dihydro-1,4-benzodioxin-2-ylmethyl ester;

3-Benzyl-1-methyl-2,4-dioxo-1,2,3,4-tetrahydro-thieno[2,3-d]pyrimidine-6-carboxylic acid 2-diethylamino-1-methyl-ethyl ester;

3-Benzyl-1-methyl-2,4-dioxo-1,2,3,4-tetrahydro-thieno[2,3-d]pyrimidine-6-carboxylic acid 4-fluoro-benzyl ester;

3-Benzyl-1-methyl-2,4-dioxo-1,2,3,4-tetrahydro-thieno[2,3-d]pyrimidine-6-carboxylic acid 4-isopropyl-benzyl ester;

3-Benzyl-1-methyl-2,4-dioxo-1,2,3,4-tetrahydro-thieno[2,3-d]pyrimidine-6-carboxylic acid 2-p-tolyl-ethyl ester;

3-Benzyl-1-methyl-2,4-dioxo-1,2,3,4-tetrahydro-thieno[2,3-d]pyrimidine-6-carboxylic acid 4-trifluoromethyl-benzyl ester;

3-Benzyl-1-methyl-2,4-dioxo-1,2,3,4-tetrahydro-thieno[2,3-d]pyrimidine-6-carboxylic acid cyclobutylmethyl ester;

3-Benzyl-1-methyl-2,4-dioxo-1,2,3,4-tetrahydro-thieno [2,3-d]pyrimidine-6-carboxylic acid 2,6-difluoro-benzyl ester;

3-Benzyl-1-methyl-2,4-dioxo-1,2,3,4-tetrahydro-thieno[2,3-d]pyrimidine-6-carboxylic acid 2-(2-hydroxy-phenyl)-ethyl ester;

3-Benzyl-1-methyl-2,4-dioxo-1,2,3,4-tetrahydro-thieno[2,3-d]pyrimidine-6-carboxylic acid 2-(2-hydroxy-phenyl)-ethyl ester;

3-Benzyl-1-methyl-2,4-dioxo-1,2,3,4-tetrahydro-thieno[2,3-d]pyrimidine-6-carboxylic acid 1-methyl-piperidin-4-yl ester;

3-Benzyl-1-methyl-2,4-dioxo-1,2,3,4-tetrahydro-thieno[2,3-d]pyrimidine-6-carboxylic acid 1-methyl-piperidin-4-yl ester;

3-Benzyl-1-methyl-2,4-dioxo-1,2,3,4-tetrahydro-thieno[2,3-d]pyrimidine-6-carboxylic acid pyridin-3-ylmethyl ester;

3-Benzyl-1-methyl-2,4-dioxo-1,2,3,4-tetrahydro-thieno[2,3-d]pyrimidine-6-carboxylic acid 3-pyridin-3-yl-propyl ester;

3-Benzyl-1-methyl-2,4-dioxo-1,2,3,4-tetrahydro-thieno [2,3-d]pyrimidine-6-carboxylic acid 2-dimethylamino-1-methyl-ethyl ester;

3-Benzyl-1-methyl-2,4-dioxo-1,2,3,4-tetrahydro-thieno[2,3-d]pyrimidine-6-carboxylic acid 4-methoxy-benzyl ester;

3-Benzyl-1-methyl-2,4-dioxo-1,2,3,4-tetrahydro-thieno[2,3-d]pyrimidine-6-carboxylic acid tetrahydro-pyran-4-yl ester;

3-Benzyl-1-methyl-2,4-dioxo-1,2,3,4-tetrahydro-thieno[2,3-d]pyrimidine-6-carboxylic acid 2,2,2-trifluoro-1-trifluoromethyl-ethyl ester;

3-Benzyl-1-methyl-2,4-dioxo-1,2,3,4-tetrahydro-thieno[2,3-d]pyrimidine-6-carboxylic acid 2-trifluoromethyl-benzyl ester;

3-Benzyl-1-methyl-2,4-dioxo-1,2,3,4-tetrahydro-thieno[2,3-d]pyrimidine-6-carboxylic acid 2-benzyloxy-ethyl ester;

3-Benzyl-1-methyl-2,4-dioxo-1,2,3,4-tetrahydro-thieno[2,3-d]pyrimidine-6-carboxylic acid 2,2,2-trichloro-ethyl ester;

3-Benzyl-1-methyl-2,4-dioxo-1,2,3,4-tetrahydro-thieno[2,3-d]pyrimidine-6-carboxylic acid phenethyl ester;

3-Benzyl-1-methyl-2,4-dioxo-1,2,3,4-tetrahydro-thieno[2,3-d]pyrimidine-6-carboxylic acid 3-ethyl-oxetan-3-ylmethyl ester;

3-Benzyl-1-methyl-2,4-dioxo-1,2,3,4-tetrahydro-thieno[2,3-d]pyrimidine-6-carboxylic acid 2-morpholin-4-yl-ethyl ester;

3-Benzyl-1-methyl-2,4-dioxo-1,2,3,4-tetrahydro-thieno[2,3-d]pyrimidine-6-carboxylic acid 2-pyrrolidin-1-yl-ethyl ester;

3-Benzyl-1-methyl-2,4-dioxo-1,2,3,4-tetrahydro-thieno[2,3-d]pyrimidine-6-carboxylic acid 2-pyrrolidin-1-yl-ethyl ester;

3-Benzyl-1-methyl-2,4-dioxo-1,2,3,4-tetrahydro-thieno [2,3-d]pyrimidine-6-carboxylic acid 2-(2-ethoxy-ethoxy)-ethyl ester;

3-Benzyl-1-methyl-2,4-dioxo-1,2,3,4-tetrahydro-thieno[2,3-d]pyrimidine-6-carboxylic acid tetrahydro-pyran-2-ylmethyl ester;

3-Benzyl-1-methyl-2,4-dioxo-1,2,3,4-tetrahydro-thieno [2,3-d]pyrimidine-6-carboxylic acid 4-nitro-benzyl ester;

3-Benzyl-1-methyl-2,4-dioxo-1,2,3,4-tetrahydro-thieno[2,3-d]pyrimidine-6-carboxylic acid pentyl ester;

3-Benzyl-1-methyl-2,4-dioxo-1,2,3,4-tetrahydro-thieno[2,3-d]pyrimidine-6-carboxylic acid 3-phenyl-propyl ester;

3-Benzyl-1-methyl-2,4-dioxo-1,2,3,4-tetrahydro-thieno[2,3-d]pyrimidine-6-carboxylic acid 3-phenoxy-benzyl ester;

3-Benzyl-1-methyl-2,4-dioxo-1,2,3,4-tetrahydro-thieno[2,3-d]pyrimidine-6-carboxylic acid 3,5-dimethoxy-benzyl ester;

3-Benzyl-1-methyl-2,4-dioxo-1,2,3,4-tetrahydro-thieno[2,3-d]pyrimidine-6-carboxylic acid 3-methyl-butyl ester;

3-Benzyl-1-methyl-2,4-dioxo-1,2,3,4-tetrahydro-thieno[2,3-d]pyrimidine-6-carboxylic acid 4-chloro-benzyl ester;

3-Benzyl-1-methyl-2,4-dioxo-1,2,3,4-tetrahydro-thieno[2,3-d]pyrimidine-6-carboxylic acid 1-ethyl-piperidin-3-yl ester;

3-Benzyl-1-methyl-2,4-dioxo-1,2,3,4-tetrahydro-thieno[2,3-d]pyrimidine-6-carboxylic acid 4-benzyloxy-benzyl ester;

3-Benzyl-1-methyl-2,4-dioxo-1,2,3,4-tetrahydro-thieno[2,3-d]pyrimidine-6-carboxylic acid isobutyl ester;

3-Benzyl-1-methyl-2,4-dioxo-1,2,3,4-tetrahydro-thieno[2,3-d]pyrimidine-6-carboxylic acid 3-(4-methoxy-phenyl)-propyl ester;

3-Benzyl-1-methyl-2,4-dioxo-1,2,3,4-tetrahydro-thieno [2,3-d]pyrimidine-6-carboxylic acid 2-chloro-6-fluoro-benzyl ester;

3-Benzyl-1-methyl-2,4-dioxo-1,2,3,4-tetrahydro-thieno[2,3-d]pyrimidine-6-carboxylic acid (S)-(tetrahydro-furan-3-yl) ester;

3-Benzyl-1-methyl-2,4-dioxo-1,2,3,4-tetrahydro-thieno[2,3-d]pyrimidine-6-carboxylic acid 3-methoxy-benzyl ester;

3-Benzyl-1-methyl-2,4-dioxo-1,2,3,4-tetrahydro-thieno[2,3-d]pyrimidine-6-carboxylic acid 3-methoxy-benzyl ester;

3-Benzyl-1-methyl-2,4-dioxo-1,2,3,4-tetrahydro-thieno[2,3-d]pyrimidine-6-carboxylic acid 3-pyridin-2-yl-propyl ester;

3-Benzyl-1-methyl-2,4-dioxo-1,2,3,4-tetrahydro-thieno[2,3-d]pyrimidine-6-carboxylic acid 2-piperidin-2-yl-ethyl ester;

3-Benzyl-1-methyl-2,4-dioxo-1,2,3,4-tetrahydro-thieno[2,3-d]pyrimidine-6-carboxylic acid 5-bromo-2-methoxy-benzyl ester;

3-Benzyl-1-methyl-2,4-dioxo-1,2,3,4-tetrahydro-thieno[2,3-d]pyrimidine-6-carboxylic acid cycloheptylmethyl ester;

3-Benzyl-1-methyl-2,4-dioxo-1,2,3,4-tetrahydro-thieno[2,3-d]pyrimidine-6-carboxylic acid 1,2,3,4-tetrahydro-naphthalen-1-yl ester;

3-Benzyl-1-methyl-2,4-dioxo-1,2,3,4-tetrahydro-thieno[2,3-d]pyrimidine-6-carboxylic acid (S)-1-pyrrolidin-2-ylmethyl ester;

3-Benzyl-1-methyl-2,4-dioxo-1,2,3,4-tetrahydro-thieno[2,3-d]pyrimidine-6-carboxylic acid 3-chloro-benzyl ester;

3-Benzyl-1-methyl-2,4-dioxo-1,2,3,4-tetrahydro-thieno[2,3-d]pyrimidine-6-carboxylic acid 1,3-benzodioxol-5-ylmethyl ester;

3-Benzyl-1-methyl-2,4-dioxo-1,2,3,4-tetrahydro-thieno[2,3-d]pyrimidine-6-carboxylic acid 4-methylsulfanyl-benzyl ester;

3-Benzyl-1-methyl-2,4-dioxo-1,2,3,4-tetrahydro-thieno[2,3-d]pyrimidine-6-carboxylic acid 4-methylsulfanyl-benzyl ester;

3-Benzyl-1-methyl-2,4-dioxo-1,2,3,4-tetrahydro-thieno[2,3-d]pyrimidine-6-carboxylic acid 3,4-dichloro-benzyl ester;

3-Benzyl-1-methyl-2,4-dioxo-1,2,3,4-tetrahydro-thieno[2,3-d]pyrimidine-6-carboxylic acid 3,3-diphenyl-propyl ester;

3-Benzyl-1-methyl-2,4-dioxo-1,2,3,4-tetrahydro-thieno[2,3-d]pyrimidine-6-carboxylic acid 2-pyridin-2-yl-ethyl ester;

3-Benzyl-1-methyl-2,4-dioxo-1,2,3,4-tetrahydro-thieno[2,3-d]pyrimidine-6-carboxylic acid furan-3-ylmethyl ester;

3-Benzyl-1-methyl-2,4-dioxo-1,2,3,4-tetrahydro-thieno[2,3-d]pyrimidine-6-carboxylic acid but-3-enyl ester;

3-Benzyl-1-methyl-2,4-dioxo-1,2,3,4-tetrahydro-thieno[2,3-d]pyrimidine-6-carboxylic acid 2-cyano-ethyl ester;

3-Benzyl-1-methyl-2,4-dioxo-1,2,3,4-tetrahydro-thieno[2,3-d]pyrimidine-6-carboxylic acid 2-ethoxy-ethyl ester;

3-Benzyl-1-methyl-2,4-dioxo-1,2,3,4-tetrahydro-thieno[2,3-d]pyrimidine-6-carboxylic acid cyano-phenyl-methyl ester;

3-Benzyl-1-methyl-2,4-dioxo-1,2,3,4-tetrahydro-thieno[2,3-d]pyrimidine-6-carboxylic acid 4-trifluoromethyl-benzylamide;

3-Benzyl-1-methyl-2,4-dioxo-1,2,3,4-tetrahydro-thieno[2,3-d]pyrimidine-6-carboxylic acid 4-methyl-benzylamide;

3-Benzyl-1-methyl-2,4-dioxo-1,2,3,4-tetrahydro-thieno[2,3-d]pyrimidine-6-carboxylic acid phenethyl-amide;

3-Benzyl-1-methyl-2,4-dioxo-1,2,3,4-tetrahydro-thieno[2,3-d]pyrimidine-6-carboxylic acid cyclopropylamide;

1-Methyl-1,2,3,4-tetrahydro-thieno[2,3-d]pyrimidine-6-carboxylic acid 4-methoxy-benzylamide;

1-Methyl-2,4-dioxo-3-(4-sulfamoyl-benzyl)-1,2,3,4-tetrahydro-thieno[2,3-d]pyrimidine-6-carboxylic acid 4-methoxy-benzylamide;

1-Methyl-2,4-dioxo-1,2,3,4-tetrahydro-thieno[2,3-d]pyrimidine-6-carboxylic acid 3-methoxy-benzylamide;

1-Methyl-2,4-dioxo-3-(3-oxo-3-phenyl-propyl)-1,2,3,4-tetrahydro-[2,3-d]pyrimidine-6-carboxylic acid 3-methoxy-benzylamide;

3-[4-(N-Hydroxycarbamimidoyl)-benzyl]-11-methyl-2,4-dioxo-1,2,3,4-tetrahydro-thieno[2,3-d]pyrimidine-6-carboxylic acid 4-methoxy-benzylamide;

1-Methyl-2,4-dioxo-3-[4-(5-oxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;

1-Methyl-2,4-dioxo-3-[4-(5-thioxo-4,5-dihydro-1,2,4-oxadiazol-3-yl)-benzyl]-1,2,3,4-tetrahydro-thieno[2,3-d]pyrimidine-6-carboxylic acid 4-methoxy-benzylamide;

4-(5-Isopropyl-2H-pyrazol-3-yl)-pyridine;

3-Cyanomethyl-1-methyl-2,4-dioxo-1,2,3,4-tetrahydro-thieno[2,3-d]pyrimidine-6-carboxylic acid 3-methoxy-benzylamide;

(E)-4-[6-(4-Methoxy-benzylcarbamoyl)-1-methyl-2,4-dioxo-1,4-dihydro-2H-thieno[2,3-d]pyrimidin-3-yl]-but-2-enoic acid methyl ester;

(E)-4-[6-(4-Methoxy-benzylcarbamoyl)-1-methyl-2,4-dioxo-1,4-dihydro-2H-thieno[2,3-d]pyrimidin-3-yl]-but-2-enoic acid;

3-(2-Benzenesulfonyl-ethyl)-1-methyl-2,4-dioxo-1,2,3,4-tetrahydro-[2,3-d]pyrimidine-6-carboxylic acid 4-methoxy-benzylamide;

2-Methoxy-4-[6-(4-methoxy-benzylcarbamoyl)-1-methyl-2,4-dioxo-1,4-dihydro-2H-thieno[2,3-d]pyrimidin-3-ylmethyl]-benzoic acid methyl ester;

3-(2-Methoxymethyl-1,1,3-trioxo-2,3-dihydro-1H-11⁶-1,2-benzisothiazol-6-ylmethyl)-1-methyl-2,4-dioxo-1,2,3,4-tetrahydro-thieno[2,3-d]pyrimidine-6-carboxylic acid 4-methoxy-benzylamide;

1-Methyl-3-oct-2-ynyl-2,4-dioxo-1,2,3,4-tetrahydro-thieno[2,3-d]pyrimidine-6-carboxylic acid 4-methoxy-benzylamide;

3-[2-(4-Chloro-benzenesulfonylamino)-ethyl]-1-methyl-2,4-dioxo-1,2,3,4-tetrahydro-thieno[2,3-d]pyrimidine-6-carboxylic acid 4-methoxy-benzylamide;

3-[2-(4-Bromo-phenoxy)-ethyl]-1-methyl-2,4-dioxo-1,2,3,4-tetrahydro-[2,3-d]pyrimidine-6-carboxylic acid 4-methoxy-benzylamide;

3-[2-(4-Bromo-phenoxy)-ethyl]-1-methyl-2,4-dioxo-1,2,3,4-tetrahydro-[2,3-d]pyrimidine-6-carboxylic acid 3-methoxy-benzylamide;

3-[2-(4-Fluoro-phenoxy)-ethyl]-1-methyl-2,4-dioxo-1,2,3,4-tetrahydro-[2,3-d]pyrimidine-6-carboxylic acid 4-methoxy-benzylamide;

3-[2-(4-Chloro-benzenesulfonyl)-ethyl]-1-methyl-2,4-dioxo-1,2,3,4-tetrahydro-thieno[2,3-d]pyrimidine-6-carboxylic acid 4-methoxy-benzylamide;

3-[2-(4-Fluoro-phenoxy)-ethyl]-1-methyl-2,4-dioxo-1,2,3,4-tetrahydro-[2,3-d]pyrimidine-6-carboxylic acid 3-methoxy-benzylamide;

1-Methyl-2,4-dioxo-1,2,3,4-tetrahydro-thieno[2,3-d]pyrimidine-6-carboxylic acid (2-methoxy-pyridin-4-ylmethyl)-amide;

3-Cyclohexylmethyl-1-methyl-2,4-dioxo-1,2,3,4-tetrahydro-thieno[2,3-d]pyrimidine-6-carboxylic acid 3-methoxy-benzylamide;

4-[6-(4-Methoxy-benzylcarbamoyl)-1-methyl-2,4-dioxo-1,4-dihydro-2H-[2,3-d]pyrimidin-3-ylmethyl]-2-methyl-benzoic acid methyl ester;

4-[6-(4-Methoxy-benzylcarbamoyl)-1-methyl-2,4-dioxo-1,4-dihydro-2H-[2,3-d]pyrimidin-3-ylmethyl]-benzoic acid methyl ester;

2-Methoxy-4-[6-(3-methoxy-benzylcarbamoyl)-1-methyl-2,4-dioxo-1,4-dihydro-2H-thieno[2,3-d]pyrimidin-3-ylmethyl]-benzoic acid methyl ester;

4-[6-(3-Methoxy-benzylcarbamoyl)-1-methyl-2,4-dioxo-1,4-dihydro-2H-[2,3-d]pyrimidin-3-ylmethyl]-2-methyl-benzoic acid methyl ester;

1-Methyl-2,4-dioxo-3-(3-oxo-3-phenyl-propyl)-1,2,3,4-tetrahydro-[2,3-d]pyrimidine-6-carboxylic acid 4-methoxy-benzylamide;

3-[2-(4-Chloro-phenoxy)-ethyl]-1-methyl-2,4-dioxo-1,2,3,4-tetrahydro-[2,3-d]pyrimidine-6-carboxylic acid 3-methoxy-benzylamide;

1-Methyl-2,4-dioxo-3-[2-(3-trifluoromethyl-benzenesulfonyl)-ethyl]-1,2,3,4-tetrahydro-thieno[2,3-d]pyrimidine-6-carboxylic acid 3-methoxy-benzylamide;

1-Methyl-2,4-dioxo-3-[2-(3-trifluoromethyl-benzenesulfonyl)-ethyl]-1,2,3,4-tetrahydro-thieno[2,3-d]pyrimidine-6-carboxylic acid 4-methoxy-benzylamide;

3-[2-(4-Chloro-benzenesulfonyl)-ethyl]-1-methyl-2,4-dioxo-1,2,3,4-tetrahydro-thieno[2,3-d]pyrimidine-6-carboxylic acid 3-methoxy-benzylamide; and

3-(2-Amino-ethyl)-1-methyl-2,4-dioxo-1,2,3,4-tetrahydro-thieno[2,3-d]pyrimidine-6-carboxylic acid 4-methoxy-benzylamide; or a pharmaceutically acceptable salt thereof.

37. The combination according to Embodiment 23, wherein the compound of Formula IB is selected from:

38. The combination according to Embodiment 23, wherein the compound of Formula IB is selected from:

1-Methyl-2,4-dioxo-1,2,3,4-tetrahydro-thieno[2,3-d]pyrimidine-6-carboxylic acid;

4-(6-Carbamoyl-1-methyl-2,4-dioxo-1,4-dihydro-2H-thieno[2,3-d]pyrimidin-3-ylmethyl)-2-methyl-benzoic acid;

4-(6-Carbamoyl-1-methyl-2,4-dioxo-1,4-dihydro-2H-thieno[2,3-d]pyrimidin-3-ylmethyl)-2-methyl-benzoic acid methyl ester;

4-[6-(3-Hydroxy-benzylcarbamoyl)-1-methyl-2,4-dioxo-1,4-dihydro-2H-[2,3-d]pyrimidin-3-ylmethyl]-2-methyl-benzoic acid;

4-(6-Carbamoyl-1-methyl-2,4-dioxo-1,4-dihydro-2H-thieno[2,3-d]pyrimidin-3-ylmethyl)-2-hydroxy-benzoic acid;

3-(2-Amino-ethyl)-1-methyl-2,4-dioxo-1,2,3,4-tetrahydro-thieno[2,3-d]pyrimidine-6-carboxylic acid 3-methoxy-benzylamide; and

4-(2,5-Di-pyridin-4-yl-thiophen-3-yl)-benzaldehyde.

39. The combination according to Embodiment 23, wherein the compound of Formula IB is selected from:

1-Methyl-2,4-dioxo-3-(1-phenyl-ethyl)-1,2,3,4-tetrahydro-thieno[2,3-d]pyrimidine-6-carboxylic acid 4-methoxy-benzylamide;

1-Methyl-2,4-dioxo-3-(3-oxo-3-phenyl-propyl)-1,2,3,4-tetrahydro-[2,3-d]pyrimidine-6-carboxylic acid 4-methoxy-benzylamide;

3-((S)-3,7-Dimethyl-oct-6-enyl)-1-methyl-2,4-dioxo-1,2,3,4-tetrahydro-[2,3-d]pyrimidine-6-carboxylic acid 4-methoxy-benzylamide;

3-(2-Ethyl-hexyl)-1-methyl-2,4-dioxo-1,2,3,4-tetrahydro-thieno[2,3-d]pyrimidine-6-carboxylic acid 4-methoxy-benzylamide;

3-(5-Cyano-pentyl)-1-methyl-2,4-dioxo-1,2,3,4-tetrahydro-thieno[2,3-d]pyrimidine-6-carboxylic acid 4-methoxy-benzylamide;

3-(E)-But-2-enyl-1-methyl-2,4-dioxo-1,2,3,4-tetrahydro-thieno[2,3-d]pyrimidine-6-carboxylic acid 4-methoxy-benzylamide;

1-Methyl-3-(2-naphthalen-1-yl-ethyl)-2,4-dioxo-1,2,3,4-tetrahydro-[2,3-d]pyrimidine-6-carboxylic acid 4-methoxy-benzylamide;

1-Methyl-2,4-dioxo-3-(E)-pent-2-enyl-1,2,3,4-tetrahydro-thieno[2,3-d]pyrimidine-6-carboxylic acid 4-methoxy-benzylamide;

1-Methyl-2,4-dioxo-3-(2-phenylsulfanyl-ethyl)-1,2,3,4-tetrahydro-[2,3-d]pyrimidine-6-carboxylic acid 4-methoxy-benzylamide;

3-sec-Butyl-1-methyl-2,4-dioxo-1,2,3,4-tetrahydro-thieno [2,3-d]pyrimidine-6-carboxylic acid 4-methoxy-benzylamide;

1-Methyl-3-(2-methyl-allyl)-2,4-dioxo-1,2,3,4-tetrahydro-thieno[2,3-d]pyrimidine-6-carboxylic acid 4-methoxy-benzylamide;

3-(1-Ethyl-propyl)-1-methyl-2,4-dioxo-1,2,3,4-tetrahydro-thieno[2,3-d]pyrimidine-6-carboxylic acid 4-methoxy-benzylamide;

1-Methyl-2,4-dioxo-3-pent-2-ynyl-1,2,3,4-tetrahydro-thieno [2,3-d]pyrimidine-6-carboxylic acid 4-methoxy-benzylamide;

3-(2-Benzenesulfonyl-ethyl)-1-methyl-2,4-dioxo-1,2,3,4-tetrahydro-[2,3-d]pyrimidine-6-carboxylic acid 4-methoxy-benzylamide;

1-Methyl-3-(3-methyl-but-2-enyl)-2,4-dioxo-1,2,3,4-tetrahydro-[2,3-d]pyrimidine-6-carboxylic acid 4-methoxy-benzylamide;

3-[2-(4-Fluoro-benzenesulfonyl)-ethyl]-1-methyl-2,4-dioxo-1,2,3,4-tetrahydro-thieno[2,3-d]pyrimidine-6-carboxylic acid 4-methoxy-benzylamide;

1-Methyl-2,4-dioxo-3-[2-(toluene-4-sulfonyl)-ethyl]-1,2,3,4-tetrahydro-[2,3-d]pyrimidine-6-carboxylic acid 4-methoxy-benzylamide;

3-[3-(4-Fluoro-phenyl)-3-oxo-propyl]-1-methyl-2,4-dioxo-1,2,3,4-tetrahydro-thieno[2,3-d]pyrimidine-6-carboxylic acid 4-methoxy-benzylamide;

3-[3-(4-Chloro-phenyl)-3-oxo-propyl]-11-methyl-2,4-dioxo-1,2,3,4-tetrahydro-thieno[2,3-d]pyrimidine-6-carboxylic acid 4-methoxy-benzylamide;

3-(2-Benzoylamino-ethyl)-1-methyl-2,4-dioxo-1,2,3,4-tetrahydro-[2,3-d]pyrimidine-6-carboxylic acid 4-methoxy-benzylamide;

3-[2-(4-Bromo-phenoxy)-ethyl]-1-methyl-2,4-dioxo-1,2,3,4-tetrahydro-[2,3-d]pyrimidine-6-carboxylic acid 4-methoxy-benzylamide;

3-Benzofurazan-5-ylmethyl-1-methyl-2,4-dioxo-1,2,3,4-tetrahydro-[2,3-d]pyrimidine-6-carboxylic acid 4-methoxy-benzylamide;

1-Methyl-2,4-dioxo-3-(2-phenoxy-ethyl)-1,2,3,4-tetrahydro-thieno[2,3-d]pyrimidine-6-carboxylic acid 4-methoxy-benzylamide;

{5-[6-(4-Methoxy-benzylcarbamoyl)-1-methyl-2,4-dioxo-1,4-dihydro-2H-[2,3-d]pyrimidin-3-ylmethyl]-isoxazol-3-yl}-carbamic acid methyl ester;

3-Benzyloxycarbonylamino-5-[6-(4-methoxy-benzylcarbamoyl)-1-methyl-2,4-dioxo-1,4-dihydro-2H-thieno[2,3-d]pyrimidin-3-yl]-4-oxo-pentanoic acid tert-butyl ester;

3-[2-(4-Chloro-phenylsulfanyl)-ethyl]-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-(1-phenyl-ethyl)-1,2,3,4-tetrahydro-thieno[2,3-d]pyrimidine-6-carboxylic acid 3-methoxy-benzylamide;

1-Methyl-2,4-dioxo-3-(E)-pent-2-enyl-1,2,3,4-tetrahydro-thieno[2,3-d]pyrimidine-6-carboxylic acid 3-methoxy-benzylamide;

3-(2-Ethyl-hexyl)-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-(2-phenylmethanesulfonyl-ethyl)-1,2,3,4-tetrahydro-thieno[2,3-d]pyrimidine-6-carboxylic acid 3-methoxy-benzylamide;

3-(5-Cyano-pentyl)-1-methyl-2,4-dioxo-1,2,3,4-tetrahydro-thieno[2,3-d]pyrimidine-6-carboxylic acid 3-methoxy-benzylamide;

3-(E)-But-2-enyl-1-methyl-2,4-dioxo-1,2,3,4-tetrahydro-thieno[2,3-d]pyrimidine-6-carboxylic acid 3-methoxy-benzylamide;

1-Methyl-3-(2-naphthalen-1-yl-ethyl)-2,4-dioxo-1,2,3,4-tetrahydro-[2,3-d]pyrimidine-6-carboxylic acid 3-methoxy-benzylamide;

1-Methyl-2,4-dioxo-3-(E)-pent-2-enyl-1,2,3,4-tetrahydro-thieno[2,3-d]pyrimidine-6-carboxylic acid 3-methoxy-benzylamide;

1-Methyl-2,4-dioxo-3-(2-phenylsulfanyl-ethyl)-1,2,3,4-tetrahydro-[2,3-d]pyrimidine-6-carboxylic acid 3-methoxy-benzylamide;

3-sec-Butyl-1-methyl-2,4-dioxo-1,2,3,4-tetrahydro-thieno[2,3-d]pyrimidine-6-carboxylic acid 3-methoxy-benzylamide;

1-Methyl-3-(2-methyl-allyl)-2,4-dioxo-1,2,3,4-tetrahydro-thieno[2,3-d]pyrimidine-6-carboxylic acid 3-methoxy-benzylamide;

3-(1-Ethyl-propyl)-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-pent-2-ynyl-1,2,3,4-tetrahydro-thieno[2,3-d]pyrimidine-6-carboxylic acid 3-methoxy-benzylamide;

1-Methyl-3-(3-methyl-but-2-enyl)-2,4-dioxo-1,2,3,4-tetrahydro-[2,3-d]pyrimidine-6-carboxylic acid 3-methoxy-benzylamide;

1-Methyl-2,4-dioxo-3-[2-(toluene-4-sulfonyl)-ethyl]-1,2,3,4-tetrahydro-[2,3-d]pyrimidine-6-carboxylic acid 3-methoxy-benzylamide;

3-(2-Benzoylamino-ethyl)-1-methyl-2,4-dioxo-1,2,3,4-tetrahydro-[2,3-d]pyrimidine-6-carboxylic acid 3-methoxy-benzylamide;

3-[2-(4-Bromo-phenoxy)-ethyl]-1-methyl-2,4-dioxo-1,2,3,4-tetrahydro-[2,3-d]pyrimidine-6-carboxylic acid 3-methoxy-benzylamide;

3-Benzofurazan-5-ylmethyl-1-methyl-2,4-dioxo-1,2,3,4-tetrahydro-[2,3-d]pyrimidine-6-carboxylic acid 3-methoxy-benzylamide;

1-Methyl-2,4-dioxo-3-(2-phenoxy-ethyl)-1,2,3,4-tetrahydro-thieno[2,3-d]pyrimidine-6-carboxylic acid 3-methoxy-benzylamide;

{5-[6-(3-Methoxy-benzylcarbamoyl)-1-methyl-2,4-dioxo-1,4-dihydro-2H-[2,3-d]pyrimidin-3-ylmethyl]-isoxazol-3-yl}-carbamic acid methyl ester; and

3-Benzyloxycarbonylamino-5-[6-(3-methoxy-benzylcarbamoyl)-1-methyl-2,4-dioxo-1,4-dihydro-2H-thieno[2,3-d]pyrimidin-3-yl]-4-oxo-pentanoic acid tert-butyl ester.

40. The combination according to Embodiment 23, wherein the compound of Formula IB is selected from:

3-Benzyl-1-methyl-2,4-dioxo-1,2,3,4-tetrahydro-thieno[2,3-d]pyrimidine-6-carboxylic acid methyl ester;

3-(4-Bromo-benzyl)-5-methyl-2,4-dioxo-1,2,3,4-tetrahydro-thieno[2,3-d]pyrimidine-6-carboxylic acid benzyl ester;

3-(4-Fluoro-benzyl)-5-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno [2,3-d]pyrimidine-6-carboxylic acid pyridin-4-ylmethyl ester;

3-Benzyl-1-methyl-2,4-dioxo-1,2,3,4-tetrahydro-thieno[2,3-d]pyrimidine-6-carboxylic acid benzo[b]thiophen-2-ylmethyl ester;

3-Benzyl-1-methyl-2,4-dioxo-1,2,3,4-tetrahydro-thieno[2,3-d]pyrimidine-6-carboxylic acid 1-methyl-1H-indol-5-ylmethyl ester;

3-Benzyl-2,4-dioxo-1,2,3,4-tetrahydro-thieno[2,3-d]pyrimidine-6-carboxylic acid thiophen-3-ylmethyl ester;

3-1,3-Benzodioxol-5-ylmethyl-1-methyl-2,4-dioxo-1,2,3,4-tetrahydro-[2,3-d]pyrimidine-6-carboxylic acid benzyl ester;

1-Methyl-2,4-dioxo-3-pyridin-4-ylmethyl-1,2,3,4-tetrahydro-thieno[2,3-d]pyrimidine-6-carboxylic acid benzyl ester;

3-(4-tert-Butyl-benzyl)-1-methyl-2,4-dioxo-1,2,3,4-tetrahydro-thieno[2,3-d]pyrimidine-6-carboxylic acid benzyl ester;

3-(3,4-Dichloro-benzyl)-5-methyl-2,4-dioxo-1,2,3,4-tetrahydro-thieno[2,3-d]pyrimidine-6-carboxylic acid benzyl ester;

1-Methyl-2,4-dioxo-3-(4-trifluoromethoxy-benzyl)-1,2,3,4-tetrahydro-[2,3-d]pyrimidine-6-carboxylic acid benzyl ester;

1-Methyl-3-naphthalen-2-ylmethyl-2,4-dioxo-1,2,3,4-tetrahydro-[2,3-d]pyrimidine-6-carboxylic acid benzyl ester;

3-(4-Cyano-benzyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzofuran-5-ylmethyl ester;

3-(3,5-Dimethoxy-benzyl)-1-methyl-2,4-dioxo-1,2,3,4-tetrahydro-[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid benzyl ester;

3-(3,5-Dinitro-benzyl)-1-methyl-2,4-dioxo-1,2,3,4-tetrahydro-thieno[2,3-d]pyrimidine-6-carboxylic acid benzyl ester;

3-Benzyl-1-methyl-2,4-dioxo-1,2,3,4-tetrahydro-thieno[2,3-d]pyrimidine-6-carboxylic acid; and

3-(4-Carboxy-benzyl)-1-methyl-2,4-dioxo-1,2,3,4-tetrahydro-thieno[2,3-d]pyrimidine-6-carboxylic acid 2-ethoxy-benzyl ester.

41. The combination according to Embodiment 23, wherein the compound of Formula IB is selected from:

3-Benzyl-1-methyl-2,4-dioxo-1,2,3,4-tetrahydro-thieno[2,3-d]pyrimidine-6-carboxylic acid [2-(3,4-dimethoxy-phenyl)-ethyl]-amide;

3-Benzyl-1-methyl-2,4-dioxo-1,2,3,4-tetrahydro-thieno[2,3-d]pyrimidine-6-carboxylic acid 4-amino-benzylamide;

3-Benzyl-1-methyl-2,4-dioxo-1,2,3,4-tetrahydro-thieno[2,3-d]pyrimidine-6-carboxylic acid [2-(4-chloro-phenyl)-ethyl]-amide;

3-Benzyl-1-methyl-2,4-dioxo-1,2,3,4-tetrahydro-thieno[2,3-d]pyrimidine-6-carboxylic acid (biphenyl-2-ylmethyl)-amide;

3-Benzyl-1-methyl-2,4-dioxo-1,2,3,4-tetrahydro-thieno[2,3-d]pyrimidine-6-carboxylic acid 3,4-dimethoxy-benzylamide;

3-Benzyl-1-methyl-2,4-dioxo-1,2,3,4-tetrahydro-thieno[2,3-d]pyrimidine-6-carboxylic acid (2-pyridin-4-yl-ethyl)-amide;

3-Benzyl-1-methyl-2,4-dioxo-1,2,3,4-tetrahydro-thieno[2,3-d]pyrimidine-6-carboxylic acid 2-difluoromethoxy-benzylamide;

3-Benzyl-1-methyl-2,4-dioxo-1,2,3,4-tetrahydro-thieno[2,3-d]pyrimidine-6-carboxylic acid [2-(3-ethoxy-phenyl)-ethyl]-amide;

3-Benzyl-1-methyl-2,4-dioxo-1,2,3,4-tetrahydro-thieno[2,3-d]pyrimidine-6-carboxylic acid 3-chloro-4-fluoro-benzylamide;

3-Benzyl-1-methyl-2,4-dioxo-1,2,3,4-tetrahydro-thieno[2,3-d]pyrimidine-6-carboxylic acid 2,4-dichloro-benzylamide;

3-Benzyl-1-methyl-2,4-dioxo-1,2,3,4-tetrahydro-thieno[2,3-d]pyrimidine-6-carboxylic acid (2-phenyl-propyl)-amide;

3-Benzyl-1-methyl-2,4-dioxo-1,2,3,4-tetrahydro-thieno[2,3-d]pyrimidine-6-carboxylic acid 3,4,5-trimethoxy-benzylamide;

3-Benzyl-1-methyl-2,4-dioxo-1,2,3,4-tetrahydro-thieno[2,3-d]pyrimidine-6-carboxylic acid 3-chloro-benzylamide;

3-Benzyl-1-methyl-2,4-dioxo-1,2,3,4-tetrahydro-thieno[2,3-d]pyrimidine-6-carboxylic acid 3,5-dimethoxy-benzylamide;

3-Benzyl-1-methyl-2,4-dioxo-1,2,3,4-tetrahydro-thieno[2,3-d]pyrimidine-6-carboxylic acid 2,3-dimethoxy-benzylamide;

3-Benzyl-1-methyl-2,4-dioxo-1,2,3,4-tetrahydro-thieno[2,3-d]pyrimidine-6-carboxylic acid 3-trifluoromethyl-benzylamide;

3-Benzyl-1-methyl-2,4-dioxo-1,2,3,4-tetrahydro-thieno[2,3-d]pyrimidine-6-carboxylic acid 2-methoxy-benzylamide;

3-Benzyl-1-methyl-2,4-dioxo-1,2,3,4-tetrahydro-thieno[2,3-d]pyrimidine-6-carboxylic acid 2-methyl-benzylamide;

3-Benzyl-1-methyl-2,4-dioxo-1,2,3,4-tetrahydro-thieno[2,3-d]pyrimidine-6-carboxylic acid (4-phenyl-butyl)-amide;

3-Benzyl-1-methyl-2,4-dioxo-1,2,3,4-tetrahydro-thieno[2,3-d]pyrimidine-6-carboxylic acid (pyridin-3-ylmethyl)-amide;

3-Benzyl-1-methyl-2,4-dioxo-1,2,3,4-tetrahydro-thieno[2,3-d]pyrimidine-6-carboxylic acid 4-methoxy-benzylamide;

3-Benzyl-1-methyl-2,4-dioxo-1,2,3,4-tetrahydro-thieno[2,3-d]pyrimidine-6-carboxylic acid ((S)-2,2-dimethyl-4-phenyl-1,3-dioxinan-5-yl)-amide;

3-Benzyl-1-methyl-2,4-dioxo-1,2,3,4-tetrahydro-thieno[2,3-d]pyrimidine-6-carboxylic acid [2-(3-methoxy-phenyl)-ethyl]-amide;

3-Benzyl-1-methyl-2,4-dioxo-1,2,3,4-tetrahydro-thieno[2,3-d]pyrimidine-6-carboxylic acid 3-methoxy-benzylamide;

3-Benzyl-1-methyl-2,4-dioxo-1,2,3,4-tetrahydro-thieno[2,3-d]pyrimidine-6-carboxylic acid (thiophen-2-ylmethyl)-amide;

3-Benzyl-1-methyl-2,4-dioxo-1,2,3,4-tetrahydro-thieno[2,3-d]pyrimidine-6-carboxylic acid 2-chloro-benzylamide;

3-Benzyl-1-methyl-2,4-dioxo-1,2,3,4-tetrahydro-thieno[2,3-d]pyrimidine-6-carboxylic acid (5-methyl-furan-2-ylmethyl)-amide;

3-Benzyl-1-methyl-2,4-dioxo-1,2,3,4-tetrahydro-thieno[2,3-d]pyrimidine-6-carboxylic acid (2,2-diphenyl-ethyl)-amide;

3-Benzyl-1-methyl-2,4-dioxo-1,2,3,4-tetrahydro-thieno[2,3-d]pyrimidine-6-carboxylic acid [2-(2-methoxy-phenyl)-ethyl]-amide;

3-Benzyl-1-methyl-2,4-dioxo-1,2,3,4-tetrahydro-thieno[2,3-d]pyrimidine-6-carboxylic acid [2-(3-trifluoromethyl-phenyl)-ethyl]-amide;

3-Benzyl-1-methyl-2,4-dioxo-1,2,3,4-tetrahydro-thieno[2,3-d]pyrimidine-6-carboxylic acid 4-bromo-benzylamide;

3-Benzyl-1-methyl-2,4-dioxo-1,2,3,4-tetrahydro-thieno[2,3-d]pyrimidine-6-carboxylic acid [2-(1H-indol-3-yl)-ethyl]-amide;

3-Benzyl-1-methyl-2,4-dioxo-1,2,3,4-tetrahydro-thieno[2,3-d]pyrimidine-6-carboxylic acid 3,5-dichloro-benzylamide;

3-Benzyl-1-methyl-2,4-dioxo-1,2,3,4-tetrahydro-thieno[2,3-d]pyrimidine-6-carboxylic acid indan-1-ylamide;

3-Benzyl-1-methyl-2,4-dioxo-1,2,3,4-tetrahydro-thieno[2,3-d]pyrimidine-6-carboxylic acid (furan-2-ylmethyl)-amide;

3-Benzyl-1-methyl-2,4-dioxo-1,2,3,4-tetrahydro-thieno[2,3-d]pyrimidine-6-carboxylic acid [2-(4-methoxy-phenyl)-ethyl]-amide;

3-Benzyl-1-methyl-2,4-dioxo-1,2,3,4-tetrahydro-thieno[2,3-d]pyrimidine-6-carboxylic acid 2,4-dimethoxy-benzylamide;

3-Benzyl-1-methyl-2,4-dioxo-1,2,3,4-tetrahydro-thieno[2,3-d]pyrimidine-6-carboxylic acid 4-chloro-benzylamide;

3-Benzyl-1-methyl-2,4-dioxo-1,2,3,4-tetrahydro-thieno[2,3-d]pyrimidine-6-carboxylic acid (1-phenyl-ethyl)-amide;

3-Benzyl-1-methyl-2,4-dioxo-1,2,3,4-tetrahydro-thieno[2,3-d]pyrimidine-6-carboxylic acid 3,4-dichloro-benzylamide;

3-Benzyl-1-methyl-2,4-dioxo-1,2,3,4-tetrahydro-thieno[2,3-d]pyrimidine-6-carboxylic acid 4-fluoro-3-trifluoromethyl-benzylamide;

3-Benzyl-1-methyl-2,4-dioxo-1,2,3,4-tetrahydro-thieno[2,3-d]pyrimidine-6-carboxylic acid (2-pyridin-2-yl-ethyl)-amide;

3-Benzyl-1-methyl-2,4-dioxo-1,2,3,4-tetrahydro-thieno[2,3-d]pyrimidine-6-carboxylic acid [2-(2,4-dimethyl-phenyl)-ethyl]-amide;

3-Benzyl-1-methyl-2,4-dioxo-1,2,3,4-tetrahydro-thieno[2,3-d]pyrimidine-6-carboxylic acid [2-(2,4-dichloro-phenyl)-ethyl]-amide;

1,3-Dimethyl-2,4-dioxo-1,2,3,4-tetrahydro-thieno[2,3-d]pyrimidine-6-carboxylic acid 3-methoxy-benzylamide;

3-Cyanomethyl-1-methyl-2,4-dioxo-1,2,3,4-tetrahydro-thieno[2,3-d]pyrimidine-6-carboxylic acid 4-methoxy-benzylamide;

3-Benzyl-1-methyl-2,4-dioxo-1,2,3,4-tetrahydro-thieno[2,3-d]pyrimidine-6-carboxylic acid 3-methoxy-benzylamide;

3-(4-Cyclopropylsulfamoyl-benzyl)-1-methyl-2,4-dioxo-1,2,3,4-tetrahydro-thieno[2,3-d]pyrimidine-6-carboxylic acid 3-methoxy-benzylamide;

1-Methyl-3-(6-nitro-pyridin-3-ylmethyl)-2,4-dioxo-1,2,3,4-tetrahydro-[2,3-d]pyrimidine-6-carboxylic acid 3-methoxy-benzylamide;

1-Methyl-3-(6-nitro-pyridin-3-ylmethyl)-2,4-dioxo-1,2,3,4-tetrahydro-[2,3-d]pyrimidine-6-carboxylic acid 4-methoxy-benzylamide;

1-Methyl-3-(6-nitro-pyridin-3-ylmethyl)-2,4-dioxo-1,2,3,4-tetrahydro-[2,3-d]pyrimidine-6-carboxylic acid (2-methoxy-pyridin-4-ylmethyl)-amide;

3-Cyclohexylmethyl-1-methyl-2,4-dioxo-1,2,3,4-tetrahydro-thieno[2,3-d]pyrimidine-6-carboxylic acid (2-methoxy-pyridin-4-ylmethyl)-amide;

3-{2-[(1H-Benzimidazole-5-carbonyl)-aniino]-ethyl}-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-[2-(3-piperidin-1-yl-propionylamino)-ethyl]-1,2,3,4-tetrahydro-thieno[2,3-d]pyrimidine-6-carboxylic acid 3-methoxy-benzylamide;

1-Methyl-2,4-dioxo-3-{2-[(6-pyrazol-1-yl-pyridine-3-carbonyl)-amino]-ethyl}-1,2,3,4-tetrahydro-thieno[2,3-d]pyrimidine-6-carboxylic acid 3-methoxy-benzylamide;

3-[2-(4-Diethylamino-benzoylamino)-ethyl]-1-methyl-2,4-dioxo-1,2,3,4-tetrahydro-thieno[2,3-d]pyrimidine-6-carboxylic acid 3-methoxy-benzylamide;

3-{2-[(6-Chloro-pyridine-3-carbonyl)-amino]-ethyl}-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-{2-[(1H-pyrrole-2-carbonyl)-amino]-ethyl}-1,2,3,4-tetrahydro-thieno[2,3-d]pyrimidine-6-carboxylic acid 3-methoxy-benzylamide;

3-[2-(2-Dimethylamino-acetylamino)-ethyl]-1-methyl-2,4-dioxo-1,2,3,4-tetrahydro-thieno[2,3-d]pyrimidine-6-carboxylic acid 3-methoxy-benzylamide;

1-Methyl-2,4-dioxo-3-{2-[(pyrazine-2-carbonyl)-amino]-ethyl}-1,2,3,4-tetrahydro-thieno[2,3-d]pyrimidine-6-carboxylic acid 3-methoxy-benzylamide;

1-Methyl-3-[2-(2-methyl-2-methylamino-propionylamino)-ethyl]-2,4-dioxo-1,2,3,4-tetrahydro-thieno[2,3-d]pyrimidine-6-carboxylic acid 3-methoxy-benzylamide;

1-Methyl-2,4-dioxo-3-{2-[(pyrrolidine-2-carbonyl)-amino]-ethyl}-1,2,3,4-tetrahydro-thieno[2,3-d]pyrimidine-6-carboxylic acid 3-methoxy-benzylamide;

1-Methyl-2,4-dioxo-3-{2-[3-(5-phenyl-1H-pyrrol-2-yl)-propionylamino]-ethyl}-1,2,3,4-tetrahydro-thieno[2,3-d]pyrimidine-6-carboxylic acid 3-methoxy-benzylamide;

1-Methyl-2,4-dioxo-3-{2-[2-(pyridin-4-ylsulfanyl)-acetylamino]-ethyl}-1,2,3,4-tetrahydro-thieno[2,3-d]pyrimidine-6-carboxylic acid 3-methoxy-benzylamide;

3-(6-Amino-pyridin-3-ylmethyl)-1-methyl-2,4-dioxo-1,2,3,4-tetrahydro-[2,3-d]pyrimidine-6-carboxylic acid 3-methoxy-benzylamide;

1-Methyl-2,4-dioxo-3-(3-phenyl-prop-2-ynyl)-1,2,3,4-tetrahydro-[2,3-d]pyrimidine-6-carboxylic acid 4-methoxy-benzylamide;

3-(6-Amino-pyridin-3-ylmethyl)-1-methyl-2,4-dioxo-1,2,3,4-tetrahydro-[2,3-d]pyrimidine-6-carboxylic acid 4-methoxy-benzylamide;

3-(6-Amino-pyridin-3-ylmethyl)-1-methyl-2,4-dioxo-1,2,3,4-tetrahydro-[2,3-d]pyrimidine-6-carboxylic acid (pyridin-4-ylmethyl)-amide;

1-Methyl-2,4-dioxo-3-[2-(pyridin-2-ylamino)-ethyl]-1,2,3,4-tetrahydro-[2,3-d]pyrimidine-6-carboxylic acid 4-methoxy-benzylamide;

1-Methyl-2,4-dioxo-3-[2-(pyrimidin-2-ylamino)-ethyl]-1,2,3,4-tetrahydro-[2,3-d]pyrimidine-6-carboxylic acid 4-methoxy-benzylamide; and

1-Methyl-2,4-dioxo-3-[2-(pyrimidin-2-ylamino)-ethyl]-1,2,3,4-tetrahydro-[2,3-d]pyrimidine-6-carboxylic acid 3-methoxy-benzylamide; or a pharmaceutically acceptable salt thereof.

42. A combination, comprising valdecoxib, or a pharmaceutically acceptable salt thereof, and an allosteric carboxylic inhibitor of MMP-13 of Formula IC

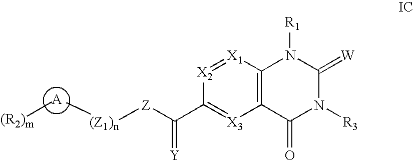

or a pharmaceutically acceptable salt thereof, or an N-oxide thereof,

in which:

R₁ represents a group selected from:

hydrogen, amino,

(C₁-C₆)alkyl, (C₃-C₆)alkenyl, (C₃-C₆)alkynyl, mono(C₁-C₆)alkylamino(C₁-C₆)alkyl, di(C₁-C₆)alkylamino(C₁-C₆)alkyl, aryl, aryl(C₁-C₆)alkyl, heterocycle, and 3- to 6-membered cycloalkyl(C₁-C₆)alkyl, these groups being unsubstituted or substituted with one or more groups, which may be identical or different, selected from amino, (C₁-C₆)alkyl, cyano, halo(C₁-C₆)alkyl, C(═O)OR₄, OR₄ and SR₄, in which R₄ represents hydrogen or (C₁-C₆)alkyl,

W represents an oxygen atom, a sulphur atom, or a group ═N—R′, in which R′ represents (C₁-C₆)alkyl, hydroxyl, or cyano,

X₁, X₂ and X₃ represent, independently of each other, a nitrogen atom or a group —C—R₆ in which R₆ represents a group selected from hydrogen, (C₁-C₆)alkyl, amino, mono(C₁-C₆)alkylamino, di(C₁-C₆)alkylamino, hydroxyl, (C₁-C₆)alkoxy, and halogen,

with the proviso that not more than two of the groups X₁, X₂ and X₃ simultaneously represent a nitrogen atom,

Y represents a group selected from oxygen atom, sulphur atom, —NH, and —N(C₁-C₆)alkyl,

Z represents:

an oxygen atom, a sulphur atom,

or a group —NR₇ in which R₇ represents a group selected from hydrogen, (C₁-C₆)alkyl, aryl(C₁-C₆)alkyl, cycloalkyl, aryl, and heteroaryl, and

when Y is an oxygen atom, a sulphur atom, or a group —N(C₁-C₆)alkyl, Z optionally represents a carbon atom which is unsubstituted or substituted with a (C₁-C₆)alkyl, an aryl, an aryl(C₁-C₆)alkyl, an aromatic or non-aromatic heterocycle or a cycloalkyl,

n is an integer from 1 to 8 inclusive,

Z₁ represents —CR₈R₉ wherein R₈ and R₉, independently of each other, represent a group selected from hydrogen, (C₁-C₆)alkyl, halo(C₁-C₆)alkyl, halogen, amino, OR₄, SR₄ or C(═O)OR₄ in which R₄ represents a hydrogen or (C₁-C₆)alkyl, and

when n is greater than or equal to 2, the hydrocarbon chain Z₁ optionally contains one or more multiple bonds,

and/or one of the carbon atoms in the hydrocarbon chain Z₁ may be replaced with an oxygen atom, a sulphur atom which is unsubstituted or substituted with one or two oxygen atoms, or a nitrogen atom which is unsubstituted or substituted with a (C₁-C₆)alkyl,

and when one of the carbon atoms in the hydrocarbon chain Z₁ is replaced with a sulphur atom which is unsubstituted or substituted with one or two oxygen atoms, then the group —C(═Y)-Z- optionally may be absent in the general formula (I),

A represents a group selected from:

aromatic or non-aromatic, 5- or 6-membered monocycle comprising from 0 to 4 heteroatoms selected from nitrogen, oxygen and sulphur, and

bicycle, composed of two aromatic or non-aromatic, 5- or 6-membered rings, which may be identical or different, comprising from 0 to 4 heteroatoms selected from nitrogen, oxygen and sulphur,

m is an integer from 0 to 7 inclusive,

the group(s) R₂, which may be identical or different, is (are) selected from (C₁-C₆)alkyl, halogen, —CN, NO₂, SCF₃, —CF₃, —OCF₃, —NR₁₀R₁₁, —OR₁₀, —SR₁₀, —SOR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:

X₅ represents a group selected from oxygen, sulphur optionally substituted by one or two oxygen atoms, and nitrogen substituted by hydrogen or (C₁-C₆)alkyl,

k is an integer from 0 to 3 inclusive,

R₁₀ and R₁₁, which may be identical or different, are selected from hydrogen and (C₁-C₆)alkyl,

X₄ represents a group selected from single bond, —CH₂—, oxygen atom, sulphur atom optionally substituted by one or two oxygen atoms, and nitrogen atom substituted by hydrogen atom or (C₁-C₆)alkyl group,

R₁₂ represents an aromatic or non-aromatic, heterocyclic or non-heterocyclic, 5- or 6-membered ring which is unsubstituted or substituted with one or more groups, which may be identical or different, selected from (C₁-C₆)alkyl, halogen, hydroxyl and amino, and when the ring is heterocyclic, it comprises from 1 to 4 beteroatoms selected from nitrogen, oxygen and sulphur;

R₃ represents a group selected from:

hydrogen,

(C₁-C₆)alkyl, (C₃-C₆)alkenyl, (C₃-C₆)alkynyl, these groups being unsubstituted or substituted with one or more groups, which may be identical or different, selected from amino, cyano, halo(C₁-C₆)alkyl, cycloalkyl, —C(═O)NR₁₀R₁₁, —C(═O)OR₁₀, OR₁₀, and SR₁₀, in which R₁₀ and R₁₁, which may be identical or different, represent hydrogen or (C₁-C₆)alkyl,

and the group of formula:

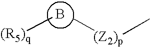

in which p is an integer from 0 to 8 inclusive,

Z₂ represents —CR₁₃R₁₄ wherein R₁₃ and R₁₄, independently of each other, represent a group selected from hydrogen, (C₁-C₆)alkyl, phenyl, halo(C₁-C₆)alkyl, halogen, amino, OR₄, SR₄ and —C(═O)OR₄ in which R₄ represents hydrogen or (C₁-C₆)alkyl, and

when p is greater than or equal to 2, the hydrocarbon chain Z₂ optionally contains one or more multiple bonds,

and/or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl, or a carbonyl group,

B represents a group selected from:

an aromatic or non-aromatic 5- or 6-membered monocycle comprising from 0 to 4 heteroatoms selected from nitrogen, oxygen and sulphur, and

a bicycle, composed of two aromatic or non-aromatic, 5- or 6-membered rings, which may be identical or different, comprising from 0 to 4 heteroatoms selected from nitrogen, oxygen and sulphur,

q is an integer from 0 to 7 inclusive,

the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, (CH₂)ₖC(═O)OR₁₅, —C(═O)O—(CH₂)ₖ₂—NR₁₅R₁₆, —C(═O)O—(CH₂)ₖ₂—C(═O)OR₁₈, —X₇(CH₂)ₖC(═O)NR₁₅R₁₆, —(CH₂)ₖC(═O)NR₁₅R₁₆, —R₁₉—C(═O)OR₁₅, —X₆—R₂₀, and —C(═O)—R₂₁—NR₁₅R₁₆ in which:

—X₇ represents a group selected from oxygen atom, sulphur atom optionally substituted by one or two oxygen atoms, and nitrogen atom substituted by a hydrogen atom or a (C₁-C₆)alkyl group,

k is an integer from 0 to 3 inclusive,

k1 is an integer from 0 to 2 inclusive,

k2 is an integer from 1 to 4 inclusive,

—R₁₅, R₁₆ and R₁₇, which may be identical or different, are selected from hydrogen and (C₁-C₆)alkyl,

R₁₈ represents a group selected from (C₁-C₆)alkyl, —R₂₁—NR₁₅R₁₆, —R₂, —NR₁₅—C(═O)—R₂₁—NR₁₆R₁₇, and —C(═O)O—R₂₁—NR₁₅R₁₆ in which R₂₁ represents a linear or branched (C₁-C₆)alkylene group, and R₁₅, R₁₆ and R₁₇ are as defined hereinbefore,

R₁₉ represents a (C₃-C₆)cycloalkyl group,

X₆ represents a group selected from single bond, —CH₂—, oxygen atom, sulphur atom optionally substituted by one or two oxygen atoms, and nitrogen atom substituted by hydrogen atom or (C₁-C₆)alkyl group,

R₂₀ represents an aromatic or non-aromatic, heterocyclic or non-heterocyclic, 5- or 6-membered ring, which is unsubstituted or substituted with one or more groups, which may be identical or different, selected from (C₁-C₆)alkyl, halogen, hydroxyl, oxo, cyano, tetrazole, amino, and —C(═O)OR4 wherein R₄ represents hydrogen or (C₁-C₆)alkyl, and, when the ring is heterocyclic, it comprises from 1 to 4 heteroatoms selected from nitrogen, oxygen and sulphur,

with the proviso that when X₁ represents a nitrogen atom, X₂ cannot represent a carbon atom substituted with a methyl group or with NH—CH₃.

43. The combination according to Embodiment 42, wherein the compound of Formula IC is selected from:

4-[6-(4-Methoxy-benzylcarbamoyl)-1-methyl-2,4-dioxo-1,4-dihydro-2H-[3,4-d]pyrimidin-3-ylmethyl]-benzoic acid;

3-Benzyl-1-methyl-2,4-dioxo-1,2,3,4-tetrahydro-pyrido[3,4-d]pyrimidine-6-carboxylic acid (1,3-benzodioxol-5-ylmethyl)-amide;

4-[6-(4-Fluoro-benzylcarbamoyl)-1-methyl-2,4-dioxo-1,4-dihydro-2H-quinazolin-3-ylmethyl]-benzoic acid;

1-Methyl-2,4-dioxo-3-[4-(5-oxo-4,5-dihydro-1,2,4-oxadiazol-3-yl)-benzyl]-1,2,3,4-tetrahydro-quinazoline-6-carboxylic acid 4-methoxy-benzylamide;

4-[6-(4-Methoxy-benzylcarbamoyl)-1-methyl-2,4-dioxo-1,4-dihydro-2H-quinazolin-3-ylmethyl]-benzoic acid hemi calcium salt;

Methyl 4-[6-(4-Methoxy-benzylcarbamoyl)-1-methyl-2,4-dioxo-1,4-dihydro-2H-pyrido[3,4-d]pyrimidin-3-ylmethyl]-benzoate;

4-[6-(3-Methoxy-benzylcarbamoyl)-1-methyl-2,4-dioxo-1,4-dihydro-2H-quinazolin-3-ylmethyl]-benzoic acid;

1-Methyl-2,4-dioxo-3-[4-(2H-tetrazol-5-yl)-benzyl]-1,2,3,4-tetrahydro-quinazoline-6-carboxylic acid 4-methoxy-benzylamide;

Methyl 2-hydroxy-4-[6-(4-methoxy-benzylcarbamoyl)-1-methyl-2,4-dioxo-1,4-dihydro-2H-quinazolin-3-ylmethyl]-benzoate;

3-(4-Chloro-benzyl)-1-methyl-2,4-dioxo-1,2,3,4-tetrahydroquinazoline-6-carboxylic acid 3-methoxy-benzylamide;

4-{6-[(1,3-Benzodioxol-5-ylmethyl)-carbamoyl]-1-methyl-2,4-dioxo-1,4-dihydro-2H-quinazolin-3-ylmethyl}-benzoic acid;

2-Hydroxy-4-[6-(4-methoxy-benzylcarbamoyl)-1-methyl-2,4-dioxo-1,4-dihydro-2H-quinazolin-3-ylmethyl]-benzoic acid;

Methyl 4-[6-(3-methoxy-benzylcarbamoyl)-1-methyl-2,4-dioxo-1,4-dihydro-2H-quinazolin-3-ylmethyl]-benzoate;

3-(3-Fluoro-benzyl)-1-methyl-2,4-dioxo-1,2,3,4-tetrahydro-quinazoline-6-carboxylic acid 3-methoxy-benzylamide;

4-Pyridylmethyl 3-benzyl-2,4-dioxo-1,2,3,4-tetrahydroquinazoline-6-carboxylate;

Methyl 4-{6-[(1,3-benzodioxol-5-ylmethyl)-carbamoyl]-1-methyl-2,4-dioxo-1,4-dihydro-2H-quinazolin-3-ylmethyl}-benzoate;

1-Methyl-3-[4-(5-methyl-1,2,4-oxadiazol-3-yl)-benzyl]-2,4-dioxo-1,2,3,4-tetrahydro-quinazoline-6-carboxylic acid 4-methoxy-benzylamide;

1-Methyl-3-[4-(3-methyl-1,2,4-oxadiazol-5-yl)-benzyl]-2,4-dioxo-1,2,3,4-tetrahydro-quinazoline-6-carboxylic acid 4-methoxy-benzylamide;

3-(3-Fluoro-benzyl)-1-methyl-2,4-dioxo-1,2,3,4-tetrahydro-quinazoline-6-carboxylic acid (2-methoxy-pyridin-4-ylmethyl)-amide;

4-[6-(4-Methoxy-benzylcarbamoyl)-1-methyl-2,4-dioxo-1,4-dihydro-2H]-quinazolin-3-ylmethyl]-benzoic acid;

1-{4-[6-(4-Methoxy-benzylcarbamoyl)-1-methyl-2,4-dioxo-1,4-dihydro-2H-quinazolin-3-ylmethyl]-phenyl}-cyclopropanecarboxylic acid;

4-Pyridylmethyl 3-benzyl-1-methyl-2,4-dioxo-1,2,3,4-tetrahydroquinazoline-6-carboxylate;

3-(4-Fluoro-benzyl)-1-methyl-2,4-dioxo-1,2,3,4-tetrahydroquinazoline-6-carboxylic acid 3-methoxy-benzylamide;

3-(3,4-Difluoro-benzyl)-1-methyl-2,4-dioxo-1,2,3,4-tetrahydro-quinazoline-6-carboxylic acid 4-methoxy-benzylamide;

3-(4-Dimethylcarbamoyl-benzyl)-1-methyl-2,4-dioxo-1,2,3,4-tetrahydroquinazoline-6-carboxylic acid 4-methoxy-benzylamide;

1-Methyl-3-[4-(2-methyl-2H-tetrazol-5-yl)-benzyl]-2,4-dioxo-1,2,3,4-tetrahydro-quinazoline-6-carboxylic acid 4-methoxy-benzylamide;

3-(4-Bromo-benzyl)-1-methyl-2,4-dioxo-1,2,3,4-tetrahydro-quinazoline-6-carboxylic acid (2-methoxy-pyridin-4-ylmethyl)-amide;

3-(3,4-Difluoro-benzyl)-1-methyl-2,4-dioxo-1,2,3,4-tetrahydro-quinazoline-6-carboxylic acid (pyridin-3-ylmethyl)-amide;

Benzo[1,3]dioxol-5-ylmethyl-3-benzyl-1-methyl-2,4-dioxo-1,2,3,4-tetrahydroquinazoline-6-carboxylate;

3-Benzyl-1-methyl-2,4-dioxo-1,2,3,4-tetrahydroquinazoline-6-carboxylic acid (benzo[1,3]dioxol-5-ylmethyl)amide;

1-Methyl-3-(4-methylcarbamoyl-benzyl)-2,4-dioxo-1,2,3,4-tetrahydro-quinazoline-6-carboxylic acid 4-methoxy-benzylamide;

3-(3-Fluoro-benzyl)-1-methyl-2,4-dioxo-1,2,3,4-tetrahydro-quinazoline-6-carboxylic acid 4-methoxy-benzylamide;

4-[6-(4-Hydroxy-benzylcarbamoyl)-1-methyl-2,4-dioxo-1,4-dihydro-2H-quinazolin-3-ylmethyl]-benzoic acid;

Methyl 4-[6-(4-fluoro-benzylcarbamoyl)-1-methyl-2,4-dioxo-1,4-dihydro-2H-quinazolin-3-ylmethyl]-benzoate;

3-(4-Chlorobenzyl)-2,4-dioxo-1,2,3,4-tetrahydroquinazoline-6-carboxylic acid (benzo[1,3]dioxol-5-ylmethyl)amide;

1-Methyl-3-[4-(1-methyl-1H-tetrazol-5-yl)-benzyl]-2,4-dioxo-1,2,3,4-tetrahydro-quinazoline-6-carboxylic acid 4-methoxy-benzylamide;

3-(4-Methoxybenzyl)-1-methyl-2,4-dioxo-1,2,3,4-tetrahydroquinazoline-6-carboxylic acid 4-methoxybenzylamide;

4-Pyridylmethyl 3-(benzo[1,3]dioxol-5-ylmethyl)-2,4-dioxo-1,2,3,4-tetrahydroquinazoline-6-carboxylate;

Methyl 4-[6-(4-methoxy-benzylcarbamoyl)-1-methyl-2,4-dioxo-1,4-dihydro-2H-quinazolin-3-ylmethyl]-benzoate;

1-Methyl-2,4-dioxo-3-pyridin-4-ylmethyl-1,2,3,4-tetrahydro-quinazoline-carboxylic acid 4-methoxy-benzylamide;

3-(4-Amino-benzyl)-1-methyl-2,4-dioxo-1,2,3,4-tetrahydro-quinazoline-6-carboxylic acid 4-methoxy-benzylamide;

1-Methyl-3-(4-nitro-benzyl)-2,4-dioxo-1,2,3,4-tetrahydro-quinazoline-6-carboxylic acid 4-methoxy-benzylamide;

2-Methoxy-4-[6-(4-methoxy-benzylcarbamoyl)-1-methyl-2,4-dioxo-1,4-dihydro-2H-quinazolin-3-ylmethyl]-benzoic acid;

1-Methyl-3-(4-methylsulfamoyl-benzyl)-2,4-dioxo-1,2,3,4-tetrahydro-quinazoline-6-carboxylic acid 4-methoxy-benzylamide;

1-Methyl-2,4-dioxo-3-(4-sulfamoyl-benzyl)-1,2,3,4-tetrahydro-quinazoline-6-carboxylic acid 4-methoxy-benzylamide;

3-(4-Fluoro-benzyl)-1-methyl-2,4-dioxo-1,2,3,4-tetrahydro-quinazoline-6-carboxylic acid 4-methoxy-benzylamide;

3-(4-Fluoro-benzyl)-1-methyl-2,4-dioxo-1,2,3,4-tetrahydro-quinazoline-6-carboxylic acid (pyridin-4-ylmethyl)-amide;

3-(4-Methoxy-benzyl)-1-methyl-2,4-dioxo-1,2,3,4-tetrahydro-quinazoline-6-carboxylic acid (pyridin-4-ylmethyl)-amide;

2-Methyl-4-[6-(4-methoxy-benzylcarbamoyl)-1-methyl-2,4-dioxo-1,4-dihydro-2H-quinazolin-3-ylmethyl]-benzoic acid;

3-(4-Cyano-benzyl)-1-methyl-2,4-dioxo-1,2,3,4-tetrahydro-quinazoline-6-carboxylic acid 4-methoxy-benzylamide;

4-{1-Methyl-2,4-dioxo-6-[(pyridin-4-ylmethyl)-carbamoyl]-1,4-dihydro-2H-quinazolin-3-ylmethyl}-benzoic acid;

3-(3-fluoro-4-methoxy-benzyl)-1-methyl-2,4-dioxo-1,2,3,4-tetrahydro-quinazoline-6-carboxylic acid 4-methoxy benzylamine;

4-[1-Ethyl-6-(4-methoxy-benzylcarbamoyl)-2,4-dioxo-1,4-dihydro-2H-quinazolin-3-ylmethyl]-benzoic acid;

3-(Benzo[1,3]dioxol-5-ylmethyl)-2,4-dioxo-1,2,3,4-tetrahydroquinazoline-6-carboxylic acid (benzo[1,3]dioxol-5-ylmethyl)amide;

3-(2′-Cyano-biphenyl-4-ylmethyl)-1-methyl-2,4-dioxo-1,2,3,4-tetrahydro-quinazoline-6-carboxylic acid 4-methoxy-benzylamide;

4-[1-Methyl-6-(4-methylsulfanyl-benzylcarbamoyl)-2,4-dioxo-1,4-dihydro-2H-quinazolin-3-ylmethyl]-benzoic acid;

4-{6-[(Benzofurazan-5-ylmethyl)-carbamoyl]-1-methyl-2,4-dioxo-1,4-dihydro-2H-quinazolin-3-ylmethyl}-benzoic acid;

Methyl 2-methyl-4-[6-(4-methoxy-benzylcarbamoyl)-1-methyl-2,4-dioxo-1,4-dihydro-2H-quinazolin-3-ylmethyl]-benzoate;

3-(4-Acetylamino-benzyl)-1-methyl-2,4-dioxo-1,2,3,4-tetrahydro-quinazoline-6-carboxylic acid 4-methoxy-benzylamide;

3-(Benzo[1,3]dioxol-5-ylmethyl)-1-methyl-2,4-dioxo-1,2,3,4-tetrahydroquinazoline-6-carboxylic acid (benzo[1,3]dioxol-5-ylmethyl)amide;

3-(4-Dimethylcarbamoylmethyl-benzyl)-1-methyl-2,4-dioxo-1,2,3,4-tetrahydro-quinazoline-6-carboxylic acid 4-methoxy-benzylamide;

Benzo[1,3]dioxol-5-ylmethyl 3-benzyl-2,4-dioxo-1,2,3,4-tetrahydroquinazoline-6-carboxylate;

{4-[6-(4-Methoxy-benzylcarbamoyl)-1-methyl-2,4-dioxo-1,4-dihydro-2H-quinazolin-3-ylmethyl]-phenyl}-acetic acid;

(4-{1-Methyl-2,4-dioxo-6-[(pyridin-4-ylmethyl)-carbamoyl]-1,4-dihydro-2H-quinazolin-3-ylmethyl}-phenyl)-acetic acid;

3-Benzyl-2,4-dioxo-1,2,3,4-tetrahydroquinazoline-6-carboxylic acid 4-methoxybenzylamide;

Methyl {4-[6-(4-methoxy-benzylcarbamoyl)-1-methyl-2,4-dioxo-1,4-dihydro-2H-quinazolin-3-ylmethyl]-phenyl}-acetate;

3-(3-Fluoro-benzyl)-1-methyl-2,4-dioxo-1,2,3,4-tetrahydro-quinazoline-6-carboxylic acid (pyridin-4-ylmethyl)-amide;

2,4-Dioxo-3-(thien-2-ylmethyl)-1,2,3,4-tetrahydroquinazoline-6-carboxylic acid (benzo[1,3]dioxol-5-ylmethyl)amide;

1-Methyl-3-(4-methylsulfamoyl-benzyl)-2,4-dioxo-1,2,3,4-tetrahydro-quinazoline-6-carboxylic acid 4-methoxy-benzylamide;

Methyl 4-{1-methyl-2,4-dioxo-6-[(pyridin-4-ylmethyl)-carbamoyl]-1,4-dihydro-2H-quinazolin-3-ylmethyl}-benzoate;

2-Fluoro-4-[6-(4-methoxy-benzylcarbamoyl)-1-methyl-2,4-dioxo-1,4-dihydro-2H-quinazolin-3-ylmethyl]-benzoic acid;

3-(4-Cyano-benzyl)-1-methyl-2,4-dioxo-1,2,3,4-tetrahydro-pyrido[3,4-d]pyrimidine-6-carboxylic acid 4-methoxy-benzylamide;

4-[6-(3-Methoxy-benzylcarbamoyl)-1-methyl-2,4-dioxo-1,4-dihydro-2H-[3,4-d]pyrimidin-3-ylmethyl]-benzoic acid;

4-[6-(4-Methoxy-benzylcarbamoyl)-1-methyl-2,4-dioxo-1,4-dihydro-2H-quinazolin-3-ylmethyl]-benzoic acid hemi magnesium salt;

4-[6-(4-Methoxy-benzylcarbamoyl)-1-methyl-2,4-dioxo-1,4-dihydro-2H-[2,3-d]pyrimidin-3-ylmethyl]-benzoic acid;

3-[4-(N-methylsulfonylamino)-benzyl]-1-methyl-2,4-dioxo-1,2,3,4-tetrahydro-quinazoline-6-carboxylic acid 4-methoxy-benzylamide;

Ethyl 2-Fluoro-4-[6-(4-methoxy-benzylcarbamoyl)-1-methyl-2,4-dioxo-1,4-dihydro-2H-quinazolin-3-ylmethyl]-benzoate;

3-(4-Dimethylsulfamoyl-benzyl)-1-methyl-2,4-dioxo-1,2,3,4-tetrahydro-quinazoline-6-carboxylic acid 4-methoxy-benzylamide; and

3-(4-Methoxybenzyl)-1-methyl-2,4-dioxo-1,2,3,4-tetrahydroquinazoline-6-carboxylic acid (benzo[1,3]dioxol-5-ylmethyl)amide; or a pharmaceutically acceptable salt thereof.

44. A combination, comprising valdecoxib, or a pharmaceutically acceptable salt thereof, and an allosteric carboxylic inhibitor of MMP-13 of Formula ID

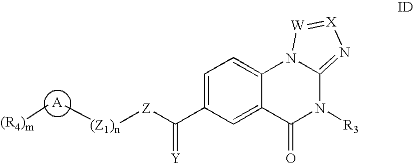

or a pharmaceutically acceptable salt thereof, or an N-oxide thereof,

in which:

W represents N or C—R₁; in which R₁ is selected from:

hydrogen atom,

OR₅, SR₅ in which R₅ is selected from hydrogen, (C₁-C₆)alkyl and aryl(C₁-C₆)alkyl,

(C₁-C₆)alkyl, cycloalkyl of 3 to 8 carbon atoms optionally interrupted with one hetero atom selected from oxygen, sulfur and nitrogen, aryl, heteroaryl and aryl(C₁-C₆)alkyl, these groups being optionally substituted by (CH₂)ₚ—OH or (CH₂)ₚ—NH₂, in which p is an integer from 0 to 4 inclusive,

X represents N or C—R₂ in which R₂ is selected from:

hydrogen atom,

NR₆R₇, OR₆, SR₆ in which R₆ and R₇, identical or different, are selected from hydrogen, (C₁-C₆)alkyl and aryl(C₁-C₆)alkyl,

(C₁-C₆)alkyl, cycloalkyl of 3 to 8 carbon atoms optionally interrupted with one hetero atom selected from oxygen, sulfur and nitrogen, aryl, heteroaryl and aryl(C₁-C₆)alkyl, these groups being optionally substituted by (CH₂)ₚ—OH or (CH₂)ₚ—NH₂, in which p is an integer from 0 to 4 inclusive,

Y represents a group selected from oxygen, sulfur, —NH, and —N(C₁-C₆)alkyl,

Z represents a group selected from:

oxygen, sulphur,

and —NR₈ in which R₈ represents a group selected from hydrogen, (C₁-C₆)alkyl, aryl(C₁-C₆)alkyl, cycloalkyl, aryl, and heteroaryl, and

when Y is oxygen, sulphur, or —N(C₁-C₆)alkyl, Z optionally represents a carbon atom which is optionally substituted by a group selected from (C₁-C₆)alkyl, aryl, aryl(C₁-C₆)alkyl, aromatic heterocycle, non-aromatic heterocycle, and cycloalkyl,

n is an integer from 0 to 8 inclusive,

Z₁ represents a group —CR₉R₁₀ wherein R₉ and R₁₀, identical or different, represent a group selected from hydrogen, (C₁-C₆)alkyl, halo(C₁-C₆)alkyl, halogen, NR₅R₁₁, OR₅, SR₅ and C(═O)OR₅ in which R₅ and R₁₁, identical or different, represents hydrogen atom or (C₁-C₆)alkyl, and

when n is greater than or equal to 2, the hydrocarbon chain Z₁ optionally contains one or more multiple bonds,

and/or one of the carbon atoms in the hydrocarbon chain Z₁ may be replaced with an oxygen atom, a sulphur atom which is optionally substituted by one or two oxygen atoms, or a nitrogen atom which is optionally substituted by (C₁-C₆)alkyl,

A represents a group selected from:

aromatic or non-aromatic, 5- or 6-membered monocycle comprising from 0 to 4 heteroatoms selected from nitrogen, oxygen and sulphur, and

bicycle, composed of two aromatic or non-aromatic, 5- or 6-membered rings, which may be identical or different, comprising from 0 to 4 heteroatoms selected from nitrogen, oxygen and sulphur,

m is an integer from 0 to 7 inclusive,

the group(s) R₄, which may be identical or different, is (are) selected from (C₁-C₆)alkyl, halogen, —CN, —NO₂, —SCF₃, —CF₃, —OCF₃, —NR₅R₁₁, —OR₅, —SR₅, —SOR₅, —SO₂R₅, —(CH₂)ₖSO₂NR₅R₁₁—X₁(CH₂)ₖC(═O)OR₅, —(CH₂)ₖC(═O)OR₅, —X₁(CH₂)ₖC(═O)NR₅R₁₁, —(CH₂)ₖC(═O)NR₅R₁₁, and —X₂—R₁₂ in which:

X₁ represents a group selected from oxygen, sulphur optionally substituted by one or two oxygen atoms, and nitrogen substituted by hydrogen or (C₁-C₆)alkyl,

k is an integer from 0 to 3 inclusive,

R₅ and R₁₁, which may be identical or different, are selected from hydrogen and (C₁-C₆)alkyl,

X₂ represents a group selected from single bond, —CH₂—, oxygen atom, sulphur atom optionally substituted by one or two oxygen atoms, and nitrogen atom substituted by hydrogen atom or (C₁-C₆)alkyl group,

R₁₂ represents an aromatic or non-aromatic, heterocyclic or non-heterocyclic, 5- or 6-membered ring which is optionally substituted by one or more groups, which may be identical or different, selected from (C₁-C₆)alkyl, halogen, hydroxyl, and amino, and when the ring is heterocyclic, it comprises from 1 to 4 heteroatoms selected from nitrogen, oxygen and sulphur,

R₃ represents a group selected from:

hydrogen,

(C₁-C₆)alkyl, (C₂-C₆)alkenyl, (C₂-C₆)alkynyl, these groups being optionally substituted by one or more groups, which may be identical or different, selected from amino, cyano, halo(C₁-C₆)alkyl, cycloalkyl, —C(═O)NR₅R₁₁, —C(═O)OR₅, —OR₅, and —SR₅, in which R₅ and R₁₁, which may be identical or different, are as defined hereinbefore,

and the group of formula:

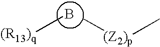

in which p is an integer from 0 to 8 inclusive,

Z₂ represents —CR₁₄R₁₅ wherein R₁₄ and R₁₅, identical or different, represent a group selected from hydrogen, (C₁-C₆)alkyl, phenyl, halo(C₁-C₆)alkyl, halogen, amino, —OR₅, —NR₅R₁₁, —SR₅ and —C(═O)OR₅ in which R₅ and R₁₁, identical or different, are as defined hereinbefore, and

when p is greater than or equal to 2, the hydrocarbon chain Z₂ optionally contains one or more multiple bonds,

and/or one of the carbon atoms in the hydrocarbon chain Z₂ may be replaced with an oxygen atom, a sulphur atom which is optionally substituted by one or two oxygen atoms, or a nitrogen atom which is optionally substituted by (C₁-C₆)alkyl,

B represents a group selected from:

aromatic or non-aromatic 5- or 6-membered monocycle comprising from 0 to 4 heteroatoms selected from nitrogen, oxygen and sulphur, and

bicycle, composed of two aromatic or non-aromatic, 5- or 6-membered rings, which may be identical or different, comprising from 0 to 4 heteroatoms selected from nitrogen, oxygen and sulphur,

q is an integer from 0 to 7 inclusive,

the group(s) R₁₃, which may be identical or different, is (are) selected from (C₁-C₆)alkyl, halogen, —CN, —NO₂, —CF₃, —OCF₃, (C₁-C₆)acyl, —(CH₂)ₖNR₁₆R₁₇, —X₃—(CH₂)ₖNR₁₆R₁₇—N(R₁₆)C(═O)R₁₇, —N(R₁₆)C(═O)OR₁₇, —N(R₁₆)SO₂R₁₇, —N(SO₂R₁₆)₂, —OR₁₆, —S(O)ₖ₁R₁₆, —(CH₂)ₖSO₂NR₁₆R₁₇, —X₃(CH₂)ₖC(═O)OR₁₆, —(CH₂)ₖC(═O)OR₁₆, —X₃(CH₂)ₖC(═O)NR₁₆R₁₇, —(CH₂)ₖC(═O)NR₁₆R₁₇, —C(═O)O—R₁₉—NR₁₆NR₁₇ and —X₄—R₁₈, in which:

X₃ represents a group selected from oxygen, sulphur optionally substituted by one or two oxygen atoms, and nitrogen substituted by a hydrogen atom or a (C₁-C₆)alkyl group,

k is an integer from 0 to 3 inclusive,

k₁ is an integer from 0 to 2 inclusive, —R₁₆ and R₁₇, which may be identical or different, are selected from hydrogen and (C₁-C₆)alkyl,

X₄ represents a group selected from single bond, —CH₂—, oxygen atom, sulphur atom optionally substituted by one or two oxygen atoms, and nitrogen atom substituted by hydrogen atom or (C₁-C₆)alkyl group,

R₁₈ represents an aromatic or non-aromatic, heterocyclic or non-heterocyclic, 5- or 6-membered ring, which is optionally substituted by one or more groups, which may be identical or different, selected from (C₁-C₆)alkyl, halogen, hydroxyl, (C₁-C₆)alkoxy, oxo, cyano, tetrazole, —NR₅R₁₁, and —C(═O)OR₅ wherein R₅ and R₁₁ are as defined hereinbefore, and, when the ring is heterocyclic, it comprises from 1 to 4 heteroatoms selected from nitrogen, oxygen and sulphur,

R₁₉ represents a (C₁-C₆)alkylene group.

45. The combination according to Embodiment 44, wherein the compound of Formula ID is selected from:

benzyl 4-benzyl-5-oxo-4H-[1,2,4]triazolo[4,3-a]quinazol-7-ylcarboxylate;

4-pyridylmethyl 4-benzyl-5-oxo-4H-[1,2,4]triazolo[4,3-a]quinazol-7-ylcarboxylate;

N-(3,4-methylenedioxybenzyl)-4-benzyl-5-oxo-4H-[1,2,4]triazolo[4,3-a]quinazol-7-ylcarboxamide;

N-(4-pyridylmethyl)-4-benzyl-5-oxo-4H-[1,2,4]triazolo [4,3-a]quinazol-7-ylcarboxamide;

N-(3,4-methylenedioxybenzyl)-4-benzyl-5-oxo-4H-imidazo[1,2-a]quinazol-7-ylcarboxamide;

N-(4-pyridylmethyl)-4-benzyl-5-oxo-4H-imidazo[1,2-a]quinazol-7-ylcarboxamide;

N-(4-methoxybenzyl)-4-benzyl-5-oxo-4,5-dihydro[1,2,4]triazolo[4,3-a]quinazoline-7-carboxamide;

N-[3-(4-pyridylsulphanyl)propyl]-4-benzyl-5-oxo-4,5-dihydro[1,2,4]triazolo-[4,3-a]quinazoline-7-carboxamide;

N-(3,4-Methylenedioxybenzyl)-4-(4-cyanobenzyl)-5-oxo-4H-[1,2,4]triazolo[4,3-a]quinazol-7-ylcarboxamide;

Methyl 4-{7-[(1,3-benzodioxol-5-ylmethyl)-carbamoyl]-5-oxo-5H-[1,2,4]triazolo[4,3-a]quinazol-4-ylmethyl}benzoate;

Methyl 4-{7-[(4-methoxybenzyl)-carbamoyl]-5-oxo-5H-[1,2,4]triazolo[4,3-a]quinazol-4-ylmethyl}benzoate;

Methyl 4-{7-[(pyridin-4-ylmethyl)-carbamoyl]-5-oxo-5H-[1,2,4]triazolo[4,3-a]quinazol-4-ylmethyl}benzoate;

(2-Dimethylamino-ethyl) 4-[7-(4-fluoro-benzylcarbamoyl)-5-oxo-5H-[1,2,4]triazolo[4,3-a]quinazol-4-ylmethyl]benzoate;

4-(4-Dimethylcarbamoyl-benzyl)-5-oxo-4,5-dihydro-[1,2,4]triazolo[4,3-ajquinazoline-7-carboxylic acid 4-methoxy-benzylamide;

N-(pyridin-4-ylmethyl)-4-(4-cyanobenzyl)-5-oxo-4H-[1,2,4]triazolo[4,3-a]quinazol-7-ylcarboxamide;

Methyl (4-{7-[(1,3-benzodioxol-5-ylmethyl)-carbamoyl]-5-oxo-5H-[1,2,4]triazolo[4,3-a]quinazolin-4-ylmethyl}-phenyl)-acetate;

Methyl (4-{7-[(4-methoxy)-benzylcarbamoyl]-5-oxo-5H-[1,2,4]triazolo[4,3-a]quinazolin-4-ylmethyl}-phenyl)-acetate;

Methyl (4-{7-[(pyridin-4-yl)-methylcarbamoyl]-5-oxo-5H-[1,2,4]triazolo[4,3-a]quinazolin-4-ylmethyl}-phenyl)-acetate;

N-(pyridin-4-ylmethyl) 4-[3-(pyridin-4-yl)-2-propen-1-yl]-5-oxo-4H-[1,2,4]triazolo[4,3-a]quinazol-7-ylcarboxamide;

4-[2-(4-Chloro-phenoxy)-ethyl]-5-oxo-4,5-dihydro-[1,2,4]triazolo[4,3-a]quinazoline-7-carboxylic acid 4-methoxy-benzylamide;

4-{7-[(4-methoxybenzyl)-carbamoyl]-5-oxo-5H-[1,2,4]triazolo[4,3-a]quinazol-4-ylmethyl}benzoic acid;

4-{7-[(1,3-benzodioxol-5-ylmethyl)-carbamoyl]-5-oxo-5H-[1,2,4]triazolo[4,3-a]quinazol-4-ylmethyl}benzoic acid;

4-{7-[(pyridin-4-ylmethyl)-carbamoyl]-5-oxo-5H-[1,2,4]triazolo[4,3-a]quinazol-4-ylmethyl}benzoic acid;

4-{7-[(4-fluoro)-benzylcarbamoyl]-5-oxo-5H-[1,2,4]triazolo[4,3-a]quinazol-4-ylmethyl}benzoic acid;

(4-{7-[(4-methoxy)-benzylcarbamoyl]-5-oxo-5H-[1,2,4]triazolo[4,3-a]quinazolin-4-ylmethyl}-phenyl)-acetic acid;

(4-{7-[(1,3-benzodioxol-5-ylmethyl)-carbamoyl]-5-oxo-5H-[1,2,4]triazolo[4,3-a]quinazolin-4-ylmethyl}-phenyl)-acetic acid; and

(4-{7-[(pyridin-4-yl)-methylcarbamoyl]-5-oxo-5H-[1,2,4]triazolo[4,3-a]quinazolin-4-ylmethyl}-phenyl)-acetic acid.

46. A combination, comprising valdecoxib, or a pharmaceutically acceptable salt thereof, and an allosteric carboxylic inhibitor of MMP-13 of Formula IE

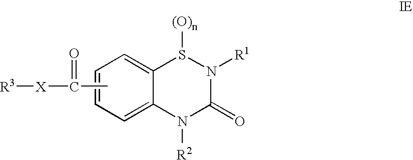

or a pharmaceutically acceptable salt thereof, wherein:

n is 0, 1, or 2;

X is O or NH;

R² is H, C₁-C₆ alkyl, or C₁-C₆ substituted alkyl;

R¹ and R³ independently are H, acyl, substituted acyl, C₁-C₆ alkyl, C₁-C₆ substituted alkyl, C₂-C₆ alkenyl, C₂-C₆ substituted alkenyl, C₂-C₆ alkynyl, C₁-C₆ substituted alkynyl, (CH₂)ₘ aryl, (CH₂)ₘ substituted aryl, (CH₂)ₘ heteroaryl, (CH₂)ₘ substituted heteroaryl, (CH₂)ₘ cycloalkyl, or (CH₂)ₘ substituted cycloalkyl; and

each m independently is an integer of from 0 to 6,

with the proviso that R³ is not (CH₂)ₘ biphenyl or (CH₂)ₘ substituted biphenyl.

47. The combination according to Embodiment 46, wherein the compound of Formula IE is a compound of Formula IIE

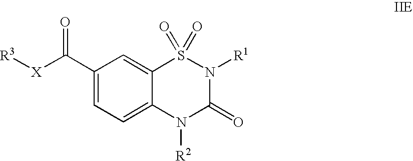

or a pharmaceutically acceptable salt thereof, wherein R¹, R², R³, and X are as defined above for Embodiment 46.

48. The combination according to Embodiment 46, wherein the compound of Formula IE is selected from:

2-Benzyl-4-methyl-1,1,3-trioxo-1,2,3,4-tetrahydro-1l⁶-benzo[1,2,4]thiadiazine-7-carboxylic acid benzyl ester;

2-Benzyl-4-methyl-1,1,3-trioxo-1,2,3,4-tetrahydro-1l⁶-benzo[1,2,4]thiadiazine-7-carboxylic acid benzylamide;

2-Benzyl-4-methyl-1,1,3-trioxo-1,2,3,4-tetrahydro-1l⁶-benzo[1,2,4]thiadiazine-7-carboxylic acid (pyridin-4-ylmethyl)-amide;

2-Benzyl-4-methyl-1,1,3-trioxo-1,2,3,4-tetrahydro-1l⁶-[1,2,4]thiadiazine-7-carboxylic acid (1H-indol-5-ylmethyl)-amide;

2-Benzyl-4-methyl-1,1,3-trioxo-1,2,3,4-tetrahydro-1l⁶-benzo[1,2,4]thiadiazine-7-carboxylic acid 4-methoxy-benzylamide;

2-Benzyl-4-methyl-1,1,3-trioxo-1,2,3,4-tetrahydro-1l⁶-[1,2,4]thiadiazine-7-carboxylic acid 4-(2-tert-butylsulfamoyl-ethyl)-benzylamide;

2-Benzyl-4-methyl-1,1,3-trioxo-1,2,3,4-tetrahydro-1l⁶-[1,2,4]thiadiazine-7-carboxylic acid (1H-indol-2-ylmethyl)-amide;

2-Benzyl-4-methyl-1,1,3-trioxo-1,2,3,4-tetrahydro-1l⁶-[1,2,4]thiadiazine-7-carboxylic acid 4-(2-sulfamoyl-ethyl)-benzylamide;

2-(4-Methanesulfonyl-benzyl)-4-methyl-1,1,3-trioxo-1,2,3,4-tetrahydro-1l⁶-benzo[1,2,4]thiadiazine-7-carboxylic acid benzylamide;

4-(7-Benzylcarbamoyl-4-methyl-1,1,3-trioxo-3,4-dihydro-1H-1l⁶-[1,2,4]thiadiazin-2-ylmethyl)-benzoic acid tert-butyl ester;

4-(7-Benzylcarbamoyl-4-methyl-1,1,3-trioxo-3,4-dihydro-1H-1l⁶-[1,2,4]thiadiazin-2-ylmethyl)-benzoic acid;

4-[7-(4-Methoxy-benzylcarbamoyl)-4-methyl-1,1,3-trioxo-3,4-dihydro-1H-1l⁶-benzo[1,2,4]thiadiazin-2-ylmethyl]-benzoic acid tert-butyl ester;

4-[7-(4-Methoxy-benzylcarbamoyl)-4-methyl-1,1,3-trioxo-3,4-dihydro-1H-1l⁶-benzo[1,2,4]thiadiazin-2-ylmethyl]-benzoic acid;

2-(4-Carbamoyl-benzyl)-4-methyl-1,1,3-trioxo-1,2,3,4-tetrahydro-1l⁶-benzo[1,2,4]thiadiazine-7-carboxylic acid 4-methoxy-benzylamide;

2-(4-Methanesulfonyl-benzyl)-4-methyl-1,1,3-trioxo-1,2,3,4-tetrahydro-1l⁶-benzo[1,2,4]thiadiazine-7-carboxylic acid 4-methoxy-benzylamide;

2-Benzyl-4-methyl-1,1,3-trioxo-1,2,3,4-tetrahydro-1l⁶-[1,2,4]thiadiazine-7-carboxylic acid 4-fluoro-benzylamide;

4-Methyl-2-(4-nitro-benzyl)-1,1,3-trioxo-1,2,3,4-tetrahydro-1l⁶-[1,2,4]thiadiazine-7-carboxylic acid 4-methoxy-benzylamide;

4-Methyl-2-(4-methylsulfamoyl-benzyl)-1,1,3-trioxo-1,2,3,4-tetrahydro-1l⁶-benzo[1,2,4]thiadiazine-7-carboxylic acid 4-methoxy-benzylamide;

4-Methyl-2-[4-(morpholine-4-sulfonyl)-benzyl]-1,1,3-trioxo-1,2,3,4-tetrahydro-1l⁶-benzo[1,2,4]thiadiazine-7-carboxylic acid 4-methoxy-benzylamide;

4-[7-(4-Fluoro-benzylcarbamoyl)-4-methyl-1,1,3-trioxo-3,4-dihydro-1H-1l⁶-benzo[1,2,4]thiadiazin-2-ylmethyl]-benzoic acid methyl ester;

2-Benzyl-4-methyl-1,1,3-trioxo-1,2,3,4-tetrahydro-1l⁶-[1,2,4]thiadiazine-7-carboxylic acid (2-methoxy-pyridin-4-ylmethyl)-amide;

4-Methyl-2-naphthalen-2-ylmethyl-1,1,3-trioxo-1,2,3,4-tetrahydro-1l⁶-[1,2,4]thiadiazine-7-carboxylic acid 4-methoxy-benzylamide;

2-Biphenyl-4-ylmethyl-4-methyl-1,1,3-trioxo-1,2,3,4-tetrahydro-1l⁶-[1,2,4]thiadiazine-7-carboxylic acid 4-methoxy-benzylamide;

2-Benzyl-4-methyl-1,1,3-trioxo-1,2,3,4-tetrahydro-1l⁶-[1,2,4]thiadiazine-7-carboxylic acid (2,1,3-benzothiadiazol-5-ylmethyl)-amide;

4-[7-(4-Fluoro-benzylcarbamoyl)-4-methyl-1,1,3-trioxo-3,4-dihydro-1H-1l⁶-benzo[1,2,4]thiadiazin-2-ylmethyl]-benzoic acid;

4-[7-(4-Methoxy-benzylcarbamoyl)-4-methyl-1,1,3-trioxo-3,4-dihydro-1H-1l⁶-benzo[1,2,4]thiadiazin-2-ylmethyl]-benzoic acid 2-dimethylamino-ethyl ester hydrochloride;

4-Methyl-1,1,3-trioxo-2-[4-(piperidine-1-carbonyl)-benzyl]-1,2,3,4-tetrahydro-1l⁶-benzo[1,2,4]thiadiazine-7-carboxylic acid 4-methoxy-benzylamide;

2-(4-[7-(4-Methoxy-benzylcarbamoyl)-4-methyl-1,1,3-trioxo-3,4-dihydro-1H-1l⁶-benzo[1,2,4]thiadiazin-2-ylmethyl]-benzoylamino}-3-methyl-butyric acid;

2-(4-Cyano-benzyl)-4-methyl-1,1,3-trioxo-1,2,3,4-tetrahydro-1l⁶-benzo[1,2,4]thiadiazine-7-carboxylic acid 4-methoxy-benzylamide;

{4-[7-(4-Methoxy-benzylcarbamoyl)-4-methyl-1,1,3-trioxo-3,4-dihydro-1H-1λ⁶-benzo[1,2,4]thiadiazin-2-ylmethyl]-phenyl}-acetic acid;

4-[7-(3-Methoxy-benzylcarbamoyl)-4-methyl-1,1,3-trioxo-3,4-dihydro-1H-1λ⁶-benzo[1,2,4]thiadiazin-2-ylmethyl]-benzoic acid;

4-Methyl-1,1,3-trioxo-2-[4-(2H-tetrazol-5-yl)-benzyl]-1,2,3,4-tetrahydro-1λ⁶-benzo[1,2,4]thiadiazine-7-carboxylic acid 4-methoxy-benzylamide;

2-(4-Amino-benzyl)-4-methyl-1,1,3-trioxo-1,2,3,4-tetrahydro-1λ⁶-[1,2,4]thiadiazine-7-carboxylic acid 4-methoxy-benzylamide;

2-Benzyl-4-methyl-1,1,3-trioxo-1,2,3,4-tetrahydro-1λ⁶-[1,2,4]thiadiazine-7-carboxylic acid 3-methoxy-benzylamide;

4-Methyl-1,1,3-trioxo-2-pent-2-ynyl-1,2,3,4-tetrahydro-1l⁶-[1,2,4]thiadiazine-7-carboxylic acid 4-methoxy-benzylamide;

4-Methyl-1,1,3-trioxo-2-(1-phenyl-ethyl)-1,2,3,4-tetrahydro-1l⁶-[1,2,4]thiadiazine-7-carboxylic acid 4-methoxy-benzylamide;

2-(5-Cyano-pentyl)-4-methyl-1,1,3-trioxo-1,2,3,4-tetrahydro-1l⁶-[1,2,4]thiadiazine-7-carboxylic acid 4-methoxy-benzylamide;

2-(E)-But-2-enyl-4-methyl-1,1,3-trioxo-1,2,3,4-tetrahydro-1l⁶-[1,2,4]thiadiazine-7-carboxylic acid 4-methoxy-benzylamide;

4-Methyl-1,1,3-trioxo-2-(E)-pent-2-enyl-1,2,3,4-tetrahydro-1l⁶-[1,2,4]thiadiazine-7-carboxylic acid 4-methoxy-benzylamide;

4-Methyl-2-(2-methyl-allyl)-1,1,3-trioxo-1,2,3,4-tetrahydro-1l⁶-benzo[1,2,4]thiadiazine-7-carboxylic acid 4-methoxy-benzylamide;

4-Methyl-2-(3-methyl-but-2-enyl)-1,1,3-trioxo-1,2,3,4-tetrahydro-1l⁶-benzo[1,2,4]thiadiazine-7-carboxylic acid 4-methoxy-benzylamide;

4-Methyl-1,1,3-trioxo-2-[2-(toluene-4-sulfonyl)-ethyl]-1,2,3,4-tetrahydro-1l⁶-benzo[1,2,4]thiadiazine-7-carboxylic acid 4-methoxy-benzylamide;

2-[3-(4-Fluoro-phenyl)-3-oxo-propyl]-4-methyl-1,1,3-trioxo-1,2,3,4-tetrahydro-1l⁶-benzo[1,2,4]thiadiazine-7-carboxylic acid 4-methoxy-benzylamide;

4-Methyl-1,1,3-trioxo-2-{2-[(1-phenyl-methanoyl)-amino]-ethyl}-1,2,3,4-tetrahydro-1l⁶-benzo[1,2,4]thiadiazine-7-carboxylic acid 4-methoxy-benzylamide;

2-Benzo[1,2,5]oxadiazol-5-ylmethyl-4-methyl-1,1,3-trioxo-1,2,3,4-tetrahydro-1l⁶-benzo[1,2,4]thiadiazine-7-carboxylic acid 4-methoxy-benzylamide;

{5-[7-(4-Methoxy-benzylcarbamoyl)-4-methyl-1,1,3-trioxo-3,4-dihydro-1H-1l⁶-benzo[1,2,4]thiadiazin-2-ylmethyl]-isoxazol-3-yl}-carbamic acid methyl ester; and

4-Methyl-1,1,3-trioxo-2-thiazol-4-ylmethyl-1,2,3,4-tetrahydro-1l⁶-[1,2,4]thiadiazine-7-carboxylic acid 4-methoxy-benzylamide; or a pharmaceutically acceptable salt thereof.

49. The combination according to Embodiment 46, wherein the compound of Formula IE is selected from:

2-Benzyl-4-methyl-1,1,3-trioxo-1,2,3,4-tetrahydro-1l⁶-[1,2,4]thiadiazine-7-carboxylic acid (pyridin-3-ylmethyl)-amide;

2-Benzyl-4-methyl-1,1,3-trioxo-1,2,3,4-tetrahydro-1l⁶-[1,2,4]thiadiazine-7-carboxylic acid 4-methoxy-benzylamide;

2-Benzyl-4-methyl-1,1,3-trioxo-1,2,3,4-tetrahydro-1l⁶-[1,2,4]thiadiazine-7-carboxylic acid 3-methoxy-benzylamide;

4-(7-Benzylcarbamoyl-4-methyl-1,1,3-trioxo-3,4-dihydro-1H-1l⁶-[1,2,4]thiadiazin-2-ylmethyl)-benzoic acid tert-butyl ester;

4-(4-Methyl-1,1,3-trioxo-7-[(pyridin-4-ylmethyl)-carbamoyl]-3,4-dihydro-1H-1l⁶-benzo[1,2,4]thiadiazin-2-ylmethyl)-benzoic acid tert-butyl ester;

4-(4-Methyl-1,1,3-trioxo-7-[(pyridin-3-ylmethyl)-carbamoyl]-3,4-dihydro-1H-1l⁶-benzo[1,2,4]thiadiazin-2-ylmethyl)-benzoic acid tert-butyl ester;

4-[7-(4-Methoxy-benzylcarbamoyl)-4-methyl-1,1,3-trioxo-3,4-dihydro-1H-1l⁶-benzo[1,2,4]thiadiazin-2-ylmethyl)-benzoic acid tert-butyl ester;

4-[7-(3-Methoxy-benzylcarbamoyl)-4-methyl-1,1,3-trioxo-3,4-dihydro-1H-1l⁶-benzo[1,2,4]thiadiazin-2-ylmethyl)-benzoic acid tert-butyl ester;

4-(7-Benzylcarbamoyl-4-methyl-1,1,3-trioxo-3,4-dihydro-1H-1l⁶-[1,2,4]thiadiazin-2-ylmethyl)-benzoic acid;

4-(4-Methyl-1,1,3-trioxo-7-[(pyridin-4-ylmethyl)-carbamoyl]-3,4-dihydro-H-1l⁶-benzo[1,2,4]thiadiazin-2-ylmethyl)-benzoic acid;

4-(4-Methyl-1,1,3-trioxo-7-[(pyridin-3-ylmethyl)-carbamoyl]-3,4-dihydro-H-1l⁶-benzo[1,2,4]thiadiazin-2-ylmethyl)-benzoic acid;

4-[7-(4-Methoxy-benzylcarbamoyl)-4-methyl-1,1,3-trioxo-3,4-dihydro-1H-1l⁶-benzo[1,2,4]thiadiazin-2-ylmethyl)-benzoic acid;

4-[7-(3-Methoxy-benzylcarbamoyl)-4-methyl-1,1,3-trioxo-3,4-dihydro-1H-1l⁶-benzo[1,2,4]thiadiazin-2-ylmethyl)-benzoic acid;

{4-(7-Benzylcarbamoyl-4-methyl-1,1,3-trioxo-3,4-dihydro-1H-1l⁶-[1,2,4]thiadiazin-2-ylmethyl)-phenyl}-acetic acid tert-butyl ester;

{4-(4-Methyl-1,1,3-trioxo-7-[(pyridin-4-ylmethyl)-carbamoyl]-3,4-dihydro-1H-1l⁶-benzo[1,2,4]thiadiazin-2-ylmethyl)-phenyl}-acetic acid tert-butyl ester;

{4-(4-Methyl-1,1,3-trioxo-7-[(pyridin-3-ylmethyl)-carbamoyl]-3,4-dihydro-1H-1l⁶-benzo[1,2,4]thiadiazin-2-ylmethyl)-phenyl}-acetic acid tert-butyl ester;

{4-[7-(4-Methoxy-benzylcarbamoyl)-4-methyl-1,1,3-trioxo-3,4-dihydro-1H-1l⁶-benzo[1,2,4]thiadiazin-2-ylmethyl)-phenyl}-acetic acid tert-butyl ester;

{4-[7-(3-Methoxy-benzylcarbamoyl)-4-methyl-1,1,3-trioxo-3,4-dihydro-1H-1l⁶-benzo[1,2,4]thiadiazin-2-ylmethyl)-phenyl}-acetic acid tert-butyl ester;

{4-(7-Benzylcarbamoyl-4-methyl-1,1,3-trioxo-3,4-dihydro-1H-1l⁶-benzo[1,2,4]thiadiazin-2-ylmethyl)-phenyl}-acetic acid;

{4-(4-Methyl-1,1,3-trioxo-7-[(pyridin-4-ylmethyl)-carbamoyl]-3,4-dihydro-1H-1l⁶-benzo[1,2,4]thiadiazin-2-ylmethyl)-phenyl}-acetic acid;

{4-(4-Methyl-1,1,3-trioxo-7-[(pyridin-3-ylmethyl)-carbamoyl]-3,4-dihydro-1H-1l⁶-benzo[1,2,4]thiadiazin-2-ylmethyl)-phenyl}-acetic acid;

{4-[7-(4-Methoxy-benzylcarbamoyl)-4-methyl-1,1,3-trioxo-3,4-dihydro-1H-1l⁶-benzo[1,2,4]thiadiazin-2-ylmethyl)-phenyl}-acetic acid;

{4-[7-(3-Methoxy-benzylcarbamoyl)-4-methyl-1,1,3-trioxo-3,4-dihydro-1H-1l⁶-benzo[1,2,4]thiadiazin-2-ylmethyl)-phenyl}-acetic acid;

2-(4-Methanesulfonyl-benzyl)-4-methyl-1,1,3-trioxo-1,2,3,4-tetrahydro-1l⁶-benzo[1,2,4]thiadiazine-7-carboxylic acid benzylamide;

2-(4-Methanesulfonyl-benzyl)-4-methyl-1,1,3-trioxo-1,2,3,4-tetrahydro-1l⁶-benzo[1,2,4]thiadiazine-7-carboxylic acid (pyridin-4-ylmethyl)-amide;

2-(4-Methanesulfonyl-benzyl)-4-methyl-1,1,3-trioxo-1,2,3,4-tetrahydro-1l⁶-benzo[1,2,4]thiadiazine-7-carboxylic acid (pyridin-3-ylmethyl)-amide;

2-(4-Methanesulfonyl-benzyl)-4-methyl-1,1,3-trioxo-1,2,3,4-tetrahydro-1l⁶-benzo[1,2,4]thiadiazine-7-carboxylic acid 4-methoxy-benzylamide;

2-(4-Methanesulfonyl-benzyl)-4-methyl-1,1,3-trioxo-1,2,3,4-tetrahydro-1l⁶-benzo[1,2,4]thiadiazine-7-carboxylic acid 3-methoxy-benzylamide;

4-Methyl-2-(4-methylsulfamoyl-benzyl)-1,1,3-trioxo-1,2,3,4-tetrahydro-1l⁶-benzo[1,2,4]thiadiazine-7-carboxylic acid benzylamide;

4-Methyl-2-(4-methylsulfamoyl-benzyl)-1,1,3-trioxo-1,2,3,4-tetrahydro-1l⁶-benzo[1,2,4]thiadiazine-7-carboxylic acid (pyridin-4-ylmethyl)-amide;

4-Methyl-2-(4-methylsulfamoyl-benzyl)-1,1,3-trioxo-1,2,3,4-tetrahydro-1l⁶-benzo[1,2,4]thiadiazine-7-carboxylic acid (pyridin-3-ylmethyl)-amide;

4-Methyl-2-(4-methylsulfamoyl-benzyl)-1,1,3-trioxo-1,2,3,4-tetrahydro-1l⁶-benzo[1,2,4]thiadiazine-7-carboxylic acid 4-methoxy-benzylamide;

4-Methyl-2-(4-methylsulfamoyl-benzyl)-1,1,3-trioxo-1,2,3,4-tetrahydro-1l⁶-benzo[1,2,4]thiadiazine-7-carboxylic acid 3-methoxy-benzylamide;

2-(4-Dimethylsulfamoyl-benzyl)-4-methyl-1,1,3-trioxo-1,2,3,4-tetrahydro-1l⁶-benzo[1,2,4]thiadiazine-7-carboxylic acid benzylamide;

2-(4-Dimethylsulfamoyl-benzyl)-4-methyl-1,1,3-trioxo-1,2,3,4-tetrahydro-1l⁶-benzo[1,2,4]thiadiazine-7-carboxylic acid (pyridin-4-ylmethyl)-amide;

2-(4-Dimethylsulfamoyl-benzyl)-4-methyl-1,1,3-trioxo-1,2,3,4-tetrahydro-1l⁶-benzo[1,2,4]thiadiazine-7-carboxylic acid (pyridin-3-ylmethyl)-amide;

2-(4-Dimethylsulfamoyl-benzyl)-4-methyl-1,1,3-trioxo-1,2,3,4-tetrahydro-1l⁶-benzo[1,2,4]thiadiazine-7-carboxylic acid 4-methoxy-benzylamide;

2-(4-Dimethylsulfamoyl-benzyl)-4-methyl-1,1,3-trioxo-1,2,3,4-tetrahydro-1l⁶-benzo[1,2,4]thiadiazine-7-carboxylic acid 3-methoxy-benzylamide;

2-Benzyl-1,1,3-trioxo-1,2,3,4-tetrahydro-1l⁶-benzo[1,2,4]thiadiazine-7-carboxylic acid benzylamide;

2-Benzyl-1,1,3-trioxo-1,2,3,4-tetrahydro-1l⁶-benzo[1,2,4]thiadiazine-7-carboxylic acid (pyridin-4-ylmethyl)-amide;

2-Benzyl-1,1,3-trioxo-1,2,3,4-tetrahydro-1l⁶-benzo[1,2,4]thiadiazine-7-carboxylic acid (pyridin-3-ylmethyl)-amide;

2-Benzyl-1,1,3-trioxo-1,2,3,4-tetrahydro-1l⁶-benzo[1,2,4]thiadiazine-7-carboxylic acid 4-methoxy-benzylamide;

2-Benzyl-1,1,3-trioxo-1,2,3,4-tetrahydro-1l⁶-benzo[1,2,4]thiadiazine-7-carboxylic acid 3-methoxy-benzylamide;

4-(7-Benzylcarbamoyl-1,1,3-trioxo-3,4-dihydro-1H-1l⁶-[1,2,4]thiadiazin-2-ylmethyl)-benzoic acid tert-butyl ester;

4-(1,1,3-Trioxo-7-[(pyridin-4-ylmethyl)-carbamoyl]-3,4-dihydro-1H-1l⁶-[1,2,4]thiadiazin-2-ylmethyl)-benzoic acid tert-butyl ester;

4-(1,1,3-Trioxo-7-[(pyridin-3-ylmethyl)-carbamoyl]-3,4-dihydro-1H-1l⁶-[1,2,4]thiadiazin-2-ylmethyl)-benzoic acid tert-butyl ester;

4-[7-(4-Methoxy-benzylcarbamoyl)-1,1,3-trioxo-3,4-dihydro-1H-1l⁶-[1,2,4]thiadiazin-2-ylmethyl)-benzoic acid tert-butyl ester;

4-[7-(3-Methoxy-benzylcarbamoyl)-1,1,3-trioxo-3,4-dihydro-1H-1l⁶-[1,2,4]thiadiazin-2-ylmethyl)-benzoic acid tert-butyl ester;

4-(7-Benzylcarbamoyl-1,1,3-trioxo-3,4-dihydro-1H-1l⁶-[1,2,4]thiadiazin-2-ylmethyl)-benzoic acid;

4-(1,1,3-Trioxo-7-[(pyridin-4-ylmethyl)-carbamoyl]-3,4-dihydro-1H-1l⁶-[1,2,4]thiadiazin-2-ylmethyl)-benzoic acid;

4-(1,1,3-Trioxo-7-[(pyridin-3-ylmethyl)-carbamoyl]-3,4-dihydro-1H-1l⁶-benzo[1,2,4]thiadiazin-2-ylmethyl)-benzoic acid;

4-[7-(4-Methoxy-benzylcarbamoyl)-1,1,3-trioxo-3,4-dihydro-1H-1l⁶-[1,2,4]thiadiazin-2-ylmethyl)-benzoic acid;

4-[7-(3-Methoxy-benzylcarbamoyl)-1,1,3-trioxo-3,4-dihydro-1H-1l⁶-[1,2,4]thiadiazin-2-ylmethyl)-benzoic acid;

{4-(7-Benzylcarbamoyl-1,1,3-trioxo-3,4-dihydro-1H-1l⁶-[1,2,4]thiadiazin-2-ylmethyl)-phenyl}-acetic acid tert-butyl ester;

{4-(1,1,3-Trioxo-7-[(pyridin-4-ylmethyl)-carbamoyl]-3,4-dihydro-1H-1l⁶-[1,2,4]thiadiazin-2-ylmethyl)-phenyl}-acetic acid tert-butyl ester;

{4-(1,1,3-Trioxo-7-[(pyridin-3-ylmethyl)-carbamoyl]-3,4-dihydro-1H-1l⁶-[1,2,4]thiadiazin-2-ylmethyl)-phenyl}-acetic acid tert-butyl ester;

{4-[7-(4-Methoxy-benzylcarbamoyl)-1,1,3-trioxo-3,4-dihydro-1H-1l⁶-[1,2,4]thiadiazin-2-ylmethyl)-phenyl}-acetic acid tert-butyl ester;

{4-[7-(3-Methoxy-benzylcarbamoyl)-1,1,3-trioxo-3,4-dihydro-1l⁶-[1,2,4]thiadiazin-2-ylmethyl)-phenyl}-acetic acid tert-butyl ester;

{4-(7-Benzylcarbamoyl-1,1,3-trioxo-3,4-dihydro-1H-1l⁶-[1,2,4]thiadiazin-2-ylmethyl)-phenyl}-acetic acid;

{4-(1,1,3-Trioxo-7-[(pyridin-4-ylmethyl)-carbamoyl]-3,4-dihydro-1H-1l⁶-benzo[1,2,4]thiadiazin-2-ylmethyl)-phenyl}-acetic acid;

{4-(1,1,3-Trioxo-7-[(pyridin-3-ylmethyl)-carbamoyl]-3,4-dihydro-1H-1l⁶-benzo[1,2,4]thiadiazin-2-ylmethyl)-phenyl}-acetic acid;

{4-[7-(4-Methoxy-benzylcarbamoyl)1,1,3-trioxo-3,4-dihydro-1H-1l⁶-benzo[1,2,4]thiadiazin-2-ylmethyl)-phenyl}-acetic acid;

{4-[7-(3-Methoxy-benzylcarbamoyl)-1,1,3-trioxo-3,4-dihydro-1H-1l⁶-[1,2,4]thiadiazin-2-ylmethyl)-phenyl}-acetic acid;

2-(4-Methanesulfonyl-benzyl)-1,1,3-trioxo-1,2,3,4-tetrahydro-1l⁶-[1,2,4]thiadiazine-7-carboxylic acid benzylamide;

2-(4-Methanesulfonyl-benzyl)-1,1,3-trioxo-1,2,3,4-tetrahydro-1l⁶-[1,2,4]thiadiazine-7-carboxylic acid (pyridin-4-ylmethyl)-amide;

2-(4-Methanesulfonyl-benzyl)-1,1,3-trioxo-1,2,3,4-tetrahydro-1l⁶-[1,2,4]thiadiazine-7-carboxylic acid (pyridin-3-ylmethyl)-amide;

2-(4-Methanesulfonyl-benzyl)-1,1,3-trioxo-1,2,3,4-tetrahydro-1l⁶-[1,2,4]thiadiazine-7-carboxylic acid 4-methoxy-benzylamide;

2-(4-Methanesulfonyl-benzyl)-1,1,3-trioxo-1,2,3,4-tetrahydro-1l⁶-[1,2,4]thiadiazine-7-carboxylic acid 3-methoxy-benzylamide;

2-(4-Methylsulfamoyl-benzyl)-1,1,3-trioxo-1,2,3,4-tetrahydro-1l⁶-[1,2,4]thiadiazine-7-carboxylic acid benzylamide;

2-(4-Methylsulfamoyl-benzyl)-1,1,3-trioxo-1,2,3,4-tetrahydro-1l⁶-[1,2,4]thiadiazine-7-carboxylic acid (pyridin-4-ylmethyl)-amide;

2-(4-Methylsulfamoyl-benzyl)-1,1,3-trioxo-1,2,3,4-tetrahydro-1l⁶-[1,2,4]thiadiazine-7-carboxylic acid (pyridin-3-ylmethyl)-amide;

2-(4-Methylsulfamoyl-benzyl)-1,1,3-trioxo-1,2,3,4-tetrahydro-1l⁶-[1,2,4]thiadiazine-7-carboxylic acid 4-methoxy-benzylamide;

2-(4-Methylsulfamoyl-benzyl)-1,1,3-trioxo-1,2,3,4-tetrahydro-1l⁶-[1,2,4]thiadiazine-7-carboxylic acid 3-methoxy-benzylamide;

2-(4-Dimethylsulfamoyl-benzyl)-1,1,3-trioxo-1,2,3,4-tetrahydro-1l⁶-[1,2,4]thiadiazine-7-carboxylic acid benzylamide;

2-(4-Dimethylsulfamoyl-benzyl)-1,1,3-trioxo-1,2,3,4-tetrahydro-1l⁶-[1,2,4]thiadiazine-7-carboxylic acid (pyridin-4-ylmethyl)-amide;

2-(4-Dimethylsulfamoyl-benzyl)-1,1,3-trioxo-1,2,3,4-tetrahydro-1l⁶-[1,2,4]thiadiazine-7-carboxylic acid (pyridin-3-ylmethyl)-amide;

2-(4-Dimethylsulfamoyl-benzyl)-1,1,3-trioxo-1,2,3,4-tetrahydro-1l⁶-[1,2,4]thiadiazine-7-carboxylic acid 4-methoxy-benzylamide; and

2-(4-Dimethylsulfamoyl-benzyl)-1,1,3-trioxo-1,2,3,4-tetrahydro-1l⁶-[1,2,4]thiadiazine-7-carboxylic acid 3-methoxy-benzylamide; or a pharmaceutically acceptable salt thereof.

50. A combination, comprising valdecoxib, or a pharmaceutically acceptable salt thereof, and an allosteric carboxylic inhibitor of MMP-13 of Formula IF

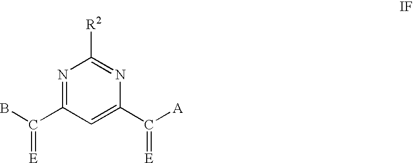

or a pharmaceutically acceptable salt thereof,

wherein:

R² is hydrogen, halo, hydroxy, C₁-C₆ alkyl, C₁-C₆ alkoxy, C₂-C₆ alkenyl, C₂-C₆ alkynyl, NO₂, NR⁴R⁵, CN, or CF₃;

E is independently O or S;

A and B independently are OR⁴ or NR⁵R⁶;

R⁴ and R⁵ independently are H, C₁-C₆ alkyl, C₂-C₆ alkenyl, C₂-C₆ alkynyl, (CH₂)ₙ aryl, (CH₂)ₙ cycloalkyl, (CH₂)ₙ heteroaryl, or R⁴ and R⁵ when taken together with the nitrogen to which they are attached complete a 3- to 8-membered ring, containing carbon atoms and optionally containing a heteroatom selected from O, S, or NH, and optionally substituted or unsubstituted; and

n is an integer from 0 to 6.

51. The combination according to Embodiment 50, wherein the compound of Formula IF is a compound of Formula IIF

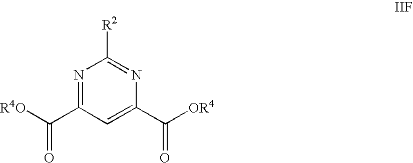

or a pharmaceutically acceptable salt thereof, wherein R² is as defined above, and each R⁴ independently is as defined above for Embodiment 50.

52. The combination according to Embodiment 50, wherein the compound of Formula IF is a compound of Formula IIIF

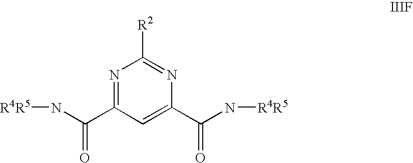

or a pharmaceutically acceptable salt thereof, wherein R² is as defined above, and each R⁴ and R⁵ independently are as defined above for Embodiment 50.

53. The combination according to Embodiment 50, wherein the compound of Formula IF is a compound of Formula IVF

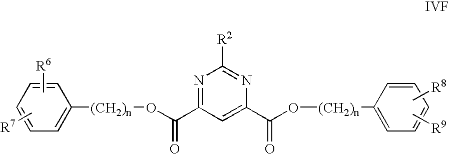

or a pharmaceutically acceptable salt thereof, wherein n and R² are as defined above for Embodiment 50, and R⁶, R⁷, R⁸, and R⁹ independently are hydrogen, halo, C₁-C₆ alkyl, C₁-C₆ alkoxy, nitro, or NH_{2.}

54. The combination according to Embodiment 50, wherein the compound of Formula IF is a compound of Formula VF

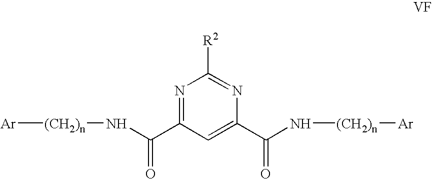

or a pharmaceutically acceptable salt thereof, wherein n and R² are as defined above for Embodiment 50, and each Ar independently is aryl or Het, wherein aryl is phenyl or substituted phenyl, and Het is an unsubstituted or substituted heteroaryl group.

55. The combination according to Embodiment 50, wherein the compound of Formula IF is selected from:

Pyrimidine-4,6-dicarboxylic acid, (4-chloro-benzylamide), [(1,3-benzodioxol-5-ylmethyl)-amide];

Pyrimidine-4,6-dicarboxylic acid, (4-carboxy-benzylamide), [(1,3-benzodioxol-5-ylmethyl)-amide];

Pyrimidine-4,6-dicarboxylic acid, (4-carboxy-benzylamide), (4-methoxy-benzylamide);

Pyrimidine-4,6-dicarboxylic acid, (4-carboxy-benzylamide), (3-methoxy-benzylamide);

Pyrimidine-4,6-dicarboxylic acid, (4-carbomethoxy-benzylamide), (3-methoxy-benzylamide);

Pyrimidine-4,6-dicarboxylic acid, (4-carboxy-benzylamide), (3-pyridylmethylamide);

Pyrimidine-4,6-dicarboxylic acid, (4-carboxy-benzylamide), (3-thiophenemethylamide);

Pyrimidine-4,6-dicarboxylic acid, (2,1,3-benzothiadiazol-5-ylmethyl) amide, [(1,3-benzodioxol-5-ylmethyl)-amide];

Pyrimidine-4,6-dicarboxylic acid, (2,1,3-benzooxadiazol-5-ylmethyl) amide, [(1,3-benzodioxol-5-ylmethyl)-amide];

Pyrimidine-4,6-dicarboxylic acid, (2,1,3-benzothiadiazol-5-ylmethyl) amide, (4-methoxy-benzylamide);

Pyrimidine-4,6-dicarboxylic acid, (2,1,3-benzothiadiazol-5-ylmethyl) amide, (3-methoxy-benzylamide);

Pyrimidine-4,6-dicarboxylic acid bis-(1,3-benzodioxol-5-ylmethyl) ester;

Pyrimidine-4,6-dicarboxylic acid, bis-(4-chloro-benzylamide);

Pyrimidine-4,6-dicarboxylic acid, bis-[(1,3-benzodioxol-5-ylmethyl)-amide];

Pyrimidine-4,6-dicarboxylic acid, bis-(4-methoxy-benzylamide);

Pyrimidine-4,6-dicarboxylic acid, bis-(3-methoxy-benzylamide);

Pyrimidine-4,6-dicarboxylic acid, bis-(4-carboxy-benzylamide); and

Pyrimidine-4,6-dicarboxylic acid, bis-(4-carbomethoxy-benzylamide); or a pharmaceutically acceptable salt thereof.

56. A combination, comprising valdecoxib, or a pharmaceutically acceptable salt thereof, and an allosteric carboxylic inhibitor of MMP-13 of Formula IG

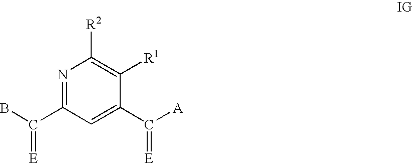

or a pharmaceutically acceptable salt thereof,

wherein:

R¹ and R² independently are hydrogen, halo, hydroxy, C₁-C₆ alkyl, C₁-C₆ alkoxy, C₂-C₆ alkenyl, C₂-C₆ alkynyl, NO₂, NR⁴R⁵, CN, or CF₃;

E is independently O or S;

A and B independently are OR⁴ or NR⁴R⁵;

R⁴ and R⁵ independently are H, C₁-C₆ alkyl, C₂-C₆ alkenyl, C₂-C₆ alkynyl, (CH₂)ₙ aryl, (CH₂)ₙ cycloalkyl, (CH₂)ₙ heteroaryl, or R⁴ and R⁵ when taken together with the nitrogen to which they are attached complete a 3- to 8-membered ring, containing carbon atoms and optionally containing a heteroatom selected from O, S, or NH, and optionally substituted or unsubstituted;

n is an integer from 0 to 6.

57. The combination according to Embodiment 56, wherein the compound of Formula IG is a compound of Formula IIG

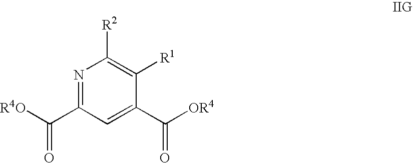

or a pharmaceutically acceptable salt thereof, wherein R¹ and R² are as defined above, and each R⁴ independently is as defined above for Embodiment 56.

58. The combination according to Embodiment 56, wherein the compound of Formula IG is a compound of Formula IIIG

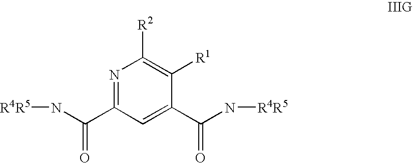

or a pharmaceutically acceptable salt thereof, wherein R¹ and R² are as defined above, and each R⁴ and R⁵ independently are as defined above for Embodiment 56.

59. The combination according to Embodiment 56, wherein the compound of Formula IG is a compound of Formula IVG

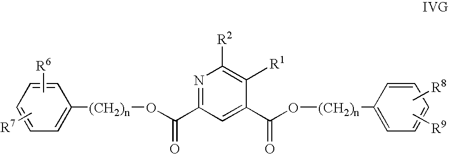

or a pharmaceutically acceptable salt thereof, wherein n, R¹, and R² are as defined above, and R⁶, R⁷, R⁸, and R⁹ independently are hydrogen, halo, C₁-C₆ alkyl, C₁-C₆ alkoxy, nitro, or NH₂.

60. The combination according to Embodiment 56, wherein the compound of Formula IG is a compound of Formula VG

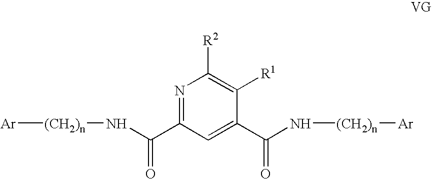

or a pharmaceutically acceptable salt thereof, wherein n, R¹, and R² are as defined above for Embodiment 56, and each Ar independently is aryl or Het, wherein aryl is phenyl or substituted phenyl, and Het is an unsubstituted or substituted heteroaryl group.

61. The combination according to Embodiment 56, wherein the compound of Formula IG is selected from:

Pyridine-3,5-dicarboxylic acid, (4-chloro-benzylamide), [(1,3-benzodioxol-5-ylmethyl)-amide];

Pyridine-3,5-dicarboxylic acid, (4-carboxy-benzylamide), [(1,3-benzodioxol-5-ylmethyl)-amide];

Pyridine-3,5-dicarboxylic acid, (4-carboxy-benzylamide), (4-methoxy-benzylamide);

Pyridine-3,5-dicarboxylic acid, (4-carboxy-benzylamide), (3-methoxy-benzylamide);

Pyridine-3,5-dicarboxylic acid, (4-carbomethoxy-benzylamide), (3-methoxy-benzylamide);

Pyridine-3,5-dicarboxylic acid, (4-carboxy-benzylamide), (3-pyridylmethylamide);

Pyridine-3,5-dicarboxylic acid, (4-carboxy-benzylamide), (3-thiophenemethylamide);

Pyridine-3,5-dicarboxylic acid, (2,1,3-benzothiadiazol-5-ylmethyl) amide, [(1,3-benzodioxol-5-ylmethyl)-amide];

Pyridine-3,5-dicarboxylic acid, (2,1,3-benzooxadiazol-5-ylmethyl) amide, [(1,3-benzodioxol-5-ylmethyl)-amide];

Pyridine-3,5-dicarboxylic acid, (2,1,3-benzothiadiazol-5-ylmethyl) amide, (4-methoxy-benzylamide);

Pyridine-3,5-dicarboxylic acid, (2,1,3-benzothiadiazol-5-ylmethyl) amide, (3-methoxy-benzylamide);

Pyridine-3,5-dicarboxylic acid bis-(1,3-benzodioxol-5-ylmethyl) ester;

2-Methoxy-pyridine-3,5-dicarboxylic acid bis-[(1,3-benzodioxol-5-ylmethyl)-amide];

2-Ethoxy-pyridine-3,5-dicarboxylic acid bis-[(1,3-benzodioxol-5-ylmethyl)-amide];

2-Amino-pyridine-3,5-dicarboxylic acid bis-[(1,3-benzodioxol-5-ylmethyl)-amide];

2-Oxo-1,2-dihydro-pyridine-3,5-dicarboxylic acid bis-benzylamide;

2-Methoxy-pyridine-3,5-dicarboxylic acid bis-benzylamide;

(3,5-Bis-benzylcarbamoyl-pyridin-2-yloxy)-acetic acid tert-butyl ester;

(3,5-Bis-benzylcarbamoyl-pyridin-2-yloxy)-acetic acid;

Pyridine-2,4-dicarboxylic acid bis-(3-methoxy-benzylamide);

Pyridine-2,4-dicarboxylic acid bis-[(1,3-benzodioxol-5-ylmethyl)-amide];

Pyridine-2,4-dicarboxylic acid bis-(2,4-dimethoxy-benzylamide);

Pyridine-2,4-dicarboxylic acid bis-(4-chloro-benzylamide);

Pyridine-2,4-dicarboxylic acid bis-benzylamide;

Pyridine-2,4-dicarboxylic acid bis-[(naphthalen-1-ylmethyl)-amide];

Pyridine-2,4-dicarboxylic acid bis-[(2-p-tolyl-ethyl)-amide];

Pyridine-2,4-dicarboxylic acid bis-(4-methoxy-benzylamide);

Pyridine-2,4-dicarboxylic acid bis-(3-fluoro-benzylamide);

Pyridine-2,4-dicarboxylic acid bis-(benzyl-ethyl-amide);

Pyridine-2,4-dicarboxylic acid bis-{[2-(3,4-dimethoxy-phenyl)-ethyl]-amide};

Pyridine-2,4-dicarboxylic acid bis-{[2-(2-phenoxy-phenyl)-ethyl]-amide};

Pyridine-2,4-dicarboxylic acid bis-[(4-phenyl-butyl)-amide];

Pyridine-2,4-dicarboxylic acid bis-{[2-(4-methoxy-phenyl)-ethyl]-amide};

Pyridine-2,4-dicarboxylic acid bis-{[2-(2-fluoro-phenyl)-ethyl]-amide};

Pyridine-2,4-dicarboxylic acid bis-{[2-(3-chloro-phenyl)-ethyl]-amide};

Pyridine-2,4-dicarboxylic acid bis-{[2-(2,4-dimethyl-phenyl)-ethyl]-amide};

Pyridine-2,4-dicarboxylic acid bis-[(2-o-tolyl-ethyl)-amide];

Pyridine-2,4-dicarboxylic acid bis-{[2-(4-ethyl-phenyl)-ethyl]-amide};

Pyridine-2,4-dicarboxylic acid bis-[(2-phenyl-propyl)-amide];

Pyridine-2,4-dicarboxylic acid bis-[(1,2-diphenyl-ethyl)-amide];

Pyridine-2,4-dicarboxylic acid bis-(2,4-dichloro-benzylamide);

Pyridine-2,4-dicarboxylic acid bis-[(biphenyl-2-ylmethyl)-amide];

Pyridine-2,4-dicarboxylic acid bis-(3,4,5-trimethoxy-benzylamide);

Pyridine-2,4-dicarboxylic acid bis-(3-chloro-benzylamide);

Pyridine-2,4-dicarboxylic acid bis-(3,5-dimethoxy-benzylamide);

Pyridine-2,4-dicarboxylic acid bis-(3,4-dimethoxy-benzylamide);

Pyridine-2,4-dicarboxylic acid bis-(ethyl-pyridin-4-ylmethyl-amide);

Pyridine-2,4-dicarboxylic acid bis-[(2-pyridin-4-yl-ethyl)-amide];

Pyridine-2,4-dicarboxylic acid bis-[(2-pyridin-3-yl-ethyl)-amide];

Pyridine-2,4-dicarboxylic acid bis-{[2-(4-chloro-phenyl)-ethyl]-amide};

Pyridine-2,4-dicarboxylic acid bis-[(pyridin-4-ylmethyl)-amide];

Pyridine-2,4-dicarboxylic acid bis-(3,5-bis-trifluoromethyl-benzylamide);

Pyridine-2,4-dicarboxylic acid bis-(2,3-dimethoxy-benzylamide);

Pyridine-2,4-dicarboxylic acid bis-(3-trifluoromethyl-benzylamide);

Pyridine-2,4-dicarboxylic acid bis-(2-trifluoromethoxy-benzylamide);

Pyridine-2,4-dicarboxylic acid bis-(3-difluoromethoxy-benzylamide);

Pyridine-2,4-dicarboxylic acid bis-(2-difluoromethoxy-benzylamide);

Pyridine-2,4-dicarboxylic acid bis-(4-fluoro-3-trifluoromethyl-benzylamide);

Pyridine-2,4-dicarboxylic acid bis-(2-methoxy-benzylamide);

Pyridine-2,4-dicarboxylic acid bis-{[2-(3-ethoxy-phenyl)-ethyl]-amide};

Pyridine-2,4-dicarboxylic acid bis-(3-chloro-4-fluoro-benzylamide);

Pyridine-2,4-dicarboxylic acid bis-(2,4-difluoro-benzylamide);

Pyridine-2,4-dicarboxylic acid bis-(4-amino-benzylamide);

Pyridine-2,4-dicarboxylic acid bis-(2-methyl-benzylamide);

Pyridine-2,4-dicarboxylic acid bis-{[bis-(4-methoxy-phenyl)-methyl]-amide};

Pyridine-2,4-dicarboxylic acid bis-[(3,3-diphenyl-propyl)-amide];

Pyridine-2,4-dicarboxylic acid bis-[(1-methyl-3-phenyl-propyl)-amide];

Pyridine-2,4-dicarboxylic acid bis-[(3,4-dimethoxy-phenyl)-amide];

Pyridine-2,4-dicarboxylic acid bis-(2-fluoro-benzylamide);

Pyridine-2,4-dicarboxylic acid bis-[(3-imidazol-1-yl-propyl)-amide];

Pyridine-2,4-dicarboxylic acid bis-(2-chloro-benzylamide);

Pyridine-2,4-dicarboxylic acid bis-(2-trifluoromethyl-benzylamide);

Pyridine-2,4-dicarboxylic acid bis-(4-methyl-benzylamide);

Pyridine-2,4-dicarboxylic acid bis-{[2-(3-methoxy-phenyl)-ethyl]-amide};

Pyridine-2,4-dicarboxylic acid bis-[(1-phenyl-ethyl)-amide];

Pyridine-2,4-dicarboxylic acid bis-[(pyridin-3-ylmethyl)-amide];

Pyridine-2,4-dicarboxylic acid bis-[(4-ethoxy-phenyl)-amide];

Pyridine-2,4-dicarboxylic acid bis-(phenethyl-amide);

Pyridine-2,4-dicarboxylic acid bis-[(thiophen-2-ylmethyl)-amide];

Pyridine-2,4-dicarboxylic acid bis-(4-trifluoromethyl-benzylamide);

Pyridine-2,4-dicarboxylic acid bis-[(5-methyl-furan-2-ylmethyl)-amide];

Pyridine-2,4-dicarboxylic acid bis-{[1-(4-fluoro-phenyl)-ethyl]-amide};

Pyridine-2,4-dicarboxylic acid bis-(2-amino-benzylamide);

Pyridine-2,4-dicarboxylic acid bis-[(1-naphthalen-1-yl-ethyl)-amide];

Pyridine-2,4-dicarboxylic acid bis-{[2-(4-hydroxy-phenyl)-ethyl]-amide};

Pyridine-2,4-dicarboxylic acid bis-(3-trifluoromethoxy-benzylamide);

Pyridine-2,4-dicarboxylic acid bis-{[1-(3-methoxy-phenyl)-ethyl]-amide};

Pyridine-2,4-dicarboxylic acid bis-[(1-phenyl-propyl)-amide];

Pyridine-2,4-dicarboxylic acid bis-{[2-(2-methoxy-phenyl)-ethyl]-amide};

Pyridine-2,4-dicarboxylic acid bis-{[2-(3-trifluoromethyl-phenyl)-ethyl]-amide};

Pyridine-2,4-dicarboxylic acid bis-indan-1-ylamide;

Pyridine-2,4-dicarboxylic acid bis-indan-1-ylamide;

Pyridine-2,4-dicarboxylic acid bis-(3,4-dichloro-benzylamide);

Pyridine-2,4-dicarboxylic acid bis-[(2-ethoxy-ethyl)-amide];

Pyridine-2,4-dicarboxylic acid bis-{[2-(4-bromo-phenyl)-ethyl]-amide};

Pyridine-2,4-dicarboxylic acid bis-[(2-pyridin-2-yl-ethyl)-amide];

Pyridine-2,4-dicarboxylic acid bis-[(2-thiophen-2-yl-ethyl)-amide];

Pyridine-2,4-dicarboxylic acid bis-{[2-(5-methoxy-1H-indol-3-yl)-ethyl]-amide);

Pyridine-2,4-dicarboxylic acid bis-{[2-(1H-indol-3-yl)-ethyl]-amide}; and

Pyridine-2,4-dicarboxylic acid bis-(3,5-dichloro-benzylamide); or a pharmaceutically acceptable salt thereof.

62. A pharmaceutical composition, comprising a combination of valdecoxib, or a pharmaceutically acceptable salt thereof, and an allosteric carboxylic inhibitor of MMP-13, or a pharmaceutically acceptable salt thereof, and a pharmaceutically acceptable carrier, diluent, or excipient.

63. The pharmaceutical composition according to Embodiment 62, wherein the combination is the combination according to any one of Embodiments 1 to 61.

64. The pharmaceutical composition according to Embodiment 62 or 63, wherein valdecoxib, or the pharmaceutically acceptable salt thereof, is in unit dosage form in an amount of from 1 milligram to 50 milligrams, and the allosteric carboxylic inhibitor of MMP-13, or a pharmaceutically acceptable salt thereof, is in unit dosage form in an amount of from 10 milligrams to 600 milligrams.

65. The pharmaceutical composition according to Embodiment 64, wherein valdecoxib, or the pharmaceutically acceptable salt thereof, is in unit dosage form in an amount of from 5 milligrams to 50 milligrams, and the allosteric carboxylic inhibitor of MMP-13, or a pharmaceutically acceptable salt thereof, is in unit dosage form in an amount of from 10 milligrams to 300 milligrams.

66. The pharmaceutical composition according to Embodiment 65, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 300 milligrams.

67. The pharmaceutical composition according to Embodiment 66, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 200 milligrams.

68. The pharmaceutical composition according to Embodiment 67, wherein valdecoxib, or the pharmaceutically acceptable salt thereof, is in unit dosage form in an amount of from 1 milligram to 5 milligrams, and the allosteric carboxylic inhibitor of MMP-13, or a pharmaceutically acceptable salt thereof, is in unit dosage form in an amount of from 25 milligrams to 100 milligrams.

69. A method of treating cartilage damage in a mammal in need thereof, comprising administering to the mammal a therapeutically effective amount of a combination comprising valdecoxib, or a pharmaceutically acceptable salt thereof, and an allosteric carboxylic inhibitor of MMP-13, or a pharmaceutically acceptable salt thereof.

70. The method according to Embodiment 69, wherein the combination is the combination according to any one of Embodiments 1 to 61.

71. A method of treating cartilage damage in a mammal in need thereof, comprising administering to the mammal a therapeutically effective amount of a pharmaceutical composition, comprising a combination of valdecoxib, or a pharmaceutically acceptable salt thereof, and an allosteric carboxylic inhibitor of MMP-13, or a pharmaceutically acceptable salt thereof, and a pharmaceutically acceptable carrier, diluent, or excipient.

72. The method according to Embodiment 71, wherein the combination is the combination according to any one of Embodiments 1 to 61.

73. The method according to Embodiment 71 or 72, wherein valdecoxib, or the pharmaceutically acceptable salt thereof, is in unit dosage form in an amount of from 1 milligram to 50 milligrams, and the allosteric carboxylic inhibitor of MMP-13, or a pharmaceutically acceptable salt thereof, is in unit dosage form in an amount of from 10 milligrams to 600 milligrams.

74. The method according to Embodiment 73, wherein valdecoxib, or the pharmaceutically acceptable salt thereof, is in unit dosage form in an amount of from 5 milligrams to 50 milligrams, and the allosteric carboxylic inhibitor of MMP-13, or a pharmaceutically acceptable salt thereof, is in unit dosage form in an amount of from 10 milligrams to 300 milligrams.

75. The method according to Embodiment 74, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 300 milligrams.

76. The method according to Embodiment 75, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 200 milligrams.

77. The method according to Embodiment 76, wherein valdecoxib, or the pharmaceutically acceptable salt thereof, is in unit dosage form in an amount of from 1 milligram to 5 milligrams, and the allosteric carboxylic inhibitor of MMP-13, or a pharmaceutically acceptable salt thereof, is in unit dosage form in an amount of from 25 milligrams to 100 milligrams.

78. A method of treating inflammation in a mammal in need thereof, comprising administering to the mammal a therapeutically effective amount of a combination comprising valdecoxib, or a pharmaceutically acceptable salt thereof, and an allosteric carboxylic inhibitor of MMP-13, or a pharmaceutically acceptable salt thereof.

79. The method according to Embodiment 78, wherein the combination is the combination according to any one of Embodiments 1 to 61.

80. A method of treating inflammation in a mammal in need thereof, comprising administering to the mammal a therapeutically effective amount of a pharmaceutical composition, comprising a combination of valdecoxib, or a pharmaceutically acceptable salt thereof, and an allosteric carboxylic inhibitor of MMP-13, or a pharmaceutically acceptable salt thereof, and a pharmaceutically acceptable carrier, diluent, or excipient.

81. The method according to Embodiment 80, wherein the combination is the combination according to any one of Embodiments 1 to 61.

82. The method according to Embodiment 80 or 81, wherein valdecoxib, or the pharmaceutically acceptable salt thereof, is in unit dosage form in an amount of from 1 milligram to 50 milligrams, and the allosteric carboxylic inhibitor of MMP-13, or a pharmaceutically acceptable salt thereof, is in unit dosage form in an amount of from 10 milligrams to 600 milligrams.

83. The method according to Embodiment 82, wherein valdecoxib, or the pharmaceutically acceptable salt thereof, is in unit dosage form in an amount of from 5 milligrams to 50 milligrams, and the allosteric carboxylic inhibitor of MMP-13, or a pharmaceutically acceptable salt thereof, is in unit dosage form in an amount of from 10 milligrams to 300 milligrams.

84. The method according to Embodiment 83, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 300 milligrams.

85. The method according to Embodiment 84, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 200 milligrams.

86. The method according to Embodiment 85, wherein valdecoxib, or the pharmaceutically acceptable salt thereof, is in unit dosage form in an amount of from 1 milligram to 5 milligrams, and the allosteric carboxylic inhibitor of MMP-13, or a pharmaceutically acceptable salt thereof, is in unit dosage form in an amount of from 25 milligrams to 100 milligrams.

87. A method of treating osteoarthritis in a mammal in need thereof, comprising administering to the mammal a therapeutically effective amount of a combination comprising valdecoxib, or a pharmaceutically acceptable salt thereof, and an allosteric carboxylic inhibitor of MMP-13, or a pharmaceutically acceptable salt thereof.

88. The method according to Embodiment 87, wherein the combination is the combination according to any one of Embodiments 1 to 61.

89. A method of treating osteoarthritis in a mammal in need thereof, comprising administering to the mammal a therapeutically effective amount of a pharmaceutical composition, comprising a combination of valdecoxib, or a pharmaceutically acceptable salt thereof, and an allosteric carboxylic inhibitor of MMP-13, or a pharmaceutically acceptable salt thereof, and a pharmaceutically acceptable carrier, diluent, or excipient.

90. The method according to Embodiment 89, wherein the combination is the combination according to any one of Embodiments 1 to 61.

91. The method according to Embodiment 89 or 90, wherein valdecoxib, or the pharmaceutically acceptable salt thereof, is in unit dosage form in an amount of from 1 milligram to 50 milligrams, and the allosteric carboxylic inhibitor of MMP-13, or a pharmaceutically acceptable salt thereof, is in unit dosage form in an amount of from 10 milligrams to 600 milligrams.

92. The method according to Embodiment 91, wherein valdecoxib, or the pharmaceutically acceptable salt thereof, is in unit dosage form in an amount of from 5 milligrams to 50 milligrams, and the allosteric carboxylic inhibitor of MMP-13, or a pharmaceutically acceptable salt thereof, is in unit dosage form in an amount of from 10 milligrams to 300 milligrams.

93. The method according to Embodiment 92, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 300 milligrams.

94. The method according to Embodiment 93, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 200 milligrams.

95. The method according to Embodiment 94, wherein valdecoxib, or the pharmaceutically acceptable salt thereof, is in unit dosage form in an amount of from 1 milligram to 5 milligrams, and the allosteric carboxylic inhibitor of MMP-13, or a pharmaceutically acceptable salt thereof, is in unit dosage form in an amount of from 25 milligrams to 100 milligrams.

96. A method of treating rheumatoid arthritis in a mammal in need thereof, comprising administering to the mammal a therapeutically effective amount of a combination comprising valdecoxib, or a pharmaceutically acceptable salt thereof, and an allosteric carboxylic inhibitor of MMP-13, or a pharmaceutically acceptable salt thereof.

97. The method according to Embodiment 96, wherein the combination is the combination according to any one of Embodiments 1 to 61.

98. A method of treating rheumatoid arthritis in a mammal in need thereof, comprising administering to the mammal a therapeutically effective amount of a pharmaceutical composition, comprising a combination of valdecoxib, or a pharmaceutically acceptable salt thereof, and an allosteric carboxylic inhibitor of MMP-13, or a pharmaceutically acceptable salt thereof, and a pharmaceutically acceptable carrier, diluent, or excipient.

99. The method according to Embodiment 98, wherein the combination is the combination according to any one of Embodiments 1 to 61.

100. The method according to Embodiment 98 or 99, wherein valdecoxib, or the pharmaceutically acceptable salt thereof, is in unit dosage form in an amount of from 1 milligram to 50 milligrams, and the allosteric carboxylic inhibitor of MMP-13, or a pharmaceutically acceptable salt thereof, is in unit dosage form in an amount of from 10 milligrams to 600 milligrams.

101. The method according to Embodiment 100, wherein valdecoxib, or the pharmaceutically acceptable salt thereof, is in unit dosage form in an amount of from 5 milligrams to 50 milligrams, and the allosteric carboxylic inhibitor of MMP-13, or a pharmaceutically acceptable salt thereof, is in unit dosage form in an amount of from 10 milligrams to 300 milligrams.

102. The method according to Embodiment 101, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 300 milligrams.

103. The method according to Embodiment 102, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 200 milligrams.

104. The method according to Embodiment 103, wherein valdecoxib, or the pharmaceutically acceptable salt thereof, is in unit dosage form in an amount of from 1 milligram to 5 milligrams, and the allosteric carboxylic inhibitor of MMP-13, or a pharmaceutically acceptable salt thereof, is in unit dosage form in an amount of from 25 milligrams to 100 milligrams.

105. A method of treating psoriatic arthritis in a mammal in need thereof, comprising administering to the mammal a therapeutically effective amount of a combination comprising valdecoxib, or a pharmaceutically acceptable salt thereof, and an allosteric carboxylic inhibitor of MMP-13, or a pharmaceutically acceptable salt thereof.

106. The method according to Embodiment 105, wherein the combination is the combination according to any one of Embodiments 1 to 61.

107. A method of treating psoriatic arthritis in a mammal in need thereof, comprising administering to the mammal a therapeutically effective amount of a pharmaceutical composition, comprising a combination of valdecoxib, or a pharmaceutically acceptable salt thereof, and an allosteric carboxylic inhibitor of MMP-13, or a pharmaceutically acceptable salt thereof, and a pharmaceutically acceptable carrier, diluent, or excipient.

108. The method according to Embodiment 107, wherein the combination is the combination according to any one of Embodiments 1 to 61.

109. The method according to Embodiment 107 or 108, wherein valdecoxib, or the pharmaceutically acceptable salt thereof, is in unit dosage form in an amount of from 1 milligram to 50 milligrams, and the allosteric carboxylic inhibitor of MMP-13, or a pharmaceutically acceptable salt thereof, is in unit dosage form in an amount of from 10 milligrams to 600 milligrams.

110. The method according to Embodiment 109, wherein valdecoxib, or the pharmaceutically acceptable salt thereof, is in unit dosage form in an amount of from 5 milligrams to 50 milligrams, and the allosteric carboxylic inhibitor of MMP-13, or a pharmaceutically acceptable salt thereof, is in unit dosage form in an amount of from 10 milligrams to 300 milligrams.

111. The method according to Embodiment 110, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 300 milligrams.

112. The method according to Embodiment 111, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 200 milligrams.

113. The method according to Embodiment 112, wherein valdecoxib, or the pharmaceutically acceptable salt thereof, is in unit dosage form in an amount of from 1 milligram to 5 milligrams, and the allosteric carboxylic inhibitor of MMP-13, or a pharmaceutically acceptable salt thereof, is in unit dosage form in an amount of from 25 milligrams to 100 milligrams.

114. A method of treating pain in a mammal in need thereof, comprising administering to the mammal a therapeutically effective amount of a combination comprising valdecoxib, or a pharmaceutically acceptable salt thereof, and an allosteric carboxylic inhibitor of MMP-13, or a pharmaceutically acceptable salt thereof.

115. The method according to Embodiment 114, wherein the combination is the combination according to any one of Embodiments 1 to 61.

116. A method of treating pain in a mammal in need thereof, comprising administering to the mammal a therapeutically effective amount of a pharmaceutical composition, comprising a combination of valdecoxib, or a pharmaceutically acceptable salt thereof, and an allosteric carboxylic inhibitor of MMP-13, or a pharmaceutically acceptable salt thereof, and a pharmaceutically acceptable carrier, diluent, or excipient.

117. The method according to Embodiment 116, wherein the combination is the combination according to any one of Embodiments 1 to 61.

118. The method according to Embodiment 116 or 117, wherein valdecoxib, or the pharmaceutically acceptable salt thereof, is in unit dosage form in an amount of from 1 milligram to 50 milligrams, and the allosteric carboxylic inhibitor of MMP-13, or a pharmaceutically acceptable salt thereof, is in unit dosage form in an amount of from 10 milligrams to 600 milligrams.

119. The method according to Embodiment 118, wherein valdecoxib, or the pharmaceutically acceptable salt thereof, is in unit dosage form in an amount of from 5 milligrams to 50 milligrams, and the allosteric carboxylic inhibitor of MMP-13, or a pharmaceutically acceptable salt thereof, is in unit dosage form in an amount of from 10 milligrams to 300 milligrams.

120. The method according to Embodiment 119, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 300 milligrams.

121. The method according to Embodiment 120, wherein valdecoxib, or the pharmaceutically acceptable salt thereof, is in unit dosage form in an amount of from 5 milligrams to 25 milligrams, and the allosteric carboxylic inhibitor of MMP-13, or a pharmaceutically acceptable salt thereof, is in unit dosage form in an amount of from 25 milligrams to 200 milligrams.

122. The method according to Embodiment 121, wherein valdecoxib, or the pharmaceutically acceptable salt thereof, is in unit dosage form in an amount of from 1 milligram to 5 milligrams, and the allosteric carboxylic inhibitor of MMP-13, or a pharmaceutically acceptable salt thereof, is in unit dosage form in an amount of from 25 milligrams to 100 milligrams.

Another invention embodiment is a combination according to any one of Embodiments 1 to 61, except where valdecoxib, or the pharmaceutically acceptable salt thereof, is replaced by celecoxib, or a pharmaceutically acceptable salt thereof.

Another invention embodiment is use of any one of the above combination Embodiments to treat a mammalian disease in a mammal in need of treatment, wherein the disease is selected from arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease, age-related macular degeneration, and cancers.

Another invention embodiment is any of the above embodiments of a combination, comprising an allosteric carboxylic inhibitor of MMP-13, or a pharmaceutically acceptable salt thereof, wherein the allosteric carboxylic inhibitor of MMP-13 is any single compound named below in the Examples of allosteric carboxylic inhibitors of MMP-13, with celecoxib, or a pharmaceutically acceptable salt thereof, or valdecoxib, or a pharmaceutically acceptable salt thereof.

Another invention embodiment is any of the above embodiments of pharmaceutical compositions, comprising a combination containing an allosteric carboxylic inhibitor of MMP-13, or a pharmaceutically acceptable salt thereof, wherein the allosteric carboxylic inhibitor of MMP-13 is any single compound named below in the Examples of allosteric carboxylic inhibitors of MMP-13, with celecoxib, or a pharmaceutically acceptable salt thereof, or valdecoxib, or a pharmaceutically acceptable salt thereof, together with a pharmaceutically acceptable carrier, diluent, or excipient.

Another invention embodiment is any of the above embodiments of a methods of treating a disease in a mammal suffering therefrom, comprising administering to the mammal a therapeutically effective amount of a combination, comprising an allosteric carboxylic inhibitor of MMP-13, or a pharmaceutically acceptable salt thereof, wherein the allosteric carboxylic inhibitor of MMP-13 is any single compound named below in the Examples of allosteric carboxylic inhibitors of MMP-13, with celecoxib, or a pharmaceutically acceptable salt thereof, or valdecoxib, or a pharmaceutically acceptable salt thereof.

Another invention embodiment is a combination, comprising an allosteric carboxylic inhibitor of MMP-13, or a pharmaceutically acceptable salt thereof, wherein the allosteric carboxylic inhibitor of MMP-13 is any single compound named below in the Examples of allosteric carboxylic inhibitors of MMP-13, with celecoxib, or a pharmaceutically acceptable salt thereof, or valdecoxib, or a pharmaceutically acceptable salt thereof.

Another invention embodiment is a pharmaceutical composition, comprising a combination containing an allosteric carboxylic inhibitor of P-13, or a pharmaceutically acceptable salt thereof, wherein the allosteric carboxylic inhibitor of MMP-13 is any single compound named below in the Examples of allosteric carboxylic inhibitors of MMP-13, with celecoxib, or a pharmaceutically acceptable salt thereof, or valdecoxib, or a pharmaceutically acceptable salt thereof, together with a pharmaceutically acceptable carrier, diluent, or excipient.

Another invention embodiment is a method of treating a disease that is responsive to inhibition of MMP-13 and to selective inhibition of COX-2 in a mammal suffering therefrom, comprising administering to the mammal a therapeutically effective amount of the combination according to any one of Embodiments 1 to 104.

Another invention embodiment is a method of treating a disease that is responsive to inhibition of MMP-13 and to selective inhibition of COX-2 in a mammal suffering therefrom, comprising administering to the mammal a therapeutically effective amount of a combination, comprising an allosteric carboxylic inhibitor of MMP-13, or a pharmaceutically acceptable salt thereof, wherein the allosteric carboxylic inhibitor of MMP-13 is any single compound named below in the Examples of allosteric carboxylic inhibitors of MMP-13, with celecoxib, or a pharmaceutically acceptable salt thereof, or valdecoxib, or a pharmaceutically acceptable salt thereof.

Another invention embodiment is a method of treating a first disease that is responsive to inhibition of MMP-13 and a second disease that is responsive to selective inhibition of COX-2 in a mammal suffering therefrom, comprising administering to the mammal a therapeutically effective amount of the combination according to any one of Embodiments 1 to 104.

Another invention embodiment is a method of treating a first disease that is responsive to inhibition of MMP-13 and a second disease that is responsive to selective inhibition of COX-2 in a mammal suffering therefrom, comprising administering to the mammal a therapeutically effective amount of a combination, comprising an allosteric carboxylic inhibitor of MMP-13, or a pharmaceutically acceptable salt thereof, wherein the allosteric carboxylic inhibitor of MMP-13 is any single compound named below in the Examples of allosteric carboxylic inhibitors of M-13, with celecoxib, or a pharmaceutically acceptable salt thereof, or valdecoxib, or a pharmaceutically acceptable salt thereof.

Another invention embodiment is a method of treating an arthritic condition in a mammal, comprising administering to the mammal an amount of any one of the above described invention combinations, or any one of the above-described invention pharmaceutical compositions, sufficient to effectively treat the arthritic condition.

Use of a combination comprising valdecoxib, or a pharmaceutically acceptable salt thereof, and an allosteric carboxylic inhibitor of MMP-13, or a pharmaceutically acceptable salt thereof, for the preparation of a medicament for treating cartilage damage in a mammal in need thereof.

Use of a combination comprising valdecoxib, or a pharmaceutically acceptable salt thereof, and an allosteric carboxylic inhibitor of MMP-13, or a pharmaceutically acceptable salt thereof, for the preparation of a medicament for treating inflammation in a mammal in need thereof.

Use of a combination comprising valdecoxib, or a pharmaceutically acceptable salt thereof, and an allosteric carboxylic inhibitor of MMP-13, or a pharmaceutically acceptable salt thereof, for the preparation of a medicament for treating osteoarthritis in a mammal in need thereof.

Use of a combination comprising valdecoxib, or a pharmaceutically acceptable salt thereof, and an allosteric carboxylic inhibitor of MMP-13, or a pharmaceutically acceptable salt thereof, for the preparation of a medicament for treating rheumatoid arthritis in a mammal in need thereof.

Use of a combination comprising valdecoxib, or a pharmaceutically acceptable salt thereof, and an allosteric carboxylic inhibitor of MMP-13, or a pharmaceutically acceptable salt thereof, for the preparation of a medicament for treating psoriatic arthritis in a mammal in need thereof.

Use of a combination comprising valdecoxib, or a pharmaceutically acceptable salt thereof, and an allosteric carboxylic inhibitor of MMP-13, or a pharmaceutically acceptable salt thereof, for the preparation of a medicament for treating pain in a mammal in need thereof.

### DETAILED DESCRIPTION OF THE INVENTION

As noted above, the invention provides a combination, comprising an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof. This invention also provides a method of treating a disease that is responsive to inhibition of MMP-13 and cyclooxygenase-2, comprising administering to a patient suffering from such a disease the invention combination comprising an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof. This invention also provides a pharmaceutical composition, comprising the invention combination comprising an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof, and a pharmaceutically acceptable carrier, diluent, or excipient.

The invention combinations may also be further combined with other pharmaceutical agents depending on the disease being treated.

The terms are as defined below or as they otherwise occur in the specification.

Definitions of Terms Used to Define Compounds of Formula I:

In Formula I, R¹ to R⁴ include “C₁-C₆ alkyl” groups. These are straight and branched carbon chains having from 1 to 6 carbon atoms. Examples of such alkyl groups include methyl, ethyl, isopropyl, tert-butyl, neopentyl, and n-hexyl. The alkyl groups can be substituted if desired, for instance with groups such as hydroxy, amino, alkyl, and dialkylamino, halo, trifluoromethyl, carboxy, nitro, and cyano.

Examples of NR⁴R⁵ groups include amino, methylamino, di-isopropylamino, acetyl amino, propionyl amino, 3-aminopropyl amino, 3-ethylaminobutyl amino, 3-di-n-propylamino-propyl amino, 4-diethylaminobutyl amino, and 3-carboxypropionyl amino. R⁴ and R⁵ can be taken together with the nitrogen to which they are attached to form a ring having 3 to 7 carbon atoms and 1, 2, or 3 heteroatoms selected from the group consisting of nitrogen, substituted nitrogen, oxygen, and sulfur. Examples of such cyclic NR⁴R⁵ groups include pyrrolidinyl, piperazinyl, 4-methylpiperazinyl, 4-benzylpiperazinyl, pyridinyl, piperidinyl, pyrazinyl, morpholinyl, and the like.

“Halo” includes fluoro, chloro, bromo, and iodo. It should be appreciated that invention compounds do not include compounds containing an N-halo group.

“Alkenyl” means straight and branched hydrocarbon radicals having from 2 to 6 carbon atoms and one double bond and includes ethenyl, 3-buten-1-yl, 2-ethenylbutyl, 3-hexen-1-yl, and the like.

“Alkynyl” means straight and branched hydrocarbon radicals having from 2 to 10 carbon atoms and one triple bond and includes ethynyl, 3-butyn-1-yl, propynyl, 2-butyn-1-yl, 3-pentyn-1-yl, 1-hexyn-1-yl, 7,7-dimethyl-1-octyn-1-yl, and the like.

“Cycloalkyl” means a monocyclic or polycyclic hydrocarbyl group such as cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, and the like. Examples of substituted cycloalkyl include 4-carboxycyclohexyl, 4-oxo-cyclohexyl, 4-(carboxymethyl)-cyclobutyl, 3-methyl-cyclopentyl, and 3-(carboxymethyl)cyclopentyl. Also included are rings in which 1 to 3 heteroatoms replace carbons. Such groups are termed “heterocycle” or “heterocyclyl”, which mean a cycloalkyl group also bearing at least one heteroatom selected from O, S, or NR², examples being oxiranyl, pyrrolidinyl, 4-methylpiperazinyl, piperidyl, tetrahydropyranyl, and morpholinyl. The group R² here is as defined above for Formula I, except where R² contains the functional group “NR⁵R⁶”, the groups R⁵ and R⁶ are not taken together with the nitrogen atom to which they are attached to complete a 3- to 7-membered ring.

“Alkoxy” refers to the alkyl groups mentioned above bound through oxygen, examples of which include methoxy, ethoxy, isopropoxy, tert-butoxy, and the like. In addition, alkoxy refers to polyethers such as —O—(CH₂)₂—O—CH₃, and the like.

“Alkanoyl” groups are alkyl linked through a carbonyl, i.e., C₁-C₅—C(O)—. Such groups include formyl, acetyl, propionyl, butyryl, and isobutyryl.

“Acyl” means an alkyl or aryl (Ar) group bonded through a carbonyl group, i.e., R—C(O)—. For example, acyl includes a C₁-C₆ alkanoyl, including substituted alkanoyl, wherein the alkyl portion can be substituted by NR⁴R⁵ or a carboxylic or heterocyclic group. Typical acyl groups include acetyl, benzoyl, and the like.

The alkyl, alkenyl, alkoxy, and alkynyl groups described above are optionally substituted, preferably by 1 to 3 groups selected from NR⁴R⁵, phenyl, substituted phenyl, heteroaryl, substituted heteroaryl, heterocycle, thio C₁-C₆ alkyl, C₁-C₆ alkoxy, hydroxy, carboxy, C₁-C₆ alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₙPh where n is 1, 2, or 3. Perhalo and polyhalo substitution is also embraced.

Examples of substituted alkyl groups include 2-aminoethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, benzyl(Bₙ), 3-morpholinopropyl, piperazinylmethyl, pyridyl-4-methyl(Py-4-me), 3-(pyridyl-4-thio)propyl, and 2-(4-methylpiperazinyl)ethyl.

Examples of substituted alkynyl groups include 2-methoxyethynyl, 2-ethylsulfanyethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include dimethylaminomethyl, carboxymethyl, 4-dimethylamino-3-buten-1-yl, 5-ethylmethylamino-3-pentyn-1-yl, 3-(3-methoxyphenyl)-propyn-1-yl, 3-(3,4-difluorophenyl)-propyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The terms “Ar” and “aryl” refer to unsubstituted and substituted aromatic groups. Heteroaryl groups have from 4 to 10 ring atoms, from 1 to 4 of which are independently selected from the group consisting of O, S, and N. Preferred heteroaryl groups have 1 or 2 heteroatoms in a 5- or 6-membered aromatic ring. Mono- and bicyclic aromatic ring systems are included in the definition of aryl and heteroaryl. Typical aryl groups include phenyl and naphthyl. Typical substituted aryl groups include 3,4-difluorophenyl, 4-carboxyphenyl, 3,4-methylenedioxyphenyl, 4-carboxymethylphenyl, 3-methoxyphenyl, and 7-fluoro-1-naphthyl. Typical heteroaryl groups include pyridyl, thienyl, benzothienyl, indolyl, furanyl, thiazolyl, isothiazolyl, indazolyl, 2-oxo-2H-1-benzopyranyl, and imidazolyl. Typical substituted heteroaryl groups include 3-methoxy-isothiazolyl, 3-methoxypyridin-4-yl, 4-ethylbenzothienyl, 4-thiopyridyl, 2-methoxy-pyridin-4-yl, 1-methylpyrazol-4-yl, and 2-methyl-pyridin-3-yl.

Preferred Ar groups are phenyl and phenyl substituted by 1, 2, or 3 groups independently selected from alkyl, alkoxy, alkoxycarbonyl, thio, thioalkyl, (C₁-C₆ alkyl)sulfanyl, (C₁-C₆ alkyl)sulfonyl, halo, hydroxy, (CH₂)₀₋₆CO₂R⁷, trifluoromethyl, trifluoromethoxy, nitro, amino of the formula —NR⁴R⁵, C(═O)NR⁵R⁶, N(R⁴)C(═O)OR⁵, and T(CH₂)ₘQR⁴ or T(CH₂)ₘCO₂R⁴, wherein m is 1 to 6, T is O, S, NR⁴, N(O)R⁴, NR⁴R⁶Y, or CR⁴R⁵, Q is O, S, NR5, N(O)R⁵, or NR⁵R⁶Y, wherein R⁴—R⁶ are as described above, and R⁷ is hydrogen, alkyl, or substituted alkyl, for example, methyl, trichloroethyl, diphenylmethyl, and the like. The alkyl and alkoxy groups can be substituted as defined above. For example, typical groups are carboxyalkyl, alkoxycarbonylalkyl, hydroxyalkyl, hydroxyalkoxy, and alkoxyalkyl. Examples of substituted phenyl are 3-methoxyphenyl, 2,6-dichlorophenyl, 3-nitrophenyl, 4-dimethylaminophenyl, and biphenyl.

Preferred heteroaryl groups include thienyl, furanyl, pyrrolyl, isoxazolyl, isothiazolyl, imidazolyl, pyrazolyl, oxazolyl, thiazolyl, 1,2,4-oxadiazolyl, 1,2,4,-thiadiazolyl, 1,2,4-triazolyl, tetrazolyl, benzofuranyl, benzothienyl, indolyl, benzimidazolyl, benzotriazolyl, benzoxazolyl, benzthiazolyl, pyridinyl, pyrimidinyl, quinolinyl, isoquinolinyl, and 2-oxo-2H-1-benzopyranyl.

Preferred heteroaryl groups may be substituted on a carbon atom as described above for substituted phenyl, and may further be substituted on a ring nitrogen atom (i.e., by replacing a hydrogen from a ring nitrogen atom, which is an NH group) with (C₁-C₆ alkyl) C(═O), C₁-C₆ alkyl, C₂-C₆ alkenyl, C₂-C₁₀alkynyl, or benzyl.

Definitions of Terms Used to Define Compounds of Formula IA:

In Formula IA, R¹ to R⁹ include “C₁-C₆ alkyl” groups. Alkyl groups are straight and branched carbon chains having from 1 to 6 carbon atoms. Examples of such alkyl groups include methyl, ethyl, isopropyl, tert-butyl, neopentyl, and n-hexyl. The alkyl groups can be substituted if desired, for instance with groups such as hydroxy, amino, alkyl, and dialkylamino, halo, trifluoromethyl, carboxy, nitro, and cyano.

Examples of NR⁴R⁵ groups include amino, methylamino, di-isopropylamino, acetyl amino, propionyl amino, 3-aminopropyl amino, 3-ethylaminobutyl amino, 3-di-n-propylamino-propyl amino, 4-diethylaminobutyl amino, and 3-carboxypropionyl amino. R⁴ and R⁵ can be taken together with the nitrogen to which they are attached to form a ring containing from 3 to 7 carbon atoms and 1, 2, or 3 heteroatoms selected from the group consisting of nitrogen, substituted nitrogen, oxygen, and sulfur. Examples of such cyclic NR⁴R⁵ groups include pyrrolidinyl, piperazinyl, 4-methylpiperazinyl, 4-benzylpiperazinyl, pyridinyl, piperidinyl, pyrazinyl, morpholinyl, and the like.

“Halo” includes fluoro, chloro, bromo, and iodo.

“Alkenyl” means straight and branched hydrocarbon radicals having from 2 to 6 carbon atoms and one double bond and includes ethenyl, 3-buten-1-yl, 2-ethenylbutyl, 3-hexen-1-yl, and the like.

“Alkynyl” means straight and branched hydrocarbon radicals having from 2 to 6 carbon atoms and one triple bond and includes ethynyl, 3-butyn-1-yl, propynyl, 2-butyn-1-yl, 3-pentyn-1-yl, and the like.

“Cycloalkyl” means a monocyclic or polycyclic hydrocarbyl group such as cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, and the like. Also included are rings in which 1 to 3 heteroatoms replace carbons. Such groups are termed “heterocyclyl,” which means a cycloalkyl group also bearing at least one heteroatom selected from O, S, or NR², examples being oxiranyl, pyrrolidinyl, piperidyl, tetrahydropyran, and morpholine.

“Alkoxy” refers to the alkyl groups mentioned above bound through oxygen, examples of which include methoxy, ethoxy, isopropoxy, tert-butoxy, and the like. In addition, alkoxy refers to polyethers such as —O—(CH₂)₂—O—OH₃, and the like.

“Acyl” means an R group that is a C₁-C₆ alkyl or aryl (Ar) group bonded through a carbonyl group, i.e., R—C(O)—, where R is alkyl or aryl. For example, acyl includes a C₁-C₆ alkanoyl, including substituted alkanoyl, wherein the alkyl portion can be substituted by NR⁴R⁵ or a carboxylic or heterocyclic group. Typical acyl groups include acetyl, benzoyl, isonicotinoyl, and the like.

The alkyl, alkenyl, alkoxy, and alkynyl groups described above are optionally substituted, preferably by 1 to 3 groups selected from NR⁴R⁵, phenyl, substituted phenyl, thio C₁-C₆ alkyl, C₁-C₆ alkoxy, hydroxy, carboxy, C₁-C₆ alkoxycarbonyl, acyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₙPh where n is 1, 2, or 3. Perhalo and polyhalo substitution is also embraced.

Examples of substituted alkyl groups include 2-aminoethyl, acetylmethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, 4-benzoylbutyl, and 2-(4-methylpiperazinyl)ethyl.

Examples of substituted alkynyl groups include 2-methoxyethynyl, 2-benzoylethylyl, 2-ethylsulfanyethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, acetoxymethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The terms “Ar” and “aryl” refer to unsubstituted and substituted aromatic groups. Heteroaryl groups have from 4 to 10 ring atoms, from 1 to 4 of which are independently selected from the group consisting of O, S, and N. Preferred heteroaryl groups have 1 or 2 heteroatoms in a 5- or 6-membered aromatic ring. Mono- and bicyclic aromatic ring systems are included in the definition of aryl and heteroaryl. A bicyclic aryl group is naphthyl for example. Bicyclic heteroaryl groups include indolyl and benzothienyl, to name a few. Preferred substituent groups include alkyl, alkoxy, halo, amino, alkylamino, dialkylamino, CN, CF₃, thioalkyl, acyl and hydroxy. Typical aryl and heteroaryl groups include phenyl, 3-chlorophenyl, 2,6-dibromophenyl, pyridyl, 3-methylpyridyl, benzothienyl, 2,4,6-tribromophenyl, 4-ethylbenzothienyl, furanyl, 3,4-diethylfuranyl, naphthyl, 4,7-dichloronaphthyl, morpholinyl, indolyl, benzotriazolyl, indazolyl, pyrrole, pyrazole, imidazole, thiazole, methylenedioxyphenyl, benzo-2,1,3-thiadiazole, benzo-2,1,3-oxadiazole, and the like.

Preferred Ar groups are phenyl and phenyl substituted by 1, 2, or 3 groups independently selected from the group consisting of alkyl, alkoxy, thio, thioalkyl, halo, hydroxy, —COOR⁷, trifluoromethyl, nitro, amino of the formula —NR⁴R⁵, and T(CH₂)ₘQR⁴ or T(CH₂)ₘCO₂R⁴ wherein m is 1 to 6, T is O, S, NR⁴, N(O)R⁴, NR⁴R⁶Y, or CR⁴R⁵, Q is O, S, NR⁵, N(O)R⁵, or NR⁵R⁶Y wherein R⁴ and R⁵ are as described above, and R⁷ is H, alkyl or substituted alkyl, for example, methyl, trichloroethyl, diphenylmethyl, and the like. The alkyl and alkoxy groups can be substituted as defined above. For example, typical groups are carboxyalkyl, alkoxycarbonylalkyl, hydroxyalkyl, hydroxyalkoxy, and alkoxyalkyl. Typical substituted aryl groups include 2,6-dichlorophenyl, 3-hydroxyphenyl, 1,3-benzodioxolyl, 4-dimethylaminophenyl, 2,4,6-triethoxyphenyl, 3-cyanophenyl, 4-methylthiophenyl, and 3,5-dinitrophenyl.

The term “substituted”, unless otherwise defined, includes from 1 to 3 substituents selected from:

C₁-C₆ alkyl; C₂-C₆ alkenyl; C₂-C₆ alkynyl; C₁-C₆ alkoxy; phenyl;

(C₁-C₆ alkoxy)carbonyl; (C₁-C₆ alkyl)sulfanyl; (C₁-C₆ alkyl)carbonyl;

OH; NH₂; N(H)R⁴, wherein R⁴ is as defined above for Formula IA;

NR⁴R⁵, wherein R⁴ and R⁵ are as defined above for Formula IA, or R⁴ and R⁵ are taken together with the nitrogen atom to which they are attached to form a 3- to 7-membered saturated ring containing carbon atoms and optionally from 1 or 2 heteroatoms selected from O, S, S(O), S(O)₂, N(H), and N(C₁-C₆ alkyl), wherein the ring may be optionally substituted on a carbon atom with 1 oxo (i.e., ═O) group;

C(═O)NR⁴R⁵, wherein R⁴ and R⁵ are as defined immediately above, or R⁴ and R⁵ are taken together with the nitrogen atom to which they are attached to form a 3- to 7-membered saturated ring containing carbon atoms and optionally 1 or 2 heteroatoms selected from O, S, S(O), S(O)₂, N(H), and N(C₁-C₆ alkyl), wherein the ring may be optionally substituted on a carbon atom with 1 oxo (i.e., ═O) group;

CN; NO₂; CF₃; CO₂H; CHO; SH; (C₁-C₆alkyl) S(O);

(C₁-C₆ alkyl)sulfonyl; halo; S(O)₂NR⁴R⁵, wherein R⁴ and R⁵ are as defined above for Formula IA, or R⁴ and R⁵ are taken together with the nitrogen atom to which they are attached to form a 3- to 7-membered saturated ring containing carbon atoms and optionally 1 or 2 heteroatoms selected from O, S, S(O), S(O)₂, N(H), and N(C₁-C₆ alkyl), wherein the ring may be optionally substituted on a carbon atom with 1 oxo (i.e., ═O) group;

OCF₃; and (CH₂)ₘCO2H, wherein m is as defined above for Formula IA.

Definitions of Terms Used to Define Compounds of Formula IB:

In Formula IB, R¹—R⁴ include “C₁-C₆ alkyl” groups. These are straight and branched carbon chains having from 1 to 6 carbon atoms. Examples of such alkyl groups include methyl, ethyl, isopropyl, tert-butyl, neopentyl, and n-hexyl. The alkyl groups can be substituted if desired, for instance, with groups such as aryl-O—, wherein aryl is as defined below, heteroaryl-O—, wherein heteroaryl is as defined below, hydroxy, amino, alkyl, and dialkylamino, halo, trifluoromethyl, carboxy, nitro, and cyano. Typical substituted alkyl groups thus are aminomethyl, 2-nitroethyl, 4-cyanobutyl, 2,3-dichloropentyl, and 3-hydroxy-5-carboxyhexyl.

Examples of NR⁴R⁵ groups include amino, methylamino, di-isopropylamino, acetyl amino, propionyl amino, 3-aminopropyl amino, 3-ethylaminobutyl amino, 3-di-n-propylamino-propyl amino, 4-diethylaminobutyl amino, and 3-carboxypropionyl amino. R⁴ and R⁵ can be taken together with the nitrogen to which they are attached to form a ring having 3 to 7 carbon atoms and 1, 2, or 3 heteroatoms selected from the group consisting of nitrogen, substituted nitrogen, oxygen, and sulfur. Examples of such cyclic NR⁴R⁵ groups include pyrrolidinyl, piperazinyl, 4-methylpiperazinyl, 4-benzylpiperazinyl, pyridinyl, piperidinyl, pyrazinyl, morpholinyl, and the like.

“Halo” includes fluoro, chloro, bromo, and iodo.

“Alkenyl” means straight and branched hydrocarbon radicals having from 2 to 6 carbon atoms and one double bond and includes ethenyl, 3-buten-1-yl, 2-ethenylbutyl, 3-hexen-1-yl, and the like.

“Alkynyl” means straight and branched hydrocarbon radicals having from 2 to 6 carbon atoms and one triple bond and includes ethynyl, 3-butyn-1-yl, propynyl, 2-butyn-1-yl, 3-pentyn-1-yl, and the like.

“Carbocycle” or “Cycloalkyl” mean a monocyclic or polycyclic hydrocarbyl group such as cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, and the like. Also included are rings in which 1 to 3 heteroatoms replace carbons. Such groups are termed “heterocycle” or “heterocyclic” or “heterocyclyl,” which mean a cycloalkyl group also bearing at least one heteroatom selected from O, S, or NR₂, examples being oxiranyl, pyrrolidinyl, piperidyl, tetrahydropyran, and morpholine.

“Alkoxy” refers to the alkyl groups mentioned above bound through oxygen, examples of which include methoxy, ethoxy, isopropoxy, tert-butoxy, and the like. In addition, alkoxy refers to polyethers such as —O—(CH₂)₂—O—OH₃, and the like.

“Alkanoyl” groups are alkyl linked through a carbonyl, ie, C₁-C₅-C(O)—. Such groups include formyl, acetyl, propionyl, butyryl, and isobutyryl.

“Acyl” means an alkyl or aryl (Ar) group bonded through a carbonyl group, i.e., R—C(O)—. For example, acyl includes a C₁-C₆ alkanoyl, including substituted alkanoyl, wherein the alkyl portion can be substituted by NR⁴R⁵ or a carboxylic or heterocyclic group. Typical acyl groups include acetyl, benzoyl, and the like.

The alkyl, alkenyl, alkoxy, and alkynyl groups described above are optionally substituted, preferably by 1 to 3 groups selected from NR⁴R⁵, phenyl, substituted phenyl, thio C₁-C₆ alkyl, C₁-C₆ alkoxy, hydroxy, carboxy, aryl-O—, wherein aryl is as defined below, heteroaryl-O—, wherein heteroaryl is as defined below, C₁-C₆ alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₙPh where n is 1, 2, or 3. Perhalo and polyhalo substitution is also embraced. Oxo (═O) substitution of a CH₂ carbon group to provide a carbonyl (C═O) is also embraced.

Examples of substituted alkyl groups include 2-aminoethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, and 2-(4-methylpiperazinyl)ethyl.

Examples of substituted alkynyl groups include 2-methoxyethynyl, 2-ethylsulfanyethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The terms “Ar” and “aryl” refer to unsubstituted and substituted aromatic groups. Heteroaryl groups have from 4 to 10 ring atoms which are carbon atoms, and from 1 to 4 of which are independently selected from the group consisting of O, S, and N. Preferred heteroaryl groups have 1 or 2 heteroatoms in a 5- or 6-membered aromatic ring. Mono and bicyclic aromatic ring systems are included in the definition of aryl and heteroaryl. Typical aryl and heteroaryl groups include phenyl, 3-chlorophenyl, 2,6-dibromophenyl, pyridyl, 3-methylpyridyl, benzothienyl, 2,4,6-tribromophenyl, 4-ethylbenzothienyl, furanyl, 3,4-diethylfuranyl, naphthyl, 4,7-dichloronaphthyl, morpholinyl, indolyl, benzotriazolyl, indazolyl, pyrrole, pyrazole, imidazole, thiazole, and the like.

Preferred Ar groups are phenyl and phenyl substituted by 1, 2, or 3 groups independently selected from the group consisting of alkyl, alkoxy, thio, thioalkyl, 1H-tetrazol-5-yl, halo, hydroxy, —COOR⁶, trifluoromethyl, nitro, amino of the formula —NR⁴R⁵, and T(CH₂)ₘQR⁴ or T(CH₂)ₘCO₂R⁴ wherein m is 1 to 6, T is O, S, NR⁴, N(O)R⁴, NR⁴R⁵Y, or CR⁴R⁵, Q is O, S, NR⁵, N(O)R⁵, or NR⁴R⁵Y wherein R⁴ and R⁵ are as described above, and R⁶ is hydrogen, alkyl, or substituted alkyl, for example, methyl, trichloroethyl, diphenylmethyl, and the like. The alkyl and alkoxy groups can be substituted as defined above. For example, typical groups are carboxyalkyl, alkoxycarbonylalkyl, hydroxyalkyl, hydroxyalkoxy, and alkoxyalkyl. Typical substituted aryl groups include 2,6-dichlorophenyl, 3-methoxyphenyl, 4-trifluoromethylphenyl, 4-styrylphenyl, 3-amino-4-nitrophenyl, 3,5-dihydroxyphenyl, and the like.

Most preferred aryl is phenyl, 4- or 3-methoxy-phenyl, 4-fluorophenyl, and 3-fluorophenyl, and each of 3,4-disubstituted phenyls wherein the substituents are methoxy and fluoro.

Most preferred heteroaryl is pyridin-4-yl or 2-methoxypyridin-4-yl.

Definitions of Terms Used to Define Compounds of Formula IC:

In Formula IC,:

The term “halogen” means F, Cl, Br, or I; preferably F, Br and Cl.

The term “(C₁-C₆)alkyl” means linear or branched alkyl containing from 1 to 6, and preferably from 1 to 3 carbon atoms.

The term “(C₁-C₆)alkoxy” means linear or branched alkyl containing from 1 to 6, and preferably from 1 to 3, carbon atoms bonded through an oxygen atom.

The term “(C₃-C₆)alkenyl” means an alkenyl containing from 3 to 6, and preferably 3 or 4 carbon atoms, more particularly allyl.

The term “(C₃-C₆)alkynyl” means alkynyl containing from 3 to 6, and preferably 3 or 4, carbon atoms, more particularly propargyl.

The term “aryl” means an aromatic ring containing from 5 to 10, and preferably 5 or 6, carbon atoms.

The term “heteroaryl” means a heteroaromatic aryl group interrupted with one or several hetero atom selected from nitrogen, oxygen and sulphur. The term “interrupted” means that the hetero atom can replace a carbon atom of the ring. Examples of such groups containing a heteroatom are, inter alia, thienyl, pyridyl, benzofurazanyl.

The term “heterocycle” means an aromatic or non-aromatic, 5-or 6-membered monocycle comprising carbon atoms and from 1 to 4 heteroatoms selected from nitrogen, oxygen and sulphur.

The term “aryl(C₁-C₆)alkyl” means an aryl, as defined above, bonded through an alkyl, wherein the alkyl contains from 1 to 6, and preferably from 1 to 4, carbon atoms.

The term “cycloalkyl” means a cycloalkyl containing from 3 to 8, and preferably from 3 to 6, carbon atoms.

The term “cycloalkyl(C₁-C₆)alkyl” means a cycloalkyl group bonded through an alkyl group, wherein the alkyl contains from 1 to 6, and preferably from 1 to 3, carbon atoms and the cycloalkyl contains from 3 to 6 carbon atoms.

The phrase “multiple bond” represents a double bond or a triple bond.

Definitions of Terms Used to Define Compounds of Formula ID:

The term “halogen” means F, Cl, Br, or I; preferably F, Br and Cl.

The term “(C₁-C₆)alkyl” means linear or branched alkyl containing from 1 to 6, and preferably from 1 to 3 carbon atoms.

The term “halo(C₁-C₆)alkyl” means (C₁-C₆)alkyl as defined above substituted with one or more halogen atoms, and preferably trihalogenomethyl.

The term “(C₁-C₆)alkoxy” means linear or branched alkyl containing from 1 to 6, and preferably from 1 to 3, carbon atoms bonded through an oxygen atom.

The term “(C₃-C₆)alkenyl” means an alkenyl containing from 3 to 6, and preferably 3 or 4 carbon atoms, more particularly allyl.

The term “(C₃-C₆)alkynyl” means alkynyl containing from 3 to 6, and preferably 3 or 4, carbon atoms, more particularly propargyl.

The term “aryl” means an aromatic ring containing from 5 to 10, and preferably 5 or 6, carbon atoms.

The term “heteroaryl” means a heteroaromatic aryl group interrupted with one or several hetero atom selected from nitrogen, oxygen and sulphur. The term “interrupted” means that the hetero atom can replace a carbon atom of the ring. Examples of such groups containing a heteroatom are, inter alia, thienyl, pyridyl, benzofurazanyl.

The term “heterocycle” means an aromatic or non-aromatic, 5-or 6-membered monocycle comprising carbon atoms and from 1 to 4 heteroatoms selected from nitrogen, oxygen and sulphur.

The term “aryl(C₁-C₆)alkyl” means an aryl, as defined above, bonded through an alkyl, wherein the alkyl contains from 1 to 6, and preferably from 1 to 4, carbon atoms.

The term “cycloalkyl” means a cycloalkyl containing from 3 to 8, and preferably from 3 to 6, carbon atoms.

The term “cycloalkyl(C₁-C₆)alkyl” means a cycloalkyl group bonded through an alkyl group, wherein the alkyl contains from 1 to 6, and preferably from 1 to 3, carbon atoms and the cycloalkyl contains from 3 to 6 carbon atoms.

The phrase “multiple bond” represents a double bond or a triple bond.

Definitions of Terms Used to Define Compounds of Formula IE:

In Formula IE, R¹ to R³ include “C₁-C₆ alkyl” groups. These are straight and branched carbon chains having from 1 to 6 carbon atoms. Examples of such alkyl groups include methyl, ethyl, isopropyl, tert-butyl, neopentyl, and n-hexyl. The alkyl groups can be substituted if desired, for instance with groups such as hydroxy, alkoxy, amino, alkyl and dialkylamino, alkanoyl, acyl, halo, trifluoromethyl, carboxy, nitro, and cyano.

“Alkenyl” means straight and branched hydrocarbon radicals having from 2 to 6 carbon atoms and one double bond and includes ethenyl, 3-buten-1-yl, 2-ethenylbutyl, 3-hexen-1-yl, and the like.

“Alkynyl” means straight and branched hydrocarbon radicals having from 2 to 6 carbon atoms and one triple bond and includes ethynyl, 3-butyn-1-yl, propynyl, 2-butyn-1-yl, 3-pentyn-1-yl, and the like.

“Cycloalkyl” means a monocyclic or polycyclic hydrocarbyl group such as cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, and the like. Also included are rings in which 1 to 3 heteroatoms replace carbons. Such groups are termed “heterocycle” or “heterocyclyl”, which means a cycloalkyl group also bearing at least one heteroatom selected from O, S, or NR², examples being oxiranyl, pyrrolidinyl, piperidyl, tetrahydropyran, and morpholine.

“Alkoxy” refers to the alkyl groups mentioned above bound through oxygen, examples of which include methoxy, ethoxy, isopropoxy, tert-butoxy, and the like. In addition, alkoxy refers to polyethers such as —O—(CH₂)₂—O—CH₃, and the like. “Thioalkoxy” is an alkoxy group wherein the 0 is replaced by an S.

“Alkanoyl” groups are alkyl linked through a carbonyl, ie, C₁-C₅-C(O)—. Such groups include formyl, acetyl, propionyl, butyryl, and isobutyryl.

“Acyl” means an R group that is a C₁-C₆ alkyl or aryl (Ar) group bonded through a carbonyl group, i.e., R—C(O)—, wherein C₁-C₆ alkyl and aryl are as defined above and below, respectively. The phrase “substituted acyl” means an R group that is a substituted C₁-C₆ alkyl or a substituted aryl (substituted Ar) group bonded through a carbonyl group. For example, substituted acyl includes substituted alkanoyl, wherein the alkyl portion can be substituted by NR⁴R⁵ or a carboxylic or heterocyclic group. Typical acyl groups include acetyl, benzoyl, and the like. Typical substituted acyl groups include trifluoroacetyl, 4-carboxybenzoyl, and the like.

The alkyl, alkenyl, alkoxy, and alkynyl groups described above are optionally substituted, preferably by 1 to 3 groups selected from NR⁴R⁵, phenyl, substituted phenyl, (CH₂)ₘ—C(O) phenyl, (CH₂)ₘ C(O) substituted phenyl, (CH₂)ₘ—S(O)₀₋₂ phenyl, (CH₂)ₘ S(O)₀₋₂ substituted phenyl, (CH₂)ₘ—C(O) heteroaryl, (CH₂)ₘ C(O) substituted heteroaryl, (CH₂)ₘ—S(O)₀₋₂ heteroaryl, (CH₂)ₘ—S(O)₀₋₂ substituted heteroaryl, (CH₂)ₘ cycloalkyl, heterocycle, thio C₁-C₆ alkyl, C₁-C₆ alkoxy, hydroxy, acyl, carboxy, alkanoyl, C₁-C₆ alkoxycarbonyl, halo, nitro, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)_{y}Ph where y is 1, 2, or 3. Perhalo and polyhalo substitution is also embraced.

R⁴ and R⁵ independently are hydrogen, C₁-C₆ alkyl, C₂-C₆ alkenyl, C₂-C₆ alkynyl, acyl, (CH₂)ₘ aryl, (CH₂)ₘ heteroaryl, (CH₂)ₘ cycloalkyl, wherein these groups may be unsubstituted or substituted as described herein, or R⁴ and R⁵ are taken together with the nitrogen atom to which they are attached to form a 3- to 7-membered ring containing carbon atoms, the nitrogen atom bearing R⁴ and R⁵, and optionally 1 or 2 heteroatoms selected from O, S, NH, and NR², wherein R² is as defined above, the ring optionally may be substituted with oxo (”═O”) on a carbon atom.

Examples of NR⁴R⁵ groups include amino, methylamino, di-isopropylamino, acetyl amino, propionyl amino, 3-aminopropyl amino, 3-ethylaminobutyl amino, 3-di-n-propylamino-propyl amino, 4-diethylaminobutyl amino, and 3-carboxypropionyl amino. R⁴ and R⁵ can be taken together with the nitrogen to which they are attached to form a ring having 3 to 7 carbon atoms and 1, 2, or 3 heteroatoms selected from the group consisting of nitrogen, substituted nitrogen, oxygen, and sulfur. Examples of such cyclic NR⁴R⁵ groups include pyrrolidinyl, piperazinyl, 4-methylpiperazinyl, 4-benzylpiperazinyl, pyridinyl, piperidinyl, pyrazinyl, morpholinyl, and the like.

“Halo” includes fluoro, chloro, bromo, and iodo.

Examples of substituted alkyl groups include 2-aminoethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, benzyl(Bₙ), 3-morpholinopropyl, piperazinylmethyl, pyridyl-4-methyl(Py-4-me), 3-(pyridyl-4-thio)propyl, and 2-(4-methylpiperazinyl)ethyl.

Examples of substituted alkynyl groups include 2-methoxyethynyl, 2-ethylsulfanyethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The terms “Ar” and “aryl” refer to unsubstituted and substituted aromatic groups. Heteroaryl groups have from 4 to 10 ring atoms, from 1 to 4 of which are independently selected from the group consisting of O, S, and N. Preferred heteroaryl groups have 1 or 2 heteroatoms in a 5- or 6-membered aromatic ring. Mono- and bicyclic aromatic ring systems are included in the definition of aryl and heteroaryl. Typical aryl and heteroaryl groups include phenyl, 3-chlorophenyl, 3,4-methylenedioxyphenyl, 2,6-dibromophenyl, pyridyl, 3-methylpyridyl, 4-thiopyridyl, benzothienyl, 2,4,6-tribromophenyl, 4-ethylbenzothienyl, furanyl, 3,4-diethylfuranyl, naphthyl, 4,7-dichloronaphthyl, morpholinyl, indolyl, benzotriazolyl, indazolyl, pyrrole, pyrazole, imidazole, thiazole, and the like.

Preferred Ar groups are phenyl or naphthyl, and phenyl or naphthyl substituted by 1, 2, or 3 groups independently selected from the group consisting of alkyl, alkoxy, thio, thioalkyl, thioalkoxy, (CH₂)ₘN(R⁴)S(O)₂(C₁-C₆ alkyl), (CH₂)ₘS(O)₂NR⁴R⁵, wherein R⁴, R⁵, and m are as defined above, S(O)₂NR⁴R⁵, C(O)NR⁴R⁵, N(H)C(O)NR⁴R⁵, O—C(O)NR⁴R⁵, halo, hydroxy, —COOR⁶, trifluoromethyl, nitro, amino of the formula —NR⁴R⁵, C(O)NR⁴R⁵, S(O)C₁-C₆ alkyl, S(O)₂C₁-C₆ alkyl, 5-membered heteroaryl, N(R⁵)C(O)O(C₁-C₆ alkyl), and T(CH₂)ₚQR⁴ or T(CH₂)ₚCO₂R⁴, wherein p is 1 to 6, T is O, S, SO, SO₂, NR⁴, N(O)R⁴, NR⁴R⁶Y, or CR⁴R⁵, Q is O, S, SO, SO₂, NR⁵, N(O)R⁵, or NR⁵R⁶Y, wherein R⁴ and R⁵ are as described above, Y is a counter ion such as halo, R⁶ is H, C₁-C₆ alkyl, or substituted C₁-C₆ alkyl, for example, methyl, trichloroethyl, diphenylmethyl, and the like. The alkyl and alkoxy groups can be substituted as defined above. For example, typical groups are carboxyalkyl, alkoxycarbonylalkyl, hydroxyalkyl, hydroxyalkoxy, and alkoxyalkyl. Examples of substituted phenyl are 3-methoxyphenyl, 2,6-dichlorophenyl, 3-nitrophenyl, 4-dimethylaminophenyl, and biphenyl. Examples of quaternary ammonium groups defined by NR⁴R⁶Y are trimethylammonium chloride and triethylammonium bromide.

Heteroaryl groups may be substituted with up to 3 groups independently selected from the 1, 2, or 3 groups described above for substituted phenyl.

Definitions of Terms Used to Define Compounds of Formula IF:

In Formula IF, R¹ to R⁹ include “C₁-C₆ alkyl” groups. These are straight and branched carbon chains having from 1 to 6 carbon atoms. Examples of such alkyl groups include methyl, ethyl, isopropyl, tert-butyl, neopentyl, and n-hexyl. The alkyl groups can be substituted if desired, for instance with groups such as hydroxy, amino, alkyl, and dialkylamino, halo, trifluoromethyl, carboxy, nitro, and cyano.

“Alkenyl” means straight and branched hydrocarbon radicals having from 2 to 6 carbon atoms and one double bond and includes ethenyl, 3-buten-1-yl, 2-ethenylbutyl, 3-hexen-1-yl, and the like.

“Alkynyl” means straight and branched hydrocarbon radicals having from 2 to 6 carbon atoms and one triple bond and includes ethynyl, 3-butyn-1-yl, propynyl, 2-butyn-1-yl, 3-pentyn-1-yl, and the like.

“Cycloalkyl” means a monocyclic or polycyclic hydrocarbyl group such as cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, and the like. Also included are rings in which 1 to 3 heteroatoms replace carbons. Such groups are termed “heterocyclyl,” which means a cycloalkyl group also bearing at least one heteroatom selected from O, S, or NR², examples being oxiranyl, pyrrolidinyl, piperidyl, tetrahydropyranyl, and morpholinyl.

“Alkoxy” refers to the alkyl groups mentioned above bound through oxygen, examples of which include methoxy, ethoxy, isopropoxy, tert-butoxy, and the like. In addition, alkoxy refers to polyethers such as —O—(CH₂)₂—O—OH₃, and the like.

“Acyl” means an R group that is an alkyl or aryl (Ar) group bonded through a carbonyl group, i.e., R—C(O)—, where R is alkyl or aryl. For example, acyl includes a C₁-C₆ alkanoyl, including substituted alkanoyl, wherein the alkyl portion can be substituted by NR⁴R⁵ or a carboxylic or heterocyclic group. Typical acyl groups include acetyl, benzoyl, isonicotinoyl, and the like.

The alkyl, alkenyl, alkoxy, and alkynyl groups described above are optionally substituted, preferably by 1 to 3 groups selected from NR⁴R⁵, phenyl, substituted phenyl, thio C₁-C₆ alkyl, C₁-C₆ alkoxy, hydroxy, carboxy, C₁-C₆ alkoxycarbonyl, acyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₙPh where n is 1, 2, or 3. Perhalo and polyhalo substitution is also embraced.

Examples of substituted alkyl groups include 2-aminoethyl, acetylmethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, 4-benzoylbutyl, and 2-(4-methylpiperazinyl)ethyl.

Examples of substituted alkynyl groups include 2-methoxyethynyl, 2-benzoylethylyl, 2-ethylsulfanyethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, acetoxymethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The terms “Ar” and “aryl” refer to unsubstituted and substituted aromatic groups. Heteroaryl (Het) groups have from 4 to 9 ring atoms, from 1 to 4 ring atoms of which are independently selected from the group consisting of O, S, and N. Preferred heteroaryl groups have 1 or 2 heteroatoms in a 5- or 6-membered aromatic ring. Mono- and bicyclic aromatic ring systems are included in the definition of aryl and heteroaryl. Preferred substituent groups include alkyl, alkoxy, halo, amino, alkylamino, dialkylamino, CN, CF₃, thioalkyl, acyl and hydroxy. Typical aryl and heteroaryl groups include phenyl, 3-chlorophenyl, 2,6-dibromophenyl, pyridyl, 3-methylpyridyl, benzothienyl, 2,4,6-tribromophenyl, 4-ethylbenzothienyl, furanyl, 3,4-diethylfuranyl, naphthyl, 4,7-dichloronaphthyl, morpholinyl, indolyl, benzotriazolyl, indazolyl, pyrrole, pyrazole, imidazole, thiazole, methylenedioxyphenyl, benzo-2,1,3-thiadiazole, benzo-2,1,3-oxadiazole, and the like.

Preferred Ar groups are phenyl and phenyl substituted by 1, 2, or 3 groups independently selected from the group consisting of alkyl, alkoxy, thio, thioalkyl, halo, hydroxy, —COOR⁷, trifluoromethyl, nitro, amino of the formula —NR⁴R⁵, and T(CH₂)ₘQR⁴ or T(CH₂)ₘCO₂R⁴ wherein m is 1 to 6, T is O, S, NR⁴, N(O)R⁴, NR⁴R⁶Y, or CR⁴R⁵, Q is O, S, NR⁵, N(O)R⁵, or NR⁵R⁶Y wherein R⁴ and R⁵ are as described above, and R⁷ is hydrogen, alkyl, or substituted alkyl, for example, methyl, trichloroethyl, diphenylmethyl, and the like. The alkyl and alkoxy groups can be substituted as defined above. For example, typical groups are carboxyalkyl, alkoxycarbonylalkyl, hydroxyalkyl, hydroxyalkoxy, and alkoxyalkyl. Typical substituted aryl groups include 2,6-dichlorophenyl, 3-hydroxyphenyl, 1,3-benzodioxolyl, 4-dimethylaminophenyl, 2,4,6-triethoxyphenyl, 3-cyanophenyl, 4-methylthiophenyl, and 3,5-dinitrophenyl.

Examples of NR⁴R⁵ groups include amino, methylamino, di-isopropylamino, acetyl amino, propionyl amino, 3-aminopropyl amino, 3-ethylaminobutyl amino, 3-di-n-propylamino-propyl amino, 4-diethylaminobutyl amino, and 3-carboxypropionyl amino. R⁴ and R⁵ can be taken together with the nitrogen to which they are attached to form a ring having 3 to 7 carbon atoms and 1, 2, or 3 heteroatoms selected from the group consisting of nitrogen, substituted nitrogen, oxygen, and sulfur. Examples of such cyclic NR⁴R⁵ groups include pyrrolidinyl, piperazinyl, 4-methylpiperazinyl, 4-benzylpiperazinyl, pyridinyl, piperidinyl, pyrazinyl, morpholinyl, and the like.

“Halo” includes fluoro, chloro, bromo, and iodo.

Definitions of Terms Used to Define Compounds of Formula IG:

In Formula IG, R¹ to R⁹ include “C₁-C₆ alkyl” groups. These are straight and branched carbon chains having from 1 to 6 carbon atoms. Examples of such alkyl groups include methyl, ethyl, isopropyl, tert-butyl, neopentyl, and n-hexyl. The alkyl groups can be substituted if desired, for instance with groups such as hydroxy, amino, alkyl, aryl, and dialkylamino, halo, trifluoromethyl, carboxy, nitro, and cyano.

“Alkenyl” means straight and branched hydrocarbon radicals having from 2 to 6 carbon atoms and one double bond and includes ethenyl, 3-buten-1-yl, 2-ethenylbutyl, 3-hexen-1-yl, and the like.

“Alkynyl” means straight and branched hydrocarbon radicals having from 2 to 6 carbon atoms and one triple bond and includes ethynyl, 3-butyn-1-yl, propynyl, 2-butyn-1-yl, 3-pentyn-1-yl, and the like.

“Cycloalkyl” means a monocyclic or polycyclic hydrocarbyl group such as cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, and the like. Cycloalkyl groups can also be fused by two points of attachment to other groups such as aryl and heteroaryl groups. Also included are rings in which 1 to 3 heteroatoms replace carbons. Such groups are termed “heterocyclyl,” which means a cycloalkyl group also bearing at least one heteroatom selected from O, S, or NR², examples being oxiranyl, pyrrolidinyl, piperidyl, tetrahydropyran, and morpholine.

“Alkoxy” refers to the alkyl groups mentioned above bound through oxygen, examples of which include methoxy, ethoxy, isopropoxy, tert-butoxy, and the like. In addition, alkoxy refers to polyethers such as —O—(CH₂)₂—O—OH₃, and the like.

“Acyl” means an R group that is an alkyl or aryl (Ar) group bonded through a carbonyl group, i.e., R—C(O)—, where R is alkyl or aryl. For example, acyl includes a C₁-C₆ alkanoyl, including substituted alkanoyl, wherein the alkyl portion can be substituted by NR⁴R⁵ or a carboxylic or heterocyclic group. Typical acyl groups include acetyl, benzoyl, isonicotinoyl, and the like.

The alkyl, alkenyl, alkoxy, and alkynyl groups described above are optionally substituted, preferably by 1 to 3 groups selected from NR⁴R⁵, phenyl, substituted phenyl, naphthyl, thio C₁-C₆ alkyl, C₁-C₆ alkoxy, hydroxy, carboxy, C₁-C₆ alkoxycarbonyl, acyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₙPh where n is 1, 2, or 3. Perhalo and polyhalo substitution is also embraced.

Examples of substituted alkyl groups include 2-aminoethyl, acetylmethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, 4-benzoylbutyl, and 2-(4-methylpiperazinyl)ethyl.

Examples of substituted alkynyl groups include 2-methoxyethynyl, 2-benzoylethylyl, 2-ethylsulfanyethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, acetoxymethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The terms “Ar” and “aryl” refer to unsubstituted and substituted aromatic groups. Heteroaryl (Het) groups have from 4 to 9 ring atoms, from 1 to 4 of which are independently selected from the group consisting of O, S, and N. Preferred heteroaryl groups have 1 or 2 heteroatoms in a 5- or 6-membered aromatic ring. Mono- and bicyclic aromatic ring systems are included in the definition of aryl and heteroaryl. Preferred substituent groups include alkyl, alkoxy, aryloxy, halo, amino, alkylamino, dialkylamino, CN, CF₃, thioalkyl, acyl and hydroxy. Typical aryl and heteroaryl groups include phenyl, 3-chlorophenyl, 2,6-dibromophenyl, pyridyl, 3-methylpyridyl, benzothienyl, 2,4,6-tribromophenyl, 4-ethylbenzothienyl, furanyl, 3,4-diethylfuranyl, naphthyl, 4,7-dichloronaphthyl, morpholinyl, indolyl, benzotriazolyl, indazolyl, pyrrole, pyrazole, imidazole, thiazole, methylenedioxyphenyl, benzo-2,1,3-thiadiazole, benzo-2,1,3-oxadiazole, and the like.

Preferred Ar groups are phenyl and phenyl substituted by 1, 2, or 3 groups independently selected from the group consisting of alkyl, alkoxy, thio, thioalkyl, halo, hydroxy, —COOR⁷, trifluoromethyl, nitro, amino of the formula —NR⁴R⁵, and T(CH₂)ₘQR⁴ or T(CH₂)ₘCO₂R⁴ wherein m is 1 to 6, T is O, S, NR⁴, N(O)R⁴, NR⁴R⁶Y, or CR⁴R⁵, Q is O, S, NR⁵, N(O)R⁵, or NR⁵R⁶Y wherein R⁴ and R⁵ are as described above, and R⁷ is hydrogen, alkyl, or substituted alkyl, for example, methyl, trichloroethyl, diphenylmethyl, and the like. The alkyl and alkoxy groups can be substituted as defined above. For example, typical groups are carboxyalkyl, alkoxycarbonylalkyl, hydroxyalkyl, hydroxyalkoxy, and alkoxyalkyl. Typical substituted aryl groups include 2,6-dichlorophenyl, 3-hydroxyphenyl, 1,3-benzodioxolyl, 4-dimethylaminophenyl, 2,4,6-triethoxyphenyl, 3-cyanophenyl, 4-methylthiophenyl, and 3,5-dinitrophenyl.

Examples of NR⁴R⁵ groups include amino, methylamino, di-isopropylamino, acetyl amino, propionyl amino, 3-aminopropyl amino, 3-ethylaminobutyl amino, 3-di-n-propylamino-propyl amino, 4-diethylaminobutyl amino, and 3-carboxypropionyl amino. R⁴ and R⁵ can be taken together with the nitrogen to which they are attached to form a ring having 3 to 7 carbon atoms and 1, 2, or 3 heteroatoms selected from the group consisting of nitrogen, substituted nitrogen, oxygen, and sulfur. Examples of such cyclic NR⁴R⁵ groups include pyrrolidinyl, piperazinyl, 4-methylpiperazinyl, 4-benzylpiperazinyl, pyridinyl, piperidinyl, pyrazinyl, morpholinyl, and the like.

“Halo” includes fluoro, chloro, bromo, and iodo.

Unless moieties of a compound of the invention are defined as being unsubstituted, the moieties of the compound of the invention may be substituted. In the event where the substituents of the moieties which may be substituted are not defined above, the moieties of the compound of the invention may be optionally substituted from 1 to 3 times at any of from 1 to 3 carbon atoms, respectively, wherein each carbon atom is capable of substitution by replacement of a hydrogen atom with a group independently selected from:

C₁-C₄ alkyl;

C₂-C₄ alkenyl;

C₂-C₄ alkynyl;

CF₃;

halo;

OH;

O—(C₁-C₄ alkyl);

OCH₂F;

OCHF₂;

OCF₃;

OC(O)—(C₁-C₄ alkyl);

OC(O)O—(C₁-C₄ alkyl);

OC(O)NH—(C₁-C₄ alkyl);

OC(O)N(C₁-C₄ alkyl)₂;

OC(S)NH—(C₁-C₄ alkyl);

OC(S)N(C₁-C₄ alkyl)₂;

SH;

S—(C₁-C₄ alkyl);

S(O)—(C₁-C₄ alkyl);

S(O)₂—(C₁-C₄ alkyl);

SC(O)—(C₁-C₄ alkyl);

SC(O)O—(C₁-C₄ alkyl);

NH₂;

N(H)—(C₁-C₄ alkyl);

N(C₁-C₄ alkyl)₂;

N(H)C(O)—(C₁-C₄ alkyl);

N(CH₃)C(O)—(C₁-C₄ alkyl);

N(H)C(O)—CF₃;

N(CH₃)C(O)—CF₃;

N(H)C(S)—(C₁-C₄ alkyl);

N(CH₃)C(S)—(C₁-C₄ alkyl);

N(H)S(O)₂—(C₁-C₄ alkyl);

N(H)C(O)NH₂;

N(H)C(O)NH—(C₁-C₄ alkyl);

N(CH₃)C(O)NH—(C₁-C₄ alkyl);

N(H)C(O)N(C₁-C₄ alkyl)₂;

N(CH₃)C(O)N(C₁-C₄ alkyl)₂;

N(H)S(O)₂NH₂;

N(H)S(O)₂NH—(C₁-C₄ alkyl);

N(CH₃)S(O)₂NH—(C₁-C₄ alkyl);

N(H)S(O)₂N(C₁-C₄ alkyl)₂;

N(CH₃)S(O)₂N(C₁-C₄ alkyl)₂;

N(H)C(O)O—(C₁-C₄ alkyl);

N(CH₃)C(O)O—(C₁-C₄ alkyl);

N(H)S(O)₂O—(C₁-C₄ alkyl);

N(CH₃)S(O)₂O—(C₁-C₄ alkyl);

N(CH₃)C(S)NH—(C₁-C₄ alkyl);

N(CH₃)C(S)N(C₁-C₄ alkyl)₂;

N(CH₃)C(S)O—(C₁-C₄ alkyl);

N(H)C(S)NH₂;

NO₂;

CO₂H;

CO₂—(C₁-C₄ alkyl);

C(O)N(H)OH;

C(O)N(CH₃)OH;

C(O)N(CH₃)OH;

C(O)N(CH₃)O—(C₁-C₄ alkyl);

C(O)N(H)—(C₁-C₄ alkyl);

C(O)N(C₁-C₄ alkyl)₂;

C(S)N(H)—(C₁-C₄ alkyl);

C(S)N(C₁-C₄ alkyl)₂;

C(NH)N(H)—(C₁-C₄ alkyl);

C(NH)N(C₁-C₄ alkyl)₂;

C(NCH₃)N(H)—(C₁-C₄ alkyl);

C(NCH₃)N(C₁-C₄ alkyl)₂;

C(O)—(C₁-C₄ alkyl);

C(NH)—(C₁-C₄ alkyl);

C(NCH₃)—(C₁-C₄ alkyl);

C(NOH)—(C₁-C₄ alkyl);

C(NOCH₃)—(C₁-C₄ alkyl);

CN;

CHO;

CH₂OH;

CH₂O—(C₁-C₄ alkyl);

CH₂NH₂;

CH₂N(H)—(C₁-C₄ alkyl); and

CH₂N(C₁-C₄ alkyl)₂; wherein

“C₁-C₄ alkyl” means a straight or branched, unsubstituted alkyl chain of from 1 to 4 carbon atoms;

“C₂-C₄ alkenyl” means a straight or branched, unsubstituted alkenyl chain of from 2 to 4 carbon atoms; and

“C₂-C₄ alkynyl” means a straight or branched, unsubstituted alkynyl chain of from 2 to 4 carbon atoms.

It should be appreciated that the S1′ site of MMP-13 was previously thought to be a grossly linear channel which contained an opening at the top that allowed an amino acid side chain from a substrate molecule to enter during binding, and was closed at the bottom. Applicant has discovered that the S1′ site is actually composed of an S1′ channel angularly connected to a newly discovered pocket which applicant calls the S1″ site. The S1″ site is open to solvent at the bottom, which can expose a functional group of Applicant's allosteric carboxylic inhibitors to solvent. For illustrative purposes, the S1′ site of the MMP-13 enzyme can now be thought of as being like a sock with a hole in the toes, wherein the S1′ channel is the region from approximately the opening to the ankle, and the S1″ site is the foot region below the ankle, which foot region is angularly connected to the ankle region.

More particularly, the S1′ channel is a specific part of the S1′ site and is formed largely by Leu218, Val219, His222 and by residues from Leu239 to Tyr244. The S1″ binding site which has been newly discovered is defined by residues from Tyr246 to Pro255. The S1″ site contains at least two hydrogen bond donors and aromatic groups which interact with a compound which is an allosteric carboxylic inhibitor of MMP-13.

Without wishing to be bound by any particular theory, the inventor believes that the S1″ site could be a recognition site for triple helix collagen, the natural substrate for MMP-13. It is possible that the conformation of the S1″ site is modified only when an appropriate compound binds to MMP-13, thereby interfering with the collagen recognition process. This newly discovered pattern of binding offers the possibility of greater selectivity than what is achievable with the binding pattern of known selective inhibitors of MMP-13, wherein the known binding pattern requires ligation of the catalytic zinc atom at the active site and occupation the S1′ channel, but not the S1″ site.

The instant allosteric carboxylic inhibitors of MMP-13 are described in co-pending PCT international applications and their corresponding United States nonprovisional application Ser. Nos. 10/071,032; 10/075,918; 10/075,073; 10/075,069; 10/075,954; 10/075,654; 10/074,646; 10/075,909; and 10/071,073, and the related U.S. provisional application Nos. 60/268,780; 60/268,736; 60/268,756; 60/268,821; 60/268,861; 60/268,757; 60/268,782; 60/268,779; and 60/268,781, respectively, all provisional applications filed on Feb. 14, 2001, and from which benefit of priority is claimed. All of the these PCT International applications, United States provisional applications, and United States nonprovisional applications are incorporated herein by reference. For convenience, the allosteric inhibitors of MMP-13 patent application filing information is listed below in Table A.

TABLE A
Allosteric inhibitors of MMP-13 patent
application filing information

| | | Corresponding | |
|---|---|---|---|
| | | U.S. | Corresponding |
| U.S. Provisional | U.S. Provisional | Nonprovisional | PCT International |
| Application | Application | Application | Application |
| Number | Filing Date | Number | Number |
| 60/268,780 | Feb. 14, 2001 | 10/071,032 | PCT/IB02/00313 |
| 60/268,736 | Feb. 14, 2001 | 10/075,918 | PCT/IB02/00344 |
| 60/268,756 | Feb. 14, 2001 | 10/075,073 | PCT/IB02/00204 |
| 60/268,821 | Feb. 14, 2001 | 10/075,069 | PCT/IB02/00447 |
| 60/268,661 | Feb. 14, 2001 | 10/075,954 | PCT/EP02/01979 |
| 60/268,757 | Feb. 14, 2001 | 10/075,654 | PCT/FR02/00504 |
| 60/268,782 | Feb. 14, 2001 | 10/074,646 | PCT/IB02/00083 |
| 60/268,779 | Feb. 14, 2001 | 10/075,909 | PCT/IB02/00190 |
| 60/268,781 | Feb. 14, 2001 | 10/071,073 | PCT/IB02/00345 |

It should be appreciated that invention combinations may comprise celecoxib, or a pharmaceutically acceptable salt thereof, or valdecoxib, or a pharmaceutically acceptable salt thereof, and an allosteric carboxylic inhibitor of MMP-13, or a pharmaceutically acceptable salt thereof, wherein the allosteric carboxylic inhibitor of MMP-13, or a pharmaceutically acceptable salt thereof, may embrace any one of the compound embodiments described in co-pending PCT international applications and their corresponding United States nonprovisional application Ser. Nos. 10/071,032; 10/075,918; 10/075,073; 10/075,069; 10/075,954; 10/075,654; 10/074,646; 10/075,909; and 10/071,073, and the related U.S. provisional application Nos. 60/268,780; 60/268,736; 60/268,756; 60/268,821; 60/268,861; 60/268,757; 60/268,782; 60/268,779; and 60/268,781, respectively, including variants thereof described in the respective specifications and claims. It should be further appreciated that the above-described pharmaceutical compositions may comprise these invention combinations. It should be further appreciated that the above described methods of prevention, treatment, or inhibition may comprise administration of these invention combinations.

A compound that is an allosteric carboxylic inhibitor of MMP-13 may be readily identified by one of ordinary skill in the pharmaceutical or medical arts by assaying an carboxylic test compound for inhibition of MMP-13 as described below in Biological Methods 1 or 2, and for allosteric inhibition of MMP-13 by assaying the carboxylic test compound for inhibition of MMP-13 in the presence of an inhibitor to the catalytic zinc of MMP-13 as described below in Biological Methods 3 or 4.

Further, an allosteric carboxylic inhibitor of MMP-13 having an anti-inflammatory, an analgesic, anti-arthritic, or a cartilage damage inhibiting effect, or any combination of these effects, may be readily identified by one of ordinary skill in the pharmaceutical or medical arts by assaying the allosteric carboxylic inhibitor of MMP-13 in any number of well known assays for measuring determining the allosteric carboxylic inhibitor of MMP-13's effects on cartilage damage, arthritis, inflammation, or pain. These assays include in vitro assays that utilize cartilage samples and in vivo assays in whole animals that measure cartilage degradation, inhibition of inflammation, or pain alleviation.

For example with regard to assaying cartilage damage in vitro, an amount of an allosteric carboxylic inhibitor of MMP-13 or control vehicle may be administered with a cartilage damaging agent to cartilage, and the cartilage damage inhibiting effects in both tests studied by gross examination or histopathologic examination of the cartilage, or by measurement of biological markers of cartilage damage such as, for example, proteoglycan content or hydroxyproline content. Further, in vivo assays to assay cartilage damage may be performed as follows: an amount of an allosteric carboxylic inhibitor of MMP-13 or control vehicle may be administered with a cartilage damaging agent to an animal, and the effects of the allosteric carboxylic inhibitor of MMP-13 being assayed on cartilage in the animal may be evaluated by gross examination or histopathologic examination of the cartilage, by observation of the effects in an acute model on functional limitations of the affected joint that result from cartilage damage, or by measurement of biological markers of cartilage damage such as, for example, proteoglycan content or hydroxyproline content. Several methods of identifying an allosteric carboxylic inhibitor of MMP-13 with cartilage damage inhibiting properties are described below. The amount to be administered in an assay to identify an allosteric carboxylic inhibitor of MMP-13 is dependent upon the particular assay employed, but in any event is not higher than the well known maximum amount of a compound that the particular assay can effectively accommodate.

Similarly, allosteric carboxylic inhibitors of MMP-13 having pain-alleviating properties may be identified using any one of a number of in vivo animal models of pain.

Still similarly, allosteric carboxylic inhibitors of MMP-13 having anti-inflammatory properties may be identified using any one of a number of in vivo animal models of inflammation. For example, for an example of inflammation models, see U.S. Pat. No. 6, 329,429, which is incorporated herein by reference.

Still similarly, allosteric carboxylic inhibitors of MMP-13 having anti-arthritic properties may be identified using any one of a number of in vivo animal models of arthritis. For example, for an example of arthritis models, see also U.S. Pat. No. 6, 329,429.

Any allosteric carboxylic inhibitor of MMP-13 is readily available, either commercially, or by synthetic methodology, well known to those skilled in the art of organic chemistry. For specific syntheses, see the examples below and the preparations of allosteric carboxylic inhibitors of MMP-13 described in the above-referenced patent applications.

The term “celecoxib” means the compound named 4-(5-(4-methylphenyl)-3-(trifluoromethyl)-1H-pyrazol-1-yl)-benzenesulfonamide. Celecoxib is currently approved by the FDA for the treatment of osteoarthritis, rheumatoid arthritis, and Polyposis-familial adenomatus. Celecoxib is marketed under the tradename “Celebrex”. Celecoxib is currently in clinical trials for the treatment of bladder cancer, chemopreventative-lung cancer, and post-operative pain, and is registered for the treatment of dysmenorrhea. Celecoxib has the structure drawn below:

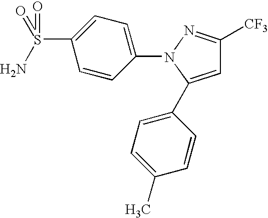

It should be appreciated that the invention combination may include celecoxib, or a pharmaceutically acceptable salt thereof. Preferred invention combinations include celecoxib.

The term “valdecoxib” means the compound named 4-(5-methyl-3-phenyl-4-isoxazolyl)-benzenesulfonamide. Valdecoxib has been approved by the FDA for treating osteoarthritis, rheumatoid arthritis, dysmenorrhea, and general pain, and is marketed under the tradename “Bextra”. Valdecoxib is in clinical trials for the treatment of migraine. Valdecoxib has the structure drawn below:

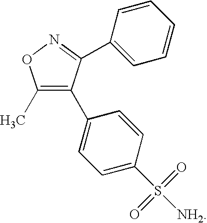

It should be appreciated that the invention combination may include valdecoxib, or a pharmaceutically acceptable salt thereof. Preferred invention combinations include valdecoxib.

It should be further appreciated that celecoxib and valdecoxib are each selective inhibitors of COX-2, which is also known as prostaglandin synthase-2 and prostaglandin PGH₂ synthase.

A selective inhibitor of COX-2 means compounds that inhibit COX-2 selectively versus COX-1 such that a ratio of IC₅₀ for a compound with COX-1 divided by a ratio of IC₅₀ for the compound with COX-2 is greater than, or equal to, 5, where the ratios are determined in one or more of the in vitro, in vivo, or ex vivo assays described below. All that is required to determine whether a compound is a selective COX-2 inhibitor is to assay a compound in one of the pairs of assays described in Biological Methods 5 to 8 below. Preferred selective COX-2 inhibitors have a selectivity greater than 5 fold versus COX-1 in the assay described in Biological Method 5 below.

The term “NSAID” is an acronym for the phrase “nonsteroidal anti-inflammatory drug”, which means any compound which inhibits cyclooxygenase-1 (“COX-1”) and cyclooxygenase-2. Most NSAIDs fall within one of the following five structural classes: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxim, peroxicam, sudoxicam, and isoxicam. Other useful NSAIDs include aspirin, acetaminophen, indomethacin, and phenylbutazone. Selective inhibitors of cyclooxygenase-2 as described above may be considered to be NSAIDs also.

For the purposes of this invention, the term “arthritis”, which is synonymous with the phrase “arthritic condition”, includes osteoarthritis, rheumatoid arthritis, degenerative joint disease, spondyloarthropathies, gouty arthritis, systemic lupus erythematosus, juvenile arthritis, and psoriatic arthritis. An allosteric carboxylic inhibitor of MMP-13 having an anti-arthritic effect is a compound as defined above that inhibits the progress, prevents further progress, or reverses progression, in part or in whole, of any one or more symptoms of any one of the arthritic diseases and disorders listed above.

Other mammalian diseases and disorders which are treatable by administration of an invention combination alone, or contained in a pharmaceutical composition as defined below, include: fever (including rheumatic fever and fever associated with influenza and other viral infections), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, bums, diabetes (including types I and II diabetes, diabetic retinopathy, neuropathy and nephropathy), tumor invasion, tumor growth, tumor metastasis, corneal scaring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock), epilepsy, convulsions, and septic shock.

The term “Thr245” means threonine 245 of an MMP-13 enzyme.

The term “Thr247” means threonine 247 of an MMP-13 enzyme.

The term “Met253” means methionine 253 of an MMP-13 enzyme.

The term “His251” means histidine 251 of an MMP-13 enzyme.

It should be appreciated that the matrix metalloproteinases include, but are not limited to, the following enzymes:

MMP-1, also known as interstitial collagenase, collagenase-1, or fibroblast-type collagenase;

MMP-2, also known as gelatinase A or 72 kDa Type IV collagenase;

MMP-3, also known as stromelysin or stromelysin-1;

MMP-7, also known as matrilysin or PUMP-1;

MMP-8, also known as collagenase-2, neutrophil collagenase or polymorphonuclear-type (“PMN-type”) collagenase;

MMP-9, also known as gelatinase B or 92 kDa Type IV collagenase;

MMP-10, also known as stromelysin-2;

MMP-11, also known as stromelysin-3;

MMP-12, also known as metalloelastase;

MMP-13, also known as collagenase-3;

MMP-14, also known as membrane-type (“MT”) 1-MMP or MT1-MMP;

MMP-15, also known as MT2-MMP;

MMP-16, also known as MT3-MMP;

MMP-17, also known as MT4-MMP;

MMP-18; and

MMP-19.

Other known MMPs include MMP-26 (Matrilysin-2).

The invention provides combinations which comprise an “allosteric carboxylic inhibitor of MMP-13”. An allosteric carboxylic inhibitor of MMP-13 is any compound that contains a carboxylic ester linker [i.e., —C(O)—O—C or C—O—C(O)-] or

carboxylic amide linker [i.e.,

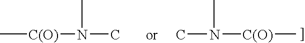

and that binds to, coordinates to, or ligates a site in an MMP-13 enzyme that is at a location other than the enzyme's catalytically active site, wherein the catalytically active site is the site where the catalytic zinc cation of the MMP-13 enzyme binds, ligates, or coordinates a natural substrate(s). Thus an allosteric carboxylic inhibitor of MMP-13 is any carboxylic-containing inhibitor of an MMP-13 that does not bind to, coordinate to, or ligate, either directly or indirectly via a bridging water molecule, the catalytic zinc cation of a MMP-13.

Further, an allosteric carboxylic inhibitor of MMP-13, as used in the present invention, is a compound that does not ligate, coordinate to, or bind to the catalytic zinc cation of MMP-13, or a truncated form thereof, and is ≧5 times more potent in vitro versus MMP-13, or a truncated form thereof, than versus at least 2 other matrix metalloproteinase enzymes, including MMP-1, MMP-2, MMP-3, MMP-7, MMP-8, MMP-9, MMP-10, MMP-11, MMP-12, MMP-14, MMP-17, MMP-18, MMP-19, MMP-21, and MMP-26, and tumor necrosis factor alpha convertase (“TACE”). A preferred aspect of the present invention is combinations comprising allosteric carboxylic inhibitors of MMP-13 that are selective inhibitors of MMP-13 over MMP-1.

Other aspects of the present invention are allosteric carboxylic inhibitors of MMP-13, or a pharmaceutically acceptable salt thereof, that are ≧10, ≧20, ≧50, ≧100, or ≧1000 times more potent versus MMP-13 than versus at least two of any other MMP enzyme or TACE.

Still other aspects of the present invention are allosteric carboxylic inhibitors of MMP-13, or a pharmaceutically acceptable salt thereof, that are selective inhibitors of MMP-13 versus 2, 3, 4, 5, 6, or 7 other MMP enzymes, or versus TACE and 1, 2, 3, 4, 5, 6, or 7 other MMP enzymes.

It should be appreciated that selectivity of an allosteric carboxylic inhibitor of MMP-13, or a pharmaceutically acceptable salt thereof, is a multidimensional characteristic that includes the number of other MMP enzymes and TACE over which selectivity for MMP-13 inhibition is present and the degree of selectivity of inhibition of MMP-13 over another particular MMP or TACE, as measured by, for example, the IC₅₀ in micromolar concentration of inhibitor for the inhibition of the other MMP enzyme or TACE divided by the IC₅₀ in micromolar concentration of inhibitor for the inhibition of MMP-13.

The term “IC₅₀” means the concentration of a compound, usually expressed as micromolar or nanomolar, required to inhibit an enzyme's catalytic activity by 50%.

The term “ED₄₀” means the concentration of a compound, usually expressed as micromolar or nanomolar, required to treat a disease in about 40% of a patient group.

The term “ED₃₀” means the concentration of a compound, usually expressed as micromolar or nanomolar, required to treat a disease in 30% of a patient group.

The phrase “pharmaceutical composition” means a composition suitable for administration in medical or veterinary use.

The term “admixed” and the phrase “in admixture” are synonymous and mean in a state of being in a homogeneous or heterogeneous mixture. Preferred is a homogeneous mixture.

As used herein, the phrase “cartilage damage” means a disorder of hyaline cartilage and subchondral bone characterized by hypertrophy of tissues in and around the involved joints, which may or may not be accompanied by deterioration of hyaline cartilage surface.

The phrase “treating”, which is related to the terms “treat” and “treated”, means administration of an invention combination as defined above that inhibits the progress, prevents further progress, or reverses progression, in part or in whole, of any one or more symptoms of any one of the diseases and disorders listed above.

The term “comprising,” which is synonymous with the terms “including,” “containing,” or “characterized by,” is inclusive or open-ended, and does not exclude additional, unrecited elements or method steps from the scope of the invention that is described following the term.

The phrase “consisting of” is closed-ended, and excludes any element, step, or ingredient not specified in the description of the invention that follows the phrase.

The phrase “consisting essentially of” limits the scope of the invention that follows to the specified elements, steps, or ingredients, and those further elements, steps, or ingredients that do not materially affect the basic and novel characteristics of the invention.

The invention combination also includes isotopically-labelled compounds, which are identical to those recited above, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention and pharmaceutically acceptable salts of said compounds which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by carrying out the procedures incorporated by reference above or disclosed in the Schemes and/or in the Examples and Preparations below, by substituting a readily available isotopically labelled reagent for a non-isotopically labelled reagent.

One of ordinary skill in the art will appreciate that the combinations of the invention are useful in treating a diverse array of diseases. One of ordinary skill in the art will also appreciate that when using the combinations of the invention in the treatment of a specific disease that the combinations of the invention may be combined with various existing therapeutic agents used for that disease.

For the treatment of rheumatoid arthritis, the combinations of the invention may be combined with agents such as TNF-α inhibitors such as anti-TNF monoclonal antibodies and TNF receptor immunoglobulin molecules (such as Enbrel®), low dose methotrexate, lefunimide, hydroxychloroquine, d-penicilamine, auranofin or parenteral or oral gold.

The combinations of the invention can also be used in combination with existing therapeutic agents for the treatment of osteoarthritis. Suitable agents to be used in combination include standard non-steroidal anti-inflammatory agents (hereinafter NSAfD's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib and rofecoxib, analgesics and intraarticular therapies such as corticosteroids and hyaluronic acids such as hyalgan and synvisc.

This invention also relates to a method of or a pharmaceutical composition for treating inflammatory processes and diseases comprising administering a combination of this invention to a mammal, including a human, cat, livestock or dog, wherein said inflammatory processes and diseases are defined as above and said inhibitory combination is used in combination with one or more other therapeutically active agents under the following conditions:

A.) where a joint has become seriously inflamed as well as infected at the same time by bacteria, fungi, protozoa and/or virus, said inhibitory combination is administered in combination with one or more antibiotic, antifungal, antiprotozoal and/or antiviral therapeutic agents;

B.) where a multi-fold treatment of pain and inflammation is desired, said inhibitory combination is administered in combination with inhibitors of other mediators of inflammation, comprising one or more members independently selected from the group consisting essentially of:

(1) NSAIDs;

(2) H₁-receptor antagonists;

(3) kinin-B₁- and B₂-receptor antagonists;

(4) prostaglandin inhibitors selected from the group consisting of PGD-, PGF- PGI₂- and PGE-receptor antagonists;

(5) thromboxane A₂ (TXA₂-) inhibitors;

(6) 5-, 12- and 15-lipoxygenase inhibitors;

(7) leukotriene LTC₄—, LTD₄/LTE₄- and LTB₄-inhibitors;

(8) PAF-receptor antagonists;

(9) gold in the form of an aurothio group together with one or more hydrophilic groups;

(10) immunosuppressive agents selected from the group consisting of cyclosporine, azathioprine and methotrexate;

(11) anti-inflammatory glucocorticoids;

(12) penicillamine;

(13) hydroxychloroquine;

(14) anti-gout agents including colchicine; xanthine oxidase inhibitors including allopurinol; and uricosuric agents selected from probenecid, sulfinpyrazone and benzbromarone;

C. where older mammals are being treated for disease conditions, syndromes and symptoms found in geriatric mammals, said inhibitory combination is administered in combination with one or more members independently selected from the group consisting essentially of:

(1) cognitive therapeutics to counteract memory loss and impairment;

(2) anti-hypertensives and other cardiovascular drugs intended to offset the consequences of atherosclerosis, hypertension, myocardial ischemia, angina, congestive heart failure and myocardial infarction, selected from the group consisting of:

a. diuretics;

b. vasodilators;

c. β-adrenergic receptor antagonists;

d. angiotensin-II converting enzyme inhibitors (ACE-inhibitors), alone or optionally together with neutral endopeptidase inhibitors;

e. angiotensin II receptor antagonists;

f. renin inhibitors;

g. calcium channel blockers;

h. sympatholytic agents;

i. α₂-adrenergic agonists;

j. α-adrenergic receptor antagonists; and

k. HMG-CoA-reductase inhibitors (anti-hypercholesterolemics);

(3) antineoplastic agents selected from:

a. antimitotic drugs selected from:

i. vinca alkaloids selected from:

[1] vinblastine and

[2] vincristine;

(4) growth hormone secretagogues;

(5) strong analgesics;

(6) local and systemic anesthetics; and

(7) H₂-receptor antagonists, proton pump inhibitors and other gastroprotective agents.

The active ingredient of the present invention may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leucotriene receptor antagonists, IL-1 processing and release inhibitors, ILra, H₁-receptor antagonists; kinin-B₁- and B₂-receptor antagonists; prostaglandin inhibitors such as PGD-, PGF- PGI₂- and PGE-receptor antagonists; thromboxane A₂ (TXA2-) inhibitors; 5- and 12-lipoxygenase inhibitors; leukotriene LTC₄-, LTD₄/LTE₄- and LTB₄-inhibitors; PAF-receptor antagonists; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The combinations of the present invention may also be used in combination with anticancer agents such as endostatin and angiostatin or cytotoxic drugs such as Adriamycin, daunomycin, cis-platinum, etoposide, taxol, taxotere and alkaloids, such as vincristine and antimetabolites such as methotrexate.

The combinations of the present invention may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The combination of the present invention may also be administered in combination with one or more antibiotic, antifungal, antiprotozoal, antiviral or similar therapeutic agents.

The combinations of the present invention may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The combinations of the present invention may also be used in combination with osteoporosis agents such as roloxifene, lasofoxifene, droloxifene or fosomax and immunosuppressant agents such as FK-506 and rapamycin.

The present invention also relates to the formulation of the combination of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

The term “drugs”, which is synonymous with the phrases “active components”, “active compounds”, and “active ingredients”, includes celecoxib, or a pharmaceutically acceptable salt thereof, valdecoxib, or a pharmaceutically acceptable salt thereof, and an allosteric carboxylic inhibitor of MMP-13, or a pharmaceutically acceptable salt thereof, and may further include one or two of the other therapeutic agents described above.

The invention method is useful in human and veterinary medicines for treating mammals suffering from one or more of the above-listed diseases and disorders.

The term “mammal” includes humans, companion animals such as cats and dogs, primates such as monkeys and chimpanzees, and livestock animals such as horses, cows, pigs, and sheep.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

All that is required to practice the method of this invention is to administer a combination of celecoxib, or a pharmaceutically acceptable salt thereof, or valdecoxib, or a pharmaceutically acceptable salt thereof, and an allosteric carboxylic inhibitor of MMP-13, or a pharmaceutically acceptable salt thereof, in an amount that is therapeutically effective for preventing, inhibiting, or reversing the condition being treated. The invention combination can be administered directly or in a pharmaceutical composition as described below.

A therapeutically effective amount, or, simply, effective amount, of an invention combination will generally be from about 1 to about 300 mg/kg of subject body weight of valdecoxib, or a pharmaceutically acceptable salt thereof, or celecoxib, or a pharmaceutically acceptable salt thereof, and from about 1 to about 300 mg/kg of subject body weight of an allosteric carboxylic inhibitor of MMP-13, or a pharmaceutically acceptable salt thereof. Typical doses will be from about 10 to about 5000 mg/day for an adult subject of normal weight for each component of the combination. In a clinical setting, regulatory agencies such as, for example, the Food and Drug Administration (“FDA”) in the U.S. may require a particular therapeutically effective amount.

In determining what constitutes an effective amount or a therapeutically effective amount of an invention combination for treating, preventing, or reversing one or more symptoms of any one of the diseases and disorders described above that are being treated according to the invention methods, a number of factors will generally be considered by the medical practitioner or veterinarian in view of the experience of the medical practitioner or veterinarian, including the Food and Drug Administration guidelines, or guidelines from an equivalent agency, published clinical studies, the subject's (e.g., mammal's) age, sex, weight and general condition, as well as the type and extent of the disease, disorder or condition being treated, and the use of other medications, if any, by the subject. As such, the administered dose may fall within the ranges or concentrations recited above, or may vary outside them, ie, either below or above those ranges, depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of the invention combination that are less than optimum for a particular subject. Thereafter, the dosage can be increased by small increments until the optimum effect under the circumstance is reached. For convenience, the total daily dosage may be divided and administered in portions during the day, if desired.

Pharmaceutical compositions, described briefly here and more fully below, of an invention combination may be produced by formulating the invention combination in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Alternatively, the active components of the invention combination may be formulated separately.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat any of the above-listed diseases and disorders.

The percentage of the active ingredients of celecoxib, or a pharmaceutically acceptable salt thereof, or valdecoxib, or a pharmaceutically acceptable salt thereof, and an allosteric carboxylic inhibitor of MMP-13, or a pharmaceutically acceptable salt thereof, combination in the foregoing compositions can be varied within wide limits, but for practical purposes it is preferably present in a total concentration of at least 10% in a solid composition and at least 2% in a primary liquid composition. The most satisfactory compositions are those in which a much higher proportion of the active ingredients are present, for example, up to about 95%.

Preferred routes of administration of an invention combination are oral or parenteral. However, another route of administration may be preferred depending upon the condition being treated. For exampled, topical administration or administration by injection may be preferred for treating conditions localized to the skin or a joint. Administration by transdermal patch may be preferred where, for example, it is desirable to effect sustained dosing.

It should be appreciated that the different routes of administration may require different dosages. For example, a useful intravenous (“IV”) dose is between 5 and 50 mg, and a useful oral dosage is between 20 and 800 mg, both for each of celecoxib, or a pharmaceutically acceptable salt thereof, or valdecoxib, or a pharmaceutically acceptable salt thereof, and an allosteric carboxylic inhibitor of MMP-13, or a pharmaceutically acceptable salt thereof. The dosage is within the dosing range used in treatment of the above-listed diseases, or as would be determined by the needs of the patient as described by the physician.

The invention combination may be administered in any form. Preferably, administration is in unit dosage form. A unit dosage form of the invention combination to be used in this invention may also comprise other compounds useful in the therapy of diseases described above. A further description of pharmaceutical formulations useful for administering the invention combinations is provided below.

The active components of the invention combination, including celecoxib, or a pharmaceutically acceptable salt thereof, or valdecoxib, or a pharmaceutically acceptable salt thereof, and an allosteric carboxylic inhibitor of MMP-13, or a pharmaceutically acceptable salt thereof, and other compounds as described above, if any, may be formulated together or separately and may be administered together or separately. The particular formulation and administration regimens used may be tailored to the particular patient and condition being treated by a practitioner of ordinary skill in the medical or pharmaceutical arts.

The advantages of using an invention combination comprising celecoxib, or a pharmaceutically acceptable salt thereof, or valdecoxib, or a pharmaceutically acceptable salt thereof, and an allosteric carboxylic inhibitor of MMP-13, or a pharmaceutically acceptable salt thereof, in a method of the instant invention include the nontoxic nature of the compounds which comprise the combination at and substantially above therapeutically effective doses, their ease of preparation, the fact that the compounds are well-tolerated, and the ease of topical, IV, or oral administration of the drugs.

Another important advantage is that the present invention combinations more effectively target a particular disease that is responsive to inhibition of MMP-13 with fewer undesirable side effects than similar combinations that contain MMP-13 inhibitors that are not allosteric carboxylic inhibitors of MMP-13. This is so because the instant allosteric carboxylic inhibitors of MMP-13, or a pharmaceutically acceptable salt thereof, do not directly, or indirectly via a bridging water molecule, ligate, coordinate to, or bind to the catalytic zinc cation of MMP-13, but instead bind at a different location from where natural substrate binds to MMP-13. The binding requirements of an allosteric MMP-13 binding site are unique to MMP-13, and account for the specificity of the instant allosteric carboxylic inhibitors of MMP-13 for inhibiting MMP-13 over any other MMP enzyme. This binding mode has not been reported in the art. Indeed, prior art inhibitors of MMP-13 bind to the catalytic zinc cations of other MMP enzymes as well as to the catalytic zinc cation of MMP-13 and, and are consequently significantly less selective inhibitors of MMP-13 enzyme.

The instant allosteric carboxylic inhibitors of MMP-13 are thus therapeutically superior to other inhibitors of MMP-13, or even tumor necrosis factor-alpha converting enzyme (“TACE”), because of fewer undesirable side effects from inhibition of the other MMP enzymes or TACE. For example, virtually all prior art MMP inhibitors tested clinically to date have exhibited an undesirable side effect known as muscoloskeletal syndrome (“MSS”). MSS is associated with administering an inhibitor of multiple MMP enzymes or an inhibitor of a particular MMP enzyme such as MMP-1. MSS will be significantly reduced in type and severity by administering the invention combination instead of any combination of a prior art MMP-13 inhibitor with celecoxib or valdecoxib, or a pharmaceutically acceptable salt thereof. The invention combinations are superior to similar combinations that include a COX-2 selective inhibitor with an MMP inhibitor that interacts with the catalytic zinc cation of the MMP-13 enzyme as discussed above, even if that inhibitor shows some selectivity for the MMP-13.

This advantage of the instant combinations will also significantly increase the likelihood that agencies which regulate new drug approvals, such as the United States Food and Drug Administration, will approve the instant combination versus a competing similar combination as discussed above even in the unlikely event that the two combinations behaved similarly in clinical trials. These regulatory agencies are increasingly aware that clinical trials, which test drug in limited population groups, do not always uncover safety problems with a drug, and thus all other things being equal, the agencies will favor the drug with the lowest odds of producing undesirable side effects.

Another important advantage is that the independent anti-inflammatory and pain reducing properties described above for valdecoxib and celecoxib and the disease modifying properties of allosteric carboxylic inhibitors of MMP-13 provide patients suffering from cartilage damage, arthritis, inflammation and/or pain with both relief of symptoms and prevention or inhibition of the underlying disease pathology such as cartilage degradation.

A further advantage of the invention combination is administration of the combination to treat a disease or disorder in a mammal may allow lower doses of valdecoxib, or a pharmaceutically acceptable salt thereof, or celecoxib, or a pharmaceutically acceptable salt thereof, and/or an allosteric carboxylic inhibitor of MMP-13 of the combination to be used than would be used if valdecoxib, or a pharmaceutically acceptable salt thereof, or celecoxib, or a pharmaceutically acceptable salt thereof, and the allosteric carboxylic inhibitor of MMP-13 were each administered alone. This advantage is a result of an unexpected synergistic therapeutically beneficial effect on inhibition of cartilage damage or alleviation of pain for the combination over the sum of the therapeutic effects for each component of the combination administered alone.

Some of the compounds utilized in an invention combination are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds useful in the invention combination.

Pharmaceutically acceptable acid addition salts of the basic compounds useful in the invention combination include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

An acid addition salt of a basic compound useful in the invention combination is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the compounds and their respective acid addition salt forms are equivalent for purposes of the present invention.

A pharmaceutically acceptable base addition salt of an acidic compound useful in the invention combination may be prepared by contacting the free acid form of the compound with a nontoxic metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an acidic compound useful in the invention combination may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the compounds useful in the invention combination differ from their respective salt forms somewhat in certain physical properties such as solubility, crystal structure, hygroscopicity, and the like, but otherwise the salts are equivalent to their respective free acid for purposes of the present invention.

Certain of the compounds useful in the invention combination can exist in unsolvated forms as well as solvated forms, including hydrated forms. In general, the solvated forms, including hydrated forms, are equivalent to unsolvated forms and are encompassed within the scope of the present invention.

Certain of the compounds useful in the invention combination possess one or more chiral centers, and each center may exist in the R or S configuration. An invention combination may utilize any diastereomeric, enantiomeric, or epimeric form of a compound useful in the invention combination, as well as mixtures thereof.

Additionally, certain compounds useful in the invention combination may exist as geometric isomers such as the entgegen (E) and zusammen (Z) isomers of 1,2-disubstituted alkenyl groups or cis and trans isomers of disubstituted cyclic groups. An invention combination may utilize any cis, trans, syn, anti, entgegen (E), or zusammen (Z) isomer of a compound useful in the invention combination, as well as mixtures thereof.

Certain compounds useful in the invention combination can exist as two or more tautomeric forms. Tautomeric forms of the compounds may interchange, for example, via enolization/de-enolization, 1,2-hydride, 1,3-hydride, or 1,4-hydride shifts, and the like. An invention combination may utilize any tautomeric form of a compound useful in the invention combination, as well as mixtures thereof.

The syntheses of valdecoxib, or a pharmaceutically acceptable salt thereof, and celecoxib, or a pharmaceutically acceptable salt thereof, are well-known in the art, and have even been carried out to produce commercial-scale quantities of compound. The synthesis of allosteric inhibitors of MMP-13 are taught in the patent applications incorporated above by reference.

Intermediates for the synthesis of valdecoxib, or a pharmaceutically acceptable salt thereof, celecoxib, or a pharmaceutically acceptable salt thereof, and an allosteric carboxylic inhibitor of MMP-13, or a pharmaceutically acceptable salt thereof, useful in the invention combination may be prepared by one of ordinary skill in the art of organic chemistry by adapting various synthetic procedures incorporated by reference above or that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods, 1989, by Wiley-Interscience; the text Advanced Organic Chemistry, 4^{th} edition, by Jerry March, Wiley-Interscience, New York,1992; or the Handbook of Heterocyclic Chemistry by Alan R. Katritzky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the compounds useful in an invention combination may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A. G., Switzerland, or Lancaster Synthesis Ltd, United Kingdom.

Syntheses of some compounds useful in the invention combination may utilize starting materials, intermediates, or reaction products that contain a reactive functional group. During chemical reactions, a reactive functional group may be protected from reacting by a protecting group that renders the reactive functional group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of valdecoxib, or a pharmaceutically acceptable salt thereof, or celecoxib, or a pharmaceutically acceptable salt thereof, or an allosteric carboxylic inhibitor of MMP-13, or a pharmaceutically acceptable salt thereof, and then later remove them. Procedures for introducing and removing protecting groups are known and referenced such as, for example, in Protective Groups in Organic Synthesis, 2^{nd} ed., Greene T. W. and Wuts P. G., John Wiley & Sons, New York: New York, 1991, which is hereby incorporated by reference.

Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

Preparations of valdecoxib, or a pharmaceutically acceptable salt thereof, or an allosteric carboxylic inhibitor of MMP-13, or a pharmaceutically acceptable salt thereof, useful in the invention combination are incorporated by reference to the patents, patent applications, and patent application publications described above.

Allosteric carboxylic inhibitors of MMP-13 useful in the present invention combinations may be prepared by one of ordinary skill in the art of synthetic organic chemistry readily adapting known literature methods. Additional methods of preparation are described in co-pending PCT international applications and their corresponding United States nonprovisional application Ser. Nos. 10/071,032; 10/075,918; 10/075,073; 10/075,069; 10/075,954; 10/075,654; 10/074,646; 10/075,909; and 10/071,073, and the related U.S. provisional application Nos. 60/268,780; 60/268,736; 60/268,756; 60/268,821; 60/268,861; 60/268,757; 60/268,782; 60/268,779; and 60/268,781, respectively, all provisional applications filed on Feb. 14, 2001, and from which benefit of priority is claimed. All of the PCT International applications, United States provisional applications, and United States nonprovisional applications have been incorporated herein by reference above.

### Examples of Allosteric Carboxylic Inhibitors of MMP-13

1. Examples of Thiazolopyrimidinedione Allosteric Inhibitors of MMP-13:

The syntheses of thiazolopyrimidinediones useful as allosteric inhibitors of MMP-13 are described in our co-pending U.S. nonprovisional application Ser. No. 10/071,032, the corresponding PCT International application number PCT/IB02/00313, and the priority application U.S. provisional application No. 60/268,780, filed on Feb. 14, 2001.

One example is named and drawn below:

6-Benzyl-5,7-dioxo-6,7-dihydro-5H-thiazolo[3,2-c]pyrimidine-2-carboxylic acid benzyl ester

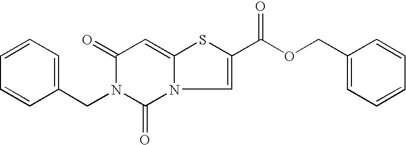

It should be appreciated that the compound drawn above has first and second hydrophobic groups and first, second and third hydrogen bond acceptors. The first hydrophobic group locates in the S1′ pocket of the enzyme and its hydrophobic aryl ring interacts with the aryl rings of His222 and Tyr244. The second hydrophobic group is open to solvent and forms hydrophobic interactions with the aryl rings of e.g. Phe252 and Tyr246. The three hydrogen bond acceptors interact respectively with Thr245, Thr247 and Met 253.

2. Examples of Isophthalic Acid Allosteric Inhibitors of MMP-13:

The syntheses of isophthalic acid derivatives are described in our co-pending United States nonprovisional application Ser. No. 10/075,918, the corresponding PCT International application number PCT/IB02/00344, and the priority application U.S. provisional application No. 60/268,736, filed on Feb. 14, 2001.

Binding to MMP-13 of a representative example of one of the isophthalic acid derivatives is as described above for Example 1. It will be observed that the compounds of this series have two hydrophobic groups and two hydrogen bond acceptors.

3. Examples of Fused Bicyclic Pyrimidone Allosteric Inhibitors of MMP-13:

The syntheses of fused bicyclic pyrimidone allosteric inhibitors of MMP-13 are described in co-pending United States nonprovisional application Ser. No. 10/075,073, the corresponding PCT International application number PCT/IB02/00204, and the priority application U.S. provisional application No. 60/268,756, filed on Feb. 14, 2001.

Binding to MMP-13 of a representative compound of the fused bicyclic pyrimidone allosteric inhibitors of MMP-13 is through two hydrophobic groups and three hydrogen bond acceptors, the third hydrogen bond acceptor binding to Met 253 and also via a bridging water molecule to the backbone carbonyl of His251.

4. Examples of Substituted Quinazoline Allosteric Inhibitors of MMP-13:

The syntheses of quinazoline allosteric inhibitors of MMP-13 are described in our co-pending United States nonprovisional application Ser. No. 10/075,954, the related PCT International application number PCT/EP02/01979, and the corresponding priority U.S. provisional application No. 60/268,661, filed on Feb. 14, 2001.

Binding to MMP-13 of the compound of Example 35 is based on two hydrophobic groups and three hydrogen bond acceptors. As in the thiazolopyrimidinediones, the third hydrogen bond acceptor binds both to Met 253 and via a bridging water molecule to the backbone carbonyl oxygen of His 251. It will also be noted from the above table that some compounds in this series do not have a second hydrophobic group, but nevertheless bind to MMP-13 and exhibit a useful inhibitory activity.

5. Examples of pyrido[2,3-d]pyrimidines:

The syntheses of pyrido[2,3-d]pyrimidine allosteric inhibitors of MMP-13 are also described in our co-pending United States nonprovisional application Ser. No. 10/075,954, the related PCT International application number PCT/EP02/01979, and the corresponding priority U.S. provisional application No. 60/268,661, filed on Feb. 14, 2001.

6. Examples of Fused Triazolo-quinazoline Allosteric Inhibitors of MMP-13:

Syntheses of fused triazolo-quinazoline allosteric inhibitors of MMP-13 are described in our co-pending United States nonprovisional application Ser. No. 10/075,654, the related PCT International application number PCT/FR02/00504, and the priority application U.S. provisional application No. 60/268,757, filed on Feb. 14, 2001.

Binding of a representative compound in the fused triazolo-quinazoline, Example 57 involves first and second hydrophobic groups and first, second and third hydrogen bond acceptors.

7. Examples of 1,1-dioxy-benzo-(1,2,4)-thiadiazine Allosteric Inhibitors of MMP-13:

The syntheses of 1,1-dioxy-benzo-(1,2,4)-thiadiazine allosteric inhibitors of MMP-13 are described in our co-pending United States nonprovisional application Ser. No. 10/074,646, the related PCT International application number PCT/IB02/00083, and the priority application U.S. provisional application No. 60/268,782, filed on Feb. 14, 2001.

For illustration purposes, examples of allosteric carboxylic inhibitors of MMP-13 are described below. The allosteric carboxylic inhibitors of MMP-13 have been evaluated in standard assays for their ability to inhibit the catalytic activity of various MMP enzymes. The assays used to evaluate the MMP biological activity of the invention compounds are well-known and routinely used by those skilled in the study of MMP inhibitors and their use to treat clinical conditions. For example, allosteric carboxylic inhibitors of MMP-13 may be readily identified by assaying a test compound for inhibition of MMP-13 according to Biological Methods 1 or 2, and further assaying the test compound for allosteric inhibition of MMP-13 according to Biological Methods 3 or 4, as described below.

Examples of allosteric carboxylic inhibitors of MMP-13 are provided below. The compounds have been shown to be potent and selective inhibitors of MMP-13 catalytic domain versus full-length MMP-1 and MMP-3 catalytic domain. Potencies with MMP-13 catalytic domain for the allosteric inhibitors of MMP-13 typically range from about 0.001 μM to about 1 μM. Some compounds were further screened with full-length MMP-2, full-length MMP-7, full-length MMP-9, and MMP-14 catalytic domain, and were found to be selective inhibitors of MMP-13 versus these other MMP enzymes also. Selectivity of the allosteric inhibitors of MMP-13 for MMP-13 catalytic domain versus another MMP enzyme (full-length or catalytic domain), as determined by dividing the IC₅₀ for the inhibitor with a comparator MMP enzyme by the IC₅₀ of the inhibitor with MMP-13 catalytic domain, typically ranged from 5 to 50,000 fold.

The allosteric carboxylic inhibitors of MMP-13 were assayed for inhibition of MMP-13 and for allosteric inhibition of MMP-13 and certain other MMP enzymes according to Biological Methods 1 to 4, which are described immediately below. The assays measure the amount by which a test compound reduces the hydrolysis of a thiopeptolide substrate catalyzed by a matrix metalloproteinase enzyme. Such assays are described in detail by Ye et al., in Biochemistry, 1992;31(45):11231-11235, which is incorporated herein by reference. One such assay is described below in Biological Method 1.

Some of the particular methods described below use the catalytic domain of the MMP-13 enzyme, namely matrix metalloproteinase-13 catalytic domain (“MMP-13CD”), rather than the corresponding full-length enzyme, MMP-13. It has been shown previously by Ye Qi-Zhuang, Hupe D., and Johnson L. (Current Medicinal Chemistry, 1996;3:407-418) that inhibitor activity against a catalytic domain of an MMP is predictive of the inhibitor activity against the respective full-length MMP enzyme.

### Biological Method 1

Thiopeptolide substrates show virtually no decomposition or hydrolysis at or below neutral pH in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain 50 mM of N-2-hydroxyethylpiperazine-N′-2-ethanesulfonic acid buffer (“HEPES,” pH 7.0), 10 mM CaCl₂, 100 μM thiopeptolide substrate, and 1 mM 5,5′-dithio-bis-(2-nitro-benzoic acid) (DTNB). The thiopeptolide substrate concentration may be varied, for example from 10 to 800 μM to obtain Kₘ and K_{cat} values. The change in absorbance at 405 nm is monitored on a Thermo Max microplate reader (molecular Devices, Menlo Park, Calif.) at room temperature (22° C.). The calculation of the amount of hydrolysis of the thiopeptolide substrate is based on E₄₁₂=13600 M⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Test compounds were evaluated at various concentrations in order to determine their respective IC₅₀ values, the micromolar concentration of compound required to cause a 50% inhibition of catalytic activity of the respective enzyme.

It should be appreciated that the assay buffer used with MMP-3CD was 50 mM N-morpholinoethane sulfonate (“MES”) at pH 6.0 rather than the HEPES buffer at pH 7.0 described above.

### Biological Method 2

The test described above for the inhibition of MMP-13 was also adapted and used to determine the ability of the allosteric carboxylic inhibitors of MMP-13 to inhibit the matrix metalloproteases MMP-1, MMP-2, MMP-3, MMP-7, MMP-9, MMP-12 and MMP-14. Allosteric carboxylic inhibitors of MMP-13 have been evaluated for their ability to inhibit MMP-13 and other MMPs using, for example, MMP-1FL, which refers to full length interstitial collagenase; MMP-2FL, which refers to full length Gelatinase A; MMP-3CD, which refers to the catalytic domain of stromelysin; MMP-7FL, which refers to full length matrilysin; MMP-9FL, which refers to full length Gelatinase B; MMP-13CD, which refers to the catalytic domain of collagenase 3; and MMP-14CD, which refers to the catalytic domain of MMP-14. Test compounds can be evaluated at various concentrations in order to determine their respective IC₅₀ values, the micromolar concentration of compound required to cause a 50% inhibition of the hydrolytic activity of the respective enzyme. The results obtained show that the allosteric carboxylic inhibitors of MMP-13 generally have IC₅₀ values for MMP-13 which are about 100 times lower than the IC₅₀ values for the same compounds with respect to the other matrix metalloproteases tested.

The results of the above assays with other MMPs establish that the allosteric carboxylic inhibitors of MMP-13 are potent and selective inhibitors of MMP enzymes. Because of this potent and selective inhibitory activity, the compounds are especially useful, in combination with a selective inhibitor of COX-2, to treat diseases mediated by the MMP enzymes and COX-2, and particularly those mediated by MMP-13 and COX-2.

Allosteric carboxylic inhibitors of MMP-13 also may be readily identified by assaying a test compound for inhibition of MMP-13 according to the methods described below in Biological Methods 3 and 4.

### Biological Method 3

Fluorigenic peptide-1 substrate based assay for identifying allosteric carboxylic inhibitors of MMP-13CD:

Final Assay Conditions:

50 mM HEPES buffer (pH 7.0)

10 mM CaCl₂

10 μM fluorigenic peptide-1 (“FP1”) substrate

0 or 15 mM acetohydroxamic acid (AcNHOH)=1 K_{d}

2% DMSO (with or without inhibitor test compound)

0.5 nM MMP-13CD enzyme

Stock Solutions:

1) 10×assay buffer: 500 mM HEPES buffer (pH 7.0) plus 100 mM CaCl₂

2) 10 mM FP1 substrate: (Mca)-Pro-Leu-Gly-Leu-(Dnp)-Dpa-Ala-Arg-NH₂ (Bachem, M-1895; “A novel coumarin-labeled peptide for sensitive continuous assays of the matrix metalloproteinases,” Knight C. G., Willenbrock F., and Murphy, G., FEBS Lett., 1992;296:263-266). Prepared 10 mM stock by dissolving 5 mg FP1 in 0.457 mL DMSO.

3) 3 M AcNHOH: Prepared by adding 4 mL H₂O and 1 mL 10×assay buffer to 2.25 g AcNHOH (Aldrich 15,903-4). Adjusted pH to 7.0 with NaOH. Diluted volume to 10 mL with H₂O. Final solution contained 3 M AcNHOH, 50 mM HEPES buffer (pH 7.0), and 10 mM CaCl₂.

4) AcNHOH dilution buffer: 50 mM HEPES buffer (pH 7.0) plus 10 mM CaCl₂

5) MMP-13CD enzyme: Stock concentration=250 nM.

6) Enzyme dilution buffer: 50 mM HEPES buffer (pH 7.0), 10 mM CaCl₂, and 0.005% BRIJ 35 detergent (Calbiochem 203728; Protein Grade, 10%)

Procedure (for one 96-well microplate):

A. Prepared assay mixture:

1100 μL 10× assay buffer

11 μL 10 mM FP1

55 μL 3 M AcNHOH or 55 μL AcNHOH dilution buffer

8500 μL H₂O

B. Diluted MMP-13CD to 5 nM working stock:

22 μL MMP-13CD (250 nM)

1078 μL enzyme dilution buffer

C. Ran kinetic assay:

1. Dispensed 2 μL inhibitor test sample (in 100% DMSO) into well.

2. Added 88 μL assay mixture and mixed well, avoiding bubbles.

3. Initiated reactions with 10 μL of 5 nM MMP-13CD; mixed well, avoiding bubbles.

4. Immediately measured the kinetics of the reactions at room temperature.

Fluorimeter: Fₘₐₓ Fluorescence Microplate Reader & SOFTMAX PRO Version 1.1 software (Molecular Devices Corporation; Sunnyvale, Calif. 94089).

Protocol menu:

| excitation: | 320 nm | emission: | 405 nm |
|---|---|---|---|
| run time: | 15 mm | interval: | 29 sec |
| RFU min: | -10 | RFU max: | 200 |
| Vₘₐₓ points: | 32/32 | | |

D. Compared % of control activity and/or IC₅₀ with inhibitor test compound ±AcNHOH.

Hydrolysis of the fluorigenic peptide-1 substrate, [(Mca)Pro-Leu-Gly-Leu-Dpa-Ala-Arg-NH₂; Bachem, catalog number M-1895], wherein “Mca” is (7-methoxy-coumarin-4-yl)acetyl and “Dpa” is (3-[2,4-dinitrophenyl]-L-2,3-diaminopropionyl), was used to screen for MMP-13 catalytic domain (CD) inhibitors. (Dpa may also be abbreviated as “Dnp”.) Reactions (100 μL) contained 0.05 M Hepes buffer (pH 7), 0.01 M calcium chloride, 0.005% polyoxyethylene (23) lauryl ether (“Brij 35”), 0 or 15 mM acetohydroxamic acid, 10 μM FP1, and 0.1 mM to 0.5 nM inhibitor in DMSO (2% final).

After recombinant human MMP-13CD (0.5 nM final) was added to initiate the reaction, the initial velocity of FP1 hydrolysis was determined by monitoring the increase in fluorescence at 405 nm (upon excitation at 320 nm) continuously for up to 30 minutes on a microplate reader at room temperature. Alternatively, an endpoint read can also be used to determine reaction velocity provided the initial fluorescence of the solution, as recorded before addition of enzyme, is subtracted from the final fluorescence of the reaction mixture. The inhibitor was assayed at different concentration values, such as, for example, 100 μM, 10 μM, 1 μM, 100 nM, 10 nM, and 1 nM. Then the inhibitor concentration was plotted on the X-axis against the percentage of control activity observed for inhibited experiments versus uninhibited experiments (i.e., (velocity with inhibitor) divided by (velocity without inhibitor)×100) on the Y-axis to determine IC₅₀ values. This determination was done for experiments done in the presence, and experiments done in the absence, of acetohydroxamic acid. Data were fit to the equation: percent control activity=100/[1+(([I]/IC₅₀)^{slope})], where [I] is the inhibitor concentration, IC₅₀ is the concentration of inhibitor where the reaction rate is 50% inhibited relative to the control, and slope is the slope of the IC₅₀ curve at the curve's inflection point, using nonlinear least-squares curve-fitting equation regression.

Results may be expressed as an IC₅₀ Ratio (+/−) ratio, which means a ratio of the IC₅₀ of the inhibitor with MMP-13 and a inhibitor to the catalytic zinc of MMP-13, divided by the IC₅₀ of the inhibitor with MMP-13 without the inhibitor to the catalytic zinc of MMP-13. Allosteric carboxylic inhibitors of MMP-13 have an IC₅₀ Ratio (+/−) ratio of less than 1, and are synergistic with the inhibitor to the catalytic zinc of MMP-13 such as, for example, AcNHOH. Compounds which are not allosteric carboxylic inhibitors of MMP-13 will be inactive in the assay or will have an IC₅₀ Ratio (+/−) of greater than 1, unless otherwise indicated. Results can be confirmed by kinetics experiments which are well known in the biochemical art.

### Biological Method 4

Fluorigenic peptide-1 based assay for identifying allosteric carboxylic inhibitors of matrix metalloproteinase-13 catalytic domain (“MMP-13CD”):

In a manner similar to Biological Method 3, an assay is run wherein 1, 10-phenanthroline is substituted for acetohydroxamic acid to identify allosteric carboxylic inhibitors of MMP-13CD.

Animal models may be used to establish that the instant allosteric carboxylic inhibitors of MMP-13, or a pharmaceutically acceptable salt thereof, or an N-oxide thereof, would be useful for preventing, treating, and inhibiting cartilage damage, and thus for treating osteoarthritis, for example.

### Biological Method 5

Selective inhibitors of COX-2 may be identified by screening a test compound in the following assays.

Human In Vitro Assays

Human Cell-Based COX-1 Assay:

Human peripheral blood obtained from healthy volunteers can be diluted to 1/10 volume with 3.8% sodium citrate solution. The platelet-rich plasma immediately obtained can be washed with 0.14 M sodium chloride containing 12 mM Tris-HCl (pH 7.4) and 1.2 mM EDTA. Platelets can then be washed with platelet buffer (Hanks buffer (Ca free) containing 0.2% BSA and 20 mM Hepes). Finally, the human washed platelets (HWP) can be suspended in platelet buffer at the concentration of 2.85×10⁸ cells/ml and stored at room temperature until use. The HWP suspension (70 μl aliquots, final 2.0×10⁷ cells/ml) can be placed in a 96-well U bottom plate and 10 μl aliquots of 12.6 mM calcium chloride added. Platelets can be incubated with A23187 (final 10 μM, Sigma) with test compound (0.1-100 μM) dissolved in DMSO (final concentration; less than 0.01%) at 37° C. for 15 minutes. The reaction can be stopped by addition of EDTA (final 7.7 mM) and TxB2 in the supernatant quantitated by using a radioimmunoassay kit (Amersham) according to the manufacturer's procedure.

Human Cell-Based COX-2 Assay:

The human cell based COX-2 assay can be carried out as previously described (Moore et al., Inflamm. Res., 45, 54, 1996). Confluent human umbilical vein endothelial cells (HUVECs, Morinaga) in a 96-well flat bottom plate can be washed with 80 ml of RPMI1640 containing 2% FBS and incubated with hIL-1β (final concentration 300 U/ml, R & D Systems) at 37° C. for 24 hours. After washing, the activated HUVECs can be incubated with test compound (final concentration; 0.1 nM-1 μM) dissolved in DMSO (final concentration; less than 0.01%) at 37° C. for 20 minutes and stimulated with A23187 (final concentration 30 mM) in Hanks buffer containing 0.2% BSA, 20 mM Hepes at 37° C. for 15 minutes. 6-Keto-PGF_{1α}, stable metabolite of PGI2, in the supernatant can be quantitated by using a radioimmunoassay method (antibody; Preseptive Diagnostics, SPA; Amersham).

Canine In Vitro Assays:

The following canine cell based COX 1 and COX-2 assays have been reported in Ricketts et al., Evaluation of Selective Inhibition of Canine Cyclooxygenase 1 and 2 by Carprofen and Other Nonsteroidal Anti-inflammatory Drugs, American Journal of Veterinary Research, 59 (11), 1441-1446.

Protocol for Evaluation of Canine COX-1 Activity:

Test compounds can be solubilized and diluted the day before the assay can be to be conducted with 0.1 mL of DMSO/9.9 mL of Hank's balanced salts solution (HBSS) and stored overnight at 4° C. On the day that the assay can be carried out, citrated blood can be drawn from a donor dog, centrifuged at 190×g for 25 minutes at room temperature and the resulting platelet-rich plasma can then be transferred to a new tube for further procedures. The platelets can be washed by centrifuging at 1500×g for 10 minutes at room temperature. The platelets can be washed with platelet buffer comprising Hank's buffer (Ca free) with 0.2% bovine serum albumin (BSA) and 20 mM HEPES. The platelet samples can then be adjusted to 1.5×10⁷/mL, after which 50 μl of calcium ionophore (A23187) together with a calcium chloride solution can be added to 50 μl of test compound dilution in plates to produce final concentrations of 1.7 μM A23187 and 1.26 mM Ca. Then, 100 μl of canine washed platelets can be added and the samples can be incubated at 37° C. for 15 minutes, after which the reaction can be stopped by adding 20 μl of 77 mM EDTA. The plates can then be centrifuged at 2000×g for 10 minutes at 4° C., after which 50 μl of supernatant can be assayed for thromboxane B₂ (TXB₂) by enzyme-immunoassay (EIA). The pg/mL of TXB₂ can be calculated from the standard line included on each plate, from which it can be possible to calculate the percent inhibition of COX-1 and the IC₅₀ values for the test compounds.

Protocol for Evaluation of Canine COX-2 Activity:

A canine histocytoma (macrophage-like) cell line from the American Type Culture Collection designated as DH82, can be used in setting up the protocol for evaluating the COX-2 inhibition activity of various test compounds. There can be added to flasks of these cells 10 μg/mL of LPS, after which the flask cultures can be incubated overnight. The same test compound dilutions as described above for the COX-1 protocol can be used for the COX-2 assay and can be prepared the day before the assay can be carried out. The cells can be harvested from the culture flasks by scraping and can then be washed with minimal Eagle's media (MEM) combined with 1% fetal bovine serum, centrifuged at 1500 rpm for 2 minutes and adjusted to a concentration of 3.2×10⁵ cells/mL. To 50 μl of test compound dilution there can be added 50 μl of arachidonic acid in MEM to give a 10 μM final concentration and there can be added as well 100 μl of cell suspension to give a final concentration of 1.6×10⁵ cells/mL. The test sample suspensions can be incubated for 1 hour and then centrifuged at 1000 rpm for 10 minutes at 4° C., after which 50 μl aliquots of each test compound sample can be delivered to EIA plates. The EIA can be performed for prostaglandin E₂ (PGE₂) and the pg/mL concentration of PGE₂ can be calculated from the standard line included on each plate. From this data it can be possible to calculate the percent inhibition of COX-2 and the IC₅₀ values for the test compounds. Repeated investigations of COX-1 and COX-2 inhibition can be conducted over the course of several months. The results are averaged and a single COX-1:COX-2 ratio is calculated.

Whole blood assays for COX-1 and COX-2 are known in the art such as the methods described in C. Brideau, et al., A Human Whole Blood Assay for Clinical Evaluation of Biochemical Efficacy of Cyclooxygenase Inhibitors, Inflammation Research, Vol. 45, pp. 68-74 (1996). These methods may be applied with feline, canine or human blood as needed.

### Biological Method 6

Carrageenan Induced Foot Edema in Rats

Male Sprague-Dawley rats (5 weeks old, Charles River Japan) can be fasted overnight. A line can be drawn using a marker above the ankle on the right hind paw and the paw volume (VO) can be measured by water displacement using a plethysmometer (Muromachi). Animals can be given orally either vehicle (0.1% methyl cellulose or 5% Tween 80) or a test compound (2.5 ml per 100 g body weight). One hour later, the animals can then be injected intradermally with □-carrageenan (0.1 ml of 1% w/v suspension in saline, Zushikagaku) into right hind paw (Winter et al., Proc. Soc. Exp. Biol. Med., 111, 544, 1962; Lombardino et al., Arzneim. Forsch., 25, 1629, 1975) and three hours later, the paw volume (V3) can be measured and the increase in volume (V3-V0) calculated. Since maximum inhibition attainable with classical NSAIDs is 60-70%, ED₃₀ values can be calculated.

### Biological Method 7

Gastric Ulceration in Rats:

The gastric ulcerogenicity of test compound can be assessed by a modification of the conventional method (Ezer et al., J. Pharm. Pharmacol., 28, 655, 1976; Cashin et al., J. Pharm. Pharmacol., 29, 330-336, 1977). Male Sprague-Dawley rats (5 weeks old, Charles River Japan), fasted overnight, can be given orally either vehicle (0.1% methyl cellulose or 5% Tween 80) or a test compound (1 ml per 100 g body weight). Six hours after, the animals can be sacrificed by cervical dislocation. The stomachs can be removed and inflated with 1% formalin solution (10 ml). Stomachs can be opened by cutting along the greater curvature. From the number of rats that showed at least one gastric ulcer or haemorrhaging erosion (including ecchymosis), the incidence of ulceration can be calculated. Animals did not have access to either food or water during the experiment.

### Biological Method 8

Canine Whole Blood Ex Vivo Determinations of COX-1 and COX-2 Activity Inhibition

The in vivo inhibitory potency of a test compound against COX-1 and COX-2 activity may be evaluated using an ex vivo procedure on canine whole blood. Three dogs can be dosed with 5 mg/kg of the test compound administered by oral gavage in 0.5% methylcellulose vehicle and three dogs can be untreated. A zero-hour blood sample can be collected from all dogs in the study prior to dosing, followed by 2- and 8-hour post-dose blood sample collections. Test tubes can be prepared containing 2 μL of either (A) calcium ionophore A23187 giving a 50 μM final concentration, which stimulates the production of thromboxane B₂ (TXB₂) for COX-1 activity determination; or of (B) lipopolysaccharide (LPS) to give a 10 μg/mL final concentration, which stimulates the production of prostaglandin E₂ (PGE₂) for COX-2 activity determination. Test tubes with unstimulated vehicle can be used as controls. A 500 μL sample of blood can be added to each of the above-described test tubes, after which they can be incubated at 37° C. for one hour in the case of the calcium ionophore-containing test tubes and overnight in the case of the LPS-containing test tubes. After incubation, 10 μL of EDTA can be added to give a final concentration of 0.3%, in order to prevent coagulation of the plasma which sometimes occurs after thawing frozen plasma samples. The incubated samples can be centrifuged at 4° C. and the resulting plasma sample of ˜200 μL can be collected and stored at −20° C. in polypropylene 96-well plates. In order to determine endpoints for this study, enzyme immunoassay (EIA) kits available from Cayman can be used to measure production of TXB₂ and PGE₂, utilizing the principle of competitive binding of tracer to antibody and endpoint determination by colorimetry. Plasma samples can be diluted to approximate the range of standard amounts which would be supplied in a diagnostic or research tools kit, i.e., 1/500 for TXB₂ and 1/750 for PGE₂.

COX inhibition is observed when the measured percent inhibition is greater than that measured for untreated controls. The percent inhibition in the above table is calculated in a straightforward manner in accordance with the following equation:

$$\% Inhibition ( 2 \text{-} hour ) = \frac{( {PGE}_{2} at t = 0 ) - ( {PGE}_{2} at t = 2 )}{( {PGE}_{2} at t = 0 )}$$

Data Analysis:

Statistical program packages, SYSTAT (SYSTAT, INC.) and StatView (Abacus Cencepts, Inc.) for Macintosh can be used. Differences between test compound treated group and control group can be tested for using ANOVA. The IC₅₀ (ED30) values can be calculated from the equation for the log-linear regression line of concentration (dose) versus percent inhibition.

The selective COX-2 inhibitors described above have been, or could have been, identified by at least one of the methods described above and show, or would show, IC₅₀ values of 0.001 μM to 3 μM with respect to inhibition of COX-2 in either the canine or human assays.

As mentioned above, COX-2 selectivity can be determined by ratio in terms of IC₅₀ value of COX-1 inhibition to COX-2 inhibition. In general, it can be said that a compound showing a COX-1/COX-2 inhibition ratio of more than 5 has sufficient COX-2 selectivity.

The newly discovered ability of an allosteric carboxylic inhibitor of MMP-13, or a pharmaceutically acceptable salt thereof, to inhibit cartilage damage, alleviate pain, and treat osteoarthritis may be established in animal models as described below. The activity of an invention combination for treating cartilage damage and pain and/or inflammation may be determined by the procedures of Biological Methods 9 or 10 as described below.

Biological Method 9

Monosodium Iodoacetate-induced Osteoarthritis in Rat Model of Cartilage Damage (“MIA Rat”):

One end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb containing the affected joint, the presence of increased amounts of proteoglycan or hydroxyproline in the joint upon biochemical analysis, or histopathological analysis of the osteoarthritic lesions.

Generally, In the MIA Rat model on Day 0, the hind-paw weight differential between the right arthritic joint and the left healthy joint of male Wistar rats (150 g) are determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Then the rats are anesthetized with isofluorine, and the right, hind leg knee joint is injected with 1.0 mg of mono-iodoacetate (“MIA”) through the infrapatellar ligament. Injection of MIA into the joint results in the inhibition of glycolysis and eventual death of surrounding chondrocytes. The rats are further administered either an invention combination such as a combination, comprising an allosteric carboxylic inhibitor of MMP-13, or a pharmaceutically acceptable salt thereof, with celecoxib, or a pharmaceutically acceptable salt thereof, or valdecoxib, or a pharmaceutically acceptable salt thereof, or vehicle (in the instant case, water) daily for 14 days or 28 days. Both the allosteric carboxylic inhibitor of MMP-13, or a pharmaceutically acceptable salt thereof, and celecoxib, or a pharmaceutically acceptable salt thereof, or valdecoxib, or a pharmaceutically acceptable salt thereof, are, each independently, typically administered at a dose of 30 mg per kilogram of rat per day (30 mg/kg/day), but each component of the combination may independently be administered at other doses such as, for example, 10 mg/kg/day, 60 mg/kg/day, 90-mg/kg/day, or 100 mg/kg/day according to the requirements of the combination being studied. It is well within the level of ordinary skill in the pharmaceutical arts to determine a proper dosage of an allosteric carboxylic inhibitor of MMP-13, or a pharmaceutically acceptable salt thereof, and celecoxib, or a pharmaceutically acceptable salt thereof, or valdecoxib, or a pharmaceutically acceptable salt thereof, in this model. Administration of the allosteric carboxylic inhibitor of MMP-13, or a pharmaceutically acceptable salt thereof, and celecoxib, or a pharmaceutically acceptable salt thereof, or valdecoxib, or a pharmaceutically acceptable salt thereof, in this model is optionally by oral administration or intravenous administration via an osmotic pump. Further, administration of the allosteric carboxylic inhibitor of MMP-13, or a pharmaceutically acceptable salt thereof, and celecoxib, or a pharmaceutically acceptable salt thereof, or valdecoxib, or a pharmaceutically acceptable salt thereof, may be simultaneous as a co-formulation of both drugs, simultaneous by way of independent formulations of each drug of the invention combination alone according to optimal drug delivery profiles, or non-simultaneous such as, sequential administration of an independent formulation of one drug followed by, after some pre-determined period of time, administration of an independent formulation of the other drug of the invention combination. After 7 and 14 days for a two-week study, or 7, 14, and 28 days for a four-week study, the hind-paw weight distribution is again determined. Typically, the animals administered vehicle alone place greater weight on their unaffected left hind paw than on their right hind paw, while animals administered an invention combination show a more normal (i.e., more like a healthy animal) weight distribution between their hind paws. This change in weight distribution was proportional to the degree of joint cartilage damage. Percent inhibition of a change in hind paw joint function is calculated as the percent change in hind-paw weight distribution for treated animals versus control animals. For example, for a two week study,

$$Percent inhibition of a in hind paw joint function = [ 1 - \frac{( Δ {W}_{G} )}{( Δ {W}_{C} )} ] \times 100$$

wherein: ΔW_{C} is the hind-paw weight differential between the healthy left limb and the arthritic limb of the control animal administered vehicle alone, as measured on Day 14; and

ΔW_{G} is the hind-paw weight differential between the healthy left limb and the arthritic limb of the animal administered an invention combination, as measured on Day 14.

In order to measure biochemical or histopathological end points in the MIA Rat model, some of the animals in the above study may be sacrificed, and the amounts of free proteoglycan in both the osteoarthritic right knee joint and the contralateral left knee joint may be determined by biochemical analysis. The amount of free proteoglycan in the contralateral left knee joint provides a baseline value for the amount of free proteoglycan in a healthy joint. The amount of proteoglycan in the osteoarthritic right knee joint in animals administered an invention combination, and the amount of proteoglycan in the osteoarthritic right knee joint in animals administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control. The percent inhibition of proteoglycan loss, may be calculated as {[(proteoglycan loss from joint (%) with vehicle)−(proteoglycan loss from joint with an invention combination)]÷(proteoglycan loss from joint (%) with vehicle)}×100.

The MIA Rat data that are expected from the analysis of proteoglycan loss would establish that an invention combination is effective for inhibiting cartilage damage and inflammation and/or alleviating pain in mammalian patients, including human.

The results of these studies with oral dosing may be presented in tabular format in the columns labelled “IJFL (%+/−SEM)”, wherein UFL means Inhibition of Joint Function Limitation, “SDCES”, wherein SDCES means Significant Decrease In Cartilage Erosion Severity, and “S WHLE”, wherein SIJWHLE means Significant Increase in Joints Without Hind Limb Erosion.

The proportion of subjects without hind limb erosions may be analyzed via an Exact Sequential Cochran-Armitage Trend test (SAS® Institute, 1999). The Cochran-Armitage Trend test is employed when one wishes to determine whether the proportion of positive or “Yes” responders increases or decreases with increasing levels of treatment. For the particular study, it is expected that the number of animals without joint erosions increased with increasing dose.

The ridit analysis may be used to determine differences in overall erosion severity. This parameter takes into account both the erosion grade (0=no erosion, I=erosion extending into the superficial or middle layers, or II=deep layer erosion), and area (small, medium and large, quantified by dividing the area of the largest erosion in each score into thirds) simultaneously. The analysis recognizes that each unit of severity is different, but does not assume a mathematical relationship between units.

Another animal model for measuring effects of an invention combination on cartilage damage and inflammation and/or pain is described below in Biological Method 10.

### Biological Method 10

Induction of Experimental Osteoarthritis in Rabbit (“EOA in Rabbit”):

Normal rabbits are anaesthetized and anteromedial incisions of the right knees performed. The anterior cruciate ligaments are visualized and sectioned. The wounds are closed and the animals are housed in individual cages, exercised, and fed ad libitum. Rabbits are given either vehicle (water) or an invention combination dosed three times per day with 30-mg/kg/dose or 10-mg/kg/dose each independently determined for the allosteric carboxylic inhibitor of MMP-13, or a pharmaceutically acceptable salt thereof, and celecoxib, or a pharmaceutically acceptable salt thereof, or valdecoxib, or a pharmaceutically acceptable salt thereof, but each drug of the combination may independently be administered at other doses such as, for example, 3 times 20 mg/kg/day or 3 times 60 mg/kg/day according to the requirements of the combination being studied. The rabbits are euthanized 8 weeks after surgery and the proximal end of the tibia and the distal end of the femur are removed from each animal.

Macroscopic Grading

The cartilage changes on the femoral condyles and tibial plateaus are graded separately under a dissecting microscope (Stereozoom, Bausch & Lomb, Rochester, N.Y.). The depth of erosion is graded on a scale of 0 to 4 as follows: grade 0=normal surface; Grade 1=minimal fibrillation or a slight yellowish discoloration of the surface; Grade 2=erosion extending into superficial or middle layers only; Grade 3=erosion extending into deep layers; Grade 4=erosion extending to subchondral bone. The surface area changes are measured and expressed in mm². Representative specimens may also be used for histologic grading (see below).

Histologic Grading

Histologic evaluation is performed on sagittal sections of cartilage from the lesional areas of the femoral condyle and tibial plateau. Serial sections (5 um) are prepared and stained with safranin-O. The severity of OA lesions is graded on a scale of 0-14 by two independent observers using the histologic-histochemical scale of Mankin et al. This scale evaluates the severity of OA lesions based on the loss of safranin-O staining (scale 0-4), cellular changes (scale 0-3), invasion of tidemark by blood vessels (scale 0-1) and structural changes (scale 0-6). On this latter scale, 0 indicates normal cartilage structure and 6 indicates erosion of the cartilage down to the subchondral bone. The scoring system is based on the most severe histologic changes in the multiple sections.

Representative specimens of synovial membrane from the medial and lateral knee compartments are dissected from underlying tissues. The specimens are fixed, embedded, and sectioned (5 um) as above, and stained with hematoxylin-eosin. For each compartment, two synovial membrane specimens are examined for scoring purposes and the highest score from each compartment is retained. The average score is calculated and considered as a unit for the whole knee. The severity of synovitis is graded on a scale of 0 to 10 by two independent observers, adding the scores of 3 histologic criteria: synovial lining cell hyperplasia (scale 0-2); villous hyperplasia (scale 0-3); and degree of cellular infiltration by mononuclear and polymorphonuclear cells (scale 0-5): 0 indicates normal structure.

Statistical Analysis

Mean values and SEM is calculated and statistical analysis was done using the Mann-Whitney U-test.

The results of these studies would be expected to show that an invention combination would reduce the size of the lesion on the tibial plateaus, and perhaps the damage in the tibia or on the femoral condyles, as well as show pain alleviating effects if measured. In conclusion, these results would show that an invention combination would have significant inhibition effects on the damage to cartilage and pain.

The foregoing studies would establish that an invention combination is effective for the inhibition of cartilage damage and inflammation and/or alleviating pain, and thus useful for the treatment of osteoarthritis or rheumatoid arthritis in human, and other mammalian disorders. Such a treatment offers a distinct advantage over existing treatments that only modify pain or inflammation or and other secondary symptoms. The effectiveness of an invention combination in this model would indicate that the invention combination will have clinically useful effects in preventing and/or treating cartilage damage, pain and/or inflammation.

Administration according to the invention method of celecoxib, or a pharmaceutically acceptable salt thereof, or valdecoxib, or a pharmaceutically acceptable salt thereof, and an allosteric carboxylic inhibitor of MMP-13, or a pharmaceutically acceptable salt thereof, to a mammal to treat the diseases listed above is preferably, although not necessarily, accomplished by administering the compound, or a salt thereof, in a pharmaceutical dosage form.

Celecoxib, or a pharmaceutically acceptable salt thereof, or valdecoxib, or a pharmaceutically acceptable salt thereof, and allosteric carboxylic inhibitors of MMP-13, or a pharmaceutically acceptable salt thereof, can be prepared and administered according to the invention method in a wide variety of oral and parenteral pharmaceutical dosage forms. Thus, celecoxib, or a pharmaceutically acceptable salt thereof, or valdecoxib, or a pharmaceutically acceptable salt thereof, and the allosteric carboxylic inhibitors of MMP-13, or a pharmaceutically acceptable salt thereof, can be administered by injection, that is, intravenously, intramuscularly, intracutaneously, subcutaneously, intraduodenally, or intraperitoneally. Also, celecoxib, or a pharmaceutically acceptable salt thereof, or valdecoxib, or a pharmaceutically acceptable salt thereof, and the allosteric carboxylic inhibitors of MMP-13, or a pharmaceutically acceptable salt thereof, can be administered by inhalation, for example, intranasally. Additionally, celecoxib, or a pharmaceutically acceptable salt thereof, or valdecoxib, or a pharmaceutically acceptable salt thereof, and the allosteric carboxylic inhibitors of MMP-13, or a pharmaceutically acceptable salt thereof, can be administered transdermally. It will be obvious to those skilled in the art that the following dosage forms may comprise as the active components celecoxib, or a pharmaceutically acceptable salt thereof, or valdecoxib, or a pharmaceutically acceptable salt thereof, and an allosteric carboxylic inhibitor of MMP-13, or a pharmaceutically acceptable salt thereof. The active compounds generally are present in a concentration of about 5% to about 95% by weight of the formulation.

For preparing pharmaceutical compositions from celecoxib, or a pharmaceutically acceptable salt thereof, or valdecoxib, or a pharmaceutically acceptable salt thereof, and the allosteric carboxylic inhibitors of MMP-13, or a pharmaceutically acceptable salt thereof, (i.e., the active components) pharmaceutically acceptable carriers can be either solid or liquid. Solid form preparations are preferred. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component. Powders suitable for intravenous administration or administration by injection may be lyophilized.

In tablets, the active component is mixed with the carrier having the necessary binding properties in suitable proportions and compacted in the shape and size desired.

The powders and tablets preferably contain from about 5% to about 70%, total, of the active component. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active component with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing an appropriate quantity of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.01 to 1000 mg, preferably 1 to 500 mg according to the particular application and the potency of the active components. The composition can, if desired, also contain other compatible therapeutic agents.

In therapeutic use as agents to treat the above-listed diseases, the allosteric carboxylic inhibitors of MMP-13, or a pharmaceutically acceptable salt thereof, or a combination of the same with valdecoxib, or a pharmaceutically acceptable salt thereof, or celecoxib, or a pharmaceutically acceptable salt thereof, are administered at a dose that is effective for treating at least one symptom of the disease or disorder being treated. The initial dosage of about 1 mg/kg to about 100 mg/kg daily of the active component will be effective. A daily dose range of about 25 mg/kg to about 75 mg/kg of the active component is preferred. The dosages, however, may be varied depending upon the requirements of the patient, the severity of the condition being treated, and the particular allosteric carboxylic inhibitor of MMP-13, or a pharmaceutically acceptable salt thereof, and valdecoxib, or a pharmaceutically acceptable salt thereof, or celecoxib, or a pharmaceutically acceptable salt thereof, being employed in the invention combination. Determination of the proper dosage for a particular situation is within the skill of the art as described above. Typical dosages will be from about 0.1 mg/kg to about 500 mg/kg, and ideally about 25 mg/kg to about 250 mg/kg, such that it will be an amount that is effective to treat the particular disease or disorder being treated.

A preferred composition for dogs comprises an ingestible liquid peroral dosage form selected from the group consisting of a solution, suspension, emulsion, inverse emulsion, elixir, extract, tincture and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

A preferred composition provides delayed-, sustained- and/or controlled-release of valdecoxib, or a pharmaceutically acceptable salt thereof, or celecoxib, or a pharmaceutically acceptable salt thereof, and the allosteric carboxylic inhibitor of MMP-13, or a pharmaceutically acceptable salt thereof. Such preferred compositions include all such dosage forms which produce ≧40% inhibition of cartilage degradation, and result in a plasma concentration of the active component of at least 3 fold the active component's ED₄₀ for at least 2 hours; preferably for at least 4 hours; preferably for at least 8 hours; more preferably for at least 12 hours; more preferably still for at least 16 hours; even more preferably still for at least 20 hours; and most preferably for at least 24 hours. Preferably, there is included within the above-described dosage forms those which produce ≧40% inhibition of cartilage degradation, and result in a plasma concentration of the active component of at least 5 fold the active component's ED₄₀ for at least 2 hours, preferably for at least 2 hours, preferably for at least 8 hours, more preferably for at least 12 hours, still more preferably for at least 20 hours and most preferably for at least 24 hours. More preferably, there is included the above-described dosage forms which produce ≧50% inhibition of cartilage degradation, and result in a plasma concentration of the active component of at least 5 fold the active component's ED₄₀ for at least 2 hours, preferably for at least 4 hours, preferably for at least 8 hours, more preferably for at least 12 hours, still more preferably for at least 20 hours and most preferably for at least 24 hours.

The following Formulation Examples 1 to 8 illustrate the invention pharmaceutical compositions wherein the allosteric carboxylic inhibitor of MMP-13, or a pharmaceutically acceptable salt thereof, and valdecoxib, or a pharmaceutically acceptable salt thereof, or celecoxib, or a pharmaceutically acceptable salt thereof, are formulated separately, each independently as described, When the formulations comprise the allosteric carboxylic inhibitor of MMP-13, or a pharmaceutically acceptable salt thereof, and a pharmaceutically acceptable carrier, diluent, or excipient, they contain a cartilage damage treating effective amount or an anti-osteoarthritic effective amount of the allosteric carboxylic inhibitor of MMP-13, or a pharmaceutically acceptable salt thereof. When the formulations comprise valdecoxib, or a pharmaceutically acceptable salt thereof, or celecoxib, or a pharmaceutically acceptable salt thereof, they contain a pain alleviating effective amount or an anti-inflammatory effective amount of valdecoxib or celecoxib. The examples are representative only, and are not to be construed as limiting the invention in any respect.

### FORMULATION EXAMPLE 1

Tablet Formulation:

| Ingredient | Amount (mg) |
|---|---|
| An allosteric carboxylic inhibitor of MMP-13, or | 25 |
| celecoxib, or valdecoxib | |
| Lactose | 50 |
| Cornstarch (for mix) | 10 |
| Cornstarch (paste) | 10 |
| Magnesium stearate (1%) | 5 |
| Total | 100 |

The allosteric carboxylic inhibitor of MMP-13, or celecoxib, or valdecoxib, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for inhibiting cartilage damage or treating osteoarthritis.

### FORMULATION EXAMPLE 2

Coated Tablets:

The tablets of Formulation Example 1 are coated in a customary manner with a coating of sucrose, potato starch, talc, tragacanth, and colorant.

### FORMULATION EXAMPLE 3

Injection vials:

The pH of a solution of 500 g of an allosteric carboxylic inhibitor of MMP-13, or celecoxib, or valdecoxib and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of the allosteric carboxylic inhibitor of MMP-13, or celecoxib, or valdecoxib.

### FORMULATION EXAMPLE 4

Suppositories:

A mixture of 25 g of an allosteric carboxylic inhibitor of MMP-13, or celecoxib, or valdecoxib, 100 g of soya lecithin, and 1400 g of cocoa butter is fused, poured into molds, and allowed to cool. Each suppository contains 25 mg of the allosteric carboxylic inhibitor of MMP-13, or celecoxib, or valdecoxib.

### FORMULATION EXAMPLE 5

Solution:

A solution is prepared from 1 g of an allosteric carboxylic inhibitor of MMP-13, or celecoxib, or valdecoxib, 9.38 g of NaH₂PO₄.12H₂O, 28.48 g of Na₂HPO₄.12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 25 mg of the allosteric carboxylic inhibitor of MMP-13, or celecoxib, or valdecoxib.

### FORMULATION EXAMPLE 6

Ointment:

500 mg of an allosteric carboxylic inhibitor of MMP-13, or celecoxib, or valdecoxib is mixed with 99.5 g of petroleum jelly under aseptic conditions. A 5 g portion of the ointment contains 25 mg of the allosteric carboxylic inhibitor of MMP-13, or celecoxib, or valdecoxib.

### FORMULATION EXAMPLE 7

Capsules:

2 kg of an allosteric carboxylic inhibitor of MMP-13, or celecoxib, or valdecoxib are filled into hard gelatin capsules in a customary manner such that each capsule contains 25 mg of the allosteric carboxylic inhibitor of MMP-13, or celecoxib, or valdecoxib.

### FORMULATION EXAMPLE 8

Ampoules:

A solution of 2.5 kg of an allosteric carboxylic inhibitor of MMP-13, or celecoxib, or valdecoxib is dissolved in 60 L of double-distilled water. The solution is sterile filtered, and the filtrate is filled into ampoules. The ampoules are lyophilized under sterile conditions and aseptically sealed. Each ampoule contains 25 mg of the allosteric carboxylic inhibitor of MMP-13, or celecoxib, or valdecoxib.

The following Formulation Examples 9 to 16 illustrate the invention pharmaceutical compositions containing an invention combination in a single formulation with a pharmaceutically acceptable carrier, diluent, or excipient. The examples are representative only, and are not to be construed as limiting the invention in any respect.

### FORMULATION EXAMPLE 9

Tablet Formulation:

| Ingredient | Amount (mg) |
|---|---|
| An allosteric carboxylic inhibitor of MMP-13 | 25 |
| Valdecoxib or celecoxib | 20 |
| Lactose | 50 |
| Cornstarch (for mix) | 10 |
| Cornstarch (paste) | 10 |
| Magnesium stearate (1%) | 5 |
| Total | 120 |

The allosteric carboxylic inhibitor of MMP-13, valdecoxib or celecoxib, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of one of the above-listed diseases.

### FORMULATION EXAMPLE 10

Coated Tablets:

The tablets of Formulation Example 9 are coated in a customary manner with a coating of sucrose, potato starch, talc, tragacanth, and colorant.

### FORMULATION EXAMPLE 11

Injection Vials:

The pH of a solution of 250 g of valdecoxib or celecoxib, 500 g of an allosteric carboxylic inhibitor of MMP-13, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 12.5 mg of valdecoxib or celecoxib and 25 mg of the allosteric carboxylic inhibitor of MMP-13.

### FORMULATION EXAMPLE 12

Suppositories:

A mixture of 50 g of valdecoxib or celecoxib, 25 g of an allosteric carboxylic inhibitor of MMP-13, 100 g of soya lecithin, and 1400 g of cocoa butter is fused, poured into molds, and allowed to cool. Each suppository contains 50 mg of valdecoxib or celecoxib and 25 mg of the allosteric carboxylic inhibitor of MMP-13.

### FORMULATION EXAMPLE 13

Solution:

A solution is prepared from 0.5 g of valdecoxib or celecoxib, 1 g of an allosteric carboxylic inhibitor of MMP-13, 9.38 g of NaH₂PO₄ 12H₂O, 28.48 g of Na₂HPO₄.12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 12.5 mg of valdecoxib or celecoxib and 25 mg of the allosteric carboxylic inhibitor of MMP-13.

### FORMULATION EXAMPLE 14

Ointment:

100 mg of valdecoxib or celecoxib, 500 mg of an allosteric carboxylic inhibitor of MMP-13 is mixed with 99.4 g of petroleum jelly under aseptic conditions. A 5 g portion of the ointment contains 5 mg of valdecoxib or celecoxib and 25 mg of the allosteric carboxylic inhibitor of MMP-13.

### FORMULATION EXAMPLE 15

Capsules:

2 kg of valdecoxib or celecoxib and 20 kg of an allosteric carboxylic inhibitor of MMP-13 are filled into hard gelatin capsules in a customary manner such that each capsule contains 25 mg of valdecoxib or celecoxib and 250 mg of the allosteric carboxylic inhibitor of MMP-13.

### FORMULATION EXAMPLE 16

Ampoules:

A solution of 2.5 kg of valdecoxib or celecoxib and 2.5 kg of an allosteric carboxylic inhibitor of MMP-13 is dissolved in 60 L of double-distilled water. The solution is sterile filtered, and the filtrate is filled into ampoules. The ampoules are lyophilized under sterile conditions and aseptically sealed. Each ampoule contains 25 mg each of valdecoxib or celecoxib and the allosteric carboxylic inhibitor of MMP-13.

While it may be desirable to formulate valdecoxib or celecoxib and an allosteric carboxylic inhibitor of MMP-13, or a pharmaceutically acceptable salt thereof, together in one capsule, tablet, ampoule, solution, and the like, for simultaneous administration, it is not necessary for the purposes of practicing the invention methods. Valdecoxib or celecoxib and an allosteric carboxylic inhibitor of MMP-13, or a pharmaceutically acceptable salt thereof, of an invention combination alternatively can each be formulated independently in any form such as, for example, those of any one Formulation Examples 1 to 16, and administered to a patient either simultaneously or at different times.

The following examples illustrate the invention pharmaceutical compositions containing discrete formulations of the active components of the invention combination and a pharmaceutically acceptable carrier, diluent, or excipient. The examples are representative only, and are not to be construed as limiting the invention in any respect.

### FORMULATION EXAMPLE 17

Tablet Formulation of an allosteric
carboxylic inhibitor of MMP-13:

| Ingredient | Amount (mg) |
|---|---|
| An allosteric carboxylic inhibitor of MMP-13 | 25 |
| Lactose | 50 |
| Cornstarch (for mix) | 10 |
| Cornstarch (paste) | 10 |
| Magnesium stearate (1%) | 5 |
| Total | 100 |

An allosteric carboxylic inhibitor of MMP-13, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet.

Injection Vial Formulation of Valdecoxib or Celecoxib:

The pH of a solution of 500 g of valdecoxib or celecoxib and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of valdecoxib or celecoxib.

Such tablets containing the allosteric carboxylic inhibitor of MMP-13 can be administered to a human from one to four times a day for treatment of the above-listed diseases, and the injection solutions containing valdecoxib or celecoxib can be administered to a human 1 or 2 times per day, wherein the administration by injection is optionally simultaneous with administration of the tablets or at different times, for the treatment of one of the above-listed diseases.

### FORMULATION EXAMPLE 18

Coated Tablets Containing an Allosteric Carboxylic Inhibitor of MMP-13:

The tablets of Formulation Example 17 are coated in a customary manner with a coating of sucrose, potato starch, talc, tragacanth, and colorant.

Capsules Containing Valdecoxib or Celecoxib:

2 kg of valdecoxib or celecoxib are filled into hard gelatin capsules in a customary manner such that each capsule contains 25 mg of valdecoxib or celecoxib.

Such coated tablets containing the allosteric carboxylic inhibitor of MMP-13 can be administered to a human from one to four times a day for treatment of the above-listed diseases, and the capsules containing valdecoxib or celecoxib can be administered to a human 1 or 2 times per day, wherein the administration of the capsules is optionally simultaneous with administration of the tablets or at different times, for the treatment of one of the above-listed diseases.

Still further, it should be appreciated that the invention methods comprising administering an invention combination to a mammal to treat diseases or disorders listed above may be used to treat different diseases simultaneously. For example, administration of valdecoxib or celecoxib in accordance with the invention combination may be carried out as described above to treat inflammation, arthritic pain, pain associated with menstrual cramping, and migraines, while an allosteric carboxylic inhibitor of MMP-13, or a pharmaceutically acceptable salt thereof, may be administered to treat OA or inhibit cartilage damage.

As shown above, the invention method offers a distinct advantage over existing treatments for diseases such as OA that comprise cartilage damage, wherein the existing treatments modify pain or secondary symptoms, but do not show a disease modifying effect.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

Having described the invention method, various embodiments of the invention are hereupon claimed.

What is claimed is:

1. A combination, comprising valdecoxib, or a pharmaceutically acceptable salt thereof, and an allosteric carboxylic inhibitor of M-13 of Formula IC 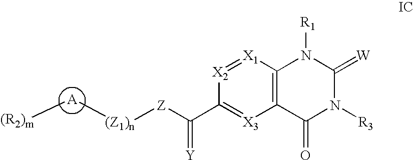
or a pharmaceutically acceptable salt thereof, or an N-oxide thereof,
in which:
R₁ represents a group selected from:
hydrogen, amino,
(C₁-C₆)alkyl, (C₃-C₆)alkenyl, (C₃-C₆)alkynyl, mono(C₁-C₆)alkylamino(C₁-C₆)alkyl, di(C₁-C₆)alkylamino(C₁-C₆)alkyl, aryl, aryl(C₁-C₆)alkyl, heterocycle, and 3- to 6-membered cycloalkyl(C₁-C₆)alkyl, these groups being unsubstituted or substituted with one or more groups, which may be identical or different, selected from amino, (C₁-C₆)alkyl, cyano, halo(C₁-C₆)alkyl, C(═O)OR₄, OR₄ and SR₄, in which R4 represents hydrogen or (C₁-C₆)alkyl,
W represents an oxygen atom, a sulphur atom, or a group ═N—R′, in which R′ represents (C₁-C₆)alkyl, hydroxyl, or cyano,
X₁, X₂ and X₃ represent, independently of each other, a nitrogen atom or a group —C—R₆ in which R₆ represents a group selected from hydrogen, (C₁-C₆)alkyl, amino, mono(C₁-C₆)alkylamino, di(C₁-C₆)alkylamino, hydroxyl, (C₁-C₆)alkoxy, and halogen,
with the proviso that not more than two of the groups X₁, X₂ and X₃ simultaneously represent a nitrogen atom,
Y represents a group selected from oxygen atom, sulphur atom, —NH, and —N(C₁-C₆)alkyl,
Z represents:
an oxygen atom, a sulphur atom,
or a group —NR₇ in which R₇ represents a group selected from hydrogen, (C₁-C₆)alkyl, aryl(C₁-C₆)alkyl, cycloalkyl, aryl, and heteroaryl, and
when Y is an oxygen atom, a sulphur atom, or a group —N(C₁-C₆)alkyl, Z optionally represents a carbon atom which is unsubstituted or substituted with a (C₁-C₆)alkyl, an aryl, an aryl(C₁-C₆)alkyl, an aromatic or non-aromatic heterocycle or a cycloalkyl,
n is an integer from 1 to 8 inclusive,
Z₁ represents —CR₈R₉ wherein R₈ and R₉, independently of each other, represent a group selected from hydrogen, (C₁-C₆)alkyl, halo(C₁-C₆)alkyl, halogen, amino, OR₄, SR₄ or C(═O)OR₄ in which R₄ represents a hydrogen or (C₁-C₆)alkyl, and
when n is greater than or equal to 2, the hydrocarbon chain Z₁ optionally contains one or more multiple bonds,
and/or one of the carbon atoms in the hydrocarbon chain Z₁ may be replaced with an oxygen atom, a sulphur atom which is unsubstituted or substituted with one or two oxygen atoms, or a nitrogen atom which is unsubstituted or substituted with a (C₁-C₆)alkyl,
and when one of the carbon atoms in the hydrocarbon chain Z₁ is replaced with a sulphur atom which is unsubstituted or substituted with one or two oxygen atoms, then the group —C(═Y)-Z- optionally may be absent in the general formula (I),
A represents a group selected from:
aromatic or non-aromatic, 5- or 6-membered monocycle comprising from 0 to 4 heteroatoms selected from nitrogen, oxygen and sulphur, and
bicycle, composed of two aromatic or non-aromatic, 5- or 6-membered rings, which may be identical or different, comprising from 0 to 4 heteroatoms selected from nitrogen, oxygen and sulphur,
m is an integer from 0 to 7 inclusive,
the group(s) R₂, which may be identical or different, is (are) selected from (C₁-C₆)alkyl, halogen, —CN, NO₂, SCF₃, —CF₃, —OCF₃, —NR₁₀R₁₁, —OR₁₀, —SR₁₀, —SOR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:
X₅ represents a group selected from oxygen, sulphur optionally substituted by one or two oxygen atoms, and nitrogen substituted by hydrogen or (C₁-C₆)alkyl,
k is an integer from 0 to 3 inclusive,
R₁₀ and R₁₁, which may be identical or different, are selected from hydrogen and (C₁-C₆)alkyl,
X₄ represents a group selected from single bond, —CH₂—, oxygen atom, sulphur atom optionally substituted by one or two oxygen atoms, and nitrogen atom substituted by hydrogen atom or (C₁-C₆)alkyl group,
R₁₂ represents an aromatic or non-aromatic, heterocyclic or non-heterocyclic, 5- or 6-membered ring which is unsubstituted or substituted with one or more groups, which may be identical or different, selected from (C₁-C₆)alkyl, halogen, hydroxyl and amino, and when the ring is heterocyclic, it comprises from 1 to 4 heteroatoms selected from nitrogen, oxygen and sulphur;
R₃ represents a group selected from:
hydrogen,
(C₁-C₆)alkyl, (C₃-C₆)alkenyl, (C₃-C₆)alkynyl, these groups being unsubstituted or substituted with one or more groups, which may be identical or different, selected from amino, cyano, halo(C₁-C₆)alkyl, cycloalkyl, —C(═O)NR₁₀R₁₁, —C(═O)OR₁₀, OR₁₀, and SR₁₀, in which R₁₀ and R₁₁, which may be identical or different, represent hydrogen or (C₁-C₆)alkyl,
and the group of formula: 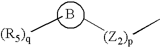
in which p is an integer from 0 to 8 inclusive,
Z₂ represents —CR₁₃R₁₄ wherein R₁₃ and R₁₄, independently of each other, represent a group selected from hydrogen, (C₁-C₆)alkyl, phenyl, halo(C₁-C₆)alkyl, halogen, amino, OR₄, SR₄ and —C(═O)OR₄ in which R₄ represents hydrogen or (C₁-C₆)alkyl, and
when p is greater than or equal to 2, the hydrocarbon chain Z₂ optionally contains one or more multiple bonds,
and/or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl, or a carbonyl group,
B represents a group selected from:
an aromatic or non-aromatic 5- or 6-membered monocycle comprising from 0 to 4 heteroatoms selected from nitrogen, oxygen and sulphur, and
a bicycle, composed of two aromatic or non-aromatic, 5- or 6-membered rings, which may be identical or different, comprising from 0 to 4 heteroatoms selected from nitrogen, oxygen and sulphur,
q is an integer from 0 to 7 inclusive,
the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ, R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, (CH₂)ₖC(═O)OR₁₅, —C(═O)O—(CH₂)ₖ₂-NR₁₅R₁₆, —C(═O)O—(CH₂)ₖ₂—C(═O)OR₁₈, —X₇(CH₂)ₖC(═O)NR₁₅R₁₆, —(CH₂)ₖC(═O)NR₁₅R₁₆, —R₁₉—C(═O)OR₁₅, —X₆—R₂₀, and —C(═O)—R₂₁—NR₁₅R₁₆ in which:
X₇ represents a group selected from oxygen atom, sulphur atom optionally substituted by one or two oxygen atoms, and nitrogen atom substituted by a hydrogen atom or a (C₁-C₆)alkyl group,
k is an integer from 0 to 3 inclusive,
k1 is an integer from 0 to 2 inclusive,
k2 is an integer from 1 to 4 inclusive,
R₁₅, R₁₆ and R₁₇, which may be identical or different, are selected from hydrogen and (C₁-C₆)alkyl,
R₁₈ represents a group selected from (C₁-C₆)alkyl, —R₂₁—NR₁₅R₁₆, —R₂₁—NR₁₅—C(═O)—R₂₁—NR₁₆R₁₇, and —C(═O)O—R₂, —NR₁₅R₁₆ in which R₂₁ represents a linear or branched (C₁-C₆)alkylene group, and R₁₅, R₁₆ and R₁₇ are as defined hereinbefore,
R₁₉ represents a (C₃-C₆)cycloalkyl group,
X₆ represents a group selected from single bond, —CH₂—, oxygen atom, sulphur atom optionally substituted by one or two oxygen atoms, and nitrogen atom substituted by hydrogen atom or (C₁-C₆)alkyl group,
R₂₀ represents an aromatic or non-aromatic, heterocyclic or non-heterocyclic, 5- or 6-membered ring, which is unsubstituted or substituted with one or more groups, which may be identical or different, selected from (C₁-C₆)alkyl, halogen, hydroxyl, oxo, cyano, tetrazole, amino, and —C(═O)OR₄ wherein R₄ represents hydrogen or (C₁-C₆)alkyl, and, when the ring is heterocyclic, it comprises from 1 to 4 heteroatoms selected from nitrogen, oxygen and sulphur,
with the proviso that when X₁ represents a nitrogen atom, X₂ cannot represent a carbon atom substituted with a methyl group or with NH—CH₃.

2. The combination according to claim 1, wherein the compound of Formula IC is selected from:
4-[6-(4-Methoxy-benzylcarbamoyl)-1-methyl-2,4-dioxo-1,4-dihydro-2H-[3,4-d]pyrimidin-3-ylmethyl]-benzoic acid;
3-Benzyl-1-methyl-2,4-dioxo-1,2,3,4-tetrahydro-pyrido[3,4-d]pyrimidine-6-carboxylic acid (1,3-benzodioxol-5-ylmethyl)-amide;
Methyl 4-[6-(4-Methoxy-benzylcarbamoyl)-1-methyl-2,4-dioxo-1,4-dihydro-2H-pyrido[3,4-d]pyrimidin-3-ylmethyl]-benzoate;
3-(4-Cyano-benzyl)-1-methyl-2,4-dioxo-1,2,3,4-tetrahydro-pyrido[3,4-d]pyrimidine-6-carboxylic acid 4-methoxy-benzylamide;
4-[6-(3-Methoxy-benzylcarbamoyl)-1-methyl-2,4-dioxo-1,4-dihydro-2H-[3,4-d]pyrimidin-3-ylmethyl]-benzoic acid;
4-[6-(4-Methoxy-benzylcarbamoyl)-1-methyl-2,4-dioxo-1,4-dihydro-2H-[2,3-d]pyrimidin-3-ylmethyl]-benzoic acid;
or a pharmaceutically acceptable salt thereof.

3. A combination, comprising valdecoxib, or a pharmaceutically acceptable salt thereof, and an allosteric carboxylic inhibitor of MMP-13 of Formula VG 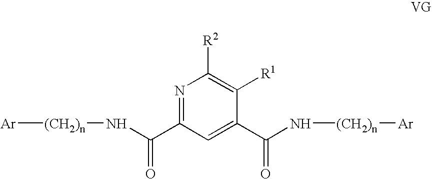
or a pharmaceutically acceptable salt thereof, wherein
R¹ and R² independently are hydrogen, halo, hydroxy, C₁-C₆ alkyl, C₁-C₆ alkoxy, C₂-C₆ alkenyl, C₂-C₆ alkynyl, NO₂, NR⁴R⁵, CN, or CF₃;
+P2
n is 1, and
Each Ar independently is aryl or Het, wherein aryl is phenyl or substituted phenyl, and Het is an unsubstituted or substituted heteroaryl group.

4. A pharmaceutical composition, comprising a combination of valdecoxib, or a pharmaceutically acceptable salt thereof, and an allosteric carboxylic inhibitor of MMP-13, or a pharmaceutically acceptable salt thereof, and a pharmaceutically acceptable carrier, diluent, or excipient.

5. A method of treating a disease or disorder selected from cartilage damage, inflammation, arthritis, and pain in a mammal, comprising administering to the mammal a therapeutically effective amount of a combination of valdecoxib, or a pharmaceutically acceptable salt thereof, and an allosteric carboxylic inhibitor of MMP-13, or a pharmaceutically acceptable salt thereof.

6. The method according to claim 5, wherein the disease or disorder is rheumatoid arthritis.

7. The method according to claim 5, wherein the disease or disorder is osteoarthritis.

8. The method according to claim 5, wherein the disease or disorder is joint inflammation.

9. The method according to claim 5, wherein the pain is joint pain.
